(19) United States
(12) Patent Application Publication
(10) Pub. No.: US 2025/0136549 A1
(43) Pub. Date: May 1, 2025

(54) AMINE COMPOUND AND ORGANIC LIGHT-EMITTING DEVICE INCLUDING THE SAME

(71) Applicant: Samsung Display Co., Ltd., Yongin-si (KR)

(72) Inventors: Chaeyeong Kim, Yongin-si (KR); Minji Kim, Yongin-si (KR); Bora Lee, Yongin-si (KR); Hanna Cha, Yongin-si (KR)

(21) Appl. No.: 18/759,204

(22) Filed: Jun. 28, 2024

(30) Foreign Application Priority Data
Oct. 25, 2023 (KR) 10-2023-0144167

(51) Int. Cl. C07D 209/88; C07D 307/91; C07D 333/76; H10K 50/15; H10K 85/60

(52) U.S. Cl. CPC C07D 209/88; C07D 307/91; C07D 333/76; H10K 50/15; H10K 85/615; H10K 85/633; H10K 85/6572; H10K 85/6574; H10K 85/6576

(57) ABSTRACT

An amine-containing compound and a light-emitting device including the same are provided. Also provided are an electronic apparatus including the light-emitting device and electronic equipment including the electronic apparatus.

### CROSS-REFERENCE TO RELATED APPLICATION

This application claims priority to and the benefit of Korean Patent Application No. 10-2023-0144167, filed on Oct. 25, 2023, in the Korean Intellectual Property Office, the entire content of which is incorporated herein by reference.

### BACKGROUND

### 1. Field

One or more aspects of embodiments of the present disclosure relate to an amine-containing compound, a light-emitting device including the same, an electronic apparatus including the light-emitting device, and electronic equipment including the light-emitting device.

### 2. Description of the Related Art

An organic light-emitting device may have wider viewing angles, higher contrast ratios, and shorter response times, compared to an inorganic light-emitting device. An organic light-emitting device may include a first electrode, a hole transport region, an emission layer, an electron transport region, and a second electrode, which are arranged sequentially in this stated order. Holes injected from the first electrode may move toward the emission layer through the hole transport region. Electrons injected from the second electrode may move to the emission layer through the electron transport region. Carriers such as the holes and the electrons may combine in the emission layer e.g., combine to generate excitons. These excitons may transition (e.g., relax) from an excited state to a ground state to generate light.

### SUMMARY

One or more aspects of embodiments of the present disclosure are directed toward an amine-containing compound with improved hole transport characteristics and a light-emitting device, having a relatively low driving voltage, a relatively high efficiency, and/or a relatively long lifespan, by employing the amine-containing compound.

One or more aspects of embodiments of the present disclosure are directed toward a high-quality electronic apparatus and electronic equipment that include the light-emitting device.

Additional aspects will be set forth in part in the description which follows and, in part, will be apparent from the description, or may be learned by practice of the presented embodiments of the disclosure.

According to one or more embodiments, an amine-containing compound may be represented by Formula 1:

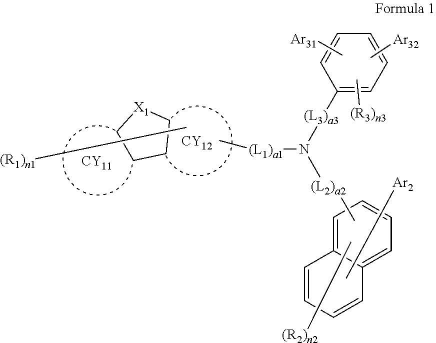

wherein, in Formula 1,
X₁ may be N(R₄), O, or S,
CY₁₁ and CY₁₂ may each independently be a C₃-C₆₀ carbocyclic group or a C₁-C₆₀ heterocyclic group,
Ar₂, Ar₃₁, and Ar₃₂ may each independently be a C₃-C₆₀ aryl group unsubstituted or substituted with at least one R₁₀ₐ or a C₁-C₆₀ heteroaryl group unsubstituted or substituted with at least one R₁₀ₐ,
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 may each independently be an integer from 0 to 5, and i) when a1 is 0, a group represented by *-(L₁)ₐ₁-*′ may be a single bond, ii) when a2 is 0, a group represented by *-(L₂)ₐ₂-*′ may be a single bond, iii) when a3 is 0, a group represented by *-(L₃)ₐ₃-*′ may be a single bond, iv) when a1 is an integer from 2 to 5, a plurality of L₁(s) may be identical to or different from each other, v) when a2 is an integer from 2 to 5, a plurality of L₂(s) may be identical to or different from each other, and vi) when a3 is an integer from 2 to 5, a plurality of L₃(s) may be identical to or different from each other,
R₁ to R₄ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
neighboring two or more of R₁ to R₃ may not be bonded or (e.g., optionally) may be bonded to each other to form (or provide) a C₅-C₆₀ carbocyclic group unsubstituted or substituted with at least one R₁₀ₐ or a C₁-C₆₀ heterocyclic group unsubstituted or substituted with at least one R₁₀ₐ,
n1 may be an integer from 0 to 20,
n2 may be an integer from 0 to 6,
n3 may be an integer from 0 to 3,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, [[or]] a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group,
a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group, and
* and *′ each indicate a binding site to a neighboring atom.

According to one or more embodiments, a light-emitting device includes the amine-containing compound.

According to one or more embodiments, an electronic apparatus includes the light-emitting device.

According to one or more embodiments, electronic equipment includes the light-emitting device.

### BRIEF DESCRIPTION OF THE DRAWINGS

The accompanying drawings are included to provide a further understanding of the preceding and other aspects, features, and advantages of certain embodiments of the disclosure are incorporated in and constitute a part of this specification. The drawings illustrate example embodiments and, together with the following description taken in conjunction with the accompanying drawings. In the drawings:

FIG. 1 is a schematic view of a light-emitting device according to one or more one or more embodiments;

FIG. 2 is a schematic view of an electronic apparatus according to one or more one or more embodiments;

FIG. 3 is a schematic view of an electronic apparatus according to one or more embodiments;

FIG. 4 is a schematic perspective view of electronic equipment including a light-emitting device according to one or more one or more embodiments;

FIG. 5 is a diagram illustrating the exterior of a vehicle as electronic equipment including a light-emitting device according to one or more one or more embodiments; and

FIGS. 6A-6C are each a diagram schematically illustrating the interior of a vehicle according to one or more embodiments.

### DETAILED DESCRIPTION

Reference will now be made in more detail to embodiments, examples of which are illustrated in the accompanying drawings, wherein like reference numerals refer to like elements throughout the specification, and duplicative descriptions thereof may not be provided. In this regard, the present embodiments may have different forms and should not be construed as being limited to the descriptions set forth herein. Accordingly, the embodiments are merely described, by referring to the drawings, to explain aspects of the present description. As utilized herein, the term “and/or” includes any and all combinations of one or more of the associated listed items. Expressions such as “at least one of,” “one of,” and “selected from,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

Unless otherwise defined, all chemical names, technical and scientific terms, and terms defined in common dictionaries should be interpreted as having meanings consistent with the context of the related art, and should not be interpreted in an ideal or overly formal sense. It will be understood that, although the terms first, second, and/or the like, may be used herein to describe various elements, these elements should not be limited by these terms. These terms are only used to distinguish one element from another element. Thus, a first element could be termed a second element without departing from the teachings of the present disclosure. Similarly, a second element could be termed a first element.

As used herein, singular forms such as “a,” “an,” and “the” are intended to include the plural forms as well, unless the context clearly indicates otherwise.

It will be further understood that the terms “comprise,” “comprises,” “comprising,” “has,” “have,” “having,” “include,” “includes,” and/or “including,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

In this context, “consisting essentially of” means that any additional components will not materially affect the chemical, physical, optical or electrical properties of the semiconductor film.

In present disclosure, “not include (or not including) a or any ‘component’”, “exclude (or excluding) a or any ‘component’”, “‘component’-free”, and/or the like refers to that the “component” not being added, selected or utilized as a component in the element/composition, but the “component” of less than a suitable amount may still be included due to other impurities and/or external factors.

Further, in this specification, the phrase “on a plane,” or “plan view,” means viewing a target portion from the top, and the phrase “on a cross-section” means viewing a cross-section formed by vertically cutting a target portion from the side.

### Light-Emitting Device

According to an aspect, provided is a light-emitting device including a first electrode, a second electrode facing the first electrode, an interlayer arranged between the first electrode and the second electrode and including an emission layer, and an amine-containing compound represented by Formula 1.

In one or more embodiments, the first electrode may be an anode. The second electrode may be a cathode. The emission layer may include a dopant and a host, and may be to emit light. The dopant and the host are described in more detail elsewhere herein.

The term “interlayer” as utilized herein refers to a single layer and/or a plurality of (e.g., all) layers between a first electrode and a second electrode of a light-emitting device.

In one or more embodiments, the interlayer may further include a hole transport region between the first electrode and the emission layer and an electron transport region between the emission layer and the second electrode. The hole transport region may include a hole injection layer, a hole transport layer, an emission auxiliary layer, an electron-blocking layer, or any combination thereof. The electron transport region may include a buffer layer, a hole-blocking layer, an electron control layer, an electron transport layer, an electron injection layer, or any combination thereof.

For example, the hole transport region may include the hole injection layer arranged on the first electrode and the hole transport layer arranged between the hole injection layer and the emission layer. The hole injection layer may have a single-layer structure or a multi-layer structure. The hole transport layer may have a single-layer structure or a multi-layer structure. For example, the hole transport layer may include a first hole transport layer, a second hole transport layer, and a third hole transport layer, which are arranged in this stated order from the hole injection layer.

For example, the electron transport region may include the electron transport layer arranged on the emission layer, and the electron injection layer arranged between the electron transport layer and the second electrode.

In one or more embodiments, the interlayer may include the amine-containing compound represented by Formula 1.

In one or more embodiments, the hole transport region may include the amine-containing compound represented by Formula 1.

In one or more embodiments, the hole transport layer may include the amine-containing compound represented by Formula 1. The hole transport layer may be in direct contact with the emission layer. For example, the amine-containing compound represented by Formula 1 may be included in the third hole transport layer. In another example, the amine-containing compound represented by Formula 1 may be included in the first hole transport layer and the third hole transport layer. In another example, the amine-containing compound represented by Formula 1 may be included in the first to third hole transport layers.

In one or more embodiments, the light-emitting device may further include a capping layer arranged outside (e.g., on) of the first electrode, and the amine-containing compound represented by Formula 1 may be included in the capping layer.

In one or more embodiments, the light-emitting device may further include a first capping layer arranged outside (e.g., on) of the first electrode and/or a second capping layer arranged outside (e.g., on) of the second electrode, and the amine-containing compound represented by Formula 1 may be included in the first capping layer or the second capping layer. For example, the amine-containing compound represented by Formula 1 may be included in the first capping layer, among the first capping layer, the first electrode, and the interlayer, which are arranged in this stated order. In another example, the amine-containing compound represented by Formula 1 may be included in the second capping layer, among the interlayer, the second electrode, and the second capping layer, which are arranged in this stated order. The amine-containing compound represented by Formula 1 may be included in both (e.g., simultaneously) of the first capping layer and the second capping layer.

According to an aspect, an electronic apparatus including the light-emitting device may be provided.

In one or more embodiments, the electronic apparatus may further include a thin-film transistor. The thin-film transistor may include a source electrode and a drain electrode, and the first electrode of the light-emitting device may be electrically connected to at least one of the source electrode and/or the drain electrode of the thin-film transistor.

According to another aspect, electronic equipment including the light-emitting device may be provided. The electronic equipment may be at least one selected from among a flat panel display, a curved display, a computer monitor, a medical monitor, a TV, a billboard, indoor or outdoor illuminations and/or signal light, a head-up display, a fully or partially transparent display, a flexible display, a rollable display, a foldable display, a stretchable display, a laser printer, a phone, a cell phone, a tablet, a phablet, a personal digital assistant (PDA), a wearable device, laptop computers, digital cameras, camcorders, viewfinders, micro displays, three-dimensional (3D) displays, virtual or augmented reality displays, vehicles, a video wall including multiple displays tiled together, a theater or stadium screen, a phototherapy device, and a signage.

According to another aspect, provided is the amine-containing compound represented by Formula 1:

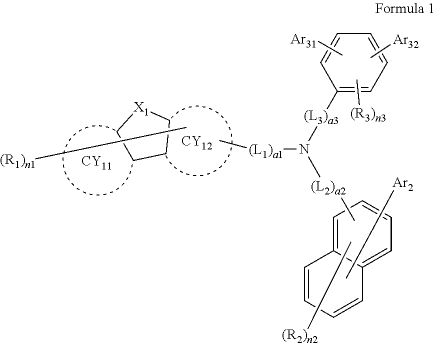

wherein, in Formula 1,
X₁ may be N(R₄), O, or S,
CY₁₁ and CY₁₂ may each independently be a C₃-C₆₀ carbocyclic group or a C₁-C₆₀ heterocyclic group,
Ar₂, Ar₃₁, and Ar₃₂ may each independently be a C₃-C₆₀ aryl group unsubstituted or substituted with at least one R₁₀ₐ or a C₁-C₆₀ heteroaryl group unsubstituted or substituted with at least one R₁₀ₐ,
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 may each independently be an integer from 0 to 5, and i) when a1 is 0, a group represented by *-(L₁)ₐ₁-*′ may be a single bond, ii) when a2 is 0, a group represented by *-(L₂)ₐ₂-*′ may be a single bond, iii) when a3 is 0, a group represented by *-(L₃)ₐ₃-*′ may be a single bond, iv) when a1 is an integer from 2 to 5, a plurality of L₁(s) may be identical to or different from each other, v) when a2 is an integer from 2 to 5, a plurality of L₂(s) may be identical to or different from each other, and vi) when a3 is an integer from 2 to 5, a plurality of L₃(s) may be identical to or different from each other,
R₁ to R₄ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
neighboring two or more of R₁ to R₃ may not be bonded or (e.g., optionally may be bonded to each other to form (or provide) a C₅-C₆₀ carbocyclic group unsubstituted or substituted with at least one R₁₀ₐ or a C₁-C₆₀ heterocyclic group unsubstituted or substituted with at least one R₁₀ₐ,
n1 may be an integer from 0 to 20,
n2 may be an integer from 0 to 6,
n3 may be an integer from 0 to 3,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group;
a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group, and
* and *′ each indicate a binding site to a neighboring atom.

In one or more embodiments, CY₁₁ and CY₁₂ may each independently be a benzene group, a naphthalene group, a phenanthrene group, a fluoranthene group, a triphenylene group, a pyrene group, a chrysene group, an indene group, a fluorene group, a spiro-bifluorene group, a benzofluorene group, a dibenzofluorene group, an indole group, a pyridine group, a pyrimidine group, a carbazole group, a benzocarbazole group, a dibenzocarbazole group, a furan group, a benzofuran group, a dibenzofuran group, a naphthofuran group, a benzonaphthofuran group, a dinaphthofuran group, a thiophene group, a benzothiophene group, a dibenzothiophene group, a naphthothiophene group, a benzonaphthothiophene group, or a dinaphthothiophene group.

In one or more embodiments, the amine-containing compound represented by Formula 1 may be a compound represented by any one selected from among Formulae 1-1 to 1-4:

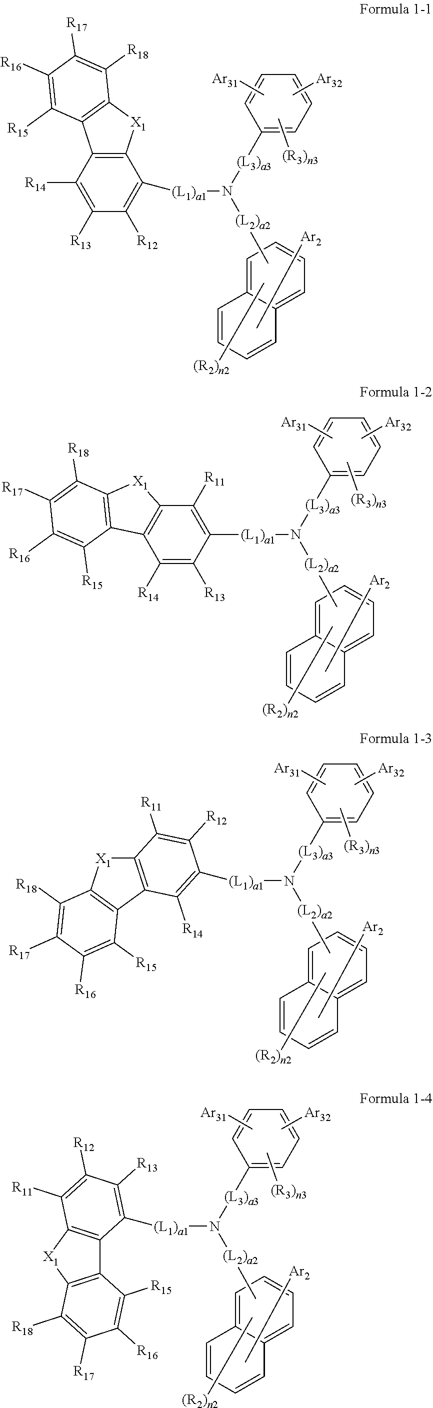

wherein, in Formulae 1-1 to 1-4,
X₁, Ar₂, Ar₃₁, Ar₃₂, L₁ to L₃, a1 to a3, R₂, R₃, n2, and n3 may each be as described herein, and
R₁₁ to R₁₈ may each be as described herein in connection with R₁.

In one or more embodiments, the amine-containing compound represented by Formula 1 may be a compound represented by Formula 1-2.

In one or more embodiments, Ar₂, Ar₃₁, and Ar₃₂ may each independently be a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a dimethylfluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indeno carbazolyl group, an indolocarbazolyl group, a naphthacenyl group, a picenyl group, a hexacenyl group, a pentaphenyl group, a rubicenyl group, a coronenyl group, an ovalenyl group, a naphthopyrrolyl group, a naphthofuranyl group, a naphthothiophenyl group, a naphthosilolyl group, a benzofurocarbazolyl group, a benzothienocarbazolyl group, a benzosilolocarbazolyl group, a triindolophenyl group, a pyrrolophenanthrenyl group, a furanophenanthrenyl group, a thienophenanthrenyl group, a benzonaphthofuranyl group, a benzonaphthothiophenyl group, an (indolo)phenanthrenyl group, a (benzofurano)phenanthrenyl group, or a (benzothieno)phenanthrenyl group, each unsubstituted or substituted with at least one R₁₀ₐ.

In one or more embodiments, Ar₂, Ar₃₁, and Ar₃₂ may each independently be a phenyl group each unsubstituted or substituted with at least one R₁₀ₐ.

In one or more embodiments, Ar₂, Ar₃₁, and Ar₃₂ may each independently be

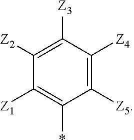

Z₁ to Z₅ may each independently be hydrogen or as described herein in connection with R₁₀ₐ, and * indicates a binding site to a neighboring atom.

In one or more embodiments, a group represented by

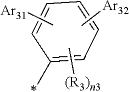

may be a group represented by any one selected from among Formulae 3A to 3F:

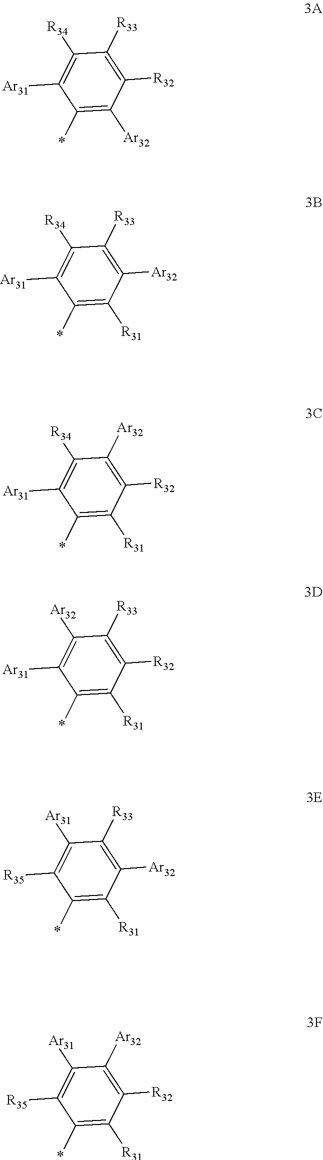

wherein, in Formulae 3A to 3F,
Ar₃₁ and Ar₃₂ are each the same as described herein,
R₃₁ to R₃₅ are each the same as described herein in connection with R₃, and
* indicates a binding site to a neighboring atom.

In one or more embodiments, a group represented by

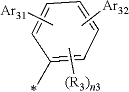

may be a group represented by any one selected from among Formulae 3A-1 to 3F-1:

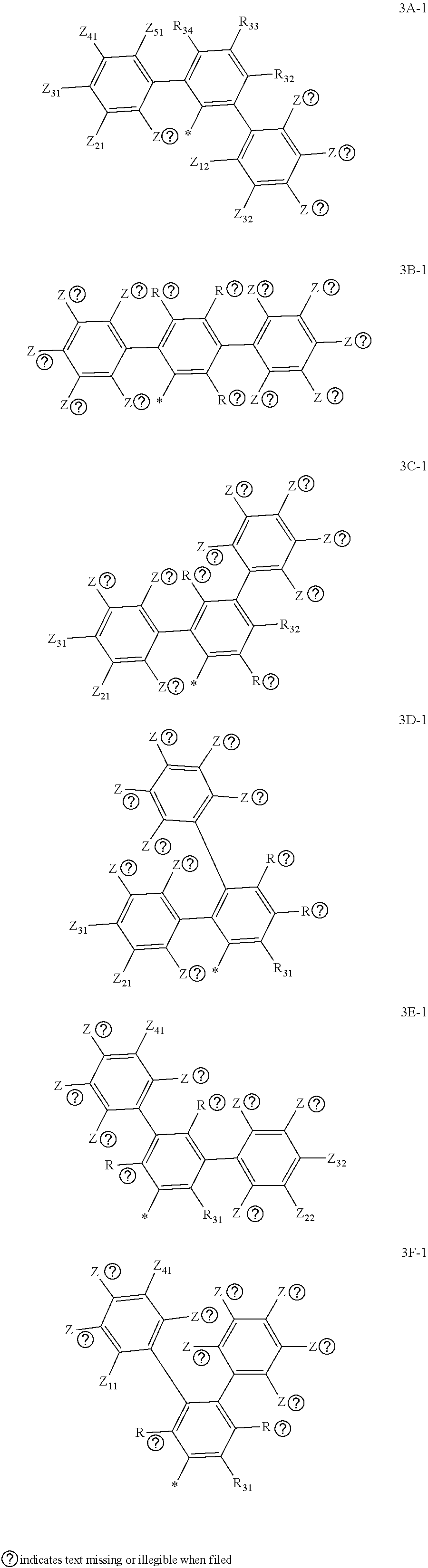

wherein, in Formulae 3A-1 to 3F-1,
Z₁₁ to Z₅₁ and Z₁₂ to Z₅₂ may each independently be hydrogen or as described herein in connection with R₁₀ₐ,
R₃₁ to R₃₅ are each as described herein in connection with R₃, and
* indicates a binding site to a neighboring atom.

In one or more embodiments, R₃ may be hydrogen or deuterium.

In one or more embodiments, a group represented by

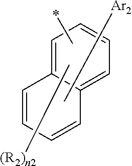

may be a group represented by any one selected from among Formulae 2A to 2N:

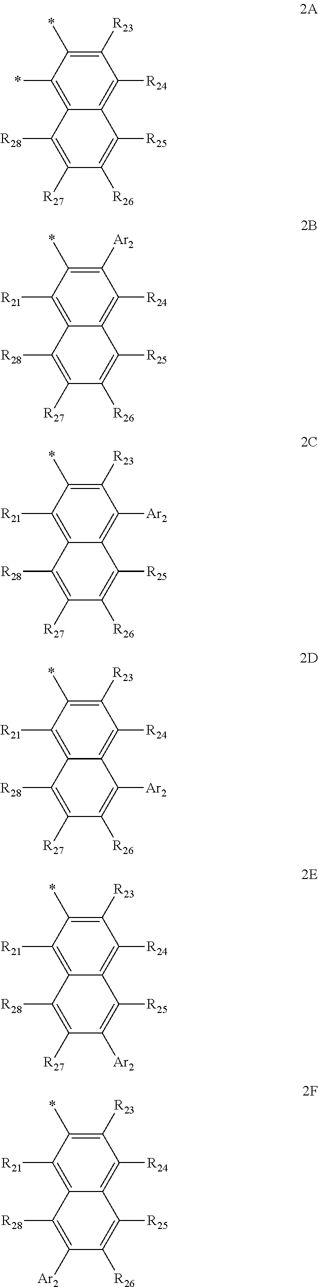

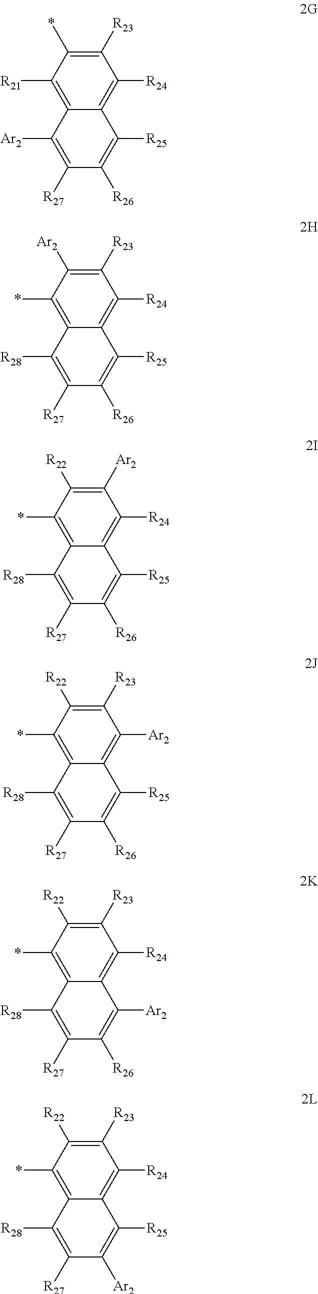

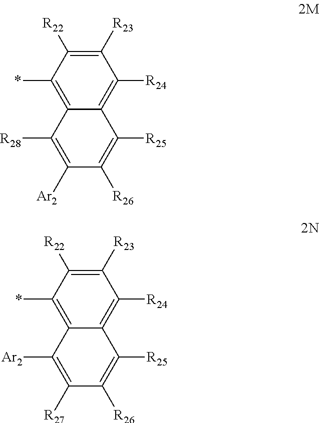

wherein, in Formulae 2A to 2N,
Ar₂ may be as described herein,
R₂₁ to R₂₈ may each be as described herein in connection with R₂, and
* indicates a binding site to a neighboring atom.

In one or more embodiments, a group represented by

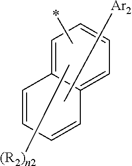

may be a group represented by any one selected from among Formulae 2A to 2G.

In one or more embodiments, a group represented by

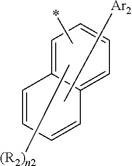

may be a group represented by Formula 2A or 2B.

In one or more embodiments, a group represented by

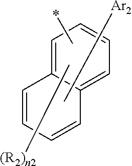

may be a group represented by any one selected from among Formulae 2A-1 to 2N-1:

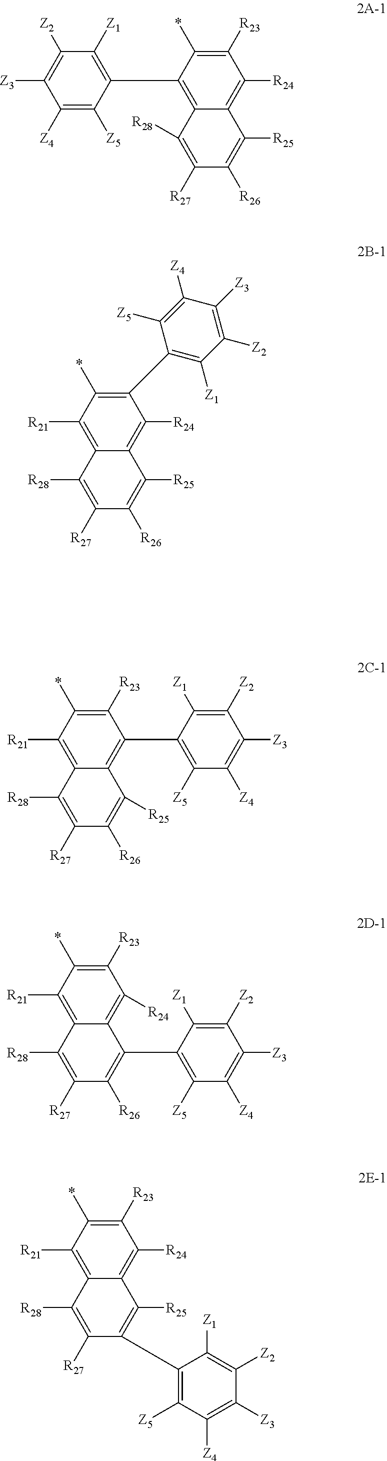

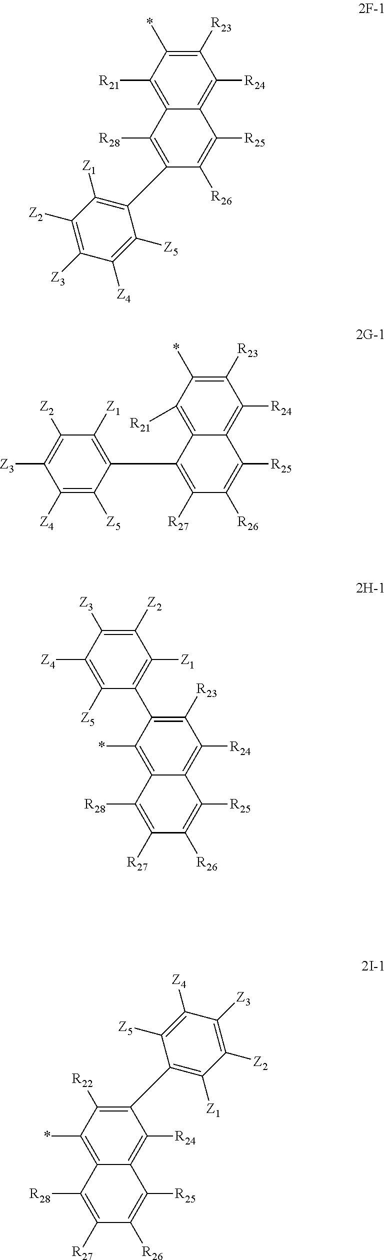

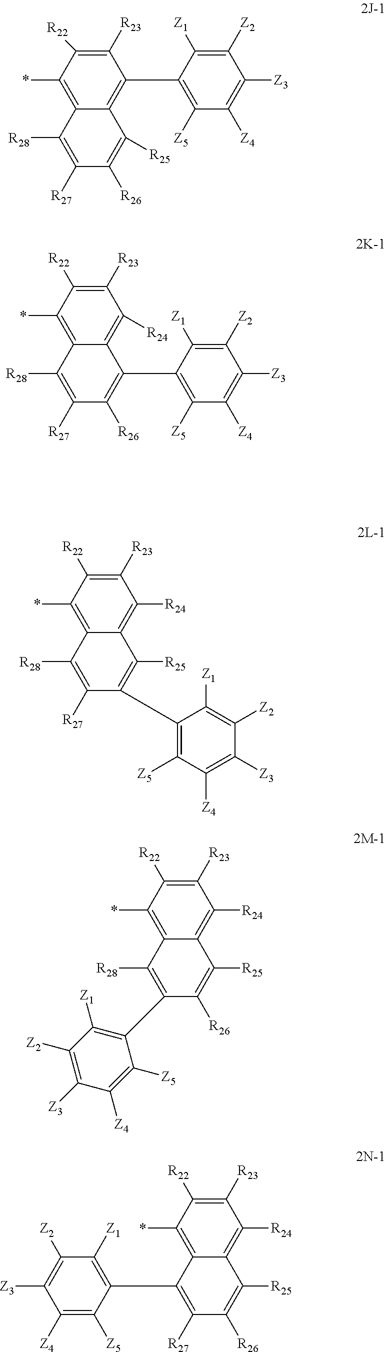

wherein, in Formulae 2A-1 to 2N-1,
Z₁ to Z₅ may each independently be hydrogen or as described herein in connection with R₁₀ₐ,
R₂₁ to R₂₈ may each be as described herein in connection with R₂, and
* indicates a binding site to a neighboring atom.

In one or more embodiments, L₁ to L₃ may each independently be a benzene group, a naphthalene group, an anthracene group, a phenanthrene group, a triphenylene group, a pyrene group, or a chrysene group, each unsubstituted or substituted with at least one R₁₀ₐ.

In one or more embodiments, L₁ to L₃ may be a group represented by one selected from among Formulae 4A to 4C:

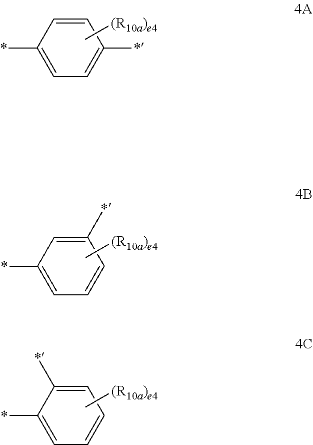

wherein, in Formulae 4A to 4C,
R₁₀ₐ may be as described herein,
e4 may be an integer from 0 to 4, and
* and *′ each indicate a binding site to a neighboring atom.

In one or more embodiments, a1 may be 0.

In one or more embodiments, a2 and a3 may each independently be 0 or 1.

In one or more embodiments, the amine-containing compound represented by Formula 1 may be any one of (e.g., one selected from among) Compounds 1 to 231:

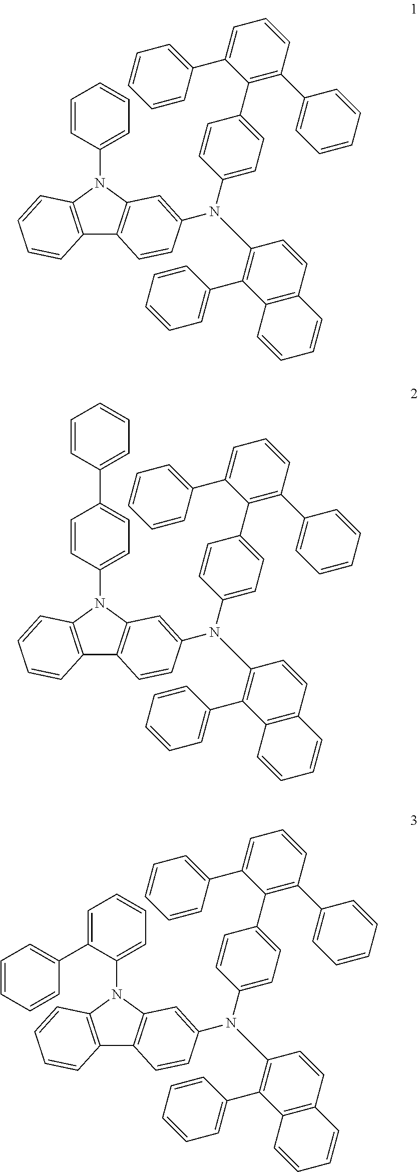 [SMILES: c1ccc(-c2cccc(-c3ccccc3)c2-c2ccc(N(c3ccc4c5ccccc5n(-c5ccccc5)c4c3)c3ccc4ccccc4c3-c3ccccc3)cc2)cc1 ; c1ccc(-c2ccc(-n3c4ccccc4c4ccc(N(c5ccc(-c6c(-c7ccccc7)cccc6-c6ccccc6)cc5)c5ccc6ccccc6c5-c5ccccc5)cc43)cc2)cc1 ; c1ccc(-c2ccccc2-n2c3ccccc3c3ccc(N(c4ccc(-c5c(-c6ccccc6)cccc5-c5ccccc5)cc4)c4ccc5ccccc5c4-c4ccccc4)cc32)cc1]

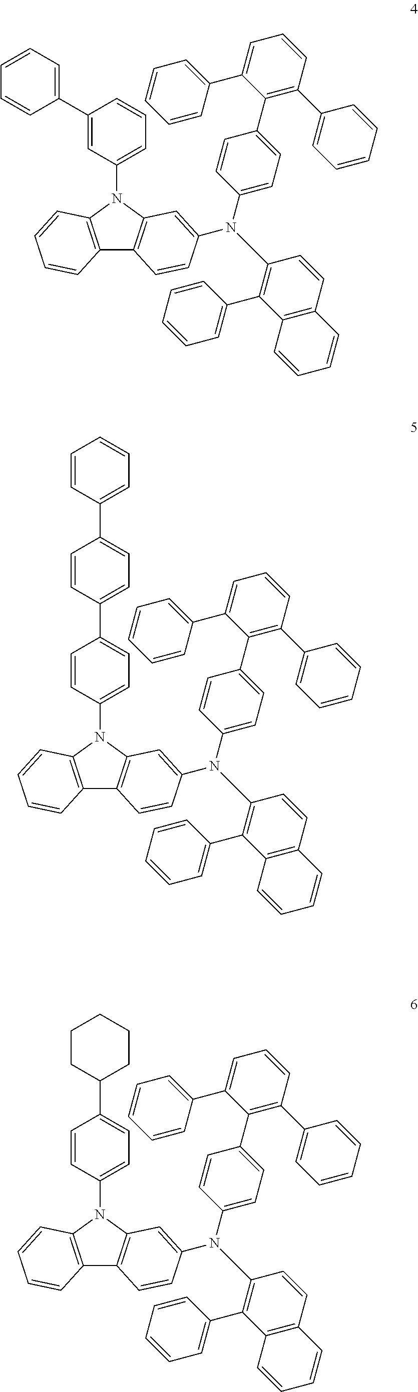 [SMILES: c1ccc(-c2cccc(-n3c4ccccc4c4ccc(N(c5ccc(-c6c(-c7ccccc7)cccc6-c6ccccc6)cc5)c5ccc6ccccc6c5-c5ccccc5)cc43)c2)cc1 ; c1ccc(-c2ccc(-c3ccc(-n4c5ccccc5c5ccc(N(c6ccc(-c7c(-c8ccccc8)cccc7-c7ccccc7)cc6)c6ccc7ccccc7c6-c6ccccc6)cc54)cc3)cc2)cc1 ; c1ccc(-c2cccc(-c3ccccc3)c2-c2ccc(N(c3ccc4c5ccccc5n(-c5ccc(C6CCCCC6)cc5)c4c3)c3ccc4ccccc4c3-c3ccccc3)cc2)cc1]

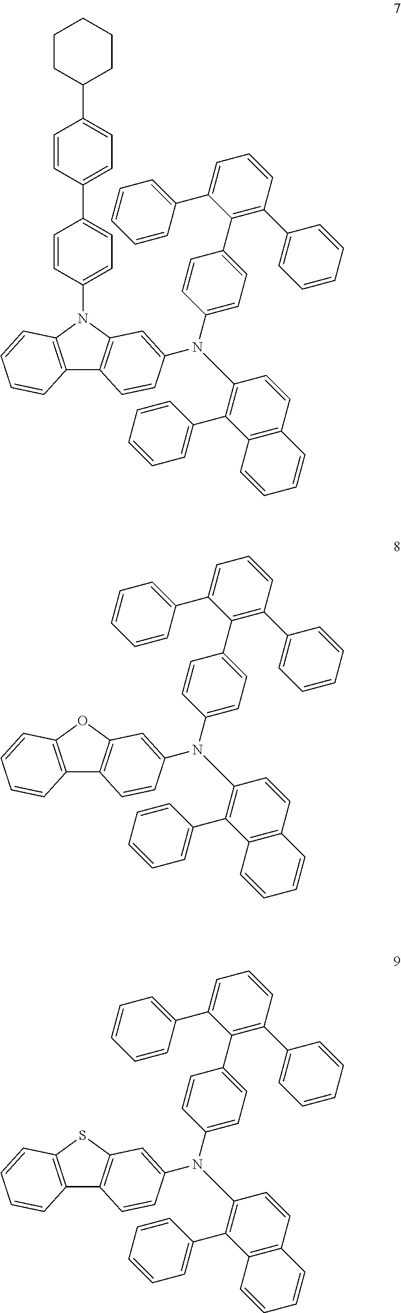 [SMILES: c1ccc(-c2cccc(-c3ccccc3)c2-c2ccc(N(c3ccc4c5ccccc5n(-c5ccc(-c6ccc(C7CCCCC7)cc6)cc5)c4c3)c3ccc4ccccc4c3-c3ccccc3)cc2)cc1 ; c1ccc(-c2cccc(-c3ccccc3)c2-c2ccc(N(c3ccc4c(c3)oc3ccccc34)c3ccc4ccccc4c3-c3ccccc3)cc2)cc1 ; c1ccc(-c2cccc(-c3ccccc3)c2-c2ccc(N(c3ccc4c(c3)sc3ccccc34)c3ccc4ccccc4c3-c3ccccc3)cc2)cc1]

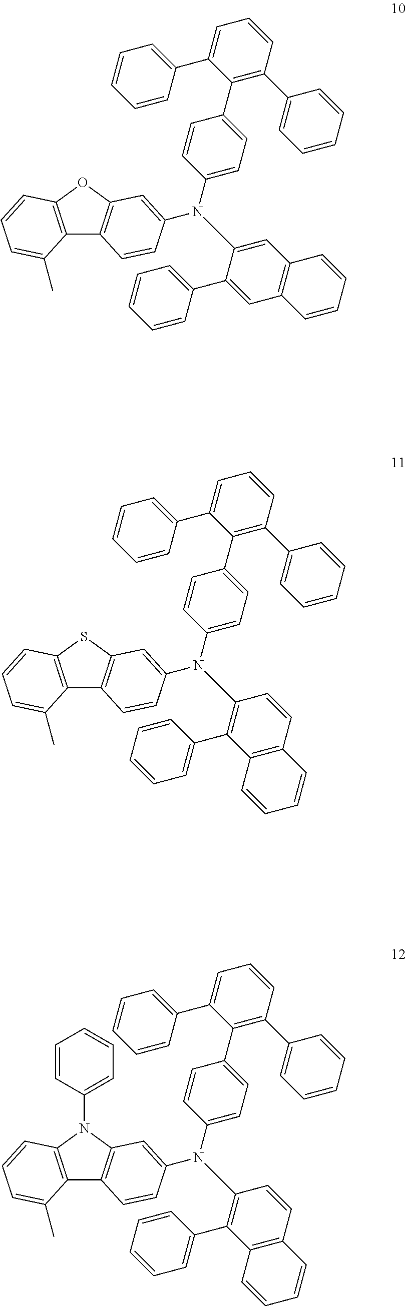 [SMILES: Cc1cccc2oc3cc(N(c4ccc(-c5c(-c6ccccc6)cccc5-c5ccccc5)cc4)c4cc5ccccc5cc4-c4ccccc4)ccc3c12 ; Cc1cccc2sc3cc(N(c4ccc(-c5c(-c6ccccc6)cccc5-c5ccccc5)cc4)c4ccc5ccccc5c4-c4ccccc4)ccc3c12 ; Cc1cccc2c1c1ccc(N(c3ccc(-c4c(-c5ccccc5)cccc4-c4ccccc4)cc3)c3ccc4ccccc4c3-c3ccccc3)cc1n2-c1ccccc1]

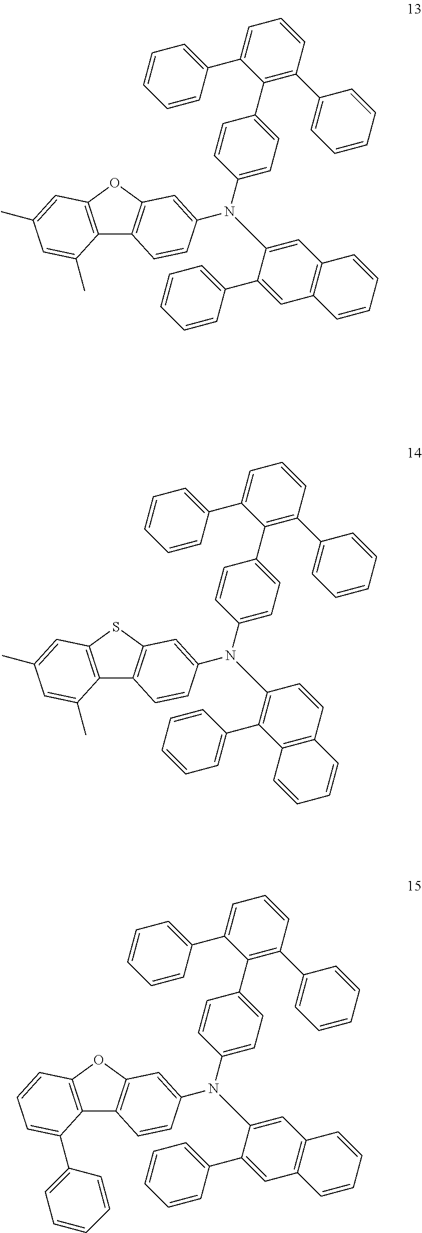 [SMILES: Cc1cc(C)c2c(c1)oc1cc(N(c3ccc(-c4c(-c5ccccc5)cccc4-c4ccccc4)cc3)c3cc4ccccc4cc3-c3ccccc3)ccc12 ; Cc1cc(C)c2c(c1)sc1cc(N(c3ccc(-c4c(-c5ccccc5)cccc4-c4ccccc4)cc3)c3ccc4ccccc4c3-c3ccccc3)ccc12 ; c1ccc(-c2cc3ccccc3cc2N(c2ccc(-c3c(-c4ccccc4)cccc3-c3ccccc3)cc2)c2ccc3c(c2)oc2cccc(-c4ccccc4)c23)cc1]

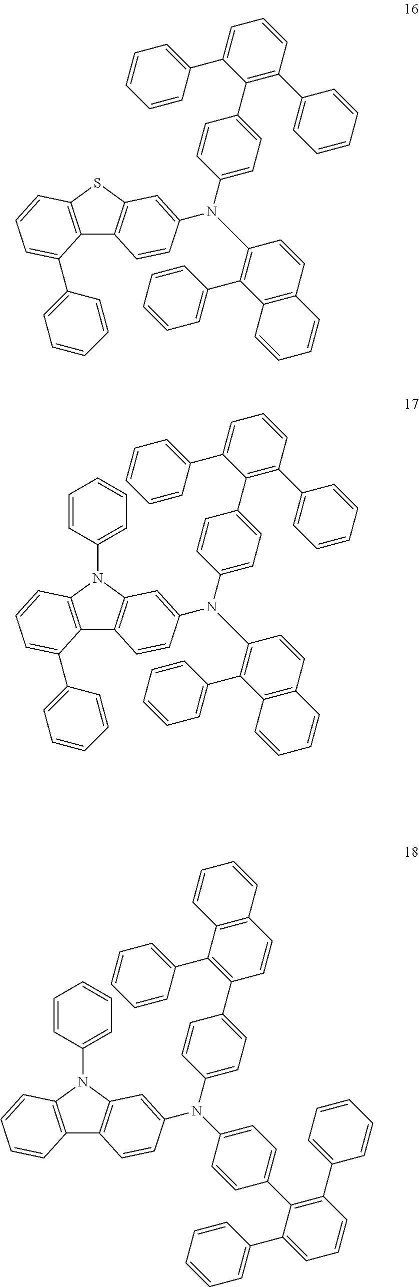 [SMILES: c1ccc(-c2cccc(-c3ccccc3)c2-c2ccc(N(c3ccc4c(c3)sc3cccc(-c5ccccc5)c34)c3ccc4ccccc4c3-c3ccccc3)cc2)cc1 ; c1ccc(-c2cccc(-c3ccccc3)c2-c2ccc(N(c3ccc4c5c(-c6ccccc6)cccc5n(-c5ccccc5)c4c3)c3ccc4ccccc4c3-c3ccccc3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccccc5)c4c3)cc2)cc1]

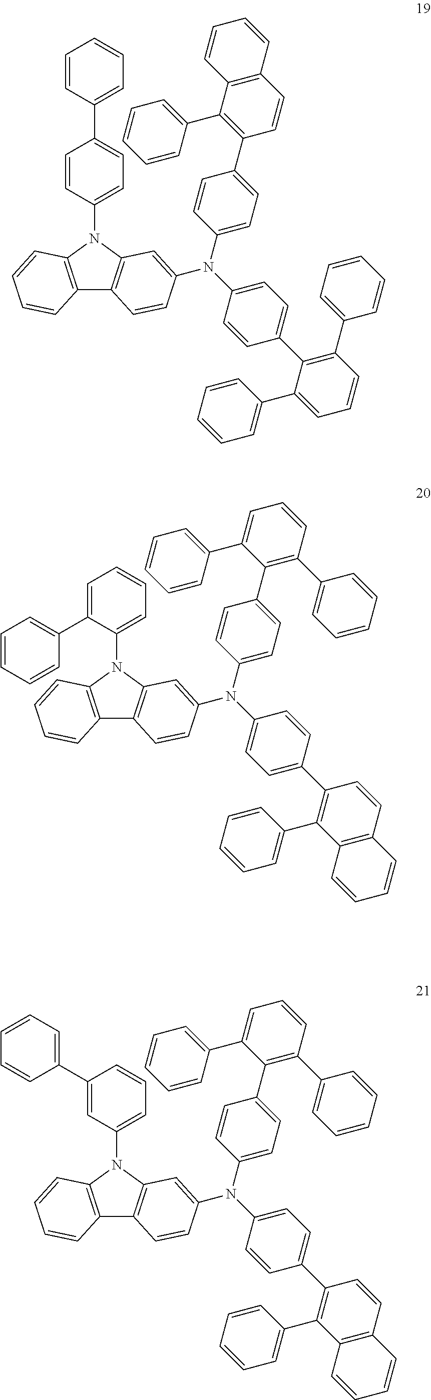 [SMILES: c1ccc(-c2ccc(-n3c4ccccc4c4ccc(N(c5ccc(-c6ccc7ccccc7c6-c6ccccc6)cc5)c5ccc(-c6c(-c7ccccc7)cccc6-c6ccccc6)cc5)cc43)cc2)cc1 ; c1ccc(-c2ccccc2-n2c3ccccc3c3ccc(N(c4ccc(-c5ccc6ccccc6c5-c5ccccc5)cc4)c4ccc(-c5c(-c6ccccc6)cccc5-c5ccccc5)cc4)cc32)cc1 ; c1ccc(-c2cccc(-n3c4ccccc4c4ccc(N(c5ccc(-c6ccc7ccccc7c6-c6ccccc6)cc5)c5ccc(-c6c(-c7ccccc7)cccc6-c6ccccc6)cc5)cc43)c2)cc1]

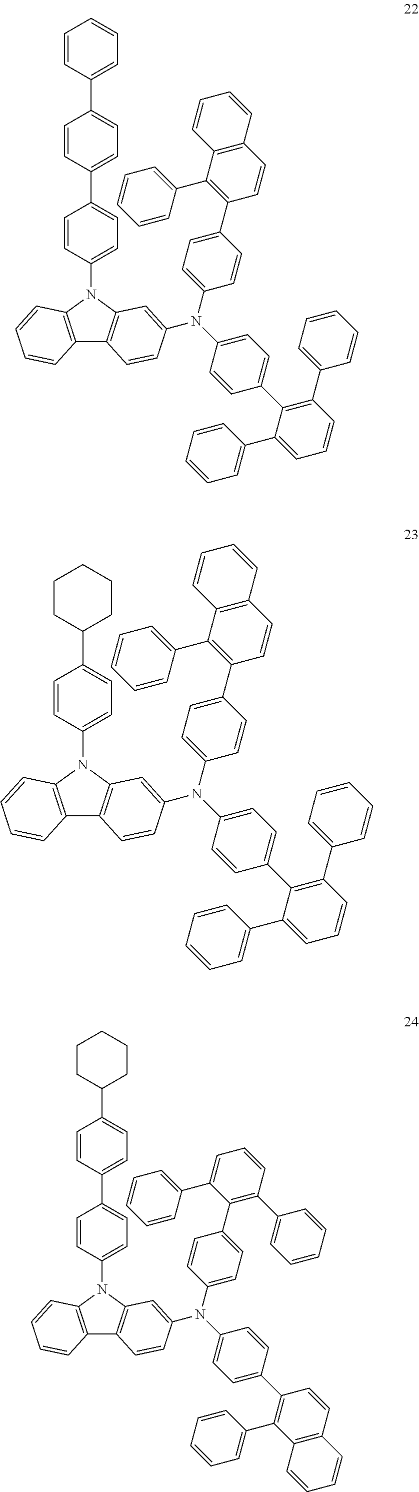 [SMILES: c1ccc(-c2ccc(-c3ccc(-n4c5ccccc5c5ccc(N(c6ccc(-c7ccc8ccccc8c7-c7ccccc7)cc6)c6ccc(-c7c(-c8ccccc8)cccc7-c7ccccc7)cc6)cc54)cc3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccc(C6CCCCC6)cc5)c4c3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccc(-c6ccc(C7CCCCC7)cc6)cc5)c4c3)cc2)cc1]

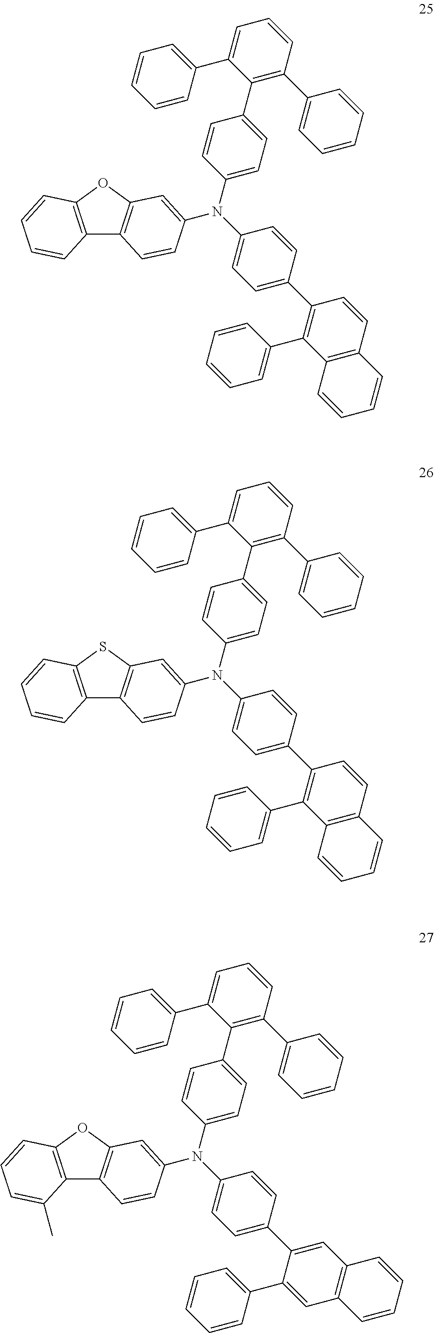 [SMILES: c1ccc(-c2cccc(-c3ccccc3)c2-c2ccc(N(c3ccc(-c4ccc5ccccc5c4-c4ccccc4)cc3)c3ccc4c(c3)oc3ccccc34)cc2)cc1 ; c1ccc(-c2cccc(-c3ccccc3)c2-c2ccc(N(c3ccc(-c4ccc5ccccc5c4-c4ccccc4)cc3)c3ccc4c(c3)sc3ccccc34)cc2)cc1 ; Cc1cccc2oc3cc(N(c4ccc(-c5cc6ccccc6cc5-c5ccccc5)cc4)c4ccc(-c5c(-c6ccccc6)cccc5-c5ccccc5)cc4)ccc3c12]

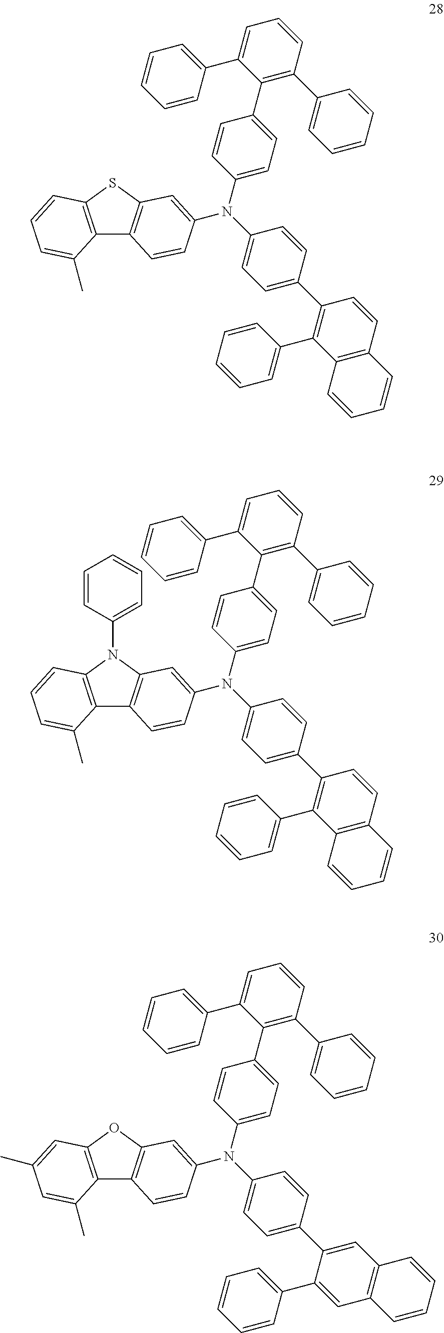 [SMILES: Cc1cccc2sc3cc(N(c4ccc(-c5ccc6ccccc6c5-c5ccccc5)cc4)c4ccc(-c5c(-c6ccccc6)cccc5-c5ccccc5)cc4)ccc3c12 ; Cc1cccc2c1c1ccc(N(c3ccc(-c4ccc5ccccc5c4-c4ccccc4)cc3)c3ccc(-c4c(-c5ccccc5)cccc4-c4ccccc4)cc3)cc1n2-c1ccccc1 ; Cc1cc(C)c2c(c1)oc1cc(N(c3ccc(-c4cc5ccccc5cc4-c4ccccc4)cc3)c3ccc(-c4c(-c5ccccc5)cccc4-c4ccccc4)cc3)ccc12]

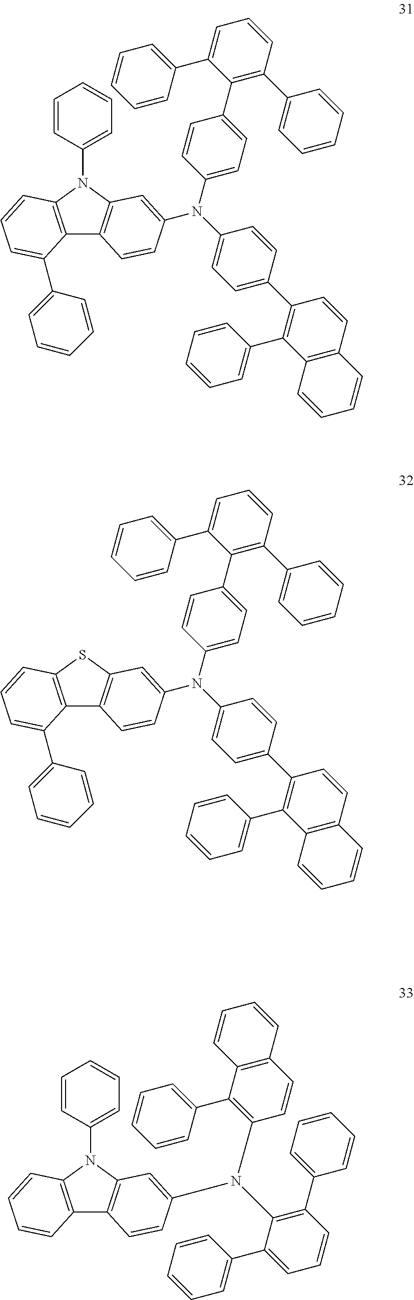 [SMILES: c1ccc(-c2cccc(-c3ccccc3)c2-c2ccc(N(c3ccc(-c4ccc5ccccc5c4-c4ccccc4)cc3)c3ccc4c5c(-c6ccccc6)cccc5n(-c5ccccc5)c4c3)cc2)cc1 ; c1ccc(-c2cccc(-c3ccccc3)c2-c2ccc(N(c3ccc(-c4ccc5ccccc5c4-c4ccccc4)cc3)c3ccc4c(c3)sc3cccc(-c5ccccc5)c34)cc2)cc1 ; c1ccc(-c2cccc(-c3ccccc3)c2N(c2ccc3c4ccccc4n(-c4ccccc4)c3c2)c2ccc3ccccc3c2-c2ccccc2)cc1]

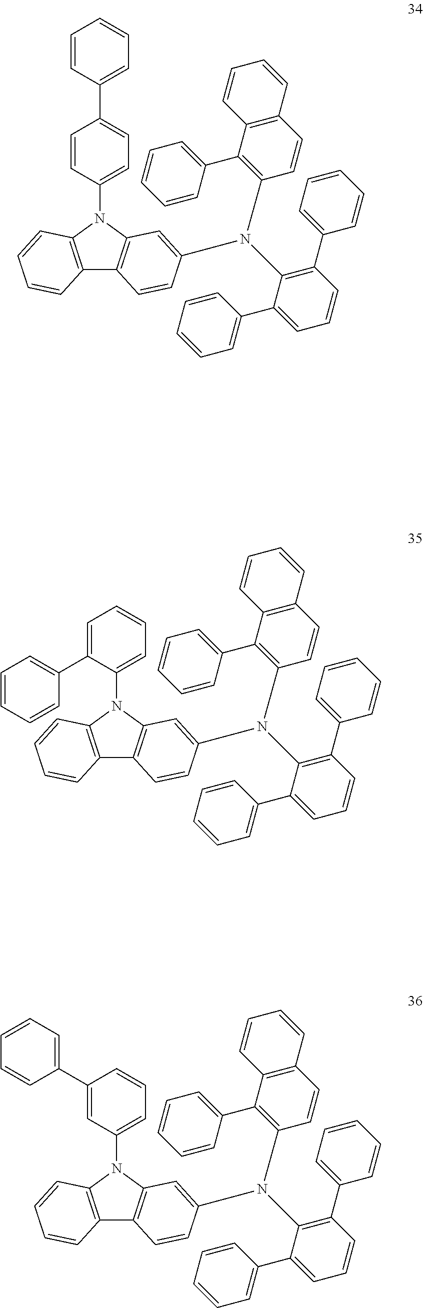 [SMILES: c1ccc(-c2ccc(-n3c4ccccc4c4ccc(N(c5ccc6ccccc6c5-c5ccccc5)c5c(-c6ccccc6)cccc5-c5ccccc5)cc43)cc2)cc1 ; c1ccc(-c2ccccc2-n2c3ccccc3c3ccc(N(c4ccc5ccccc5c4-c4ccccc4)c4c(-c5ccccc5)cccc4-c4ccccc4)cc32)cc1 ; c1ccc(-c2cccc(-n3c4ccccc4c4ccc(N(c5ccc6ccccc6c5-c5ccccc5)c5c(-c6ccccc6)cccc5-c5ccccc5)cc43)c2)cc1]

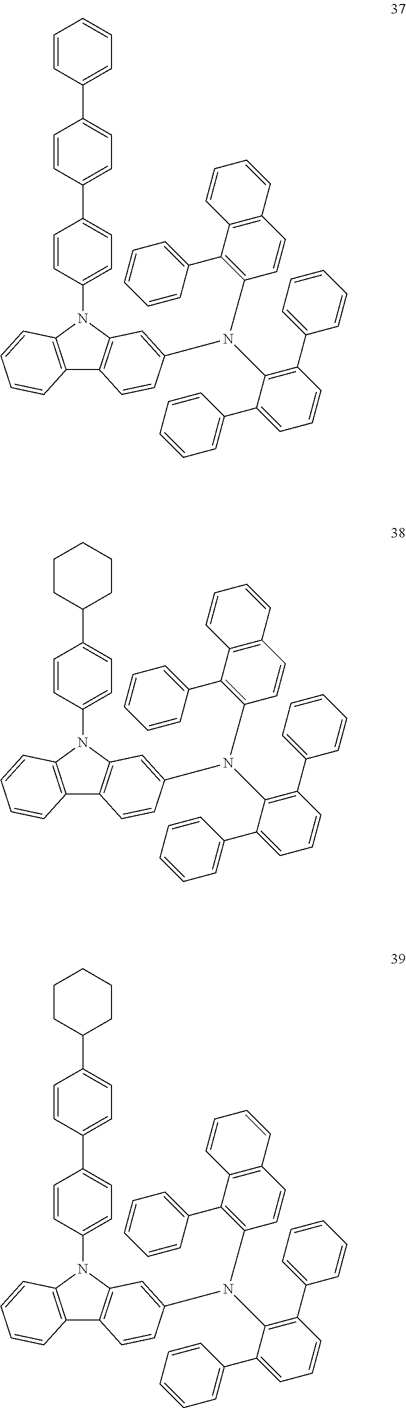 [SMILES: c1ccc(-c2ccc(-c3ccc(-n4c5ccccc5c5ccc(N(c6ccc7ccccc7c6-c6ccccc6)c6c(-c7ccccc7)cccc6-c6ccccc6)cc54)cc3)cc2)cc1 ; c1ccc(-c2cccc(-c3ccccc3)c2N(c2ccc3c4ccccc4n(-c4ccc(C5CCCCC5)cc4)c3c2)c2ccc3ccccc3c2-c2ccccc2)cc1 ; c1ccc(-c2cccc(-c3ccccc3)c2N(c2ccc3c4ccccc4n(-c4ccc(-c5ccc(C6CCCCC6)cc5)cc4)c3c2)c2ccc3ccccc3c2-c2ccccc2)cc1]

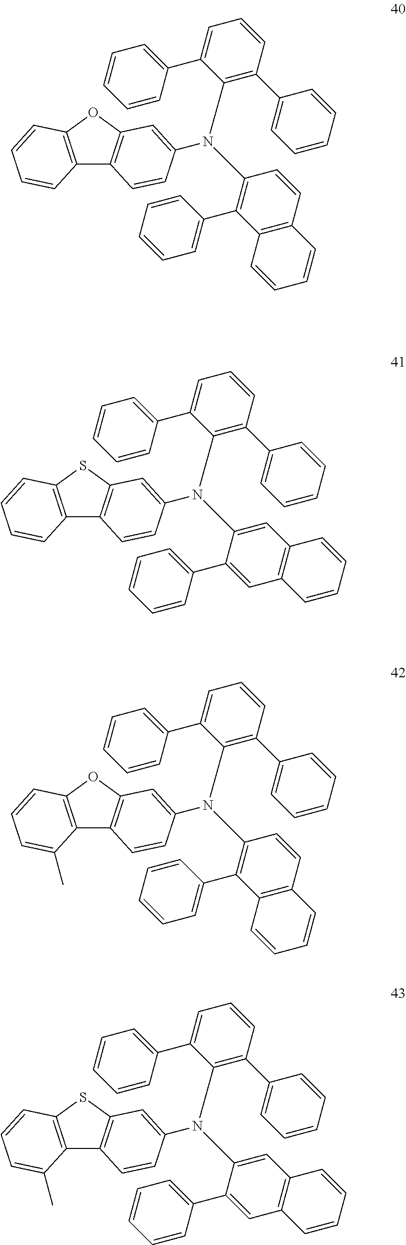 [SMILES: c1ccc(-c2cccc(-c3ccccc3)c2N(c2ccc3c(c2)oc2ccccc23)c2ccc3ccccc3c2-c2ccccc2)cc1 ; c1ccc(-c2cc3ccccc3cc2N(c2ccc3c(c2)sc2ccccc23)c2c(-c3ccccc3)cccc2-c2ccccc2)cc1 ; Cc1cccc2oc3cc(N(c4ccc5ccccc5c4-c4ccccc4)c4c(-c5ccccc5)cccc4-c4ccccc4)ccc3c12 ; Cc1cccc2sc3cc(N(c4cc5ccccc5cc4-c4ccccc4)c4c(-c5ccccc5)cccc4-c4ccccc4)ccc3c12]

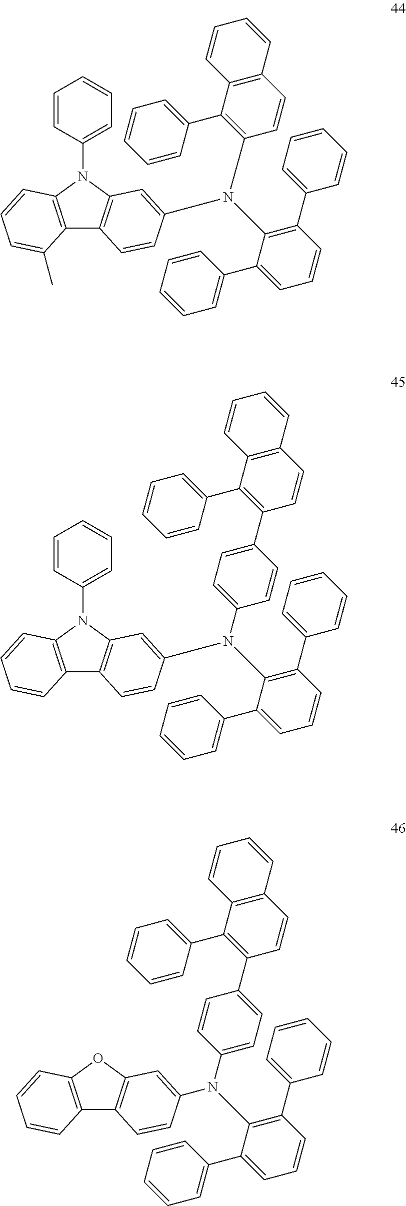 [SMILES: Cc1cccc2c1c1ccc(N(c3ccc4ccccc4c3-c3ccccc3)c3c(-c4ccccc4)cccc3-c3ccccc3)cc1n2-c1ccccc1 ; c1ccc(-c2cccc(-c3ccccc3)c2N(c2ccc(-c3ccc4ccccc4c3-c3ccccc3)cc2)c2ccc3c4ccccc4n(-c4ccccc4)c3c2)cc1 ; c1ccc(-c2cccc(-c3ccccc3)c2N(c2ccc(-c3ccc4ccccc4c3-c3ccccc3)cc2)c2ccc3c(c2)oc2ccccc23)cc1]

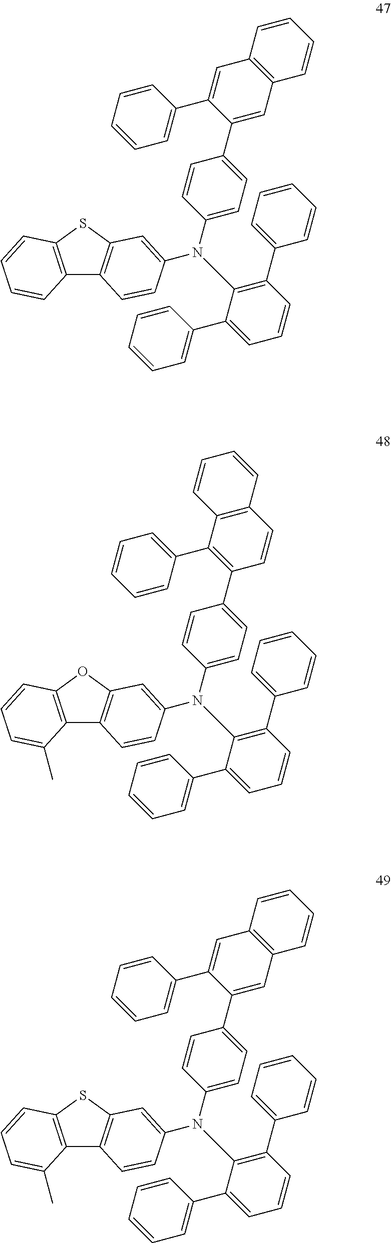 [SMILES: c1ccc(-c2cc3ccccc3cc2-c2ccc(N(c3ccc4c(c3)sc3ccccc34)c3c(-c4ccccc4)cccc3-c3ccccc3)cc2)cc1 ; Cc1cccc2oc3cc(N(c4ccc(-c5ccc6ccccc6c5-c5ccccc5)cc4)c4c(-c5ccccc5)cccc4-c4ccccc4)ccc3c12 ; Cc1cccc2sc3cc(N(c4ccc(-c5cc6ccccc6cc5-c5ccccc5)cc4)c4c(-c5ccccc5)cccc4-c4ccccc4)ccc3c12]

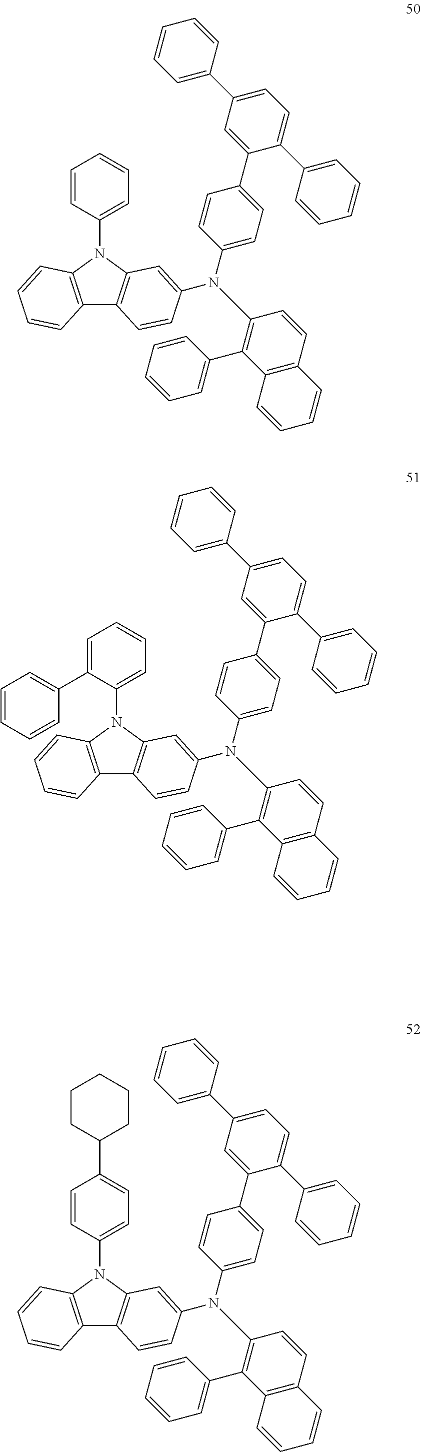 [SMILES: c1ccc(-c2ccc(-c3ccccc3)c(-c3ccc(N(c4ccc5c6ccccc6n(-c6ccccc6)c5c4)c4ccc5ccccc5c4-c4ccccc4)cc3)c2)cc1 ; c1ccc(-c2ccc(-c3ccccc3)c(-c3ccc(N(c4ccc5c6ccccc6n(-c6ccccc6-c6ccccc6)c5c4)c4ccc5ccccc5c4-c4ccccc4)cc3)c2)cc1 ; c1ccc(-c2ccc(-c3ccccc3)c(-c3ccc(N(c4ccc5c6ccccc6n(-c6ccc(C7CCCCC7)cc6)c5c4)c4ccc5ccccc5c4-c4ccccc4)cc3)c2)cc1]

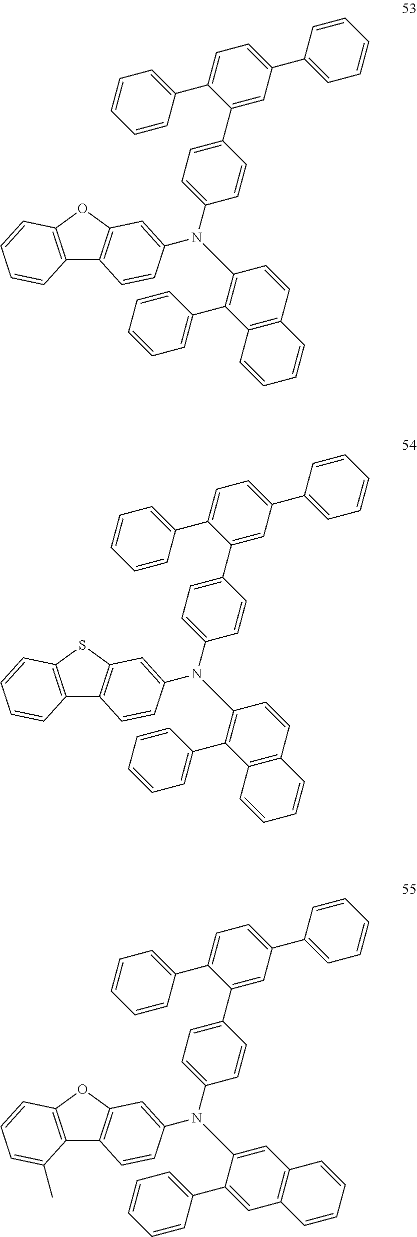 [SMILES: c1ccc(-c2ccc(-c3ccccc3)c(-c3ccc(N(c4ccc5c(c4)oc4ccccc45)c4ccc5ccccc5c4-c4ccccc4)cc3)c2)cc1 ; c1ccc(-c2ccc(-c3ccccc3)c(-c3ccc(N(c4ccc5c(c4)sc4ccccc45)c4ccc5ccccc5c4-c4ccccc4)cc3)c2)cc1 ; Cc1cccc2oc3cc(N(c4ccc(-c5cc(-c6ccccc6)ccc5-c5ccccc5)cc4)c4cc5ccccc5cc4-c4ccccc4)ccc3c12]

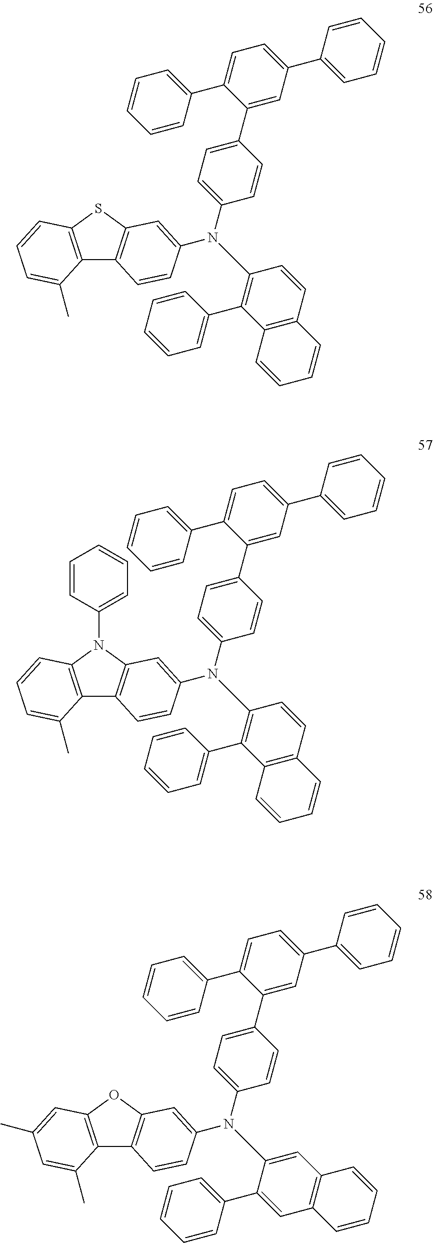 [SMILES: Cc1cccc2sc3cc(N(c4ccc(-c5cc(-c6ccccc6)ccc5-c5ccccc5)cc4)c4ccc5ccccc5c4-c4ccccc4)ccc3c12 ; Cc1cccc2c1c1ccc(N(c3ccc(-c4cc(-c5ccccc5)ccc4-c4ccccc4)cc3)c3ccc4ccccc4c3-c3ccccc3)cc1n2-c1ccccc1 ; Cc1cc(C)c2c(c1)oc1cc(N(c3ccc(-c4cc(-c5ccccc5)ccc4-c4ccccc4)cc3)c3cc4ccccc4cc3-c3ccccc3)ccc12]

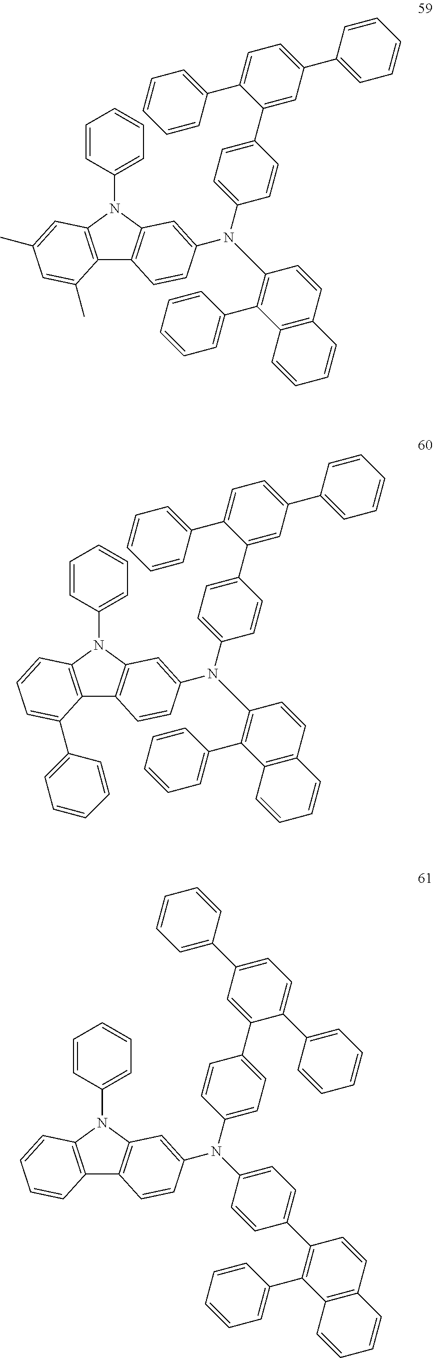 [SMILES: Cc1cc(C)c2c3ccc(N(c4ccc(-c5cc(-c6ccccc6)ccc5-c5ccccc5)cc4)c4ccc5ccccc5c4-c4ccccc4)cc3n(-c3ccccc3)c2c1 ; c1ccc(-c2ccc(-c3ccccc3)c(-c3ccc(N(c4ccc5c6c(-c7ccccc7)cccc6n(-c6ccccc6)c5c4)c4ccc5ccccc5c4-c4ccccc4)cc3)c2)cc1 ; c1ccc(-c2ccc(-c3ccccc3)c(-c3ccc(N(c4ccc(-c5ccc6ccccc6c5-c5ccccc5)cc4)c4ccc5c6ccccc6n(-c6ccccc6)c5c4)cc3)c2)cc1]

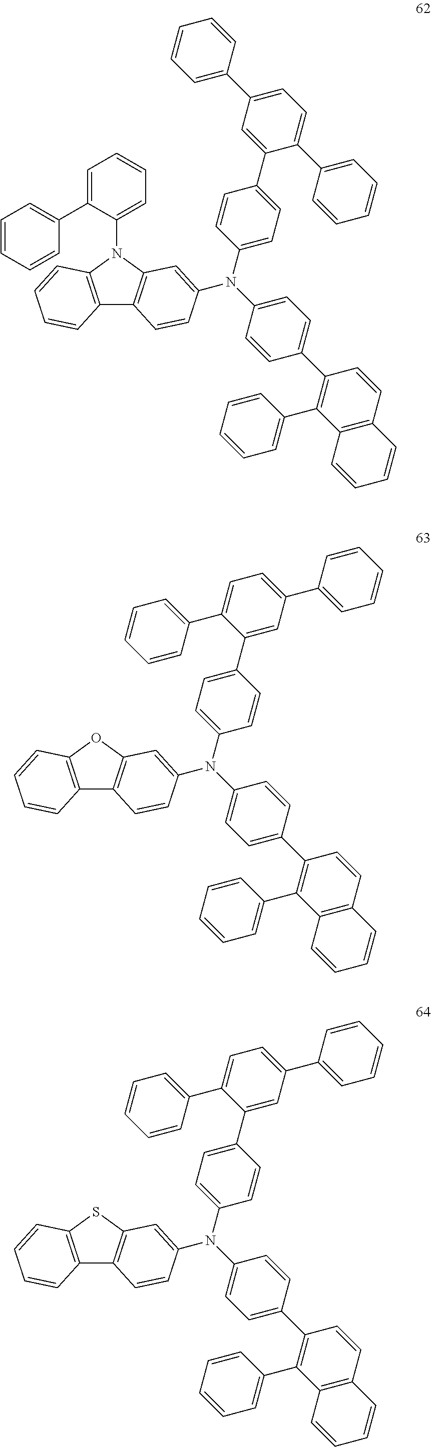 [SMILES: c1ccc(-c2ccc(-c3ccccc3)c(-c3ccc(N(c4ccc(-c5ccc6ccccc6c5-c5ccccc5)cc4)c4ccc5c6ccccc6n(-c6ccccc6-c6ccccc6)c5c4)cc3)c2)cc1 ; c1ccc(-c2ccc(-c3ccccc3)c(-c3ccc(N(c4ccc(-c5ccc6ccccc6c5-c5ccccc5)cc4)c4ccc5c(c4)oc4ccccc45)cc3)c2)cc1 ; c1ccc(-c2ccc(-c3ccccc3)c(-c3ccc(N(c4ccc(-c5ccc6ccccc6c5-c5ccccc5)cc4)c4ccc5c(c4)sc4ccccc45)cc3)c2)cc1]

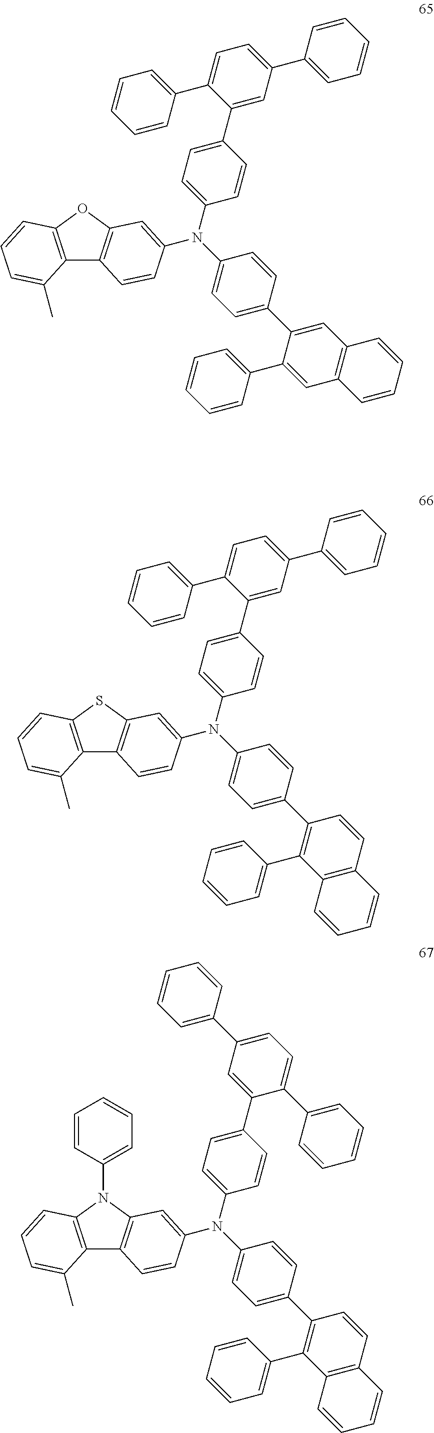 [SMILES: Cc1cccc2oc3cc(N(c4ccc(-c5cc(-c6ccccc6)ccc5-c5ccccc5)cc4)c4ccc(-c5cc6ccccc6cc5-c5ccccc5)cc4)ccc3c12 ; Cc1cccc2sc3cc(N(c4ccc(-c5cc(-c6ccccc6)ccc5-c5ccccc5)cc4)c4ccc(-c5ccc6ccccc6c5-c5ccccc5)cc4)ccc3c12 ; Cc1cccc2c1c1ccc(N(c3ccc(-c4cc(-c5ccccc5)ccc4-c4ccccc4)cc3)c3ccc(-c4ccc5ccccc5c4-c4ccccc4)cc3)cc1n2-c1ccccc1]

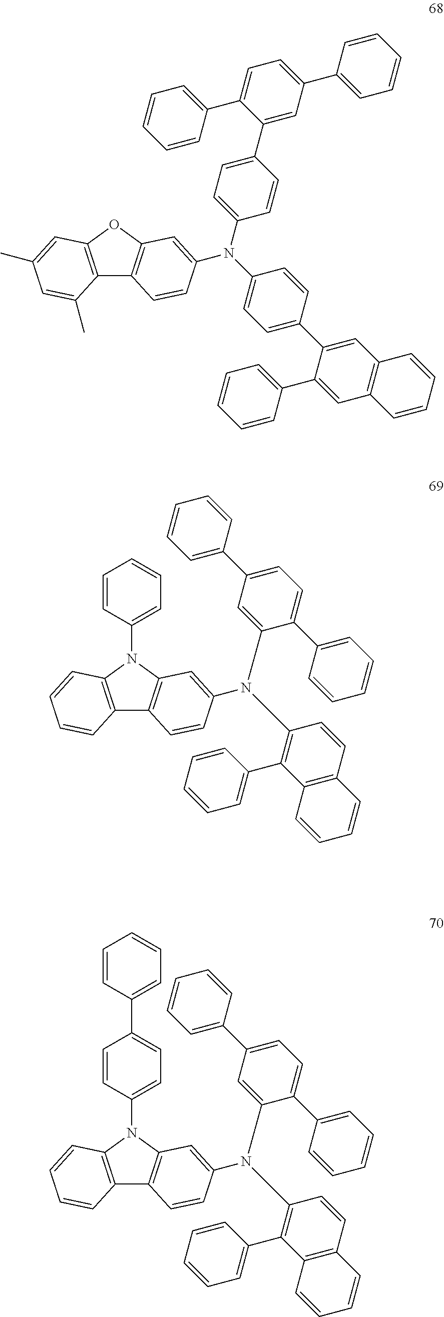 [SMILES: Cc1cc(C)c2c(c1)oc1cc(N(c3ccc(-c4cc(-c5ccccc5)ccc4-c4ccccc4)cc3)c3ccc(-c4cc5ccccc5cc4-c4ccccc4)cc3)ccc12 ; c1ccc(-c2ccc(-c3ccccc3)c(N(c3ccc4c5ccccc5n(-c5ccccc5)c4c3)c3ccc4ccccc4c3-c3ccccc3)c2)cc1 ; c1ccc(-c2ccc(-n3c4ccccc4c4ccc(N(c5cc(-c6ccccc6)ccc5-c5ccccc5)c5ccc6ccccc6c5-c5ccccc5)cc43)cc2)cc1]

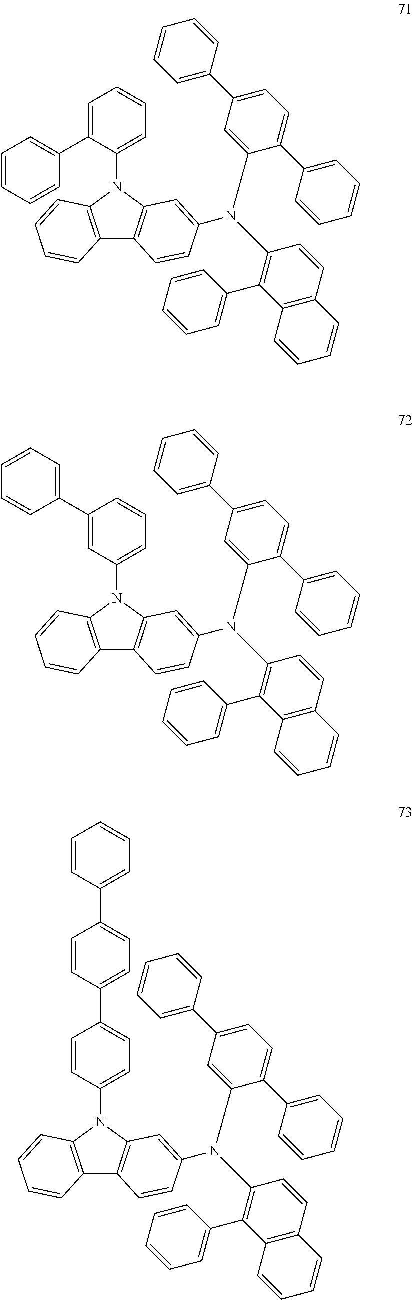 [SMILES: c1ccc(-c2ccc(-c3ccccc3)c(N(c3ccc4c5ccccc5n(-c5ccccc5-c5ccccc5)c4c3)c3ccc4ccccc4c3-c3ccccc3)c2)cc1 ; c1ccc(-c2cccc(-n3c4ccccc4c4ccc(N(c5cc(-c6ccccc6)ccc5-c5ccccc5)c5ccc6ccccc6c5-c5ccccc5)cc43)c2)cc1 ; c1ccc(-c2ccc(-c3ccc(-n4c5ccccc5c5ccc(N(c6cc(-c7ccccc7)ccc6-c6ccccc6)c6ccc7ccccc7c6-c6ccccc6)cc54)cc3)cc2)cc1]

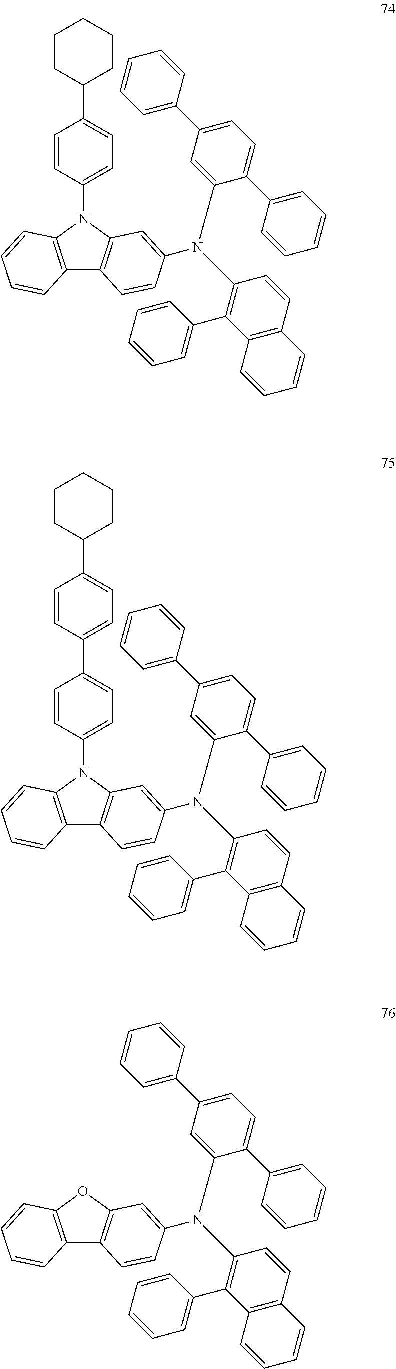 [SMILES: c1ccc(-c2ccc(-c3ccccc3)c(N(c3ccc4c5ccccc5n(-c5ccc(C6CCCCC6)cc5)c4c3)c3ccc4ccccc4c3-c3ccccc3)c2)cc1 ; c1ccc(-c2ccc(-c3ccccc3)c(N(c3ccc4c5ccccc5n(-c5ccc(-c6ccc(C7CCCCC7)cc6)cc5)c4c3)c3ccc4ccccc4c3-c3ccccc3)c2)cc1 ; c1ccc(-c2ccc(-c3ccccc3)c(N(c3ccc4c(c3)oc3ccccc34)c3ccc4ccccc4c3-c3ccccc3)c2)cc1]

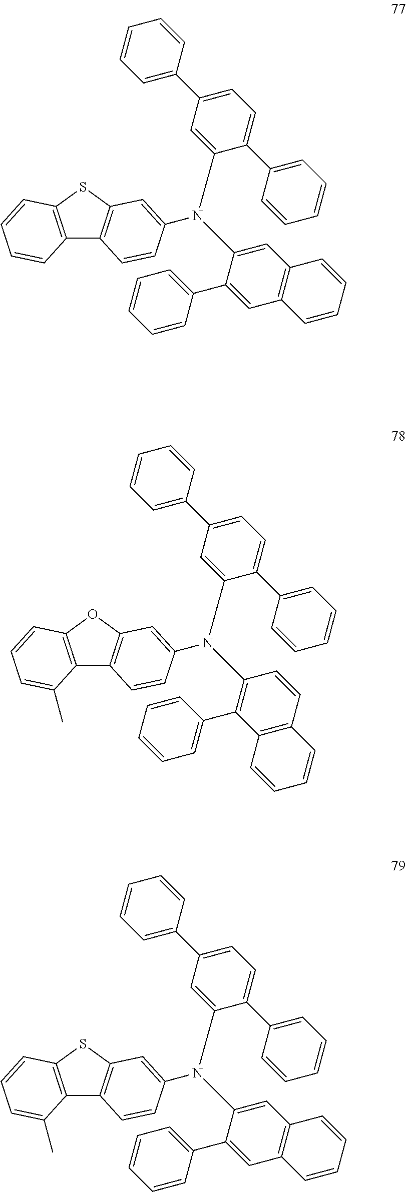 [SMILES: c1ccc(-c2ccc(-c3ccccc3)c(N(c3ccc4c(c3)sc3ccccc34)c3cc4ccccc4cc3-c3ccccc3)c2)cc1 ; Cc1cccc2oc3cc(N(c4cc(-c5ccccc5)ccc4-c4ccccc4)c4ccc5ccccc5c4-c4ccccc4)ccc3c12 ; Cc1cccc2sc3cc(N(c4cc(-c5ccccc5)ccc4-c4ccccc4)c4cc5ccccc5cc4-c4ccccc4)ccc3c12]

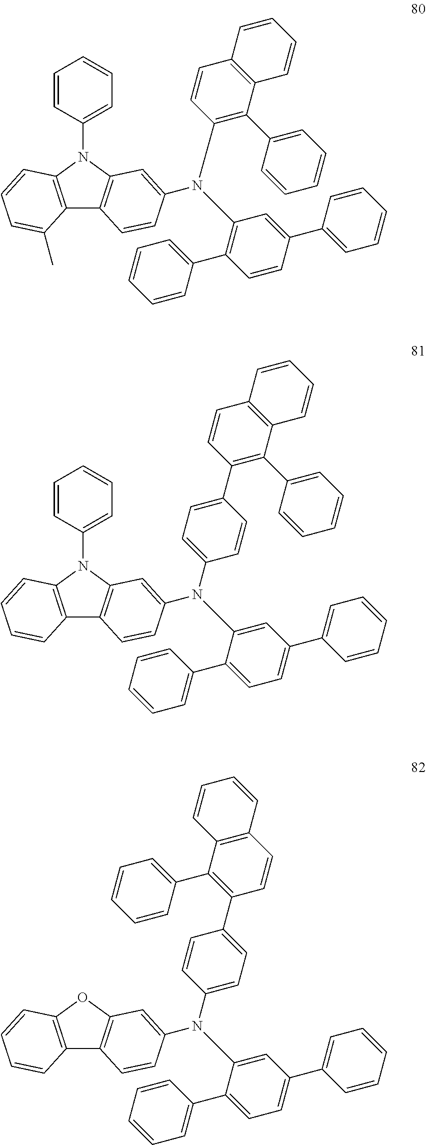 [SMILES: Cc1cccc2c1c1ccc(N(c3cc(-c4ccccc4)ccc3-c3ccccc3)c3ccc4ccccc4c3-c3ccccc3)cc1n2-c1ccccc1 ; c1ccc(-c2ccc(-c3ccccc3)c(N(c3ccc(-c4ccc5ccccc5c4-c4ccccc4)cc3)c3ccc4c5ccccc5n(-c5ccccc5)c4c3)c2)cc1 ; c1ccc(-c2ccc(-c3ccccc3)c(N(c3ccc(-c4ccc5ccccc5c4-c4ccccc4)cc3)c3ccc4c(c3)oc3ccccc34)c2)cc1]

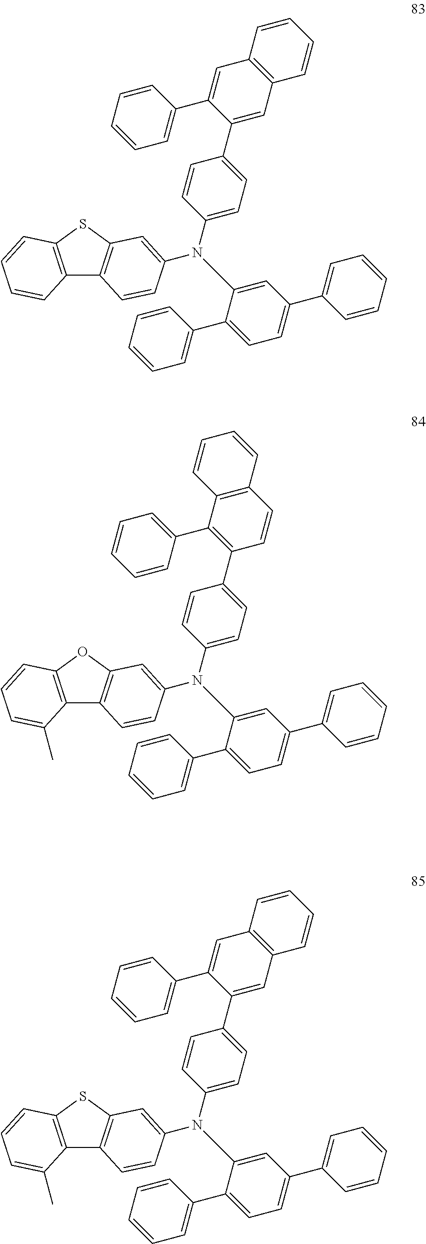 [SMILES: c1ccc(-c2ccc(-c3ccccc3)c(N(c3ccc(-c4cc5ccccc5cc4-c4ccccc4)cc3)c3ccc4c(c3)sc3ccccc34)c2)cc1 ; Cc1cccc2oc3cc(N(c4ccc(-c5ccc6ccccc6c5-c5ccccc5)cc4)c4cc(-c5ccccc5)ccc4-c4ccccc4)ccc3c12 ; Cc1cccc2sc3cc(N(c4ccc(-c5cc6ccccc6cc5-c5ccccc5)cc4)c4cc(-c5ccccc5)ccc4-c4ccccc4)ccc3c12]

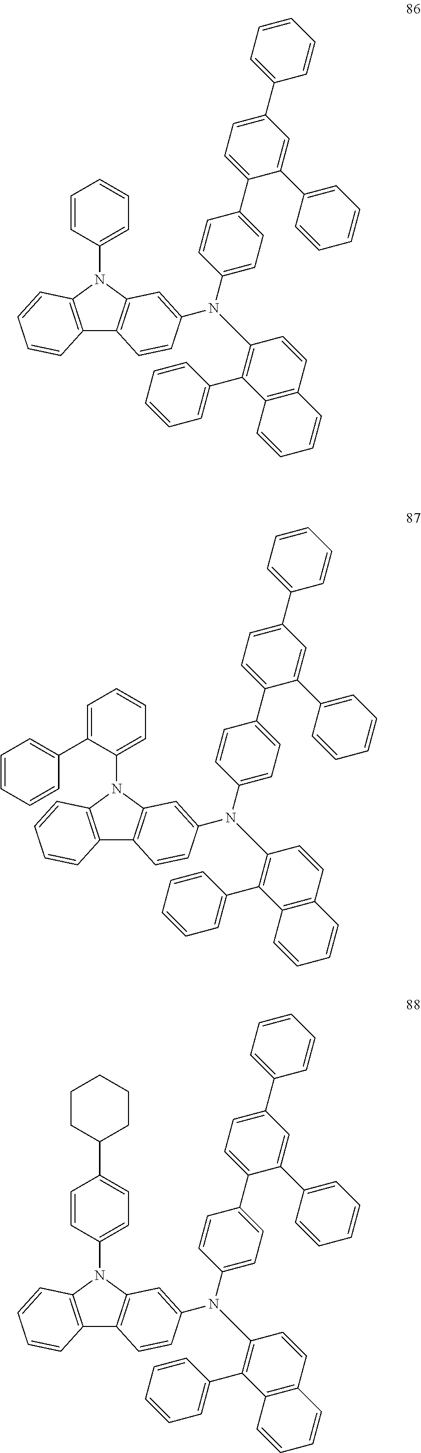 [SMILES: c1ccc(-c2ccc(-c3ccc(N(c4ccc5c6ccccc6n(-c6ccccc6)c5c4)c4ccc5ccccc5c4-c4ccccc4)cc3)c(-c3ccccc3)c2)cc1 ; c1ccc(-c2ccc(-c3ccc(N(c4ccc5c6ccccc6n(-c6ccccc6-c6ccccc6)c5c4)c4ccc5ccccc5c4-c4ccccc4)cc3)c(-c3ccccc3)c2)cc1 ; c1ccc(-c2ccc(-c3ccc(N(c4ccc5c6ccccc6n(-c6ccc(C7CCCCC7)cc6)c5c4)c4ccc5ccccc5c4-c4ccccc4)cc3)c(-c3ccccc3)c2)cc1]

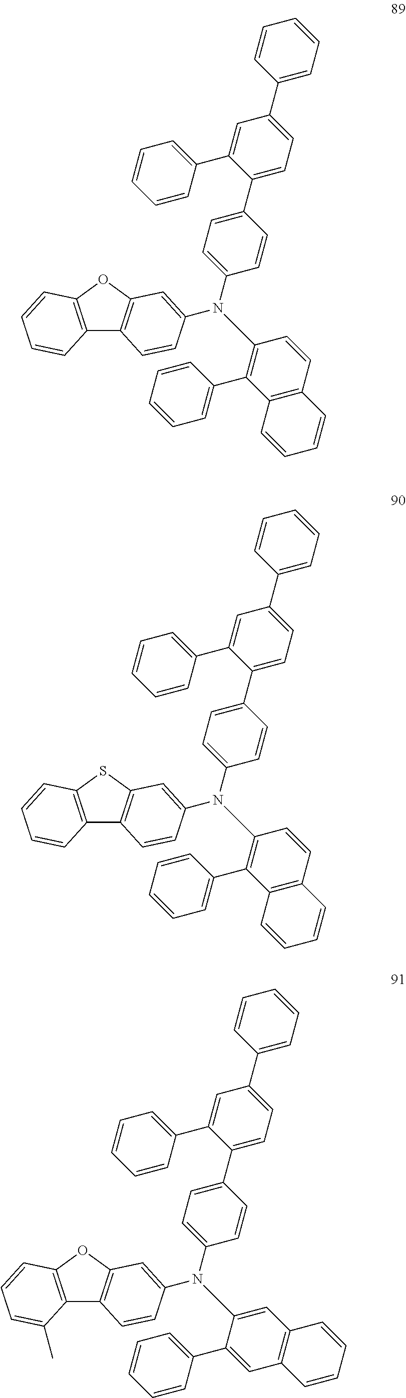

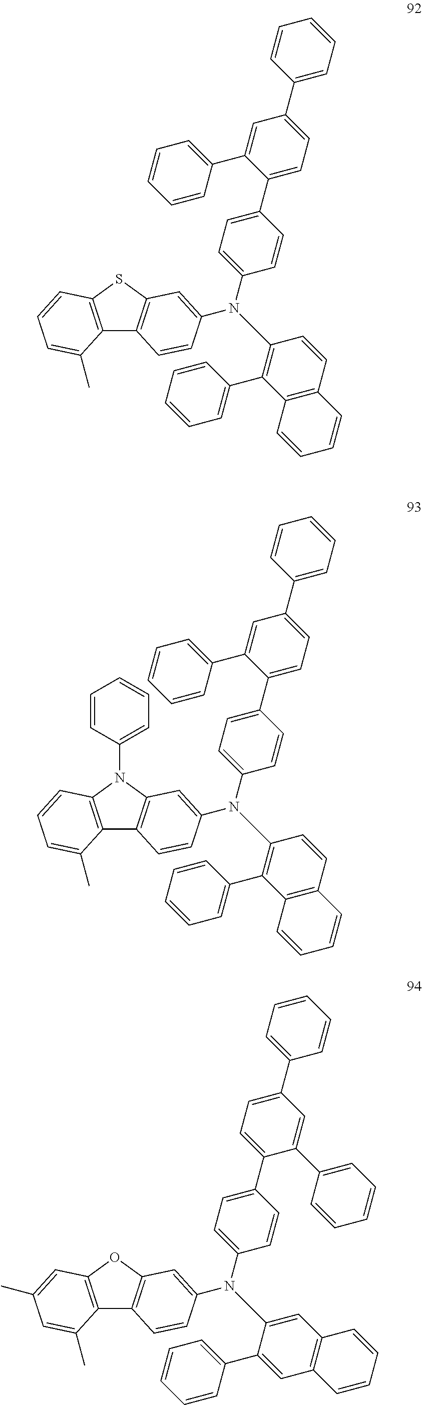

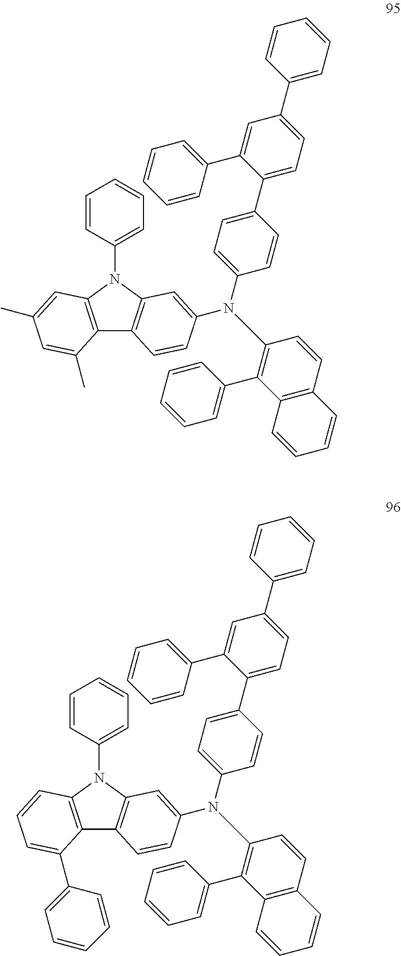 [SMILES: Cc1cc(C)c2c3ccc(N(c4ccc(-c5ccc(-c6ccccc6)cc5-c5ccccc5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)c(-c3ccccc3)c2)cc1]

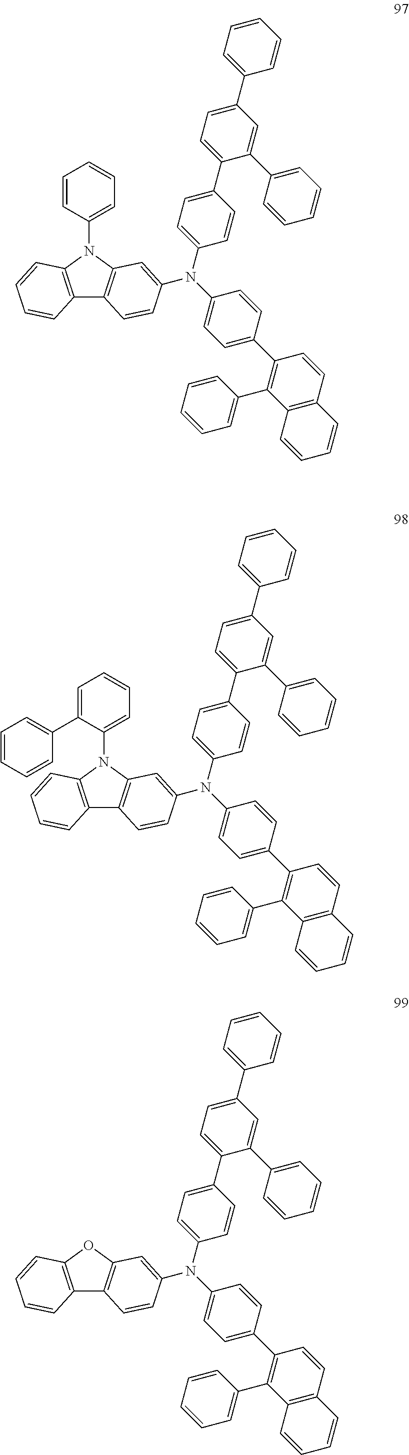 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c(c4)oc4ccccc45)cc3)c(-c3ccccc3)c2)cc1]

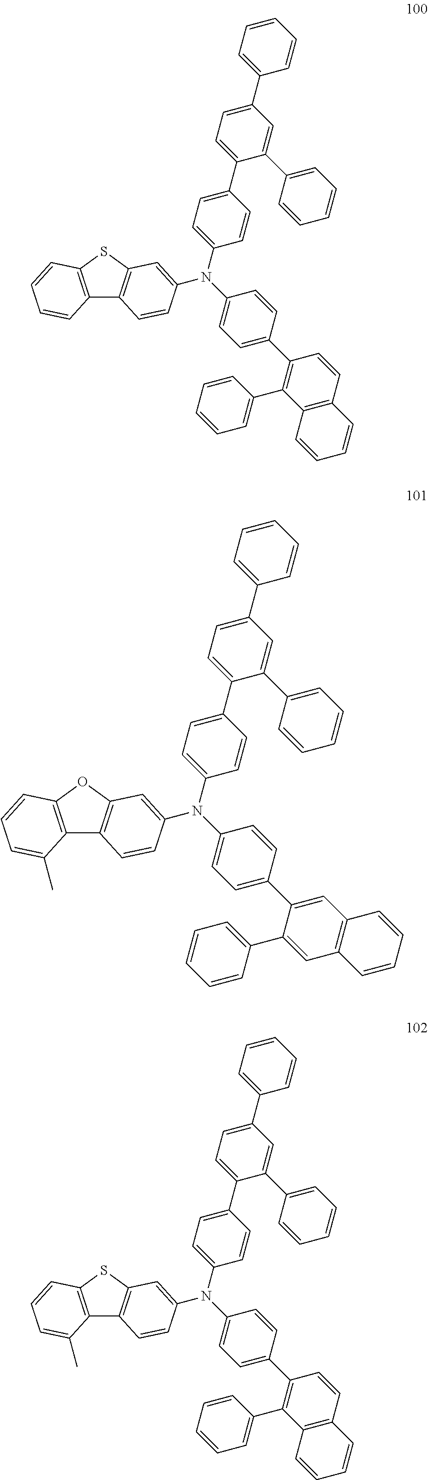 [SMILES: c1ccc(-c2ccc(-c3ccc(N(c4ccc(-c5ccc6ccccc6c5-c5ccccc5)cc4)c4ccc5c(c4)sc4ccccc45)cc3)c(-c3ccccc3)c2)cc1 ; Cc1cccc2oc3cc(N(c4ccc(-c5ccc(-c6ccccc6)cc5-c5ccccc5)cc4)c4ccc(-c5cc6ccccc6cc5-c5ccccc5)cc4)ccc3c12 ; Cc1cccc2sc3cc(N(c4ccc(-c5ccc(-c6ccccc6)cc5-c5ccccc5)cc4)c4ccc(-c5ccc6ccccc6c5-c5ccccc5)cc4)ccc3c12]

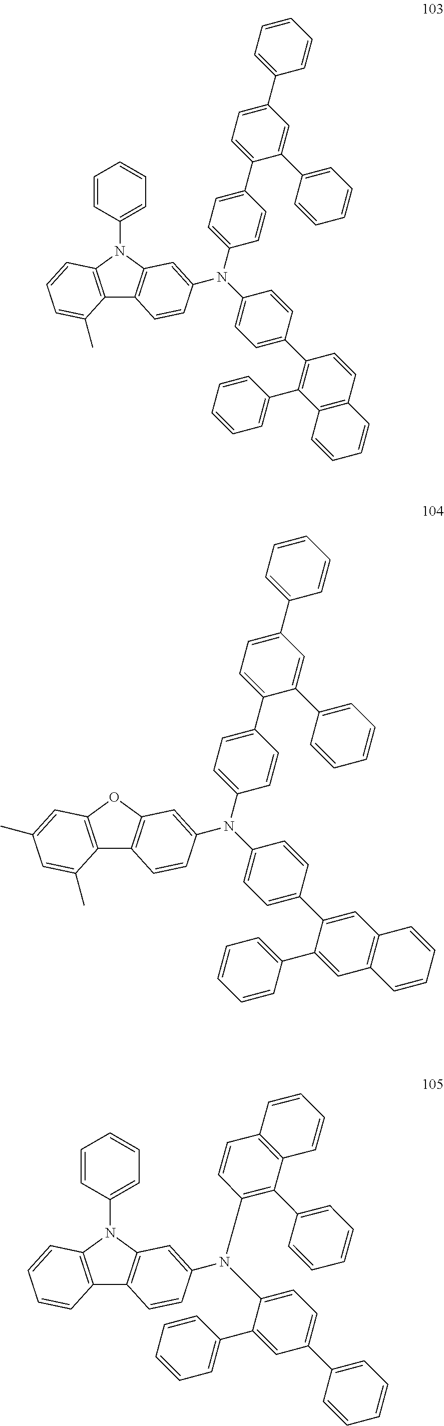 [SMILES: Cc1cccc2c1c1ccc(N(c3ccc(-c4ccc(-c5ccccc5)cc4-c4ccccc4)cc3)c3ccc(-c4ccc5ccccc5c4-c4ccccc4)cc3)cc1n2-c1ccccc1 ; Cc1cc(C)c2c(c1)oc1cc(N(c3ccc(-c4ccc(-c5ccccc5)cc4-c4ccccc4)cc3)c3ccc(-c4cc5ccccc5cc4-c4ccccc4)cc3)ccc12 ; c1ccc(-c2ccc(N(c3ccc4c5ccccc5n(-c5ccccc5)c4c3)c3ccc4ccccc4c3-c3ccccc3)c(-c3ccccc3)c2)cc1]

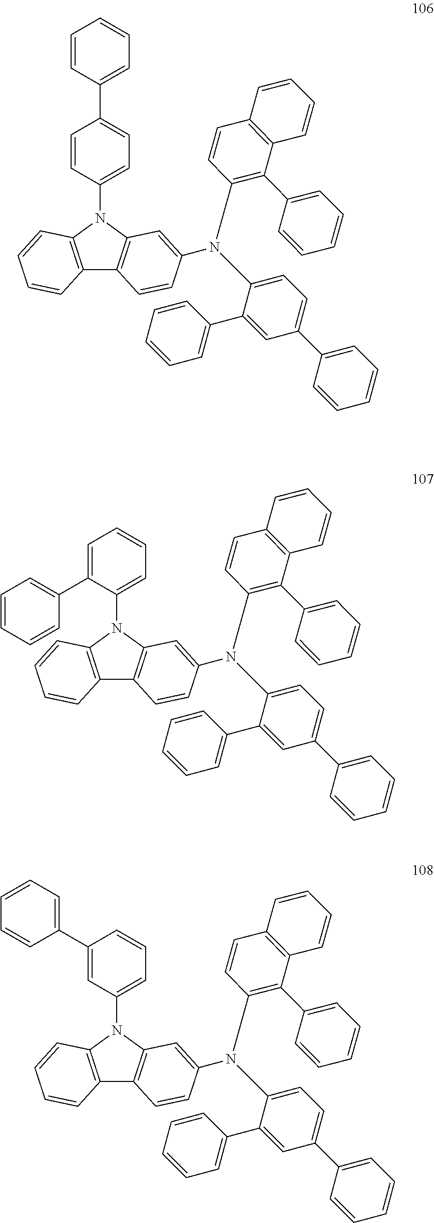 [SMILES: c1ccc(-c2ccc(-n3c4ccccc4c4ccc(N(c5ccc(-c6ccccc6)cc5-c5ccccc5)c5ccc6ccccc6c5-c5ccccc5)cc43)cc2)cc1 ; c1ccc(-c2ccc(N(c3ccc4c5ccccc5n(-c5ccccc5-c5ccccc5)c4c3)c3ccc4ccccc4c3-c3ccccc3)c(-c3ccccc3)c2)cc1 ; c1ccc(-c2cccc(-n3c4ccccc4c4ccc(N(c5ccc(-c6ccccc6)cc5-c5ccccc5)c5ccc6ccccc6c5-c5ccccc5)cc43)c2)cc1]

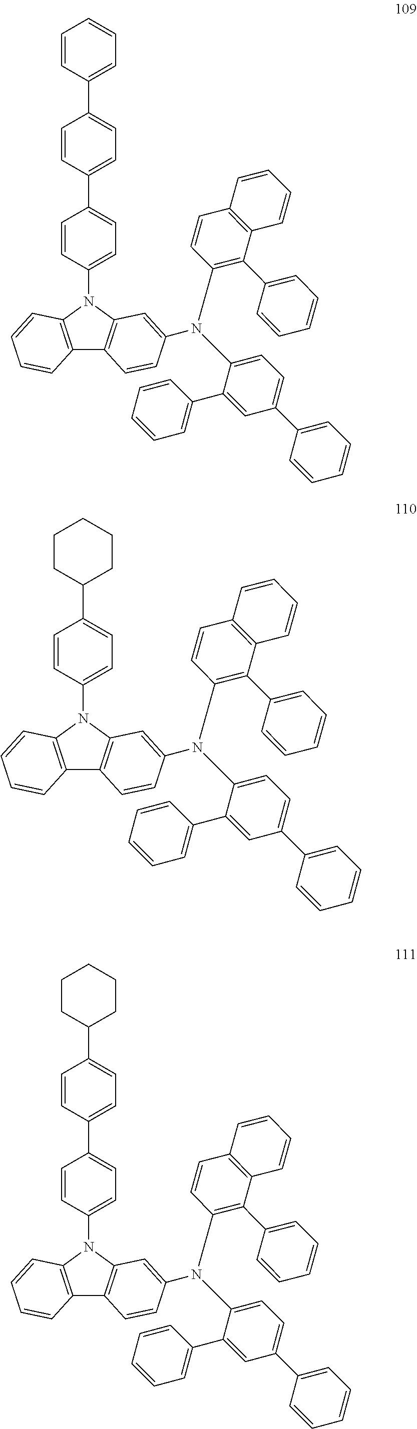 [SMILES: c1ccc(-c2ccc(-c3ccc(-n4c5ccccc5c5ccc(N(c6ccc(-c7ccccc7)cc6-c6ccccc6)c6ccc7ccccc7c6-c6ccccc6)cc54)cc3)cc2)cc1 ; c1ccc(-c2ccc(N(c3ccc4c5ccccc5n(-c5ccc(C6CCCCC6)cc5)c4c3)c3ccc4ccccc4c3-c3ccccc3)c(-c3ccccc3)c2)cc1 ; c1ccc(-c2ccc(N(c3ccc4c5ccccc5n(-c5ccc(-c6ccc(C7CCCCC7)cc6)cc5)c4c3)c3ccc4ccccc4c3-c3ccccc3)c(-c3ccccc3)c2)cc1]

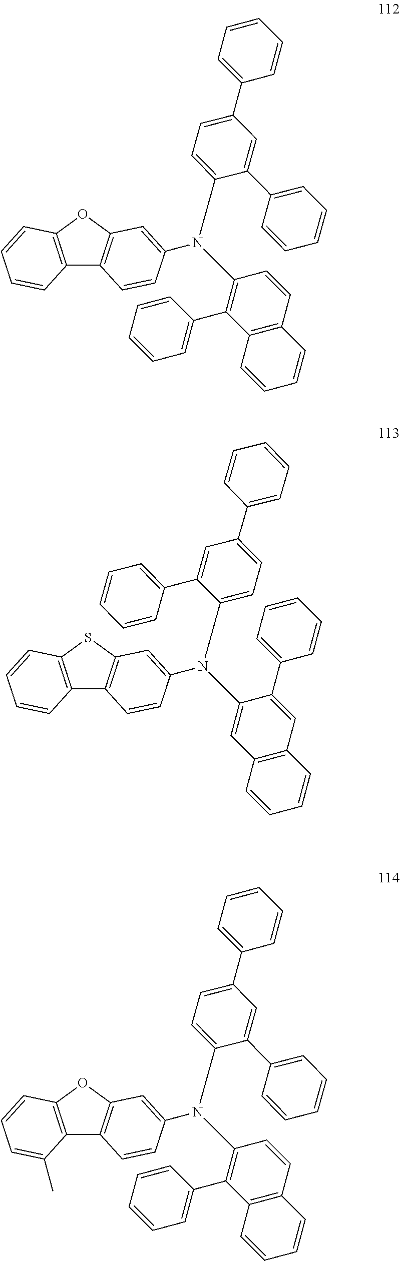 [SMILES: c1ccc(-c2ccc(N(c3ccc4c(c3)oc3ccccc34)c3ccc4ccccc4c3-c3ccccc3)c(-c3ccccc3)c2)cc1 ; c1ccc(-c2ccc(N(c3ccc4c(c3)sc3ccccc34)c3cc4ccccc4cc3-c3ccccc3)c(-c3ccccc3)c2)cc1 ; Cc1cccc2oc3cc(N(c4ccc(-c5ccccc5)cc4-c4ccccc4)c4ccc5ccccc5c4-c4ccccc4)ccc3c12]

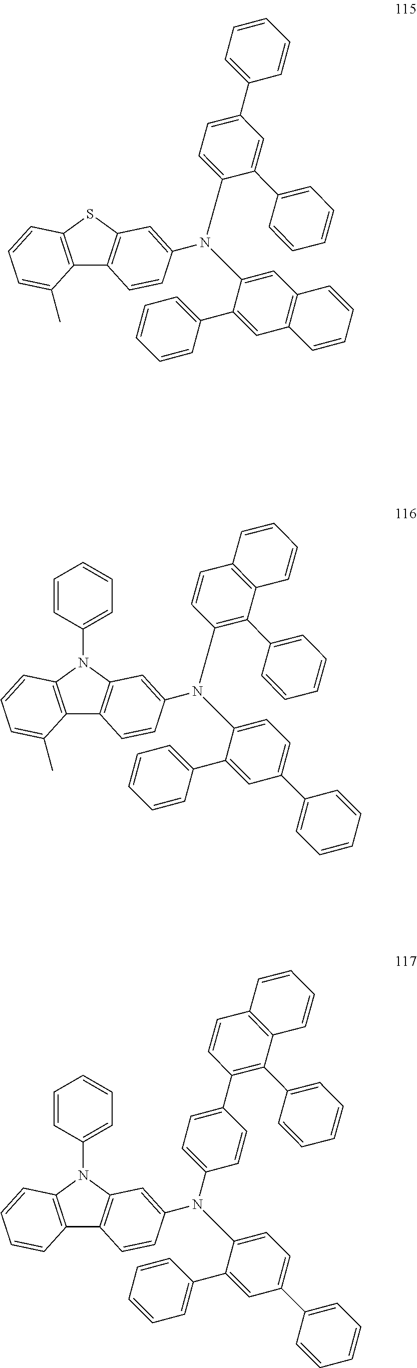 [SMILES: Cc1cccc2sc3cc(N(c4ccc(-c5ccccc5)cc4-c4ccccc4)c4cc5ccccc5cc4-c4ccccc4)ccc3c12 ; Cc1cccc2c1c1ccc(N(c3ccc(-c4ccccc4)cc3-c3ccccc3)c3ccc4ccccc4c3-c3ccccc3)cc1n2-c1ccccc1 ; c1ccc(-c2ccc(N(c3ccc(-c4ccc5ccccc5c4-c4ccccc4)cc3)c3ccc4c5ccccc5n(-c5ccccc5)c4c3)c(-c3ccccc3)c2)cc1]

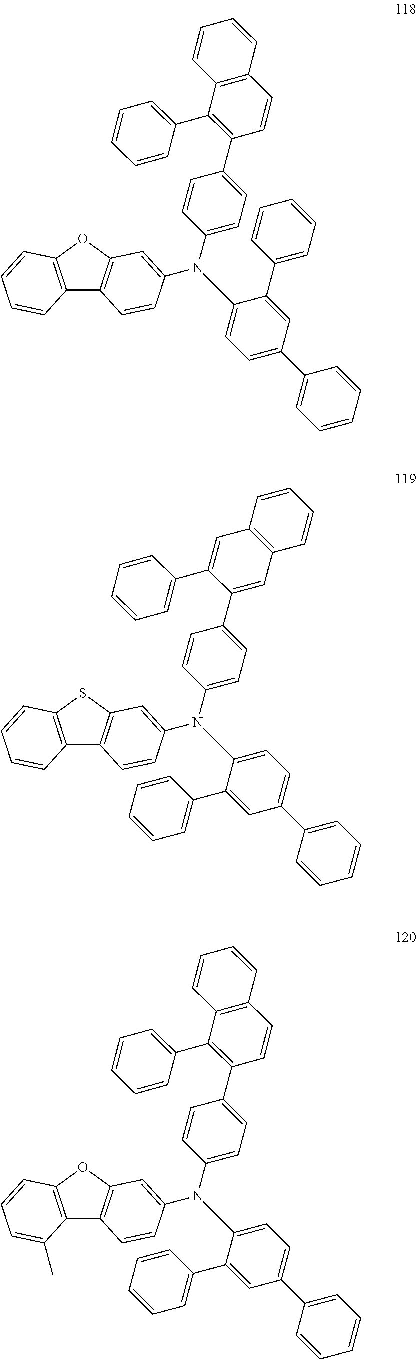 [SMILES: c1ccc(-c2ccc(N(c3ccc(-c4ccc5ccccc5c4-c4ccccc4)cc3)c3ccc4c(c3)oc3ccccc34)c(-c3ccccc3)c2)cc1 ; c1ccc(-c2ccc(N(c3ccc(-c4cc5ccccc5cc4-c4ccccc4)cc3)c3ccc4c(c3)sc3ccccc34)c(-c3ccccc3)c2)cc1 ; Cc1cccc2oc3cc(N(c4ccc(-c5ccc6ccccc6c5-c5ccccc5)cc4)c4ccc(-c5ccccc5)cc4-c4ccccc4)ccc3c12]

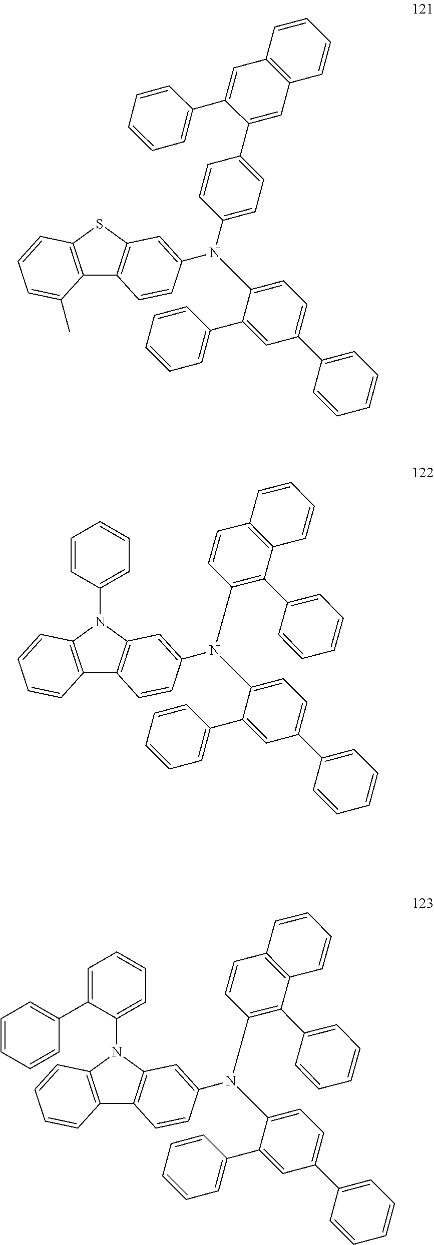 [SMILES: Cc1cccc2sc3cc(N(c4ccc(-c5cc6ccccc6cc5-c5ccccc5)cc4)c4ccc(-c5ccccc5)cc4-c4ccccc4)ccc3c12 ; c1ccc(-c2ccc(N(c3ccc4c5ccccc5n(-c5ccccc5)c4c3)c3ccc4ccccc4c3-c3ccccc3)c(-c3ccccc3)c2)cc1 ; c1ccc(-c2ccc(N(c3ccc4c5ccccc5n(-c5ccccc5-c5ccccc5)c4c3)c3ccc4ccccc4c3-c3ccccc3)c(-c3ccccc3)c2)cc1]

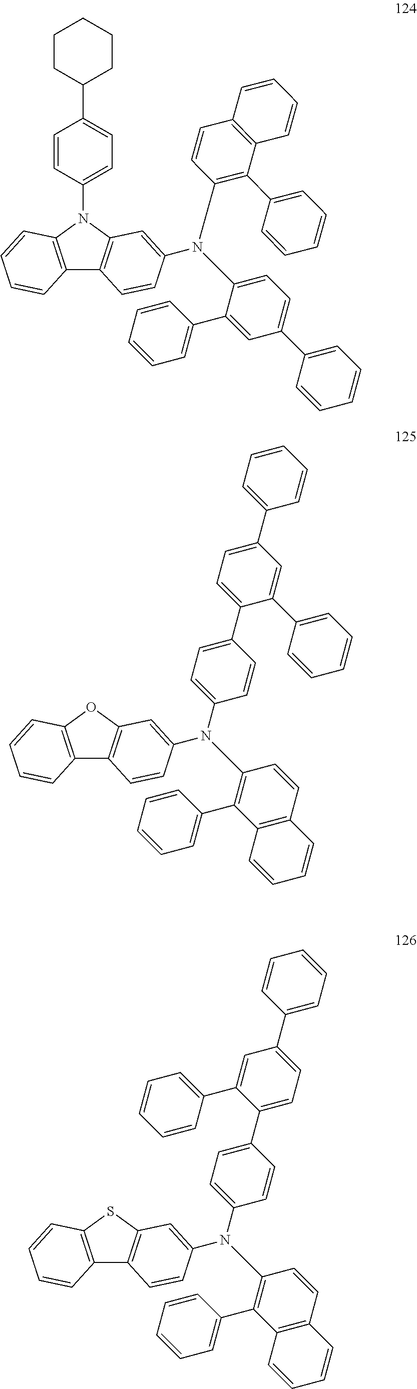 [SMILES: c1ccc(-c2ccc(N(c3ccc4c5ccccc5n(-c5ccc(C6CCCCC6)cc5)c4c3)c3ccc4ccccc4c3-c3ccccc3)c(-c3ccccc3)c2)cc1 ; c1ccc(-c2ccc(-c3ccc(N(c4ccc5c(c4)oc4ccccc45)c4ccc5ccccc5c4-c4ccccc4)cc3)c(-c3ccccc3)c2)cc1 ; c1ccc(-c2ccc(-c3ccc(N(c4ccc5c(c4)sc4ccccc45)c4ccc5ccccc5c4-c4ccccc4)cc3)c(-c3ccccc3)c2)cc1]

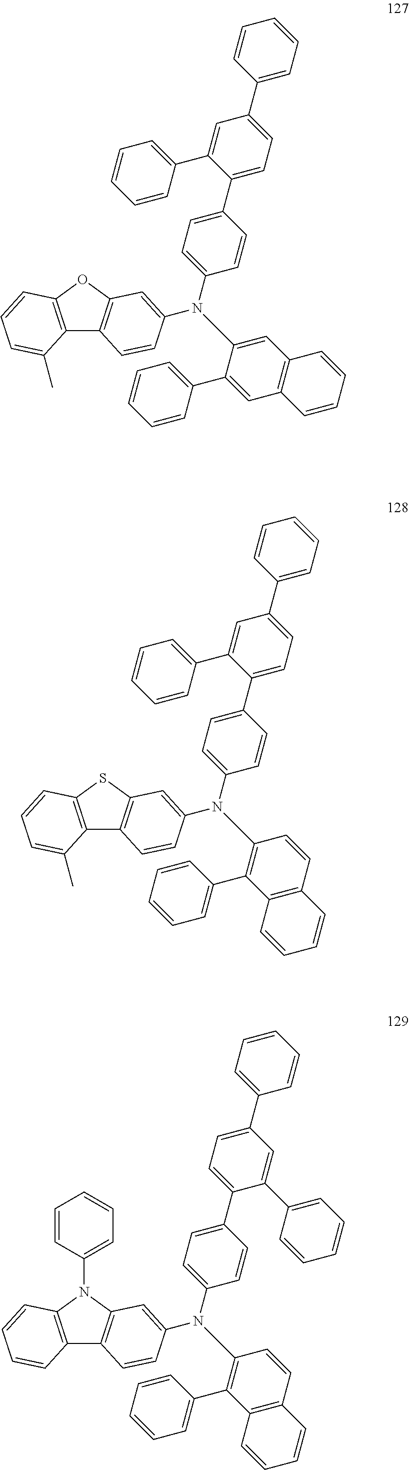 [SMILES: Cc1cccc2oc3cc(N(c4ccc(-c5ccc(-c6ccccc6)cc5-c5ccccc5)cc4)c4cc5ccccc5cc4-c4ccccc4)ccc3c12 ; Cc1cccc2sc3cc(N(c4ccc(-c5ccc(-c6ccccc6)cc5-c5ccccc5)cc4)c4ccc5ccccc5c4-c4ccccc4)ccc3c12 ; c1ccc(-c2ccc(-c3ccc(N(c4ccc5c6ccccc6n(-c6ccccc6)c5c4)c4ccc5ccccc5c4-c4ccccc4)cc3)c(-c3ccccc3)c2)cc1]

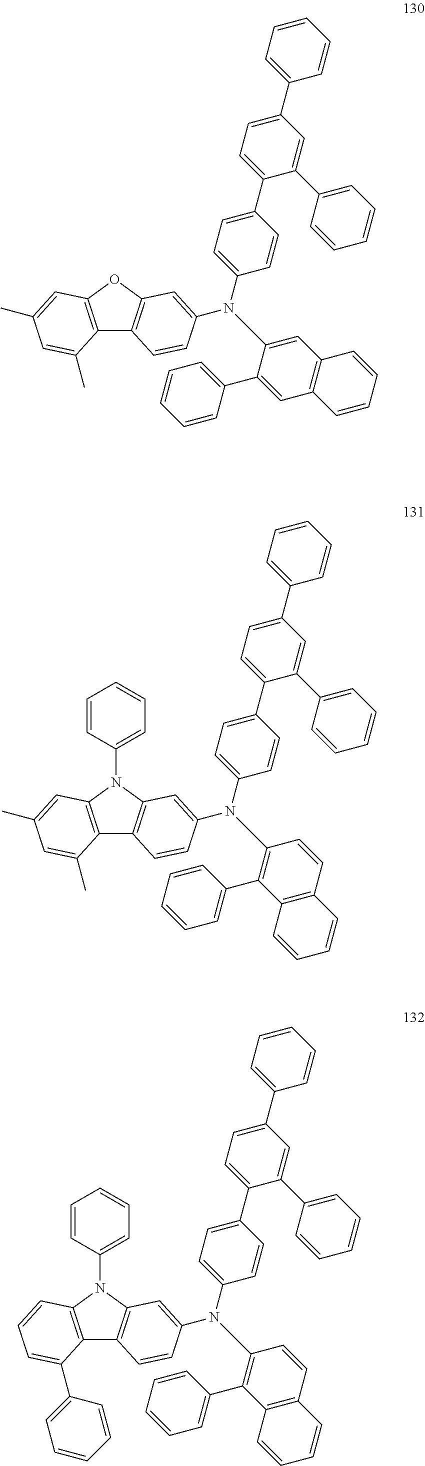 [SMILES: Cc1cc(C)c2c(c1)oc1cc(N(c3ccc(-c4ccc(-c5ccccc5)cc4-c4ccccc4)cc3)c3cc4ccccc4cc3-c3ccccc3)ccc12 ; Cc1cc(C)c2c3ccc(N(c4ccc(-c5ccc(-c6ccccc6)cc5-c5ccccc5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)c(-c3ccccc3)c2)cc1]

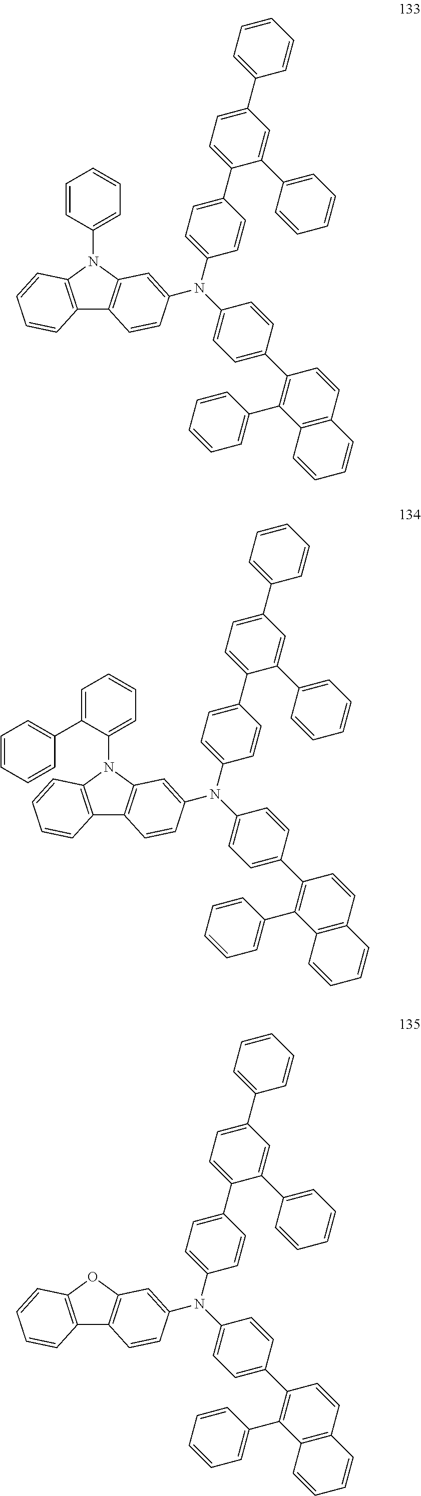 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c(c4)oc4ccccc45)cc3)c(-c3ccccc3)c2)cc1]

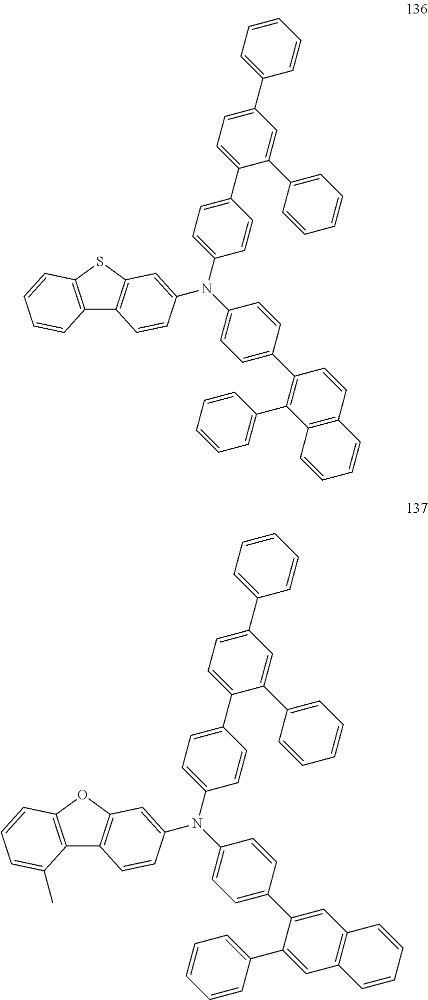 [SMILES: c1ccc(-c2ccc(-c3ccc(N(c4ccc(-c5ccc6ccccc6c5-c5ccccc5)cc4)c4ccc5c(c4)sc4ccccc45)cc3)c(-c3ccccc3)c2)cc1 ; Cc1cccc2oc3cc(N(c4ccc(-c5ccc(-c6ccccc6)cc5-c5ccccc5)cc4)c4ccc(-c5cc6ccccc6cc5-c5ccccc5)cc4)ccc3c12]

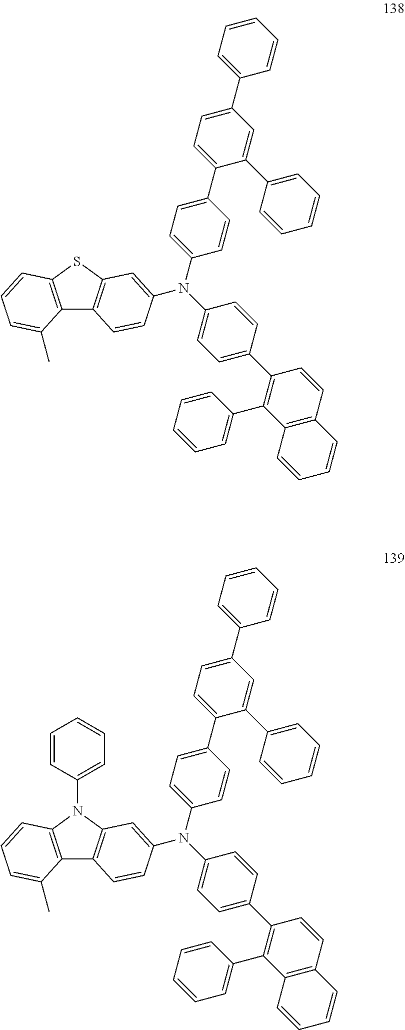 [SMILES: Cc1cccc2sc3cc(N(c4ccc(-c5ccc(-c6ccccc6)cc5-c5ccccc5)cc4)c4ccc(-c5ccc6ccccc6c5-c5ccccc5)cc4)ccc3c12 ; Cc1cccc2c1c1ccc(N(c3ccc(-c4ccc(-c5ccccc5)cc4-c4ccccc4)cc3)c3ccc(-c4ccc5ccccc5c4-c4ccccc4)cc3)cc1n2-c1ccccc1]

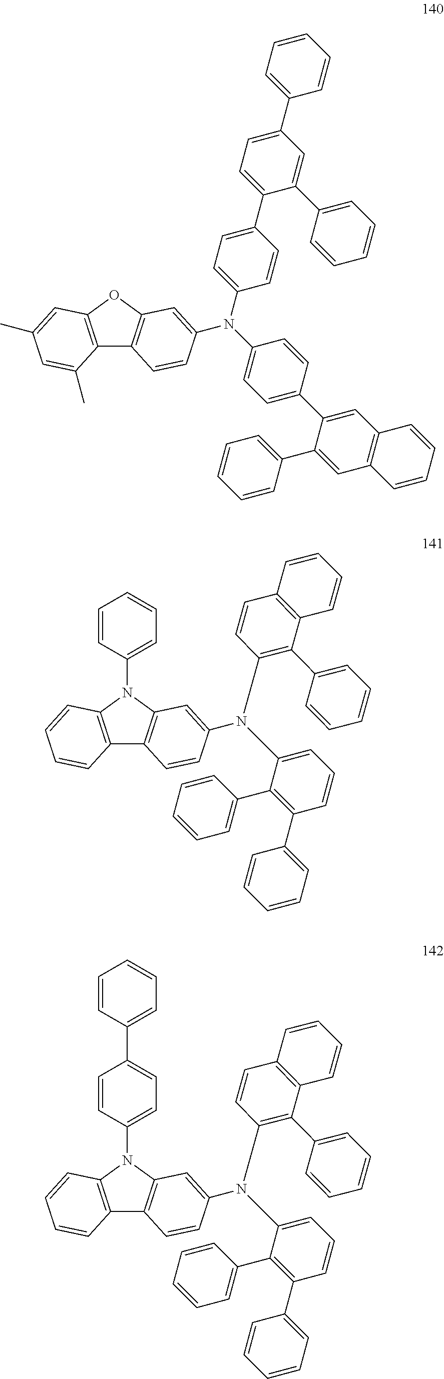 [SMILES: Cc1cc(C)c2c(c1)oc1cc(N(c3ccc(-c4ccc(-c5ccccc5)cc4-c4ccccc4)cc3)c3ccc(-c4cc5ccccc5cc4-c4ccccc4)cc3)ccc12 ; c1ccc(-c2cccc(N(c3ccc4c5ccccc5n(-c5ccccc5)c4c3)c3ccc4ccccc4c3-c3ccccc3)c2-c2ccccc2)cc1 ; c1ccc(-c2ccc(-n3c4ccccc4c4ccc(N(c5cccc(-c6ccccc6)c5-c5ccccc5)c5ccc6ccccc6c5-c5ccccc5)cc43)cc2)cc1]

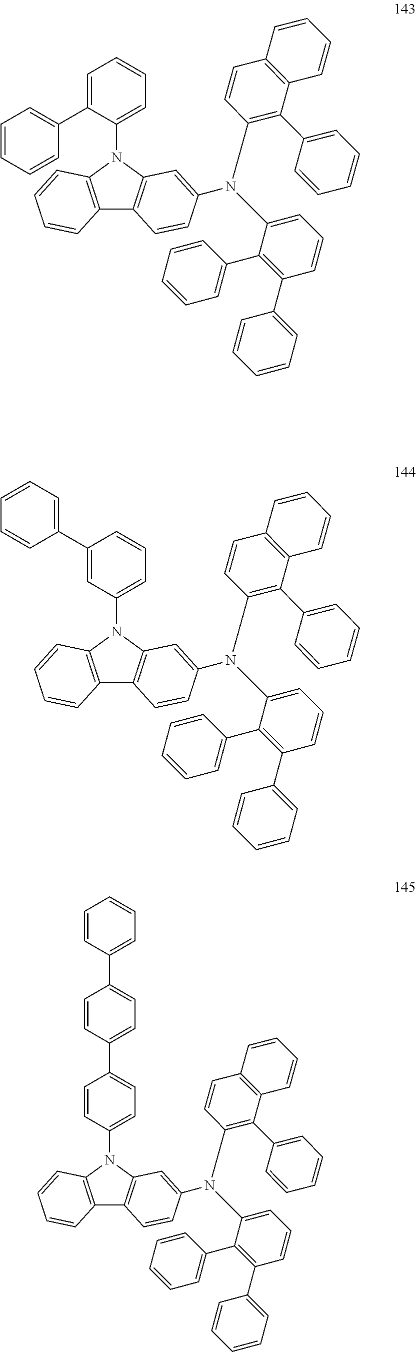 [SMILES: c1ccc(-c2ccccc2-n2c3ccccc3c3ccc(N(c4cccc(-c5ccccc5)c4-c4ccccc4)c4ccc5ccccc5c4-c4ccccc4)cc32)cc1 ; c1ccc(-c2cccc(-n3c4ccccc4c4ccc(N(c5cccc(-c6ccccc6)c5-c5ccccc5)c5ccc6ccccc6c5-c5ccccc5)cc43)c2)cc1 ; c1ccc(-c2ccc(-c3ccc(-n4c5ccccc5c5ccc(N(c6cccc(-c7ccccc7)c6-c6ccccc6)c6ccc7ccccc7c6-c6ccccc6)cc54)cc3)cc2)cc1]

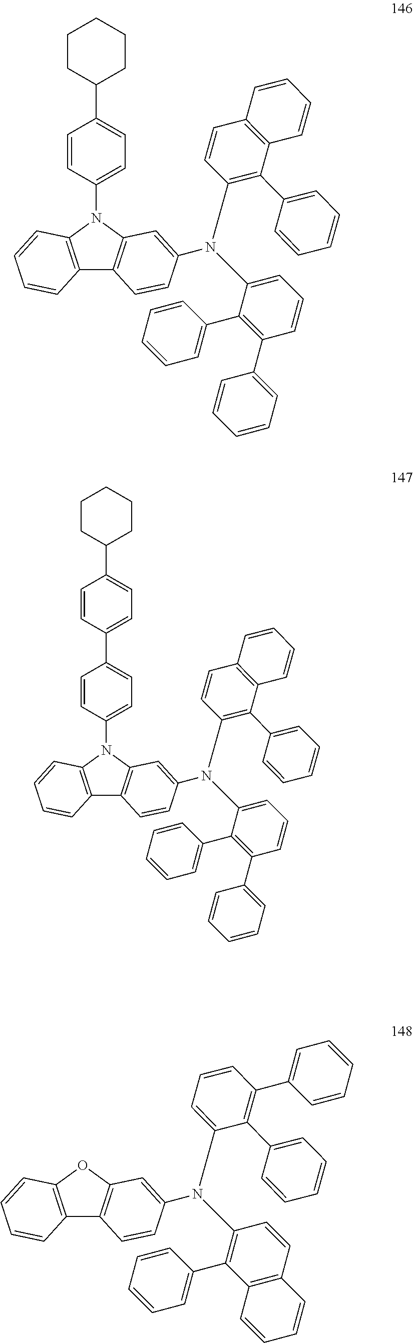 [SMILES: c1ccc(-c2cccc(N(c3ccc4c5ccccc5n(-c5ccc(C6CCCCC6)cc5)c4c3)c3ccc4ccccc4c3-c3ccccc3)c2-c2ccccc2)cc1 ; c1ccc(-c2cccc(N(c3ccc4c5ccccc5n(-c5ccc(-c6ccc(C7CCCCC7)cc6)cc5)c4c3)c3ccc4ccccc4c3-c3ccccc3)c2-c2ccccc2)cc1 ; c1ccc(-c2cccc(N(c3ccc4c(c3)oc3ccccc34)c3ccc4ccccc4c3-c3ccccc3)c2-c2ccccc2)cc1]

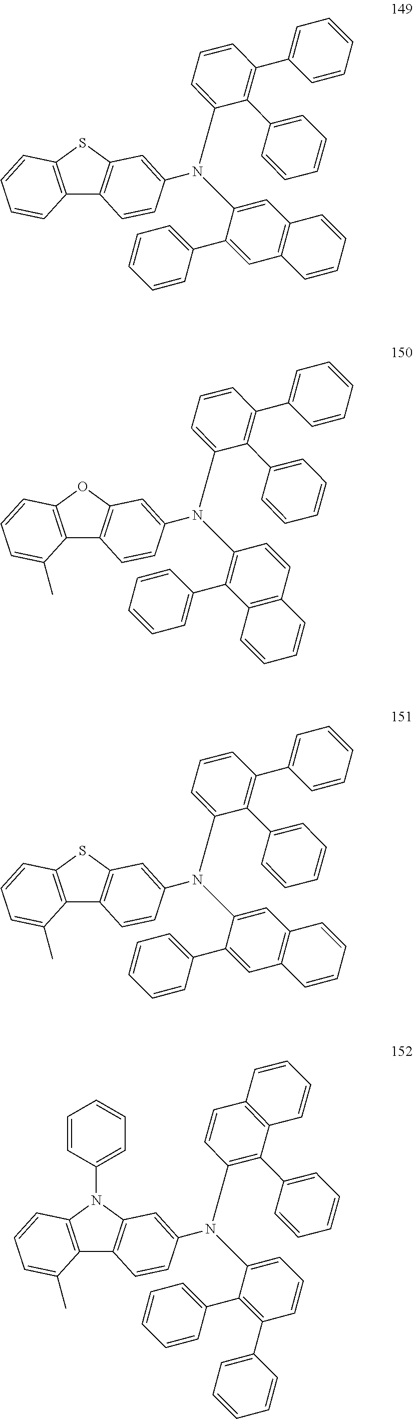 [SMILES: c1ccc(-c2cc3ccccc3cc2N(c2ccc3c(c2)sc2ccccc23)c2cccc(-c3ccccc3)c2-c2ccccc2)cc1 ; Cc1cccc2oc3cc(N(c4cccc(-c5ccccc5)c4-c4ccccc4)c4ccc5ccccc5c4-c4ccccc4)ccc3c12 ; Cc1cccc2sc3cc(N(c4cc5ccccc5cc4-c4ccccc4)c4cccc(-c5ccccc5)c4-c4ccccc4)ccc3c12 ; Cc1cccc2c1c1ccc(N(c3cccc(-c4ccccc4)c3-c3ccccc3)c3ccc4ccccc4c3-c3ccccc3)cc1n2-c1ccccc1]

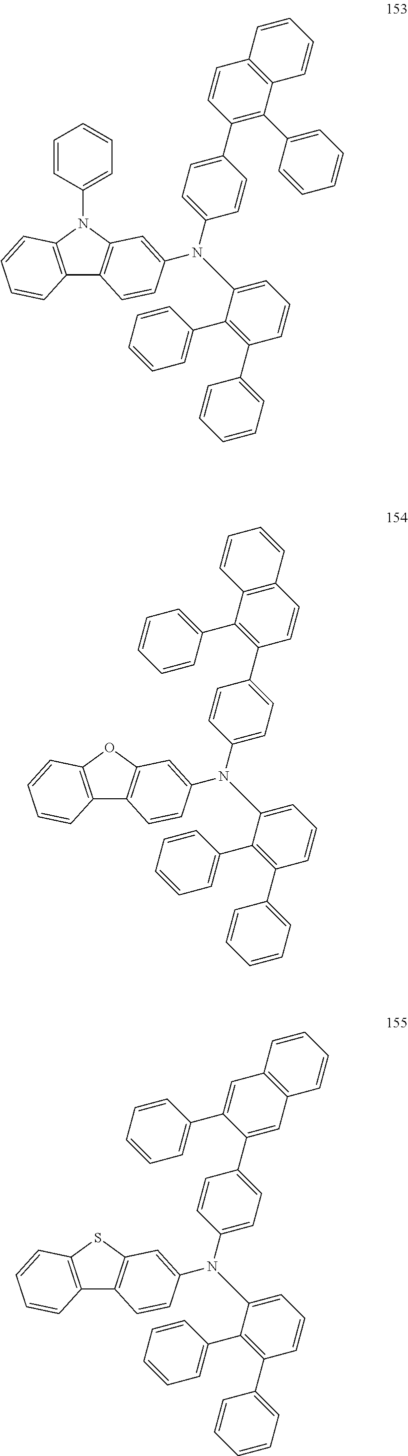 [SMILES: c1ccc(-c2cccc(N(c3ccc(-c4ccc5ccccc5c4-c4ccccc4)cc3)c3ccc4c5ccccc5n(-c5ccccc5)c4c3)c2-c2ccccc2)cc1 ; c1ccc(-c2cccc(N(c3ccc(-c4ccc5ccccc5c4-c4ccccc4)cc3)c3ccc4c(c3)oc3ccccc34)c2-c2ccccc2)cc1 ; c1ccc(-c2cc3ccccc3cc2-c2ccc(N(c3ccc4c(c3)sc3ccccc34)c3cccc(-c4ccccc4)c3-c3ccccc3)cc2)cc1]

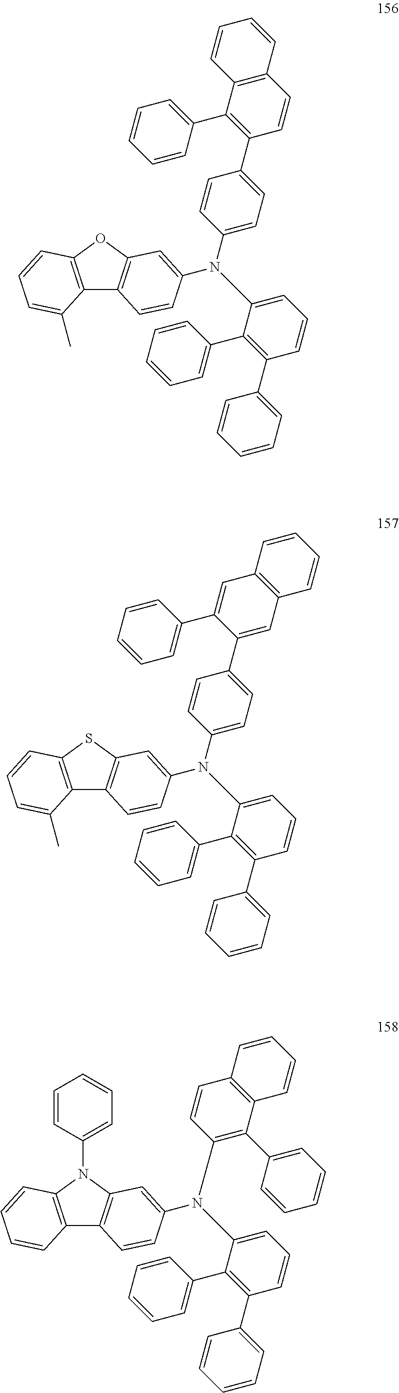 [SMILES: Cc1cccc2oc3cc(N(c4ccc(-c5ccc6ccccc6c5-c5ccccc5)cc4)c4cccc(-c5ccccc5)c4-c4ccccc4)ccc3c12 ; Cc1cccc2sc3cc(N(c4ccc(-c5cc6ccccc6cc5-c5ccccc5)cc4)c4cccc(-c5ccccc5)c4-c4ccccc4)ccc3c12 ; c1ccc(-c2cccc(N(c3ccc4c5ccccc5n(-c5ccccc5)c4c3)c3ccc4ccccc4c3-c3ccccc3)c2-c2ccccc2)cc1]

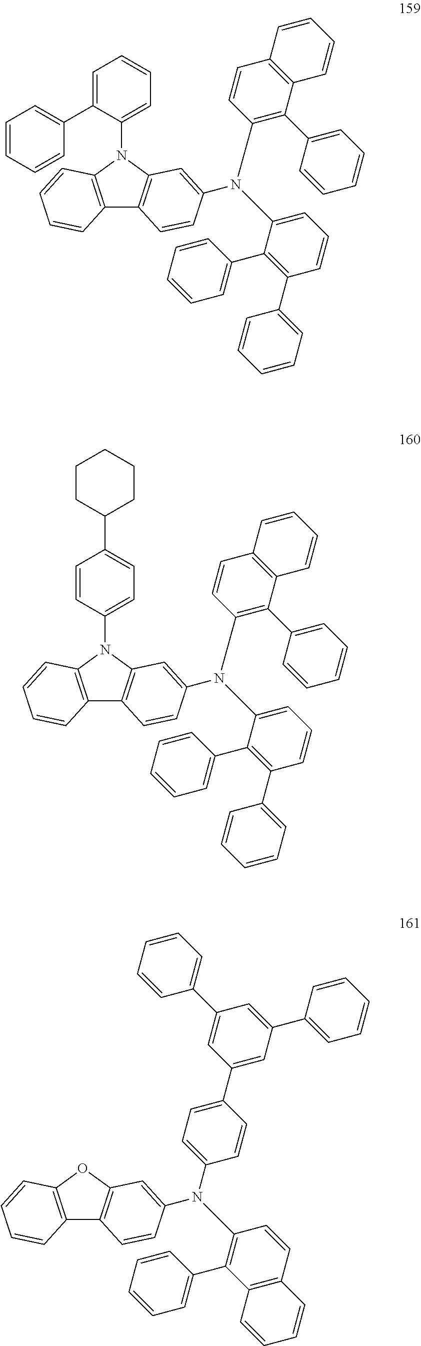 [SMILES: c1ccc(-c2ccccc2-n2c3ccccc3c3ccc(N(c4cccc(-c5ccccc5)c4-c4ccccc4)c4ccc5ccccc5c4-c4ccccc4)cc32)cc1 ; c1ccc(-c2cccc(N(c3ccc4c5ccccc5n(-c5ccc(C6CCCCC6)cc5)c4c3)c3ccc4ccccc4c3-c3ccccc3)c2-c2ccccc2)cc1 ; c1ccc(-c2cc(-c3ccccc3)cc(-c3ccc(N(c4ccc5c(c4)oc4ccccc45)c4ccc5ccccc5c4-c4ccccc4)cc3)c2)cc1]

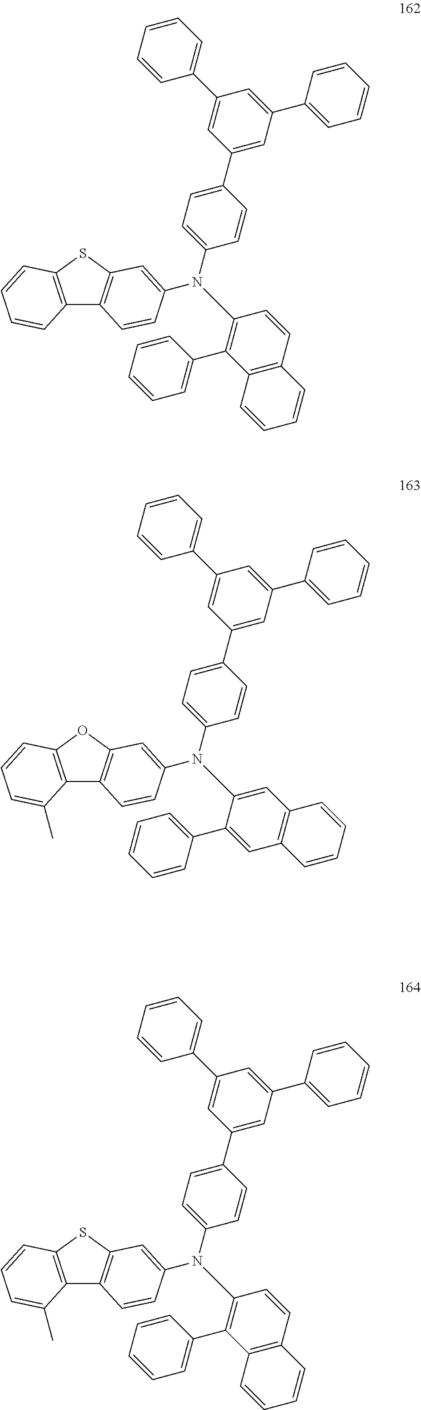 [SMILES: c1ccc(-c2cc(-c3ccccc3)cc(-c3ccc(N(c4ccc5c(c4)sc4ccccc45)c4ccc5ccccc5c4-c4ccccc4)cc3)c2)cc1 ; Cc1cccc2oc3cc(N(c4ccc(-c5cc(-c6ccccc6)cc(-c6ccccc6)c5)cc4)c4cc5ccccc5cc4-c4ccccc4)ccc3c12 ; Cc1cccc2sc3cc(N(c4ccc(-c5cc(-c6ccccc6)cc(-c6ccccc6)c5)cc4)c4ccc5ccccc5c4-c4ccccc4)ccc3c12]

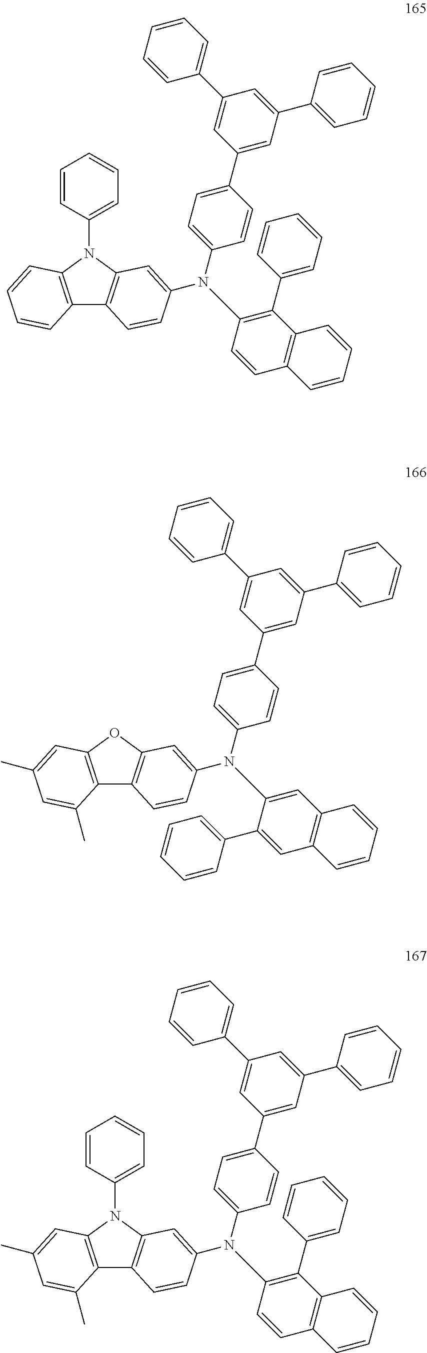 [SMILES: c1ccc(-c2cc(-c3ccccc3)cc(-c3ccc(N(c4ccc5c6ccccc6n(-c6ccccc6)c5c4)c4ccc5ccccc5c4-c4ccccc4)cc3)c2)cc1 ; Cc1cc(C)c2c(c1)oc1cc(N(c3ccc(-c4cc(-c5ccccc5)cc(-c5ccccc5)c4)cc3)c3cc4ccccc4cc3-c3ccccc3)ccc12 ; Cc1cc(C)c2c3ccc(N(c4ccc(-c5cc(-c6ccccc6)cc(-c6ccccc6)c5)cc4)c4ccc5ccccc5c4-c4ccccc4)cc3n(-c3ccccc3)c2c1]

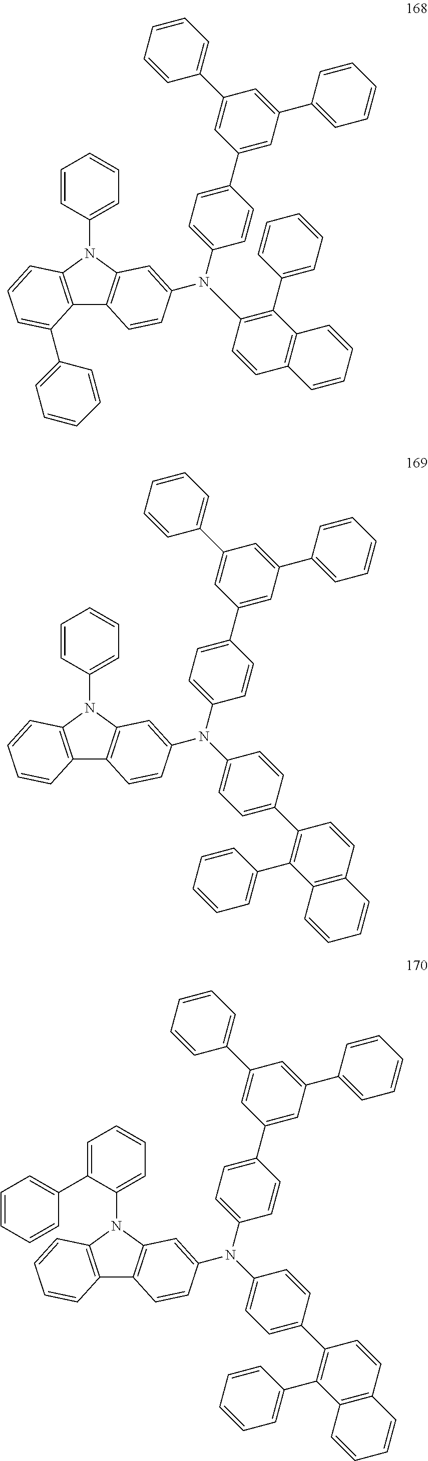 [SMILES: c1ccc(-c2cc(-c3ccccc3)cc(-c3ccc(N(c4ccc5c6c(-c7ccccc7)cccc6n(-c6ccccc6)c5c4)c4ccc5ccccc5c4-c4ccccc4)cc3)c2)cc1 ; c1ccc(-c2cc(-c3ccccc3)cc(-c3ccc(N(c4ccc(-c5ccc6ccccc6c5-c5ccccc5)cc4)c4ccc5c6ccccc6n(-c6ccccc6)c5c4)cc3)c2)cc1 ; c1ccc(-c2cc(-c3ccccc3)cc(-c3ccc(N(c4ccc(-c5ccc6ccccc6c5-c5ccccc5)cc4)c4ccc5c6ccccc6n(-c6ccccc6-c6ccccc6)c5c4)cc3)c2)cc1]

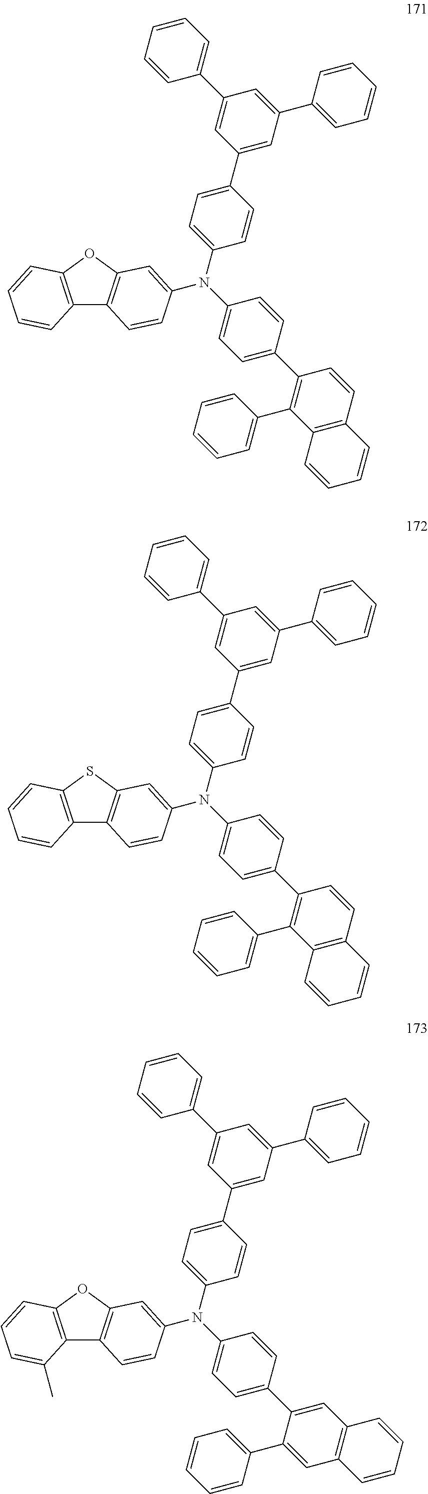 [SMILES: c1ccc(-c2cc(-c3ccccc3)cc(-c3ccc(N(c4ccc(-c5ccc6ccccc6c5-c5ccccc5)cc4)c4ccc5c(c4)oc4ccccc45)cc3)c2)cc1 ; c1ccc(-c2cc(-c3ccccc3)cc(-c3ccc(N(c4ccc(-c5ccc6ccccc6c5-c5ccccc5)cc4)c4ccc5c(c4)sc4ccccc45)cc3)c2)cc1 ; Cc1cccc2oc3cc(N(c4ccc(-c5cc(-c6ccccc6)cc(-c6ccccc6)c5)cc4)c4ccc(-c5cc6ccccc6cc5-c5ccccc5)cc4)ccc3c12]

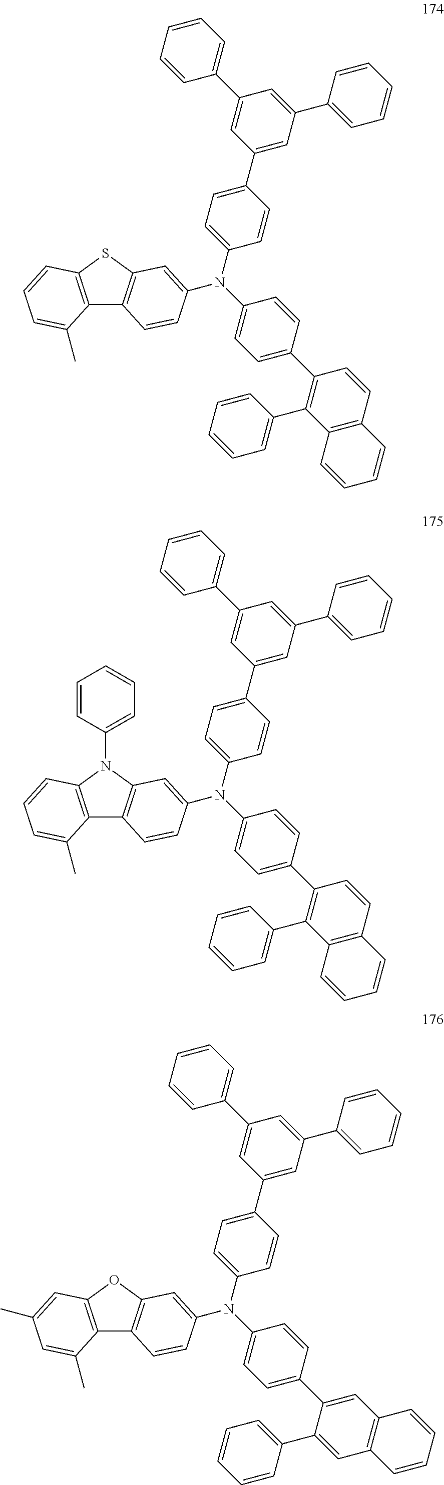 [SMILES: Cc1cccc2sc3cc(N(c4ccc(-c5cc(-c6ccccc6)cc(-c6ccccc6)c5)cc4)c4ccc(-c5ccc6ccccc6c5-c5ccccc5)cc4)ccc3c12 ; Cc1cccc2c1c1ccc(N(c3ccc(-c4cc(-c5ccccc5)cc(-c5ccccc5)c4)cc3)c3ccc(-c4ccc5ccccc5c4-c4ccccc4)cc3)cc1n2-c1ccccc1 ; Cc1cc(C)c2c(c1)oc1cc(N(c3ccc(-c4cc(-c5ccccc5)cc(-c5ccccc5)c4)cc3)c3ccc(-c4cc5ccccc5cc4-c4ccccc4)cc3)ccc12]

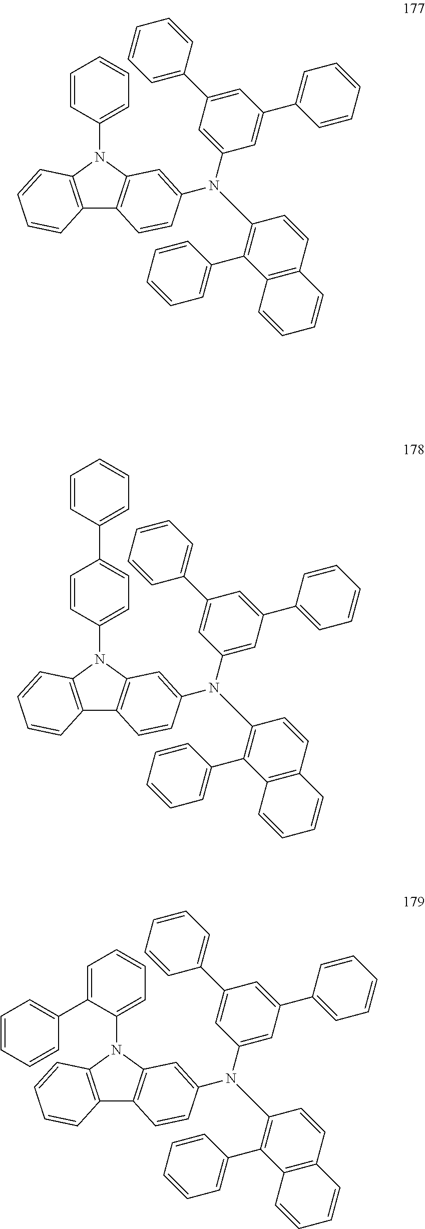 [SMILES: c1ccc(-c2cc(-c3ccccc3)cc(N(c3ccc4c5ccccc5n(-c5ccccc5)c4c3)c3ccc4ccccc4c3-c3ccccc3)c2)cc1 ; c1ccc(-c2ccc(-n3c4ccccc4c4ccc(N(c5cc(-c6ccccc6)cc(-c6ccccc6)c5)c5ccc6ccccc6c5-c5ccccc5)cc43)cc2)cc1 ; c1ccc(-c2cc(-c3ccccc3)cc(N(c3ccc4c5ccccc5n(-c5ccccc5-c5ccccc5)c4c3)c3ccc4ccccc4c3-c3ccccc3)c2)cc1]

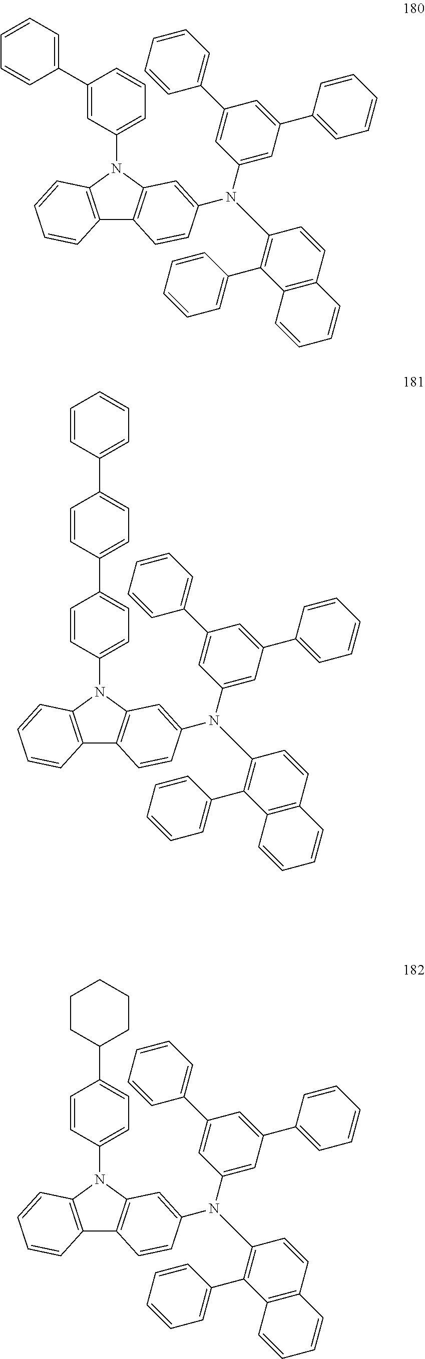 [SMILES: c1ccc(-c2cc(-c3ccccc3)cc(N(c3ccc4c5ccccc5n(-c5cccc(-c6ccccc6)c5)c4c3)c3ccc4ccccc4c3-c3ccccc3)c2)cc1 ; c1ccc(-c2ccc(-c3ccc(-n4c5ccccc5c5ccc(N(c6cc(-c7ccccc7)cc(-c7ccccc7)c6)c6ccc7ccccc7c6-c6ccccc6)cc54)cc3)cc2)cc1 ; c1ccc(-c2cc(-c3ccccc3)cc(N(c3ccc4c5ccccc5n(-c5ccc(C6CCCCC6)cc5)c4c3)c3ccc4ccccc4c3-c3ccccc3)c2)cc1]

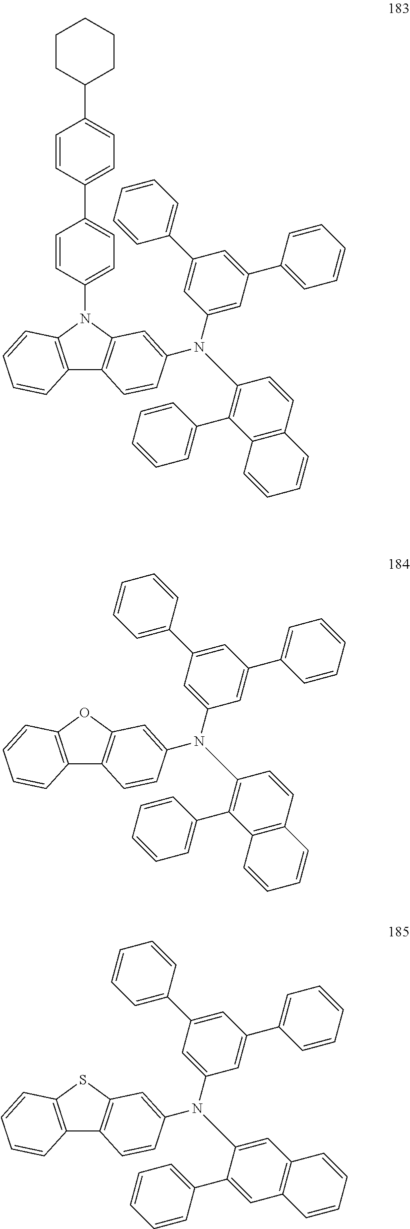 [SMILES: c1ccc(-c2cc(-c3ccccc3)cc(N(c3ccc4c5ccccc5n(-c5ccc(-c6ccc(C7CCCCC7)cc6)cc5)c4c3)c3ccc4ccccc4c3-c3ccccc3)c2)cc1 ; c1ccc(-c2cc(-c3ccccc3)cc(N(c3ccc4c(c3)oc3ccccc34)c3ccc4ccccc4c3-c3ccccc3)c2)cc1 ; c1ccc(-c2cc(-c3ccccc3)cc(N(c3ccc4c(c3)sc3ccccc34)c3cc4ccccc4cc3-c3ccccc3)c2)cc1]

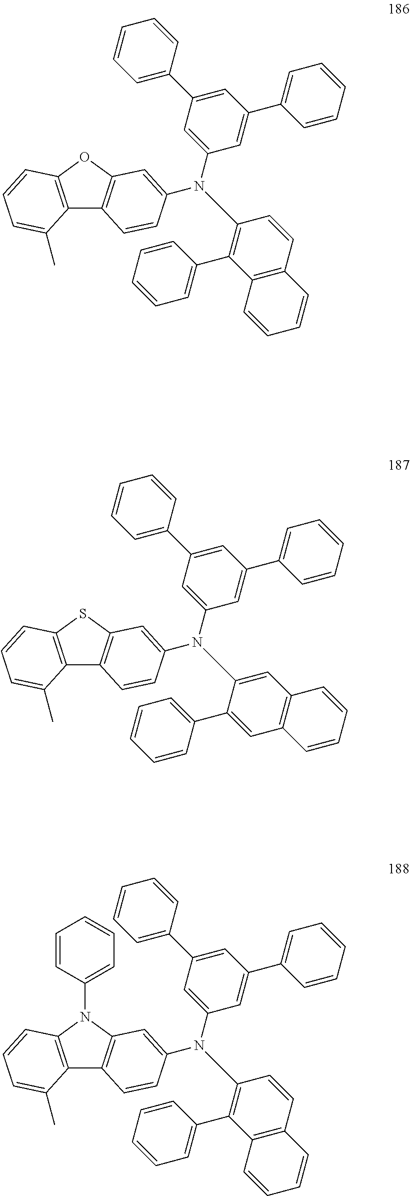 [SMILES: Cc1cccc2oc3cc(N(c4cc(-c5ccccc5)cc(-c5ccccc5)c4)c4ccc5ccccc5c4-c4ccccc4)ccc3c12 ; Cc1cccc2sc3cc(N(c4cc(-c5ccccc5)cc(-c5ccccc5)c4)c4cc5ccccc5cc4-c4ccccc4)ccc3c12 ; Cc1cccc2c1c1ccc(N(c3cc(-c4ccccc4)cc(-c4ccccc4)c3)c3ccc4ccccc4c3-c3ccccc3)cc1n2-c1ccccc1]

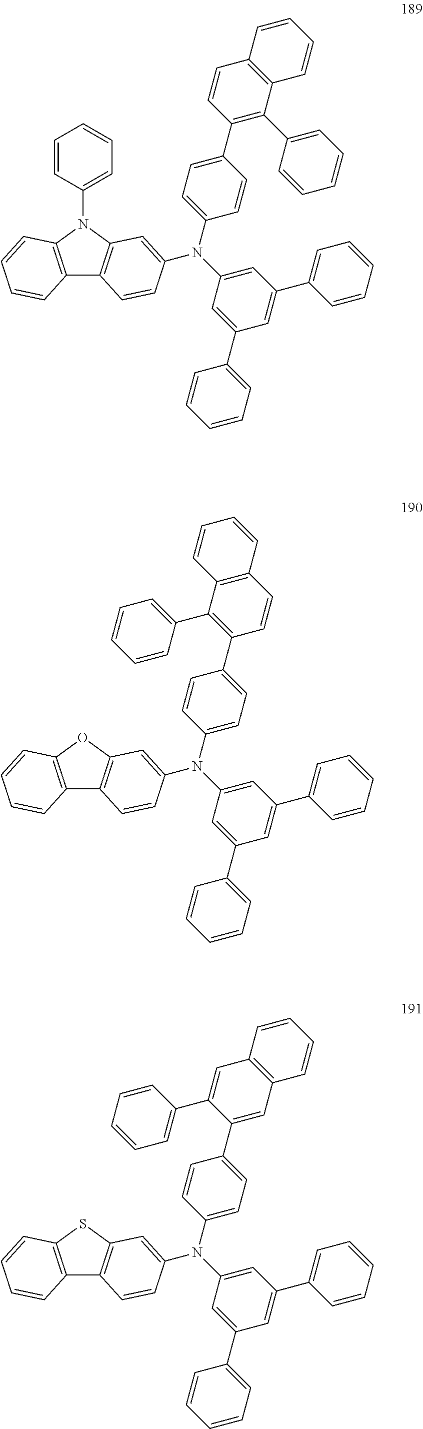 [SMILES: c1ccc(-c2cc(-c3ccccc3)cc(N(c3ccc(-c4ccc5ccccc5c4-c4ccccc4)cc3)c3ccc4c5ccccc5n(-c5ccccc5)c4c3)c2)cc1 ; c1ccc(-c2cc(-c3ccccc3)cc(N(c3ccc(-c4ccc5ccccc5c4-c4ccccc4)cc3)c3ccc4c(c3)oc3ccccc34)c2)cc1 ; c1ccc(-c2cc(-c3ccccc3)cc(N(c3ccc(-c4cc5ccccc5cc4-c4ccccc4)cc3)c3ccc4c(c3)sc3ccccc34)c2)cc1]

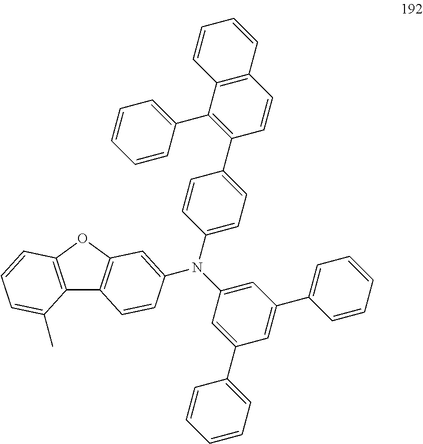 [SMILES: Cc1cccc2oc3cc(N(c4ccc(-c5ccc6ccccc6c5-c5ccccc5)cc4)c4cc(-c5ccccc5)cc(-c5ccccc5)c4)ccc3c12]

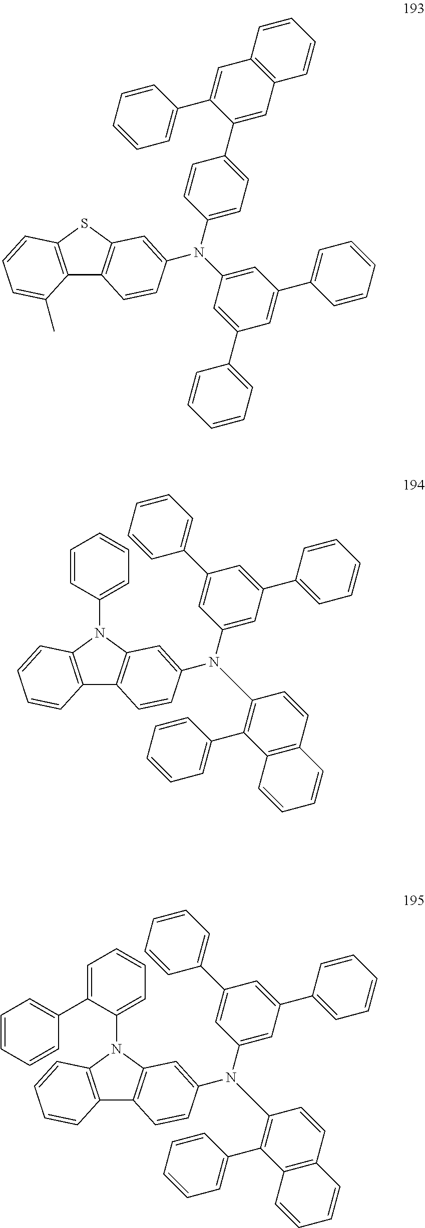 [SMILES: Cc1cccc2sc3cc(N(c4ccc(-c5cc6ccccc6cc5-c5ccccc5)cc4)c4cc(-c5ccccc5)cc(-c5ccccc5)c4)ccc3c12 ; c1ccc(-c2cc(-c3ccccc3)cc(N(c3ccc4c5ccccc5n(-c5ccccc5)c4c3)c3ccc4ccccc4c3-c3ccccc3)c2)cc1 ; c1ccc(-c2cc(-c3ccccc3)cc(N(c3ccc4c5ccccc5n(-c5ccccc5-c5ccccc5)c4c3)c3ccc4ccccc4c3-c3ccccc3)c2)cc1]

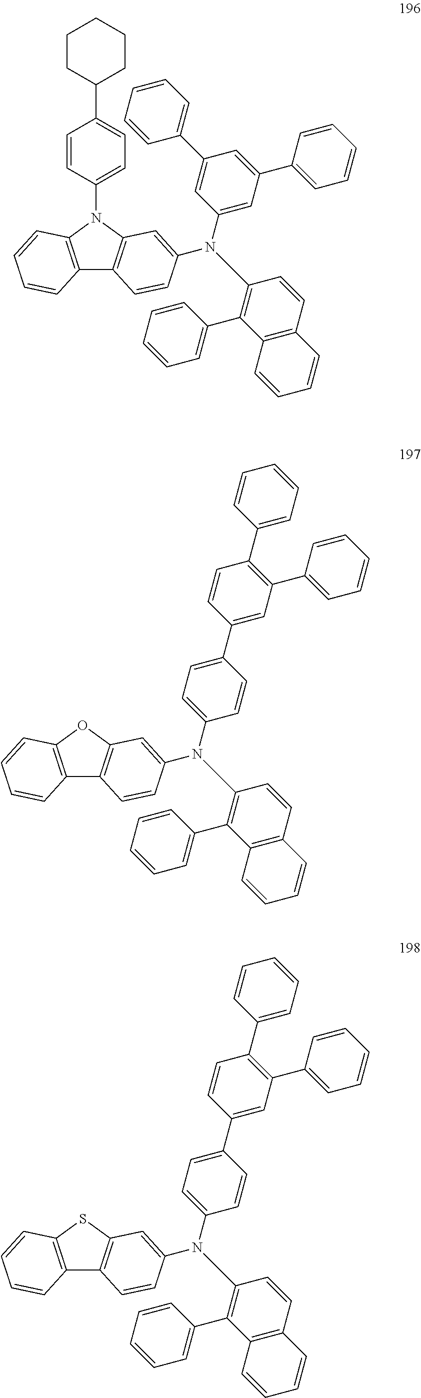 [SMILES: c1ccc(-c2cc(-c3ccccc3)cc(N(c3ccc4c5ccccc5n(-c5ccc(C6CCCCC6)cc5)c4c3)c3ccc4ccccc4c3-c3ccccc3)c2)cc1 ; c1ccc(-c2ccc(-c3ccc(N(c4ccc5c(c4)oc4ccccc45)c4ccc5ccccc5c4-c4ccccc4)cc3)cc2-c2ccccc2)cc1 ; c1ccc(-c2ccc(-c3ccc(N(c4ccc5c(c4)sc4ccccc45)c4ccc5ccccc5c4-c4ccccc4)cc3)cc2-c2ccccc2)cc1]

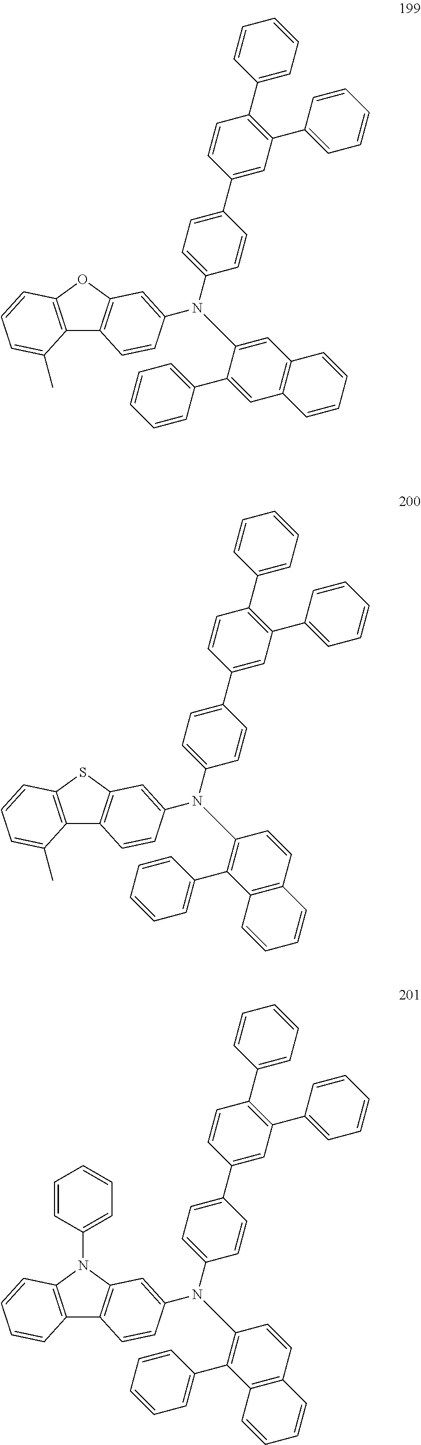 [SMILES: Cc1cccc2oc3cc(N(c4ccc(-c5ccc(-c6ccccc6)c(-c6ccccc6)c5)cc4)c4cc5ccccc5cc4-c4ccccc4)ccc3c12 ; Cc1cccc2sc3cc(N(c4ccc(-c5ccc(-c6ccccc6)c(-c6ccccc6)c5)cc4)c4ccc5ccccc5c4-c4ccccc4)ccc3c12 ; c1ccc(-c2ccc(-c3ccc(N(c4ccc5c6ccccc6n(-c6ccccc6)c5c4)c4ccc5ccccc5c4-c4ccccc4)cc3)cc2-c2ccccc2)cc1]

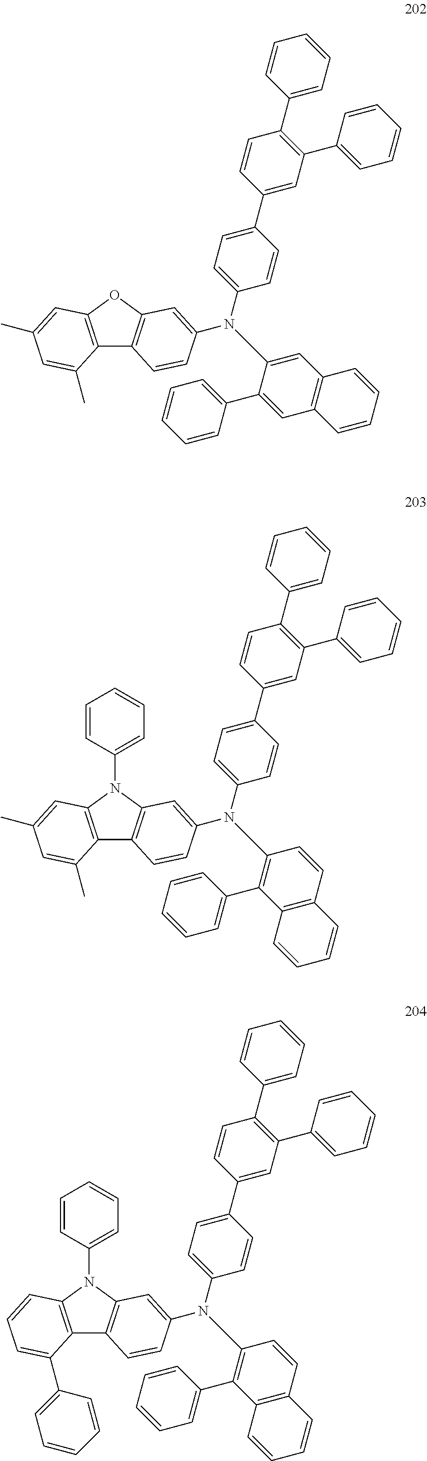 [SMILES: Cc1cc(C)c2c(c1)oc1cc(N(c3ccc(-c4ccc(-c5ccccc5)c(-c5ccccc5)c4)cc3)c3cc4ccccc4cc3-c3ccccc3)ccc12 ; Cc1cc(C)c2c3ccc(N(c4ccc(-c5ccc(-c6ccccc6)c(-c6ccccc6)c5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)cc2-c2ccccc2)cc1]

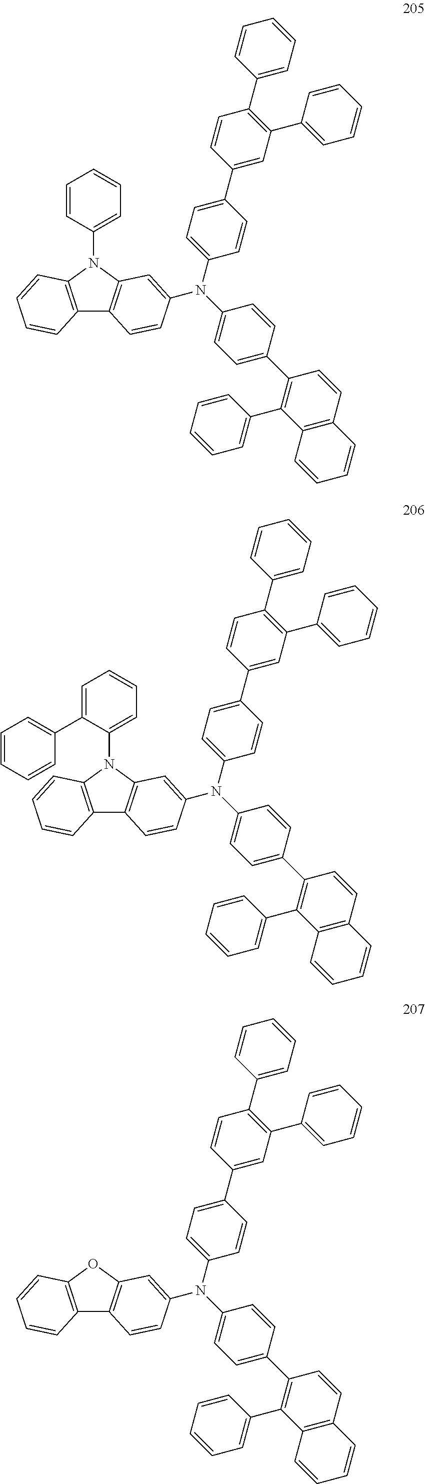 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)cc2-c2ccccc2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)cc2-c2ccccc2)cc1 ; c1ccc(-c2ccc(-c3ccc(N(c4ccc(-c5ccc6ccccc6c5-c5ccccc5)cc4)c4ccc5c(c4)oc4ccccc45)cc3)cc2-c2ccccc2)cc1]

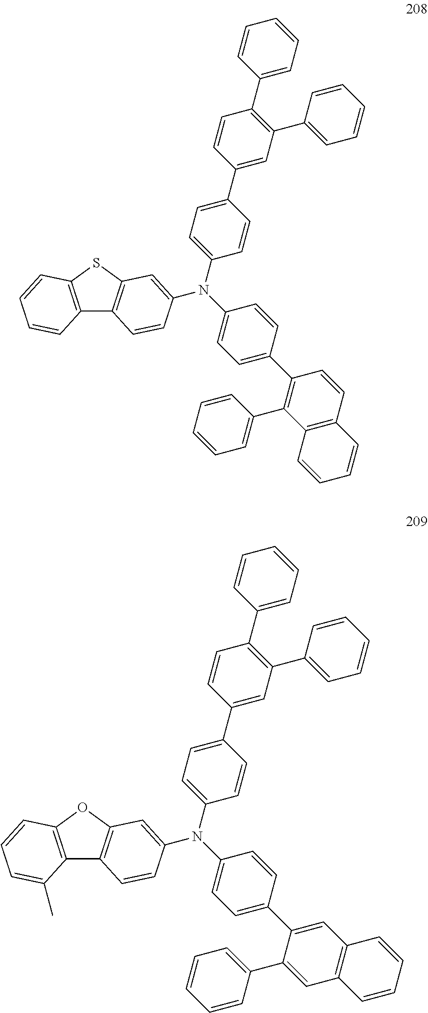 [SMILES: c1ccc(-c2ccc(-c3ccc(N(c4ccc(-c5ccc6ccccc6c5-c5ccccc5)cc4)c4ccc5c(c4)sc4ccccc45)cc3)cc2-c2ccccc2)cc1 ; Cc1cccc2oc3cc(N(c4ccc(-c5ccc(-c6ccccc6)c(-c6ccccc6)c5)cc4)c4ccc(-c5cc6ccccc6cc5-c5ccccc5)cc4)ccc3c12]

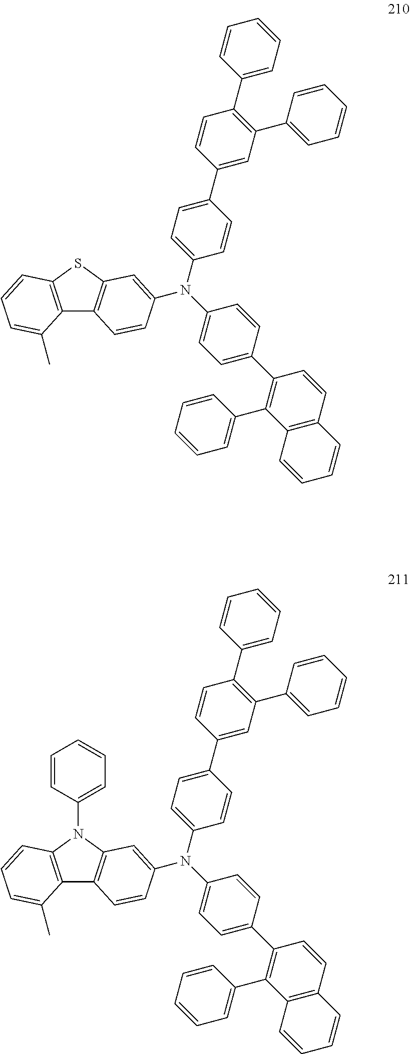 [SMILES: Cc1cccc2sc3cc(N(c4ccc(-c5ccc(-c6ccccc6)c(-c6ccccc6)c5)cc4)c4ccc(-c5ccc6ccccc6c5-c5ccccc5)cc4)ccc3c12 ; Cc1cccc2c1c1ccc(N(c3ccc(-c4ccc(-c5ccccc5)c(-c5ccccc5)c4)cc3)c3ccc(-c4ccc5ccccc5c4-c4ccccc4)cc3)cc1n2-c1ccccc1]

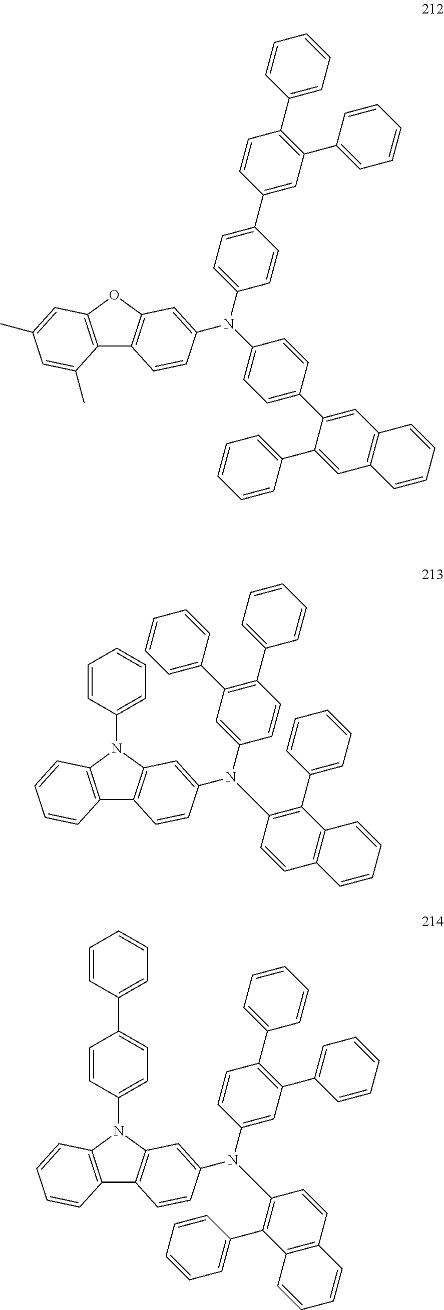 [SMILES: Cc1cc(C)c2c(c1)oc1cc(N(c3ccc(-c4ccc(-c5ccccc5)c(-c5ccccc5)c4)cc3)c3ccc(-c4cc5ccccc5cc4-c4ccccc4)cc3)ccc12 ; c1ccc(-c2ccc(N(c3ccc4c5ccccc5n(-c5ccccc5)c4c3)c3ccc4ccccc4c3-c3ccccc3)cc2-c2ccccc2)cc1 ; c1ccc(-c2ccc(-n3c4ccccc4c4ccc(N(c5ccc(-c6ccccc6)c(-c6ccccc6)c5)c5ccc6ccccc6c5-c5ccccc5)cc43)cc2)cc1]

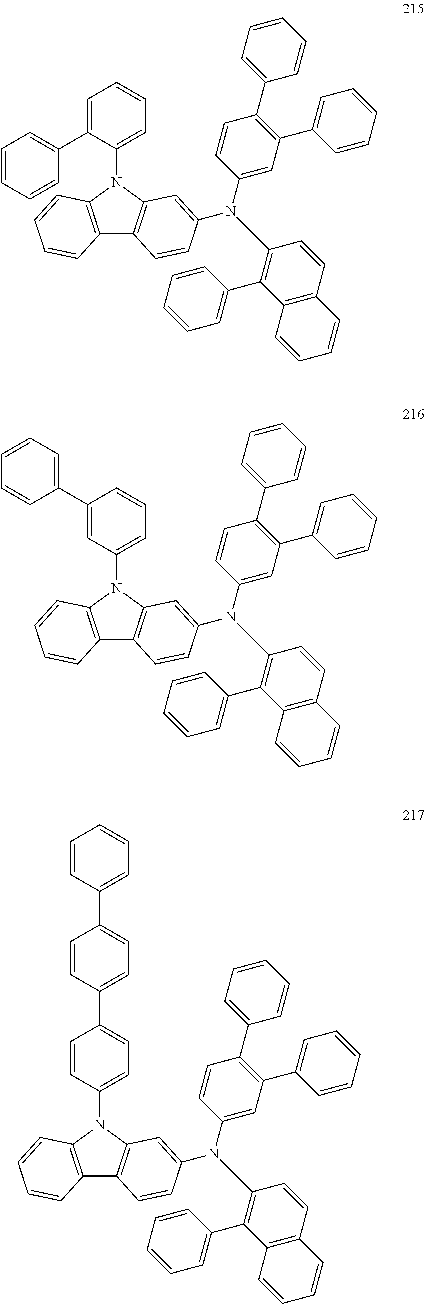 [SMILES: c1ccc(-c2ccc(N(c3ccc4c5ccccc5n(-c5ccccc5-c5ccccc5)c4c3)c3ccc4ccccc4c3-c3ccccc3)cc2-c2ccccc2)cc1 ; c1ccc(-c2cccc(-n3c4ccccc4c4ccc(N(c5ccc(-c6ccccc6)c(-c6ccccc6)c5)c5ccc6ccccc6c5-c5ccccc5)cc43)c2)cc1 ; c1ccc(-c2ccc(-c3ccc(-n4c5ccccc5c5ccc(N(c6ccc(-c7ccccc7)c(-c7ccccc7)c6)c6ccc7ccccc7c6-c6ccccc6)cc54)cc3)cc2)cc1]

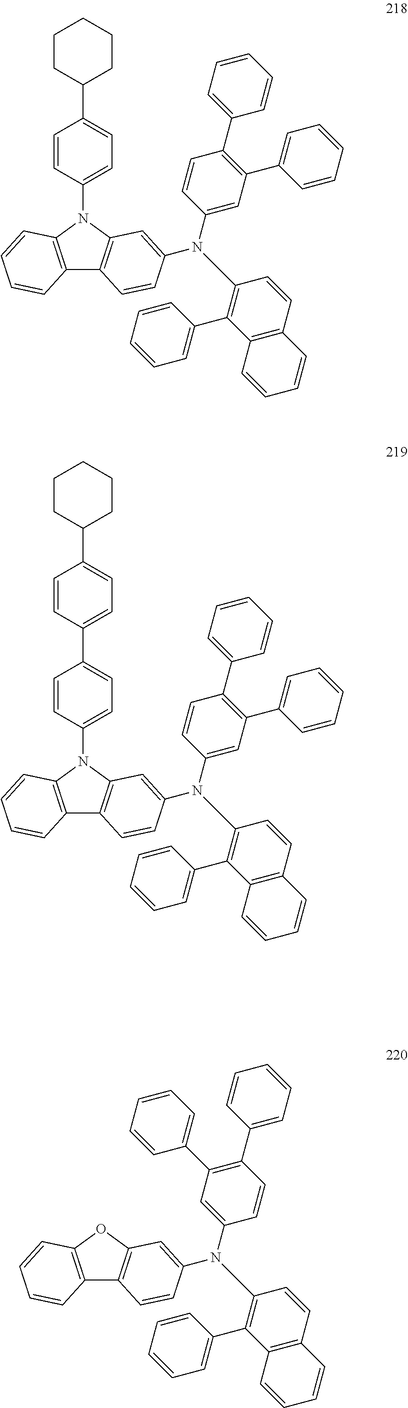 [SMILES: c1ccc(-c2ccc(N(c3ccc4c5ccccc5n(-c5ccc(C6CCCCC6)cc5)c4c3)c3ccc4ccccc4c3-c3ccccc3)cc2-c2ccccc2)cc1 ; c1ccc(-c2ccc(N(c3ccc4c5ccccc5n(-c5ccc(-c6ccc(C7CCCCC7)cc6)cc5)c4c3)c3ccc4ccccc4c3-c3ccccc3)cc2-c2ccccc2)cc1 ; c1ccc(-c2ccc(N(c3ccc4c(c3)oc3ccccc34)c3ccc4ccccc4c3-c3ccccc3)cc2-c2ccccc2)cc1]

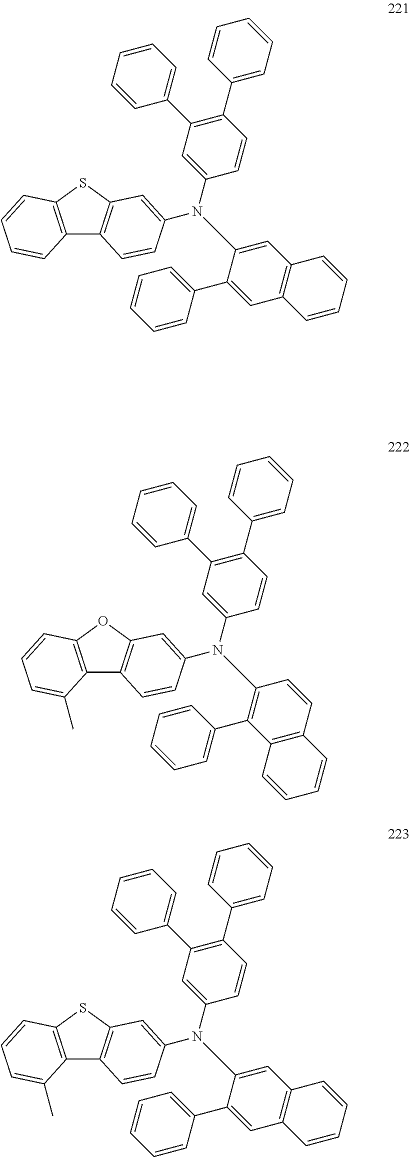 [SMILES: c1ccc(-c2ccc(N(c3ccc4c(c3)sc3ccccc34)c3cc4ccccc4cc3-c3ccccc3)cc2-c2ccccc2)cc1 ; Cc1cccc2oc3cc(N(c4ccc(-c5ccccc5)c(-c5ccccc5)c4)c4ccc5ccccc5c4-c4ccccc4)ccc3c12 ; Cc1cccc2sc3cc(N(c4ccc(-c5ccccc5)c(-c5ccccc5)c4)c4cc5ccccc5cc4-c4ccccc4)ccc3c12]

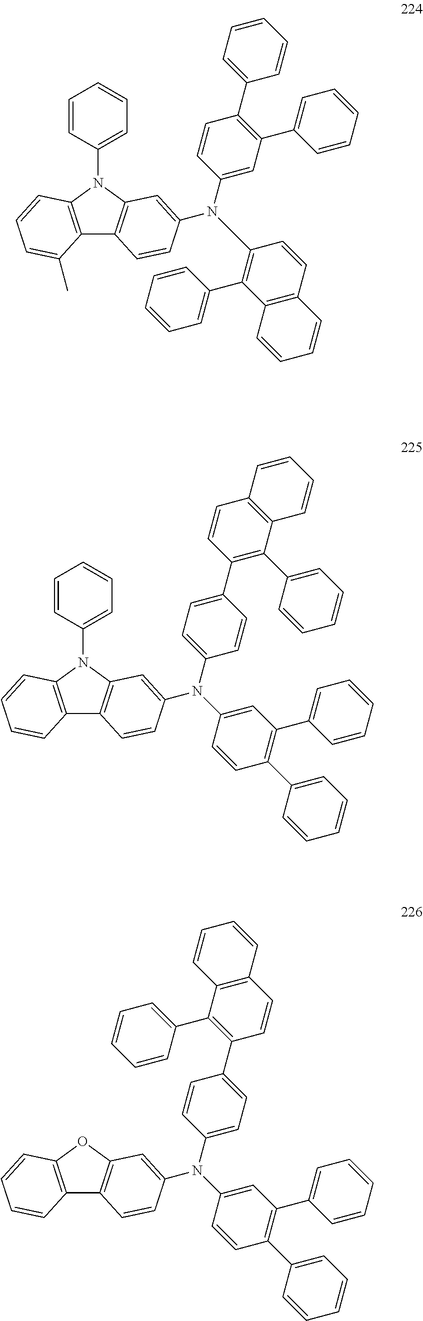 [SMILES: Cc1cccc2c1c1ccc(N(c3ccc(-c4ccccc4)c(-c4ccccc4)c3)c3ccc4ccccc4c3-c3ccccc3)cc1n2-c1ccccc1 ; c1ccc(-c2ccc(N(c3ccc(-c4ccc5ccccc5c4-c4ccccc4)cc3)c3ccc4c5ccccc5n(-c5ccccc5)c4c3)cc2-c2ccccc2)cc1 ; c1ccc(-c2ccc(N(c3ccc(-c4ccc5ccccc5c4-c4ccccc4)cc3)c3ccc4c(c3)oc3ccccc34)cc2-c2ccccc2)cc1]

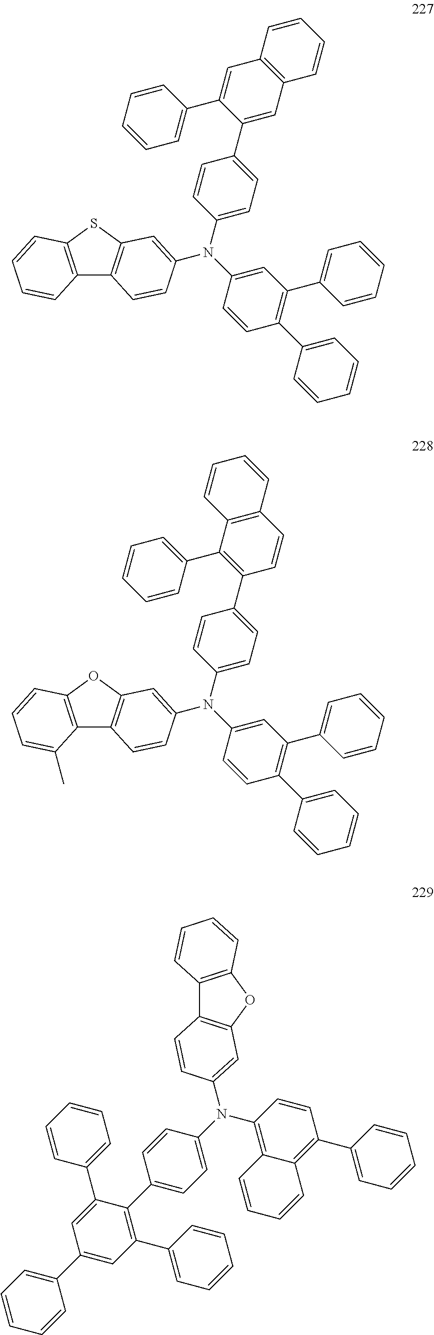 [SMILES: c1ccc(-c2ccc(N(c3ccc(-c4cc5ccccc5cc4-c4ccccc4)cc3)c3ccc4c(c3)sc3ccccc34)cc2-c2ccccc2)cc1 ; Cc1cccc2oc3cc(N(c4ccc(-c5ccc6ccccc6c5-c5ccccc5)cc4)c4ccc(-c5ccccc5)c(-c5ccccc5)c4)ccc3c12 ; c1ccc(-c2cc(-c3ccccc3)c(-c3ccc(N(c4ccc5c(c4)oc4ccccc45)c4ccc(-c5ccccc5)c5ccccc45)cc3)c(-c3ccccc3)c2)cc1]

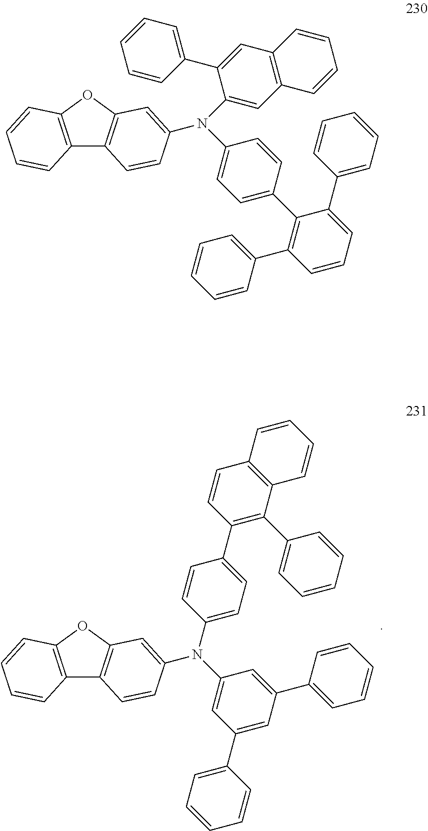 [SMILES: c1ccc(-c2cc3ccccc3cc2N(c2ccc(-c3c(-c4ccccc4)cccc3-c3ccccc3)cc2)c2ccc3c(c2)oc2ccccc23)cc1 ; c1ccc(-c2cc(-c3ccccc3)cc(N(c3ccc(-c4ccc5ccccc5c4-c4ccccc4)cc3)c3ccc4c(c3)oc3ccccc34)c2)cc1]

The amine-containing compound represented by Formula 1 may have a core structure in which “a naphthyl group of which Ar₂ is substituted,” “a phenyl group of which Ar₃₁ and Ar₃₂ are substituted,” and “a carbazole group, dibenzofuran, or dibenzothiophene” are bonded to amine. Ar₂, Ar₃₁, and Ar₃₂ may be an aryl group or a heteroaryl group. As the amine-containing compound represented by Formula 1 includes such a tricyclic group, polaron may be stabilized, which leads to a long lifespan of a light-emitting device. The term “polaron” as utilized herein is a quasiparticle that may be included in interactions between electrons and atoms in a solid material. In one or more embodiments, an increased hopping rate of charges may improve the charge mobility. Moreover, by (e.g., essentially) introducing a substituent into a naphthyl group, color coordinates may be improved to emit deep blue light, and the structural stability of the compound represented by Formula 1 may be secured. Accordingly, the amine-containing compound represented by Formula 1 may have relatively high electrical stability, high charge transport ability, and high glass transition temperature.

Also, by varying the substitution positions, and/or the like, Ar₂, Ar₃₁, and Ar₃₂, the value of a highest occupied molecular orbital (HOMO) energy level may also be suitably adjusted or changed. Accordingly, a hole injection barrier of the anode to the hole transport layer may be suitably adjusted or changed, and an appropriate or suitable value of the energy levels may be maintained between the hole transport layer and the emission layer to increase exciton generation efficiency inside the emission layer.

Therefore, the amine-containing compound represented by Formula 1 may have excellent or suitable hole transport characteristics, and the light-emitting device including the amine-containing compound represented by Formula 1 may have a relatively low driving voltage, high luminescence efficiency and long lifespan.

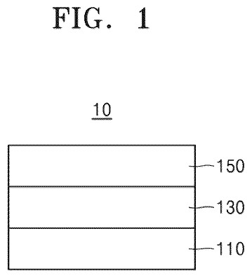

### Description of FIG. 1

FIG. 1 is a schematic cross-sectional view of a light-emitting device 10 according to one or more embodiments. The light-emitting device 10 includes a first electrode 110, an interlayer 130, and a second electrode 150.

Hereinafter, the structure of the light-emitting device 10 according to one or more embodiments and a method of manufacturing the light-emitting device 10 will be described with reference to FIG. 1.

### First Electrode 110

In FIG. 1, a substrate may be additionally located under the first electrode 110 or on the second electrode 150. As the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming (or providing) the first electrode 110 on the substrate. When the first electrode 110 is an anode, a material for forming (or providing) the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming (or providing) the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming (or providing) the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be located on the first electrode 110. The interlayer 130 may include an emission layer.

The interlayer 130 may further include a hole transport region located between the first electrode 110 and the emission layer, and an electron transport region located between the emission layer and the second electrode 150.

The interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer located between the two or more emitting units. When the interlayer 130 includes emitting units and a charge generation layer as described herein, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer including a plurality of different materials, or iii) a multi-layered structure including a plurality of layers including different materials.

The hole transport region may include a hole injection layer, a hole transport layer, an emission auxiliary layer, an electron-blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the layers of each structure being stacked sequentially from the first electrode 110.

The hole transport region may include a compound represented by Formula 201, a compound represented by Formula 202, or any combination thereof:

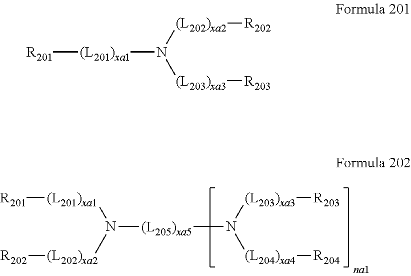

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form (or provide) a C₈-C₆₀ polycyclic group (for example, a carbazole group and/or the like) unsubstituted or substituted with at least one R₁₀ₐ (for example, Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form (or provide) a C₈-C₆₀ polycyclic group unsubstituted or substituted with at least one R₁₀ₐ, and
na1 may be an integer from 1 to 4.

For example, each of Formulae 201 and 202 may include at least one of (e.g., selected from among) groups represented by Formulae CY201 to CY217:

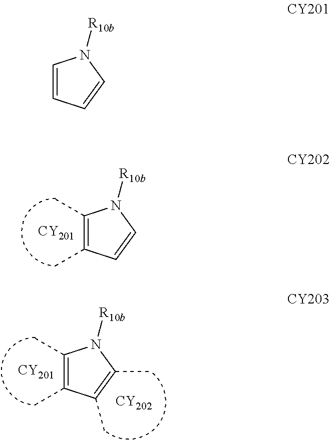

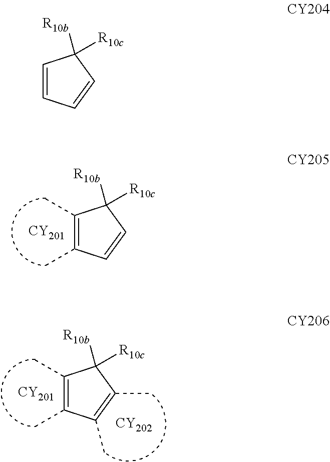

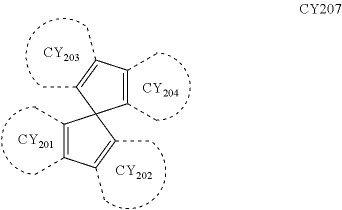

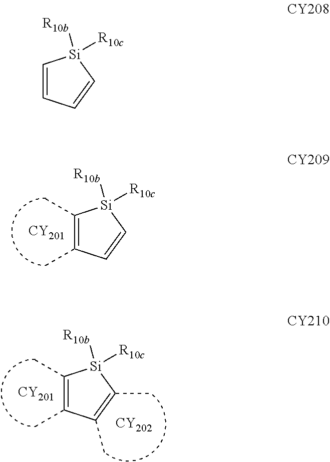

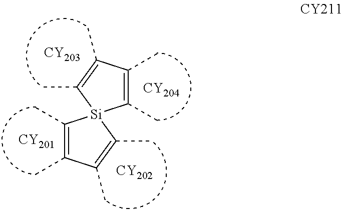

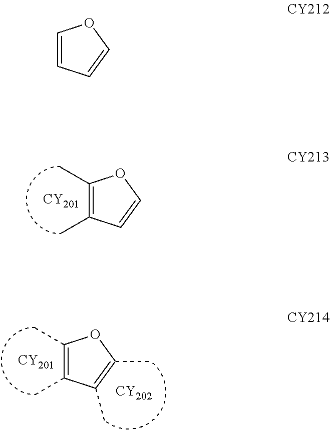

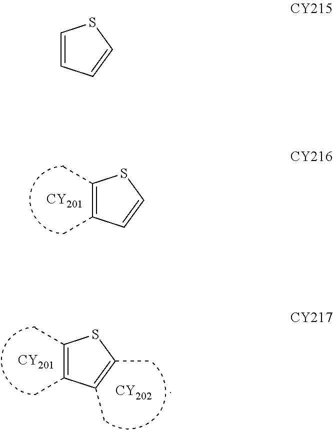

In Formulae CY201CY27 R_{10b} and R_{10c} may each be as described with Formulae CY201 to CY217 may be unsubstituted or substituted with R₁₀ₐ as described herein.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of (e.g., selected from among) groups represented by Formulae CY201 to CY203.

In one or more embodiments, Formula 201 may include at least one of (e.g., selected from among) the groups represented by Formulae CY201 to CY203 and at least one of (e.g., selected from among) the groups represented by Formulae CY204 to CY217.

In one or more embodiments, in Formula 201, xa1 may be 1, R₂₀₁ may be a group represented by one of (e.g., selected from among) Formulae CY201 to CY203, xa2 may be 0, and R₂₀₂ may be a group represented by one of (e.g., selected from among) Formulae CY204 to CY207.

In one or more embodiments, each of Formulae 201 and 202 may not include (e.g., may exclude) a group represented by one (e.g., any one) selected from among Formulae CY201 to CY203.

In one or more embodiments, each of Formulae 201 and 202 may not include (e.g., may exclude) a group represented by one (e.g., any one) selected from among Formulae CY201 to CY203, and may include at least one of (e.g., selected from among) the groups represented by Formulae CY204 to CY217.

In one or more embodiments, each of Formulae 201 and 202 may not include (e.g., may exclude) a group represented by one (e.g., any one) selected from among Formulae CY201 to CY217.

In one or more embodiments, the hole transport region may include at least one (e.g., one or more) selected from among Compounds HT1 to HT46, m-MTDATA, TDATA, 2-TNATA, NPB(NPD), β-NPB, TPD, Spiro-TPD, Spiro-NPB, methylated NPB, TAPC, HMTPD, CzSi, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

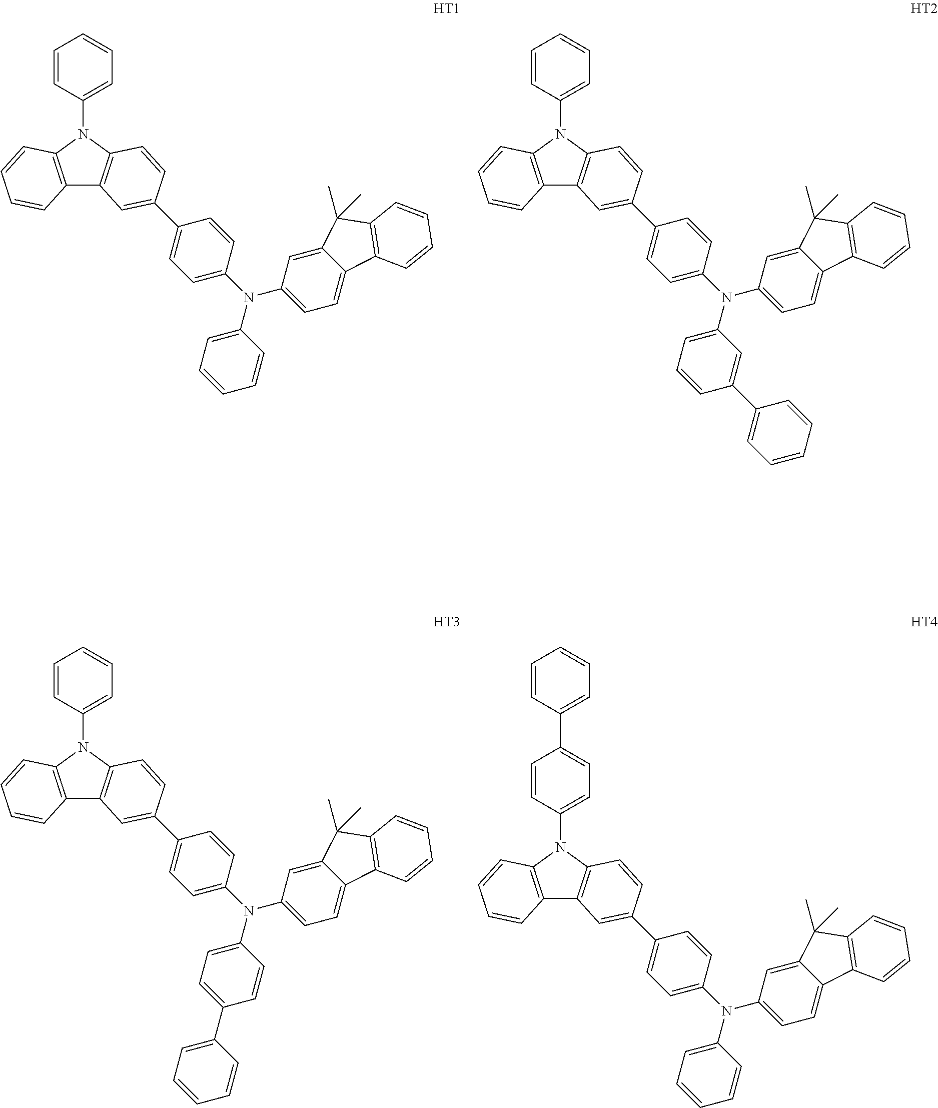 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

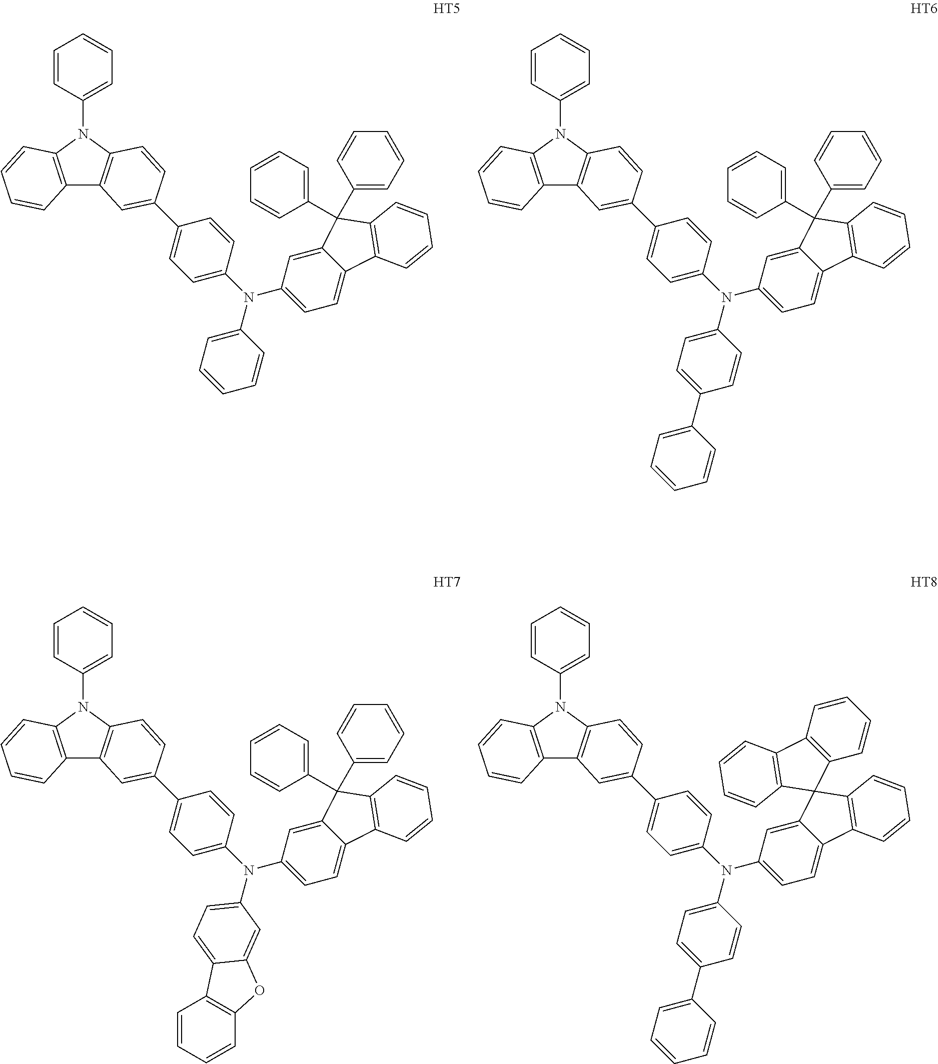 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

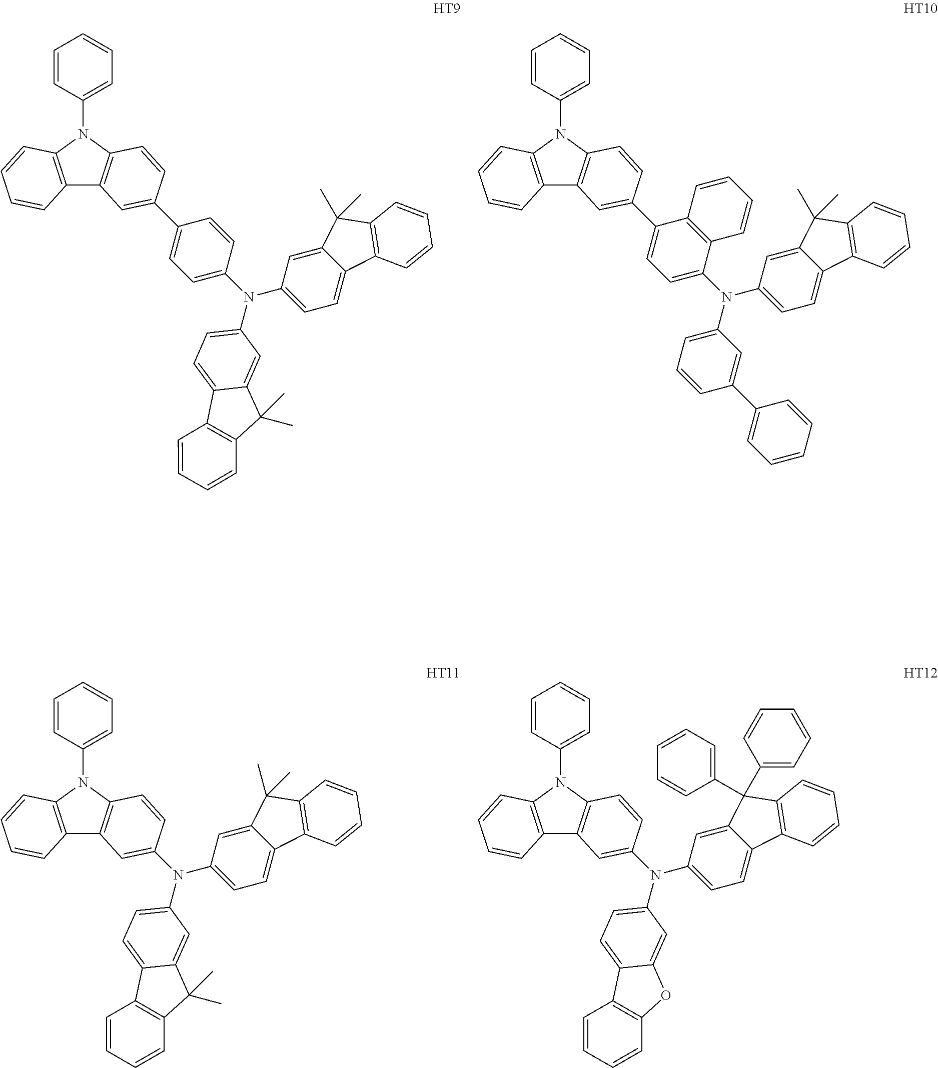 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

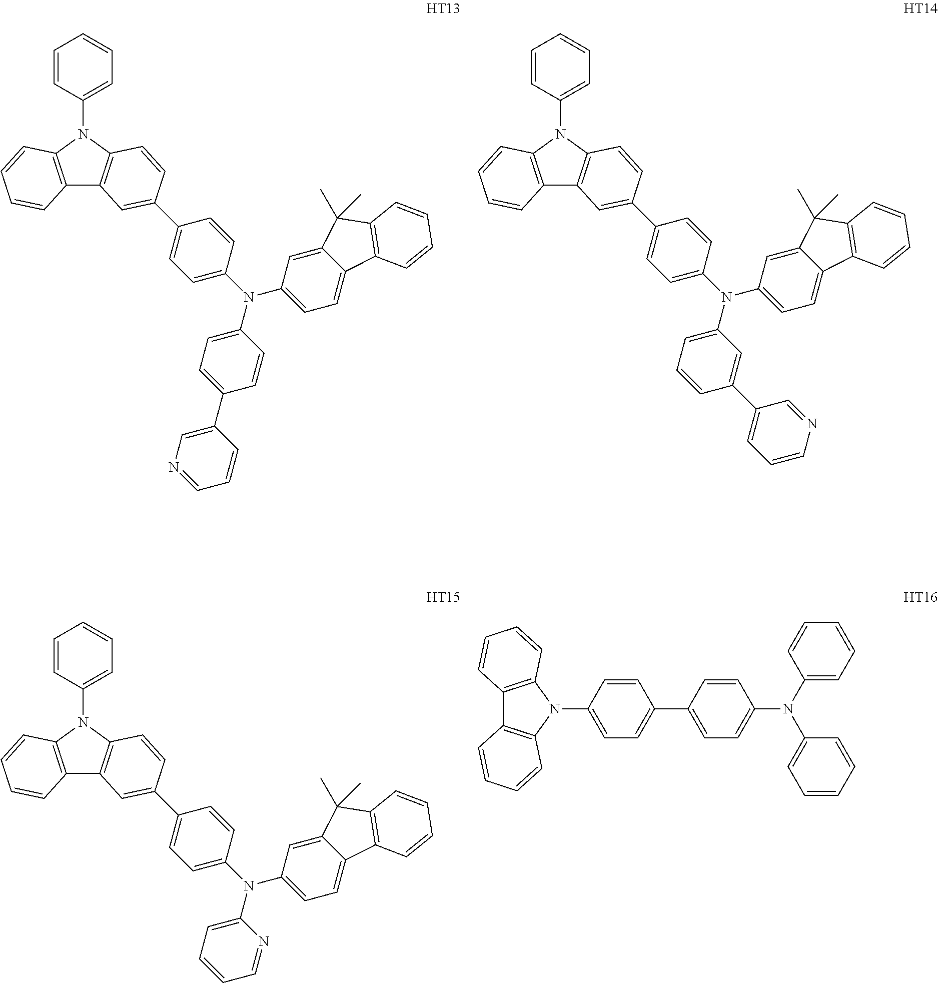 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

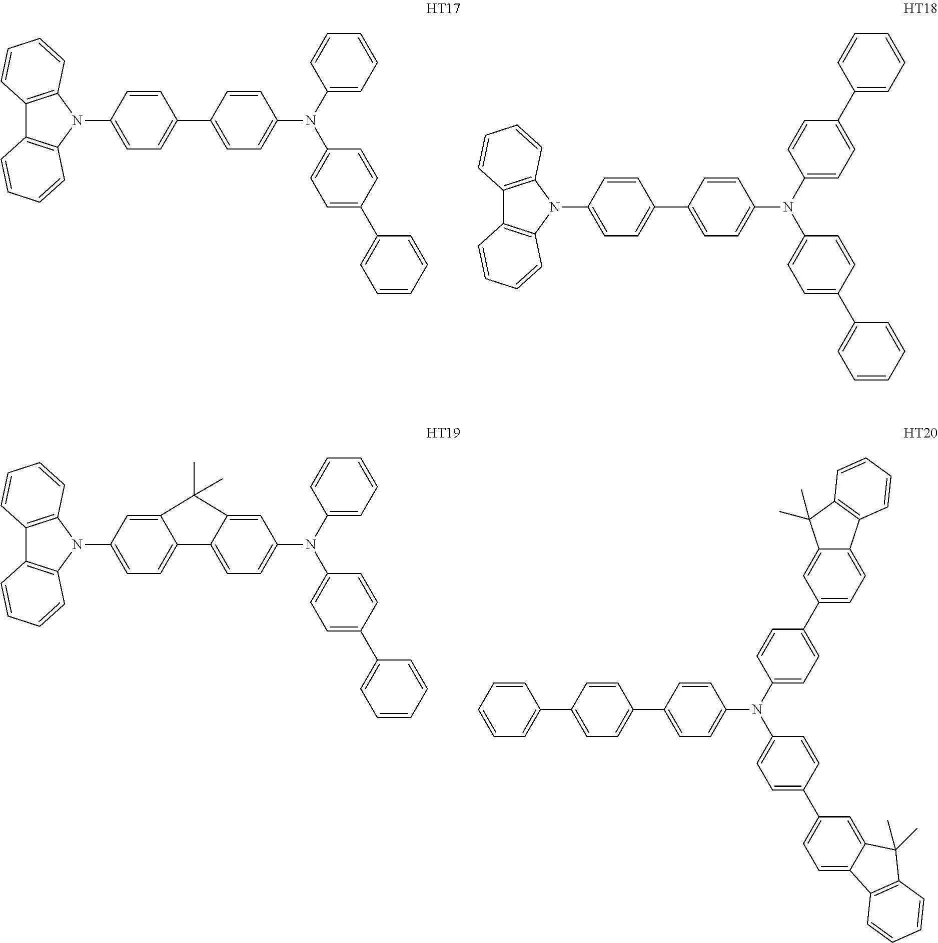 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

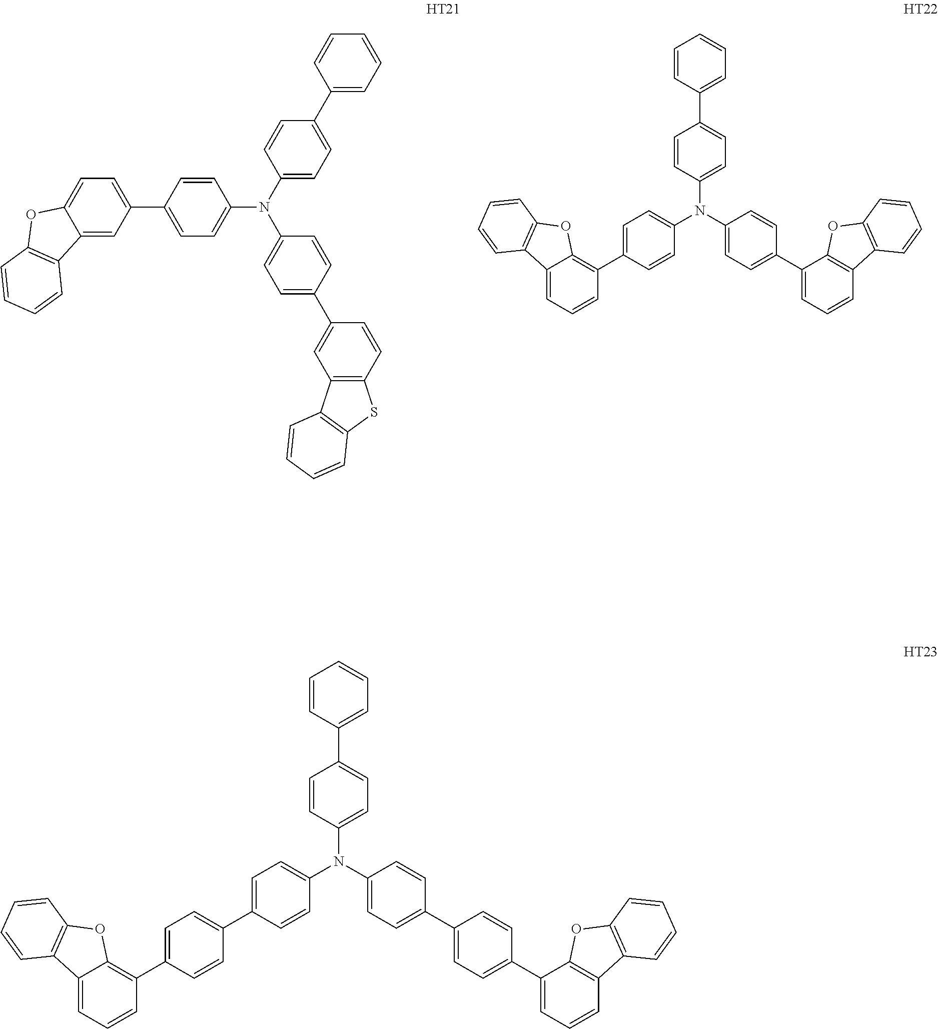 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

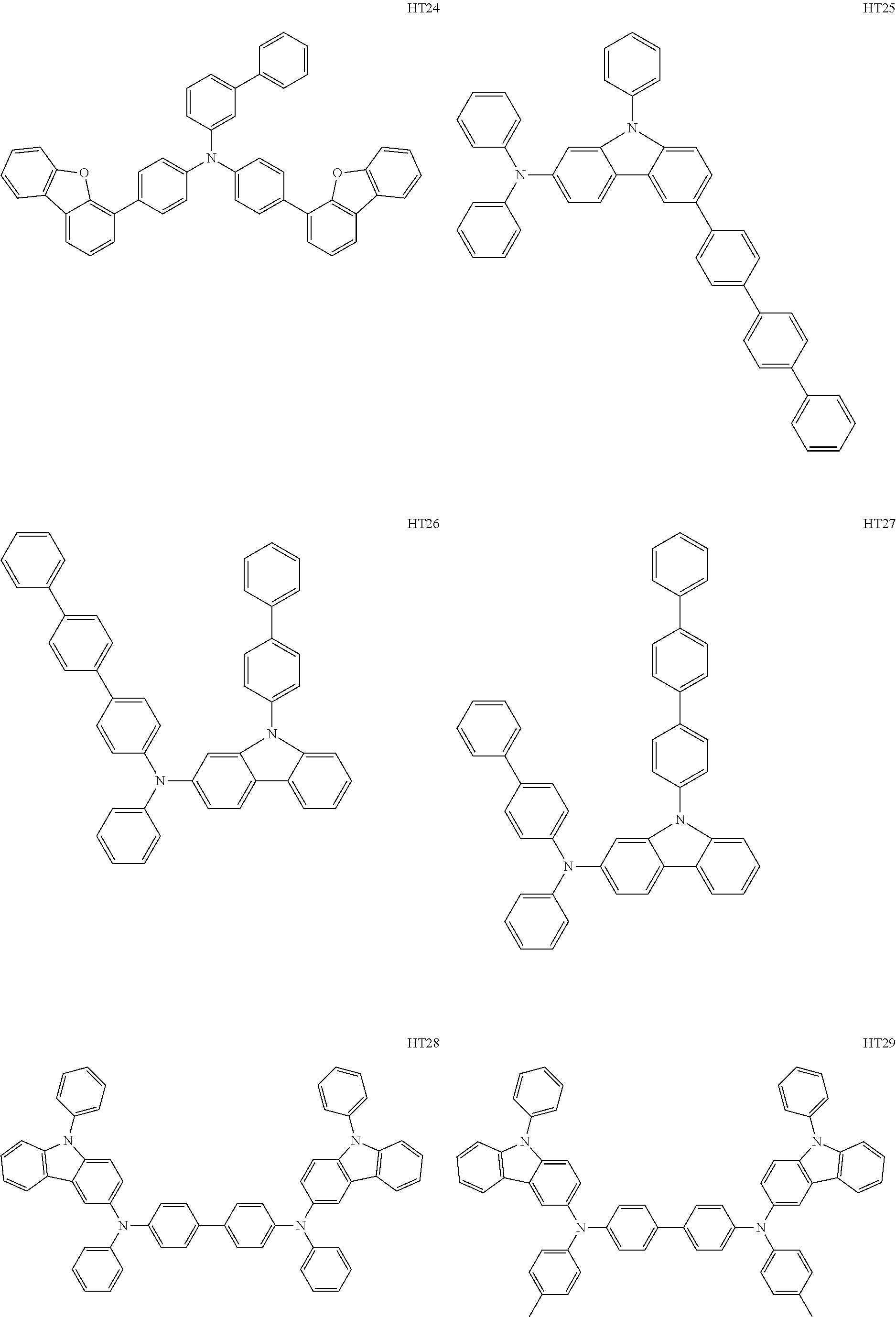

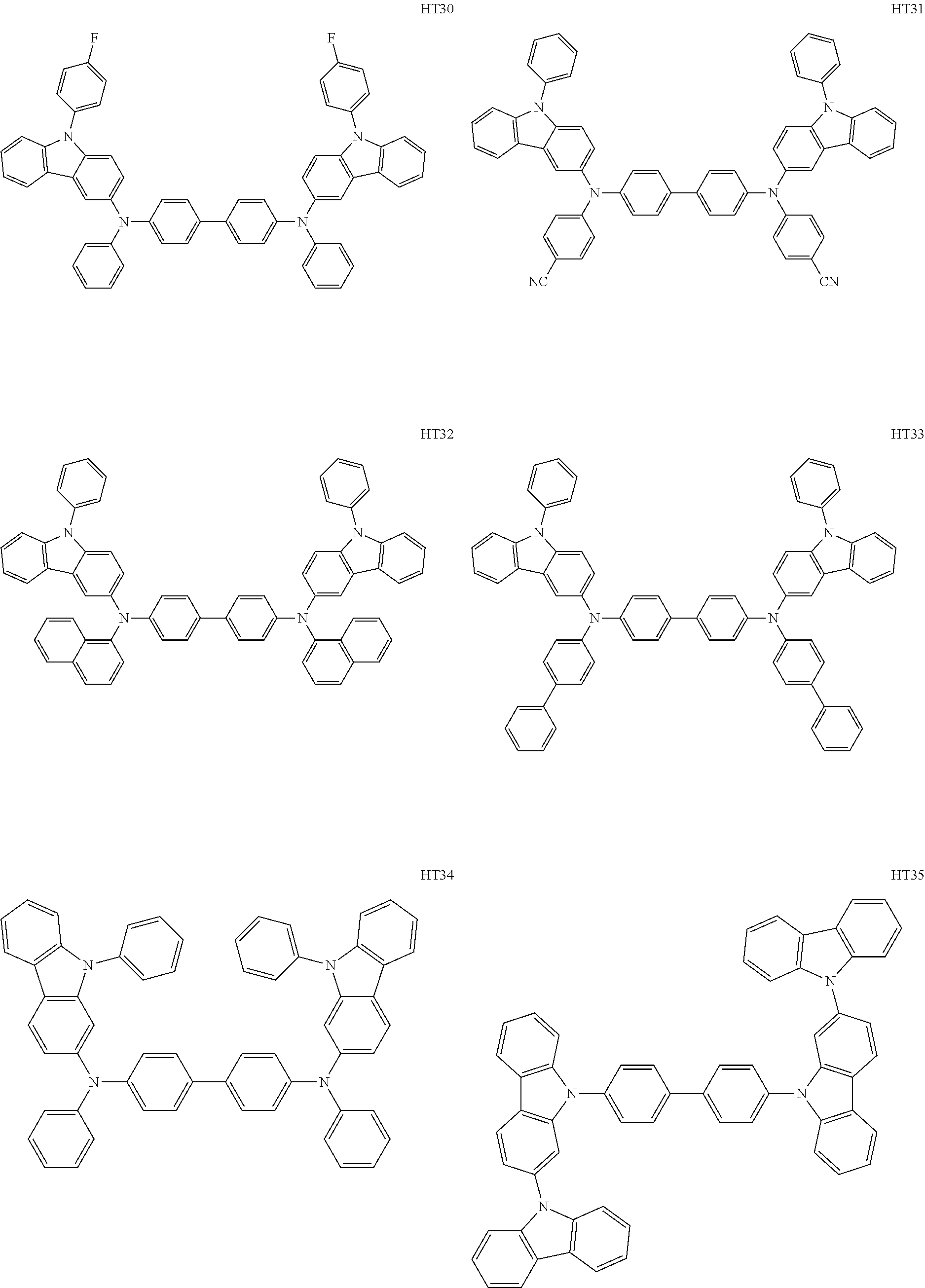

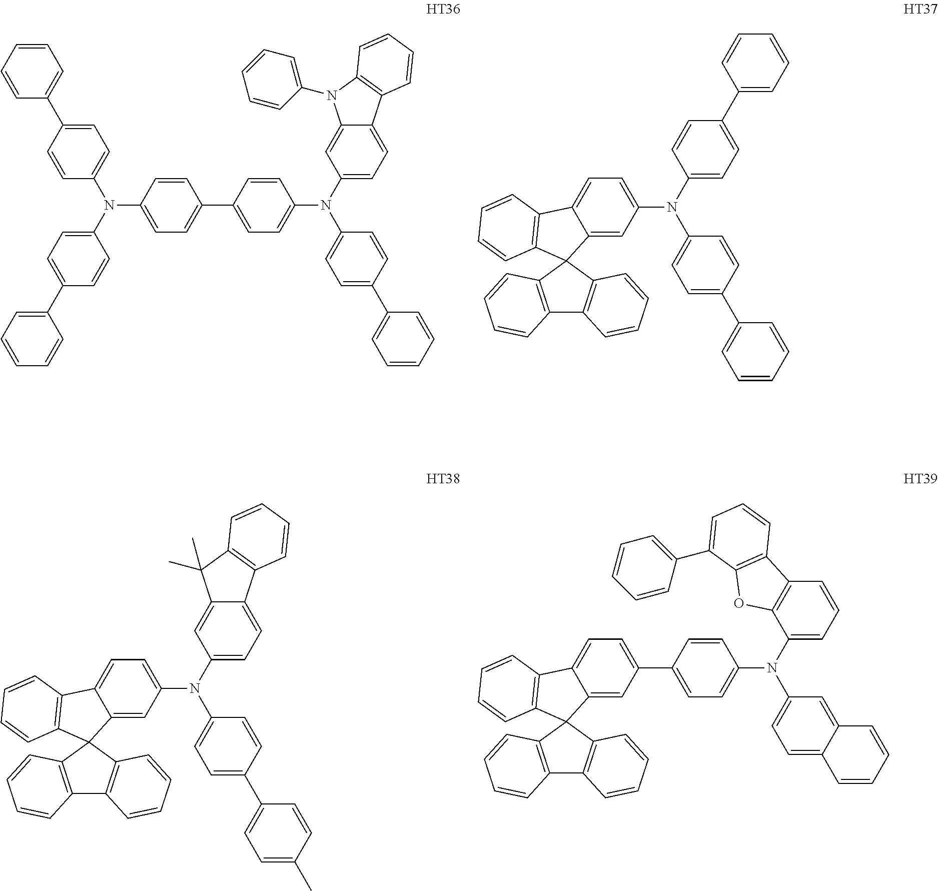 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

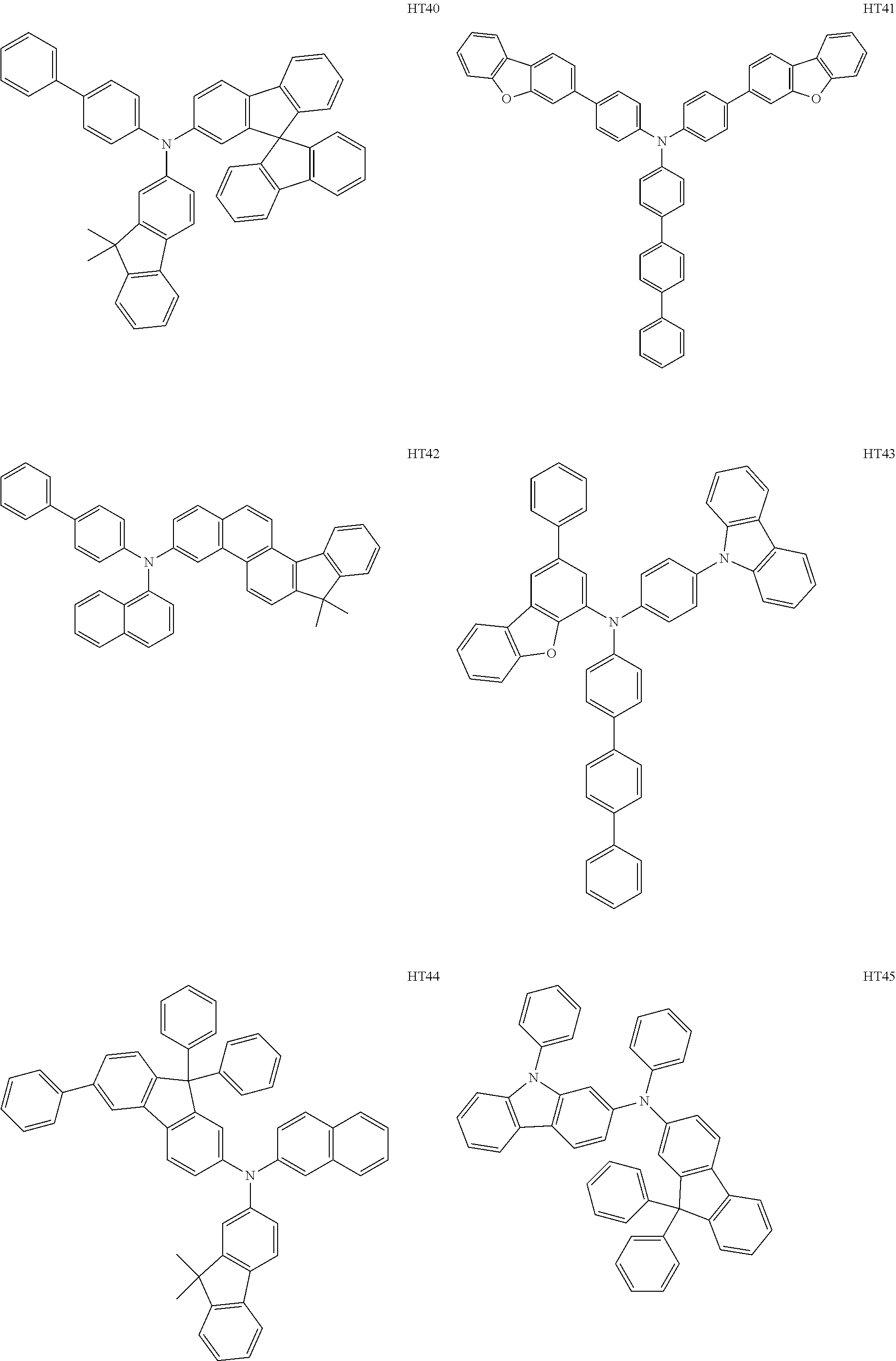

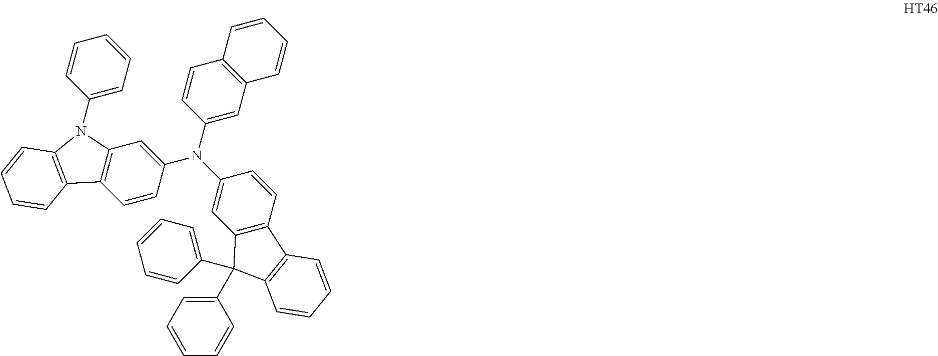 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

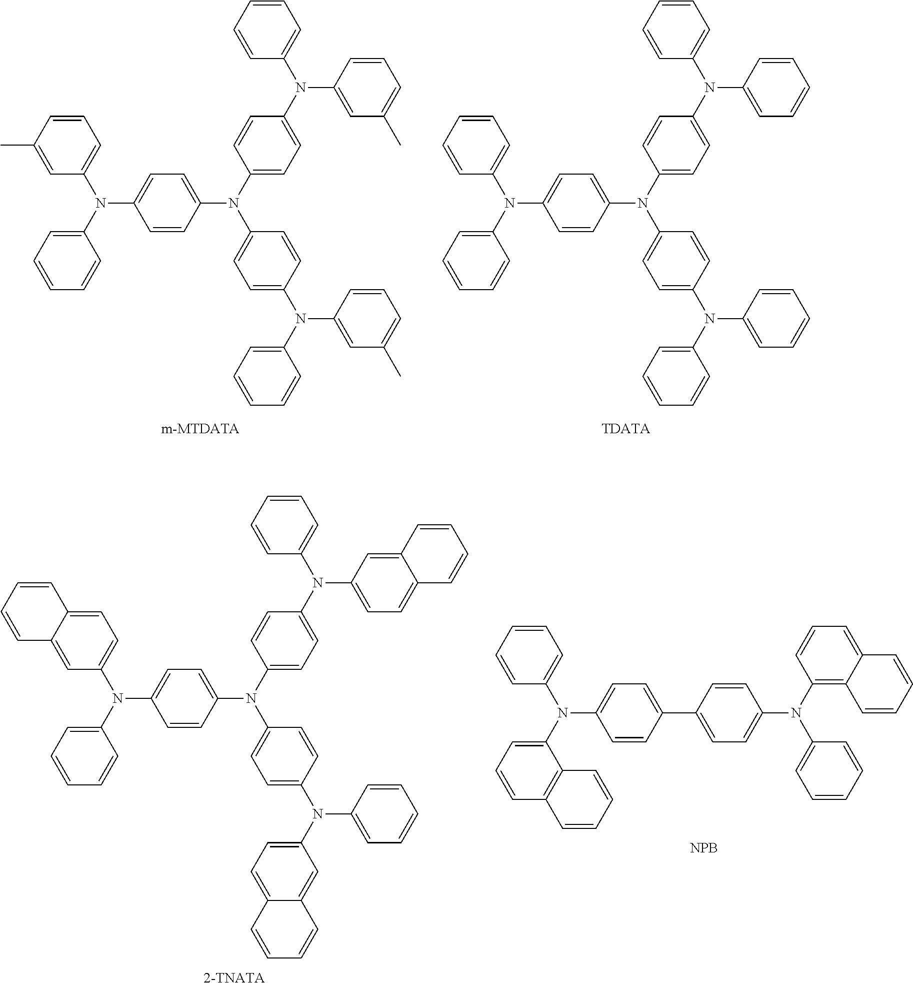

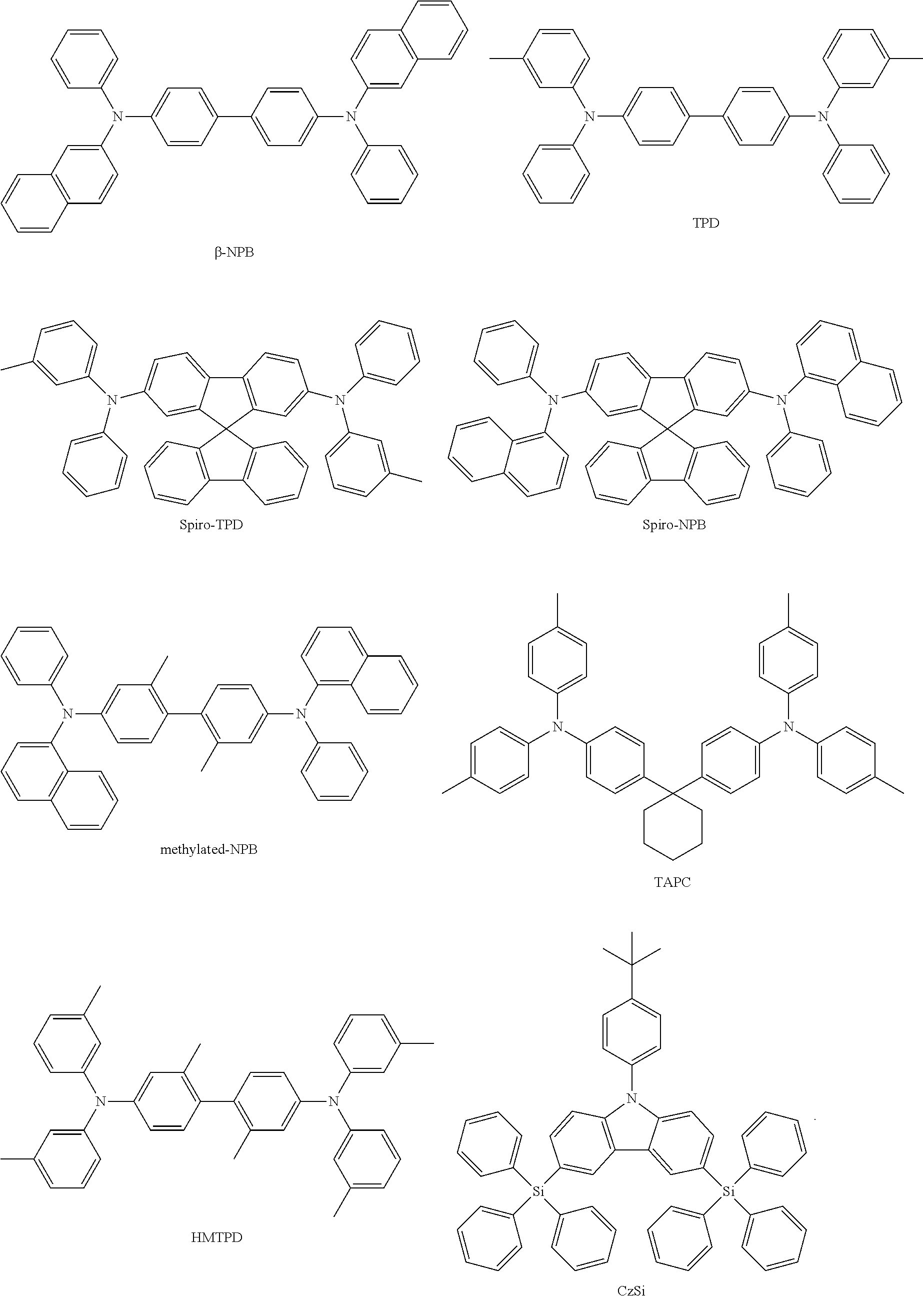

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron-blocking layer may block or reduce the leakage of electrons from an emission layer to a hole transport region. Materials that may be included in the hole transport region may be included in the emission auxiliary layer and the electron-blocking layer.

p-Dopant

The hole transport region may further include, in addition to these materials, a charge-generation material for the improvement of conductive properties. The charge-generation material may be uniformly (substantially uniformly) or non-uniformly (substantially non-uniformly) dispersed in the hole transport region (for example, in the form (or provide) of a single layer consisting of a charge-generation material).

The charge-generation material may be, for example, a p-dopant.

For example, the lowest unoccupied molecular orbital (LUMO) energy level of the p-dopant may be −3.5 electron volt (eV) or less.

In one or more embodiments, the p-dopant may include a quinone derivative, a cyano group-containing compound, a compound including element EL1 and element EL2, or any combination thereof.

Examples of the quinone derivative are TCNQ, F4-TCNQ, and/or the like.

Examples of the cyano group-containing compound are HAT-CN, and a compound represented by Formula 221:

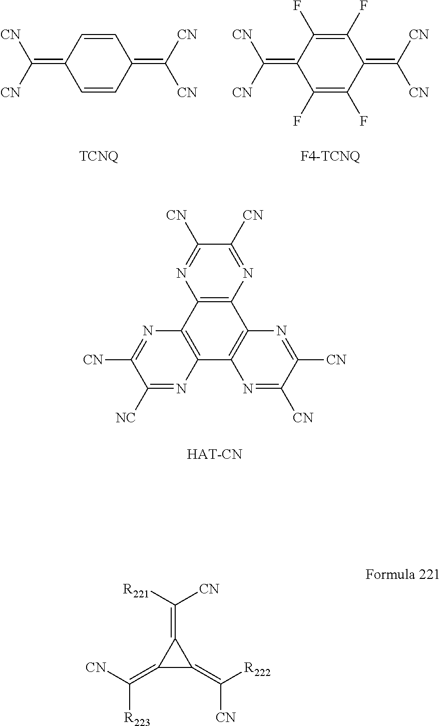

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Examples of the metal are an alkali metal (for example, lithium (Li), sodium (Na), potassium (K), rubidium (Rb), cesium (Cs), and/or the like); alkaline earth metal (for example, beryllium (Be), magnesium (Mg), calcium (Ca), strontium (Sr), barium (Ba), and/or the like); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); post-transition metal (for example, zinc (Zn), indium (In), tin (Sn), and/or the like); and lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid are silicon (Si), antimony (Sb), and tellurium (Te).

Examples of the non-metal are oxygen (O) and halogen (for example, F, Cl, Br, I, and/or the like).

Examples of the compound including element EL1 and element EL2 are metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, or any combination thereof.

Examples of the metal oxide are tungsten oxide (for example, WO, W₂O₃, WO₂, WO₃, W₂O₅, and/or the like), vanadium oxide (for example, VO, V₂O₃, VO₂, V₂O₅, and/or the like), molybdenum oxide (for example, MoO, Mo₂O₃, MoO₂, MoO₃, Mo₂O₅, and/or the like), and rhenium oxide (for example, ReO₃, and/or the like).

Examples of the metal halide are alkali metal halide, alkaline earth metal halide, transition metal halide, post-transition metal halide, and lanthanide metal halide.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide are titanium halide (for example, TiF₄, TiCl₄, TiBr₄, TiI₄, and/or the like), zirconium halide (for example, ZrF₄, ZrCl₄, ZrBr₄, ZrI₄, and/or the like), hafnium halide (for example, HfF₄, HfCl₄, HfBr₄, HfI₄, and/or the like), vanadium halide (for example, VF₃, VCl₃, VBr₃, VI₃, and/or the like), niobium halide (for example, NbF₃, NbCl₃, NbBr₃, NbI₃, and/or the like), tantalum halide (for example, TaF₃, TaCl₃, TaBr₃, TaI₃, and/or the like), chromium halide (for example, CrF₃, CrCl₃, CrBr₃, CrI₃, and/or the like), molybdenum halide (for example, MoF₃, MoCl₃, MoBr₃, MoI₃, and/or the like), tungsten halide (for example, WF₃, WCl₃, WBr₃, WI₃, and/or the like), manganese halide (for example, MnF₂, MnCl₂, MnBr₂, MnI₂, and/or the like), technetium halide (for example, TcF₂, TcCl₂, TcBr₂, TcI₂, and/or the like), rhenium halide (for example, ReF₂, ReCl₂, ReBr₂, ReI₂, and/or the like), iron halide (for example, FeF₂, FeCl₂, FeBr₂, FeI₂, and/or the like), ruthenium halide (for example, RuF₂, RuCl₂, RuBr₂, RuI₂, and/or the like), osmium halide (for example, OsF₂, OsCl₂, OsBr₂, OsI₂, and/or the like), cobalt halide (for example, CoF₂, CoCl₂, CoBr₂, CoI₂, and/or the like), rhodium halide (for example, RhF₂, RhCl₂, RhBr₂, RhI₂, and/or the like), iridium halide (for example, IrF₂, IrCl₂, IrBr₂, IrI₂, and/or the like), nickel halide (for example, NiF₂, NiCl₂, NiBr₂, NiI₂, and/or the like), palladium halide (for example, PdF₂, PdCl₂, PdBr₂, PdI₂, and/or the like), platinum halide (for example, PtF₂, PtCl₂, PtBr₂, PtI₂, and/or the like), copper halide (for example, CuF, CuCl, CuBr, CuI, and/or the like), silver halide (for example, AgF, AgCl, AgBr, AgI, and/or the like), and gold halide (for example, AuF, AuCl, AuBr, AuI, and/or the like).

Examples of the post-transition metal halide are zinc halide (for example, ZnF₂, ZnCl₂, ZnBr₂, ZnI₂, and/or the like), indium halide (for example, InI₃, and/or the like), and tin halide (for example, SnI₂, and/or the like).

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Examples of the metalloid halide are antimony halide (for example, SbCl₅ and/or the like) and/or the like.

Examples of the metal telluride are alkali metal telluride (for example, Li₂Te, Na₂Te, K₂Te, Rb₂Te, Cs₂Te, and/or the like), alkaline earth metal telluride (for example, BeTe, MgTe, CaTe, SrTe, BaTe, and/or the like), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), post-transition metal telluride (for example, ZnTe, and/or the like), lanthanide metal telluride (for example, LaTe, CeTe, PrTe, NdTe, PmTe, EuTe, GdTe, TbTe, DyTe, HoTe, ErTe, TmTe, YbTe, LuTe, and/or the like), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other to emit white light. In one or more embodiments, the emission layer may include two or more materials of a red light-emitting material, a green light-emitting material, and a blue light-emitting material, in which the two or more materials are mixed with each other in a single layer to emit white light. For example, the emission layer may be to emit blue light.

In one or more embodiments, the emission layer may include the amine-containing compound represented by Formula 1 as described in the specification.

The emission layer may include a host and a dopant.

In one or more embodiments, the dopant may include the amine-containing compound represented by Formula 1 as described in the specification. In this regard, the dopant may further include a phosphorescent dopant, a fluorescent dopant, or any combination thereof, in addition to the amine-containing compound represented by Formula 1. In addition to the amine-containing compound represented by Formula 1, the phosphorescent dopant, the fluorescent dopant, and/or the like that may be further included in the emission layer are each substantially the same as described.

The amount of the dopant in the emission layer may be from about 0.01 part by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within this range, excellent or suitable light-emission characteristics may be obtained without a substantial increase in driving voltage.

### Host

The host may include, for example, a carbazole-containing compound, an anthracene-containing compound, or any combination thereof.

In one or more embodiments, the host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be as described herein with respect to Q₁.

For example, when xb11 in Formula 301 is 2 or more, two or more of Ar₃₀₁(s) may be linked to each other via a single bond.

In one or more embodiments, the host may include at least one of (e.g., selected from among) a compound represented by Formula 301-1, a compound represented by Formula 301-2, or any combination thereof:

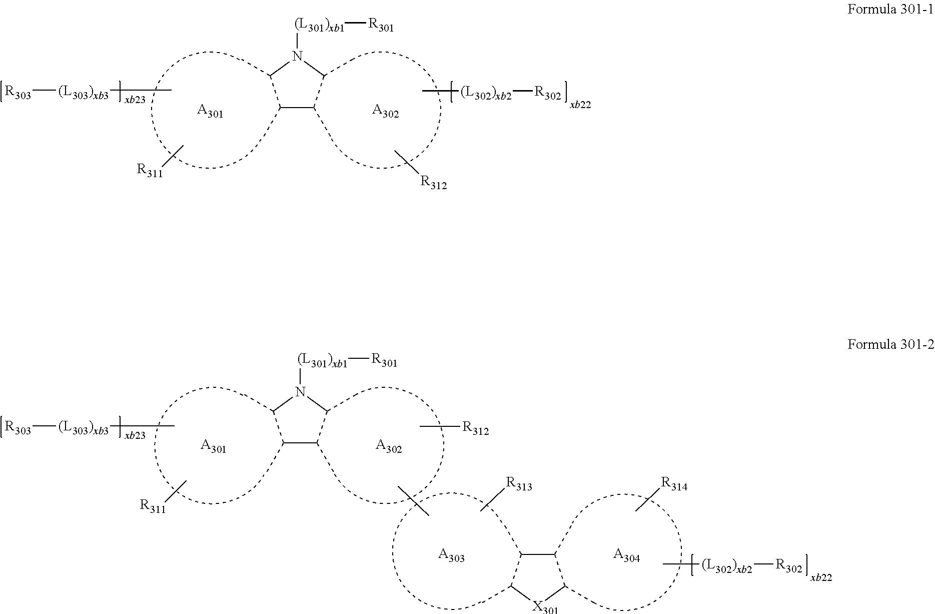

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as described herein,
L₃₀₂ to L₃₀₄ may each independently be as described herein with respect to with L₃₀₁,
xb2 to xb4 may each independently be as described herein with respect to xb1, and
R₃₀₂ to R₃₀₅ and R₃₁₁ to R₃₁₄ may each be as described herein with respect to R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In one or more embodiments, the host may include at least one (e.g., one or more) selected from among Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

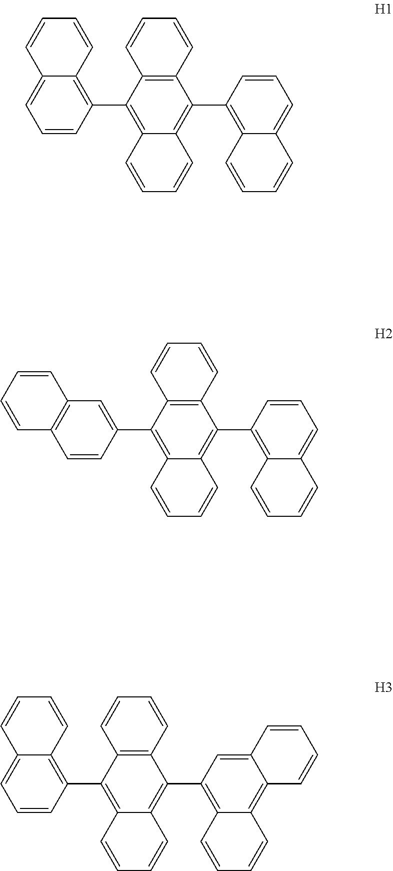

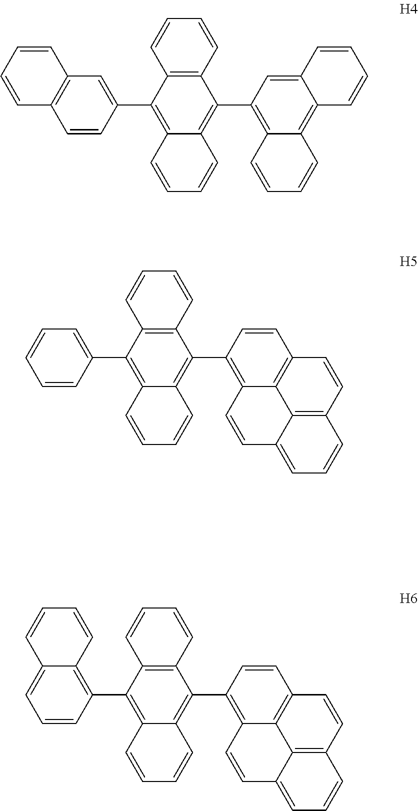

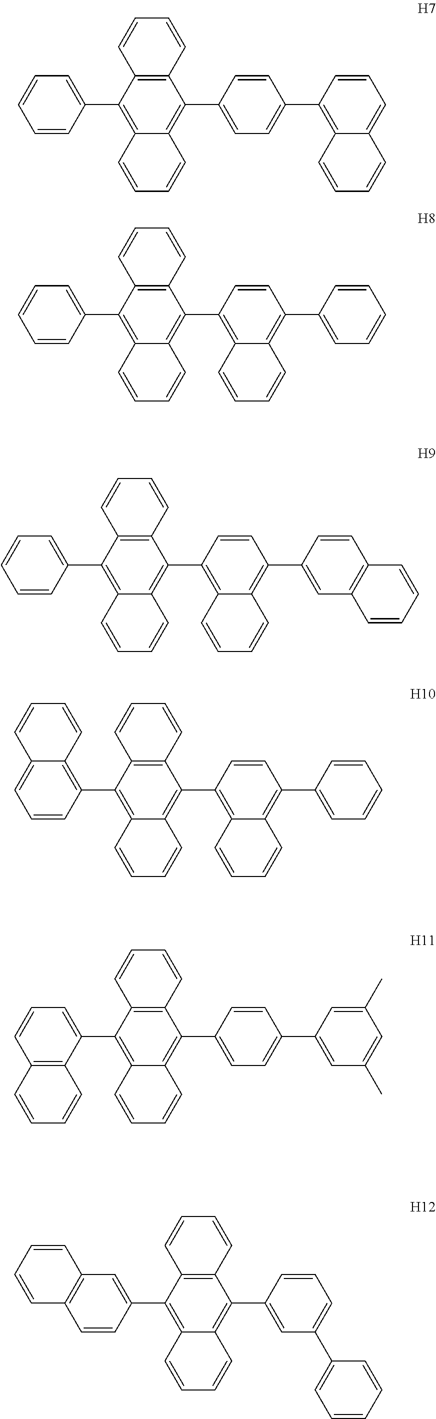

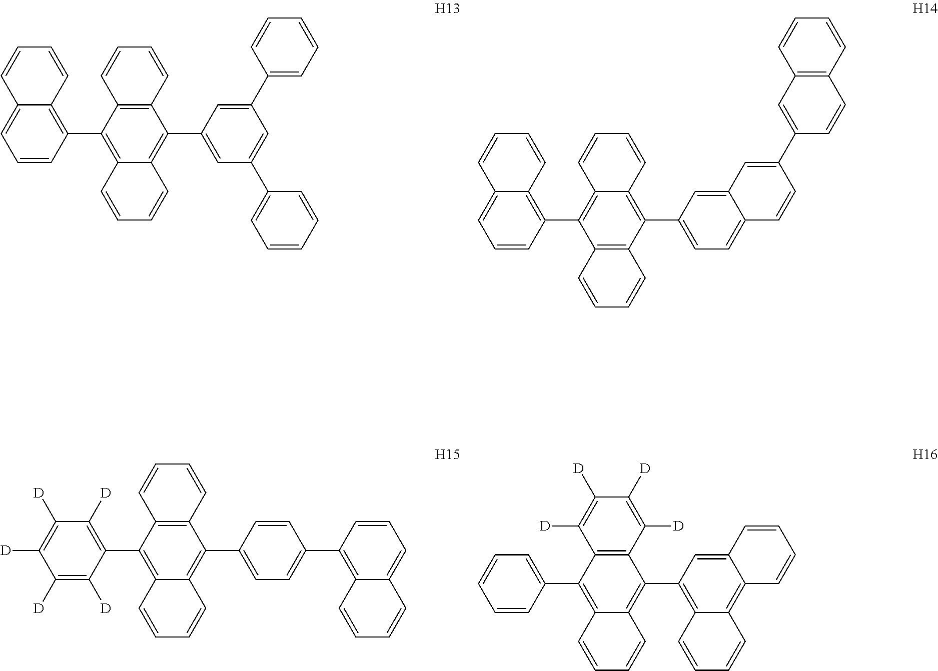

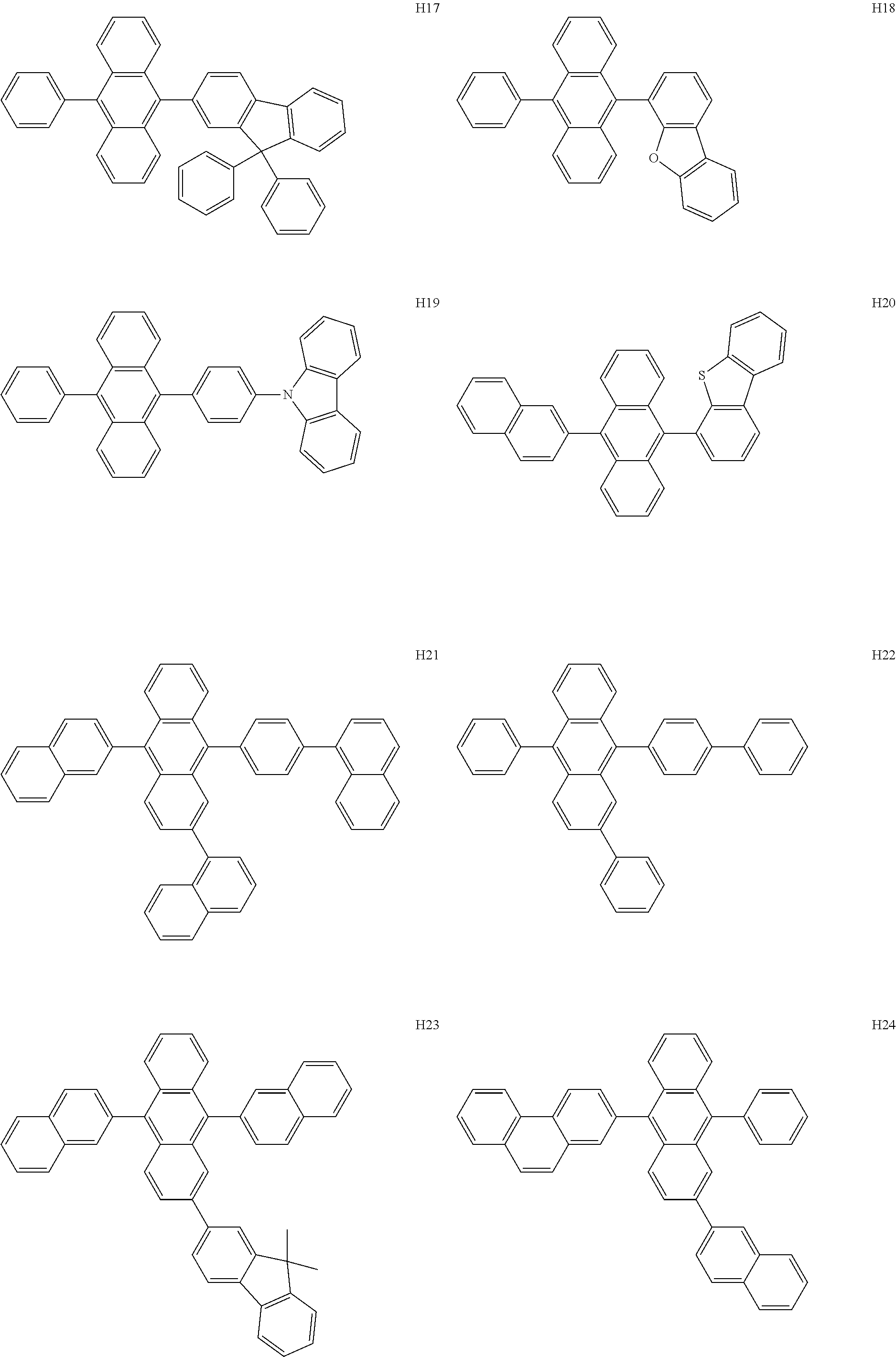

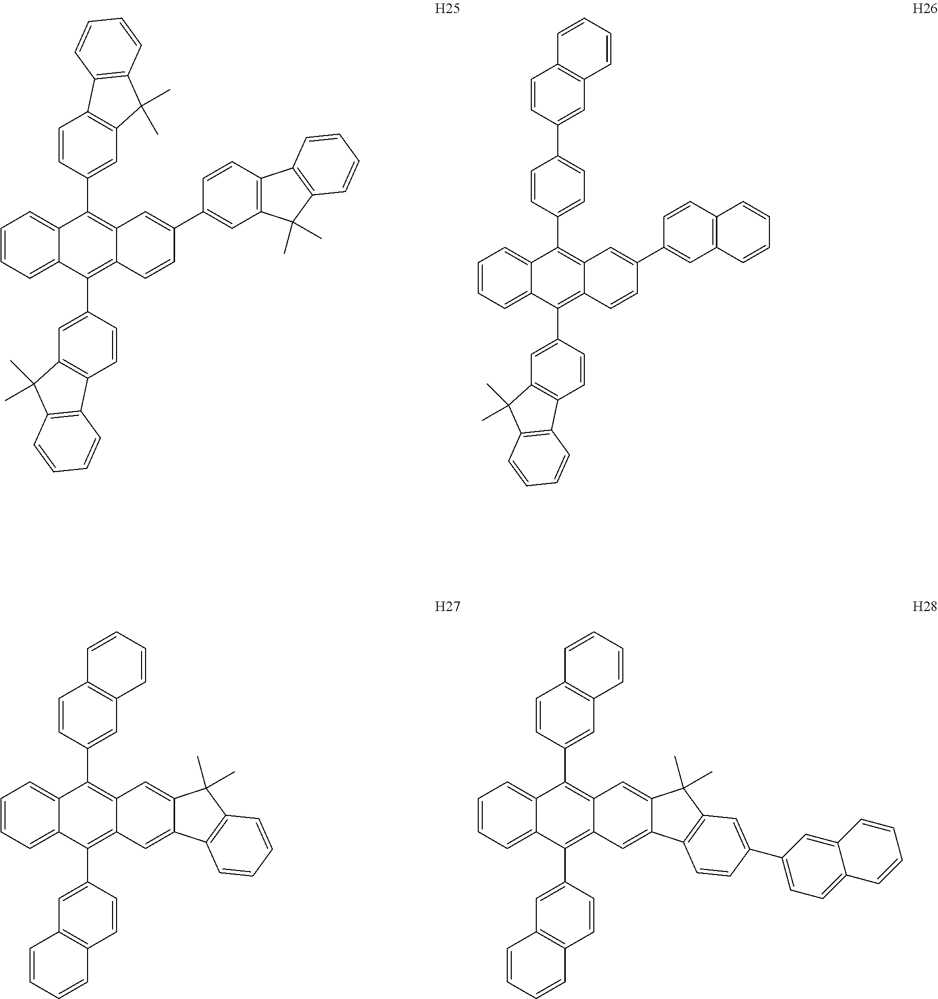

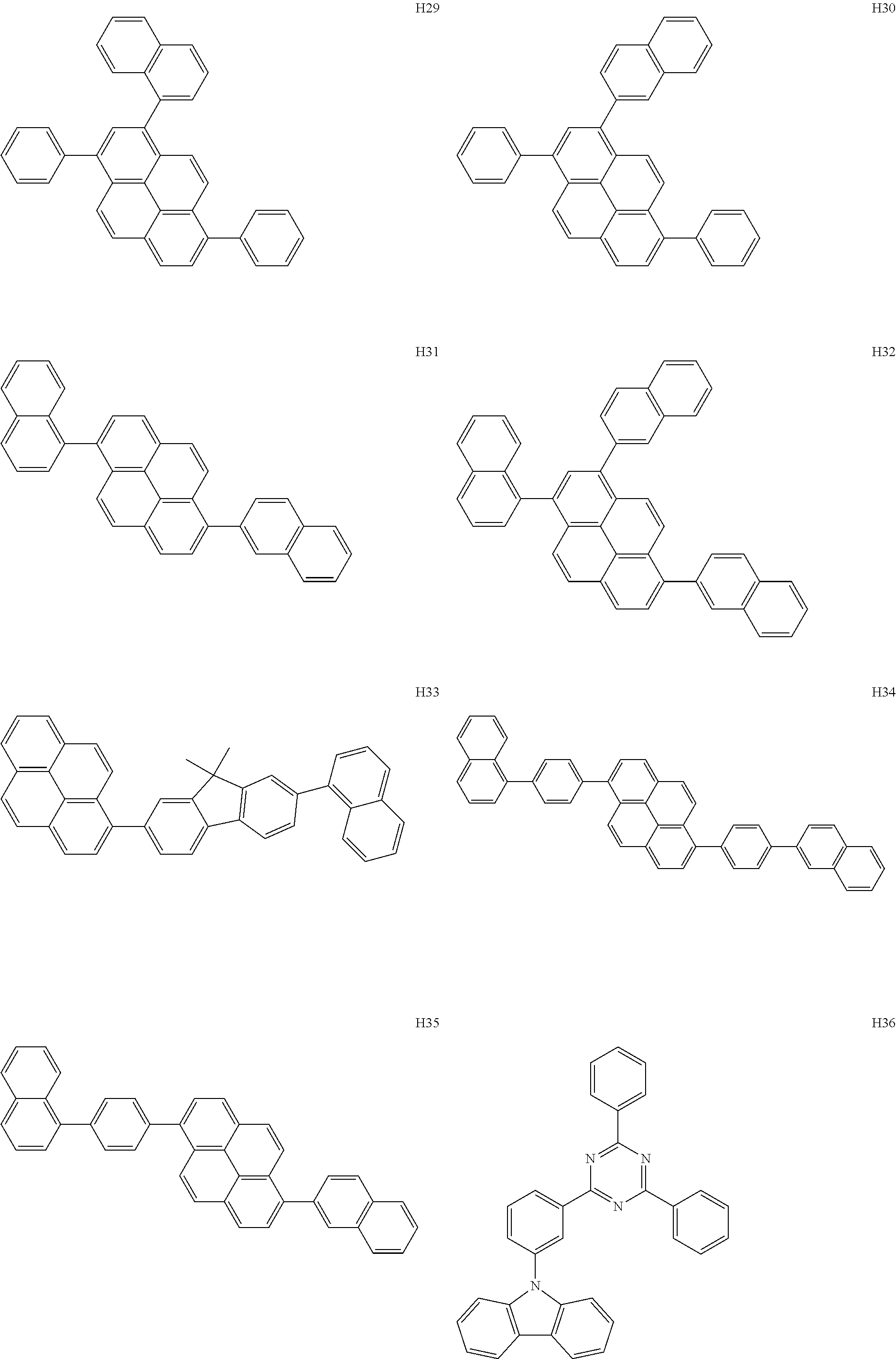

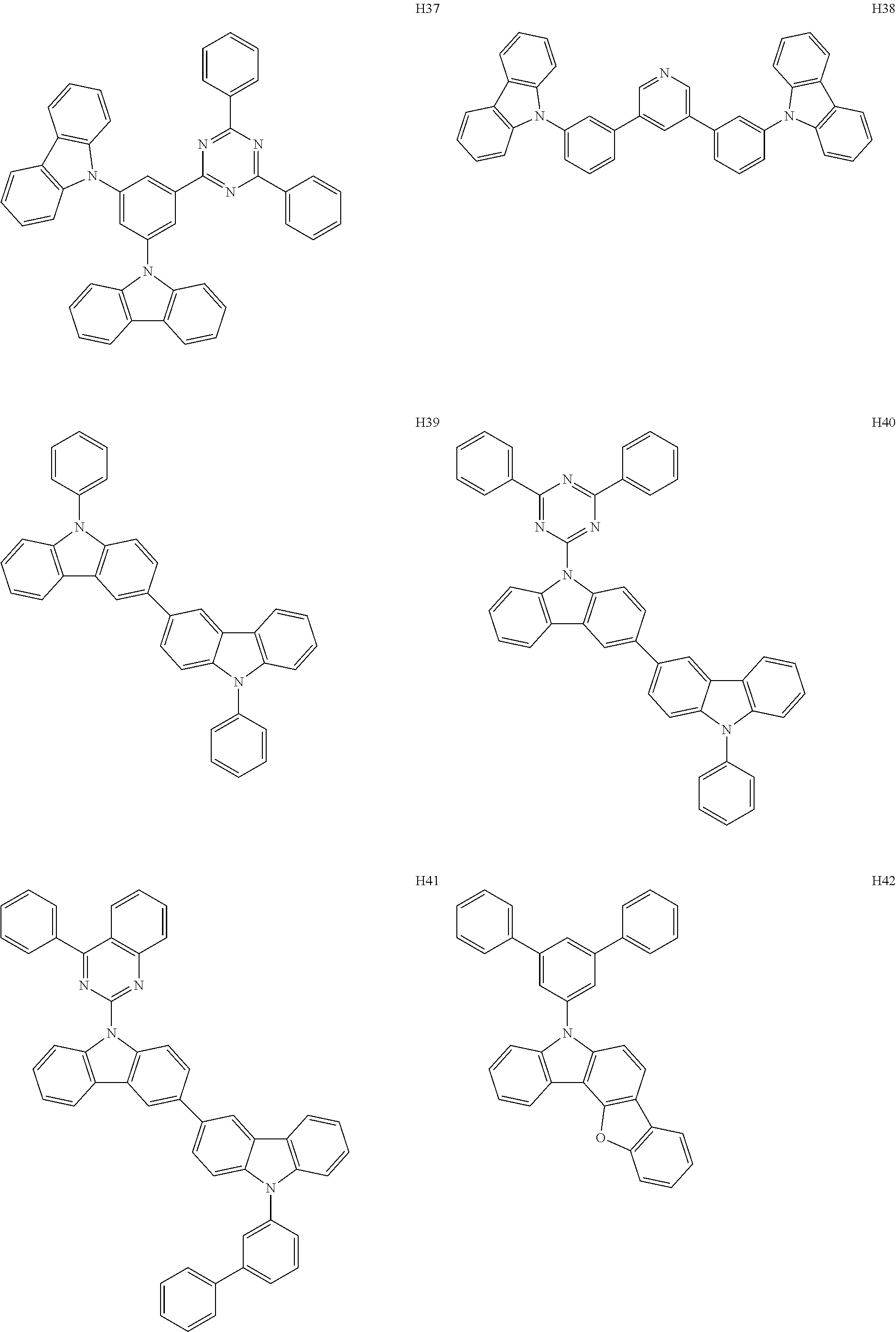 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

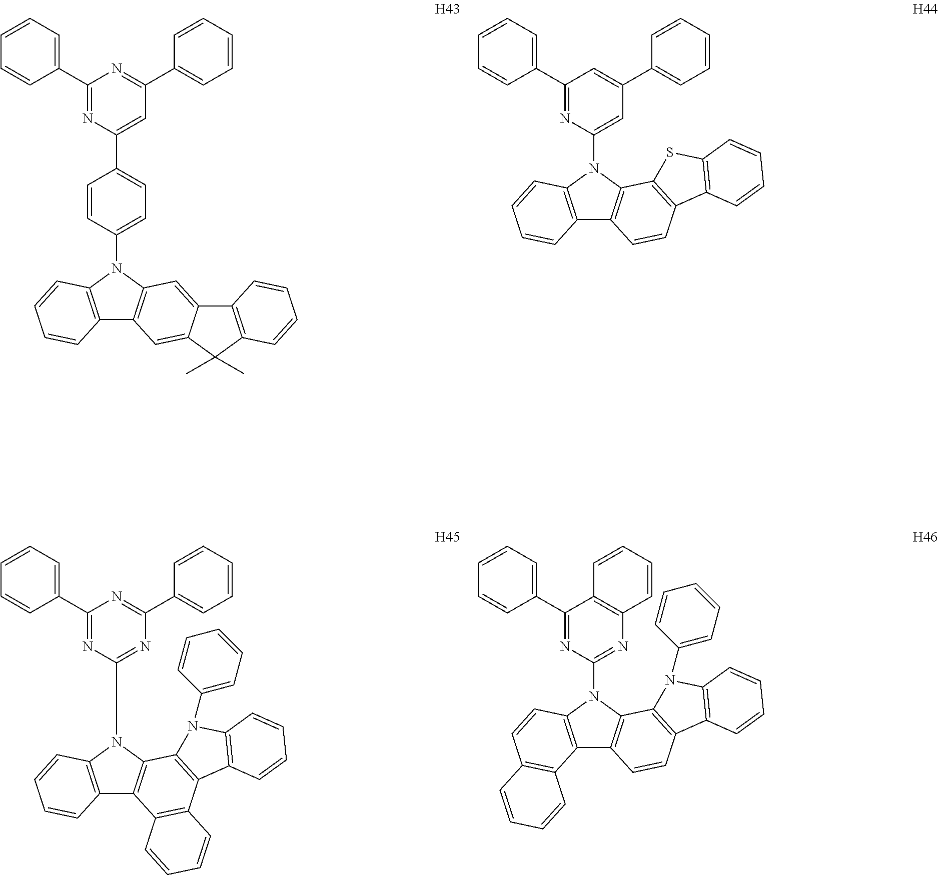 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

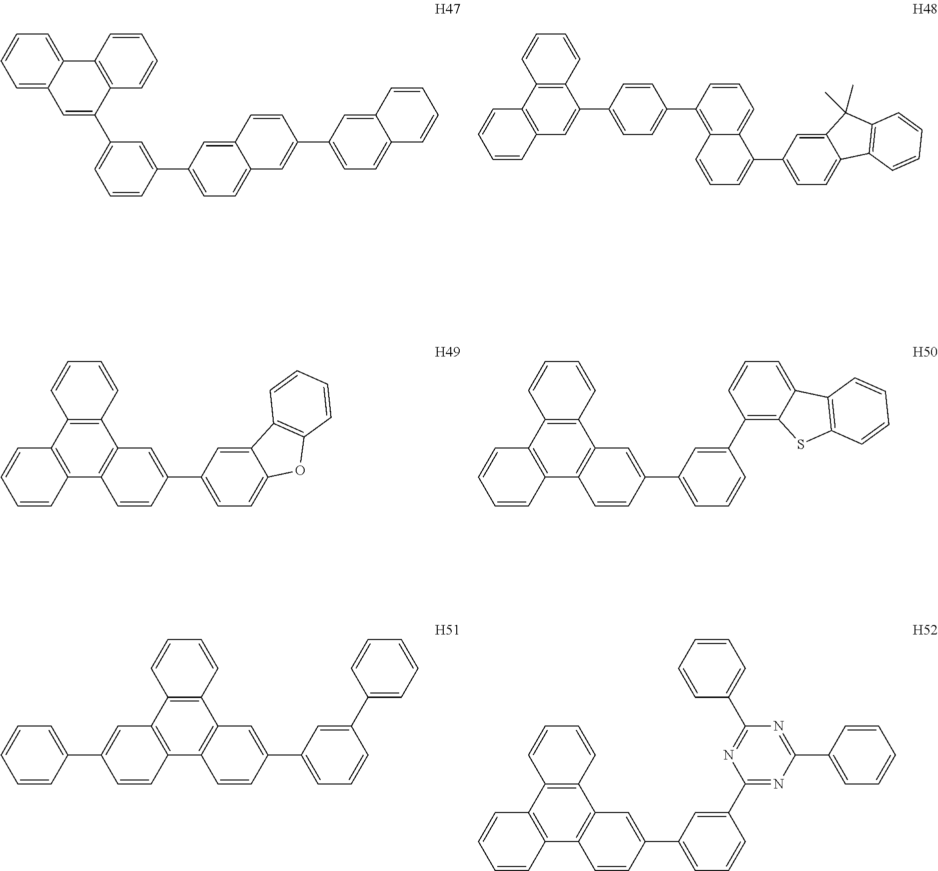

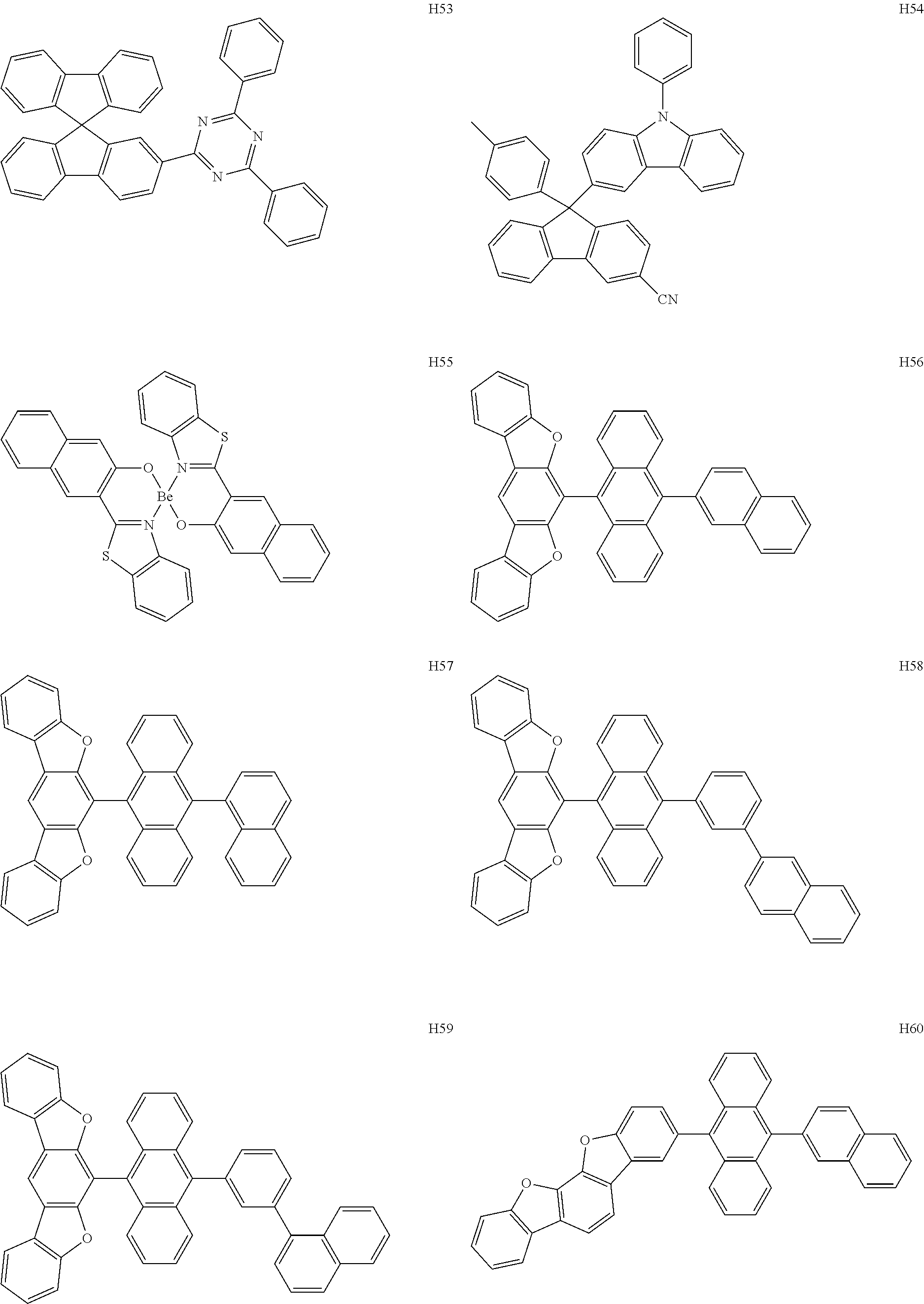

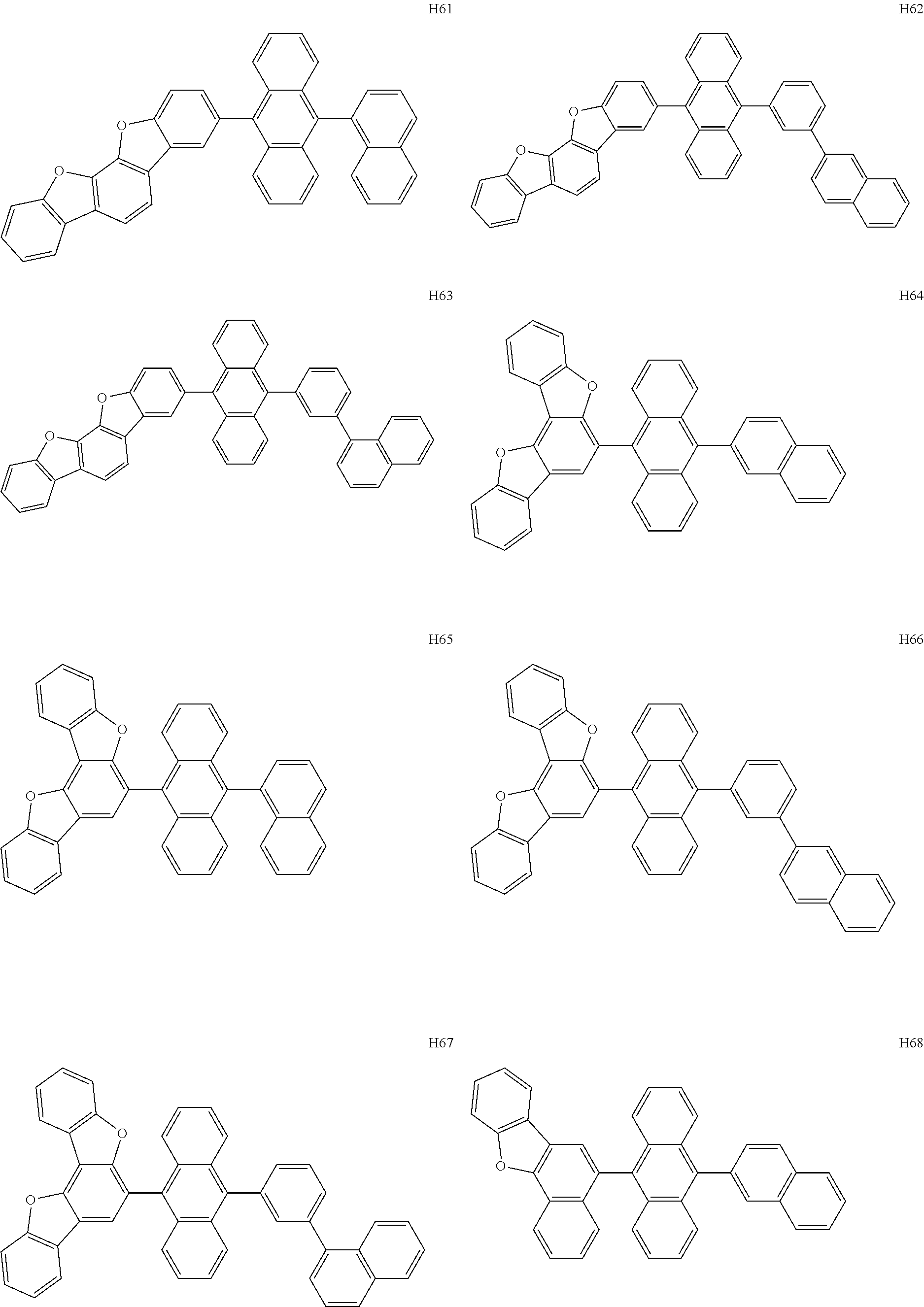

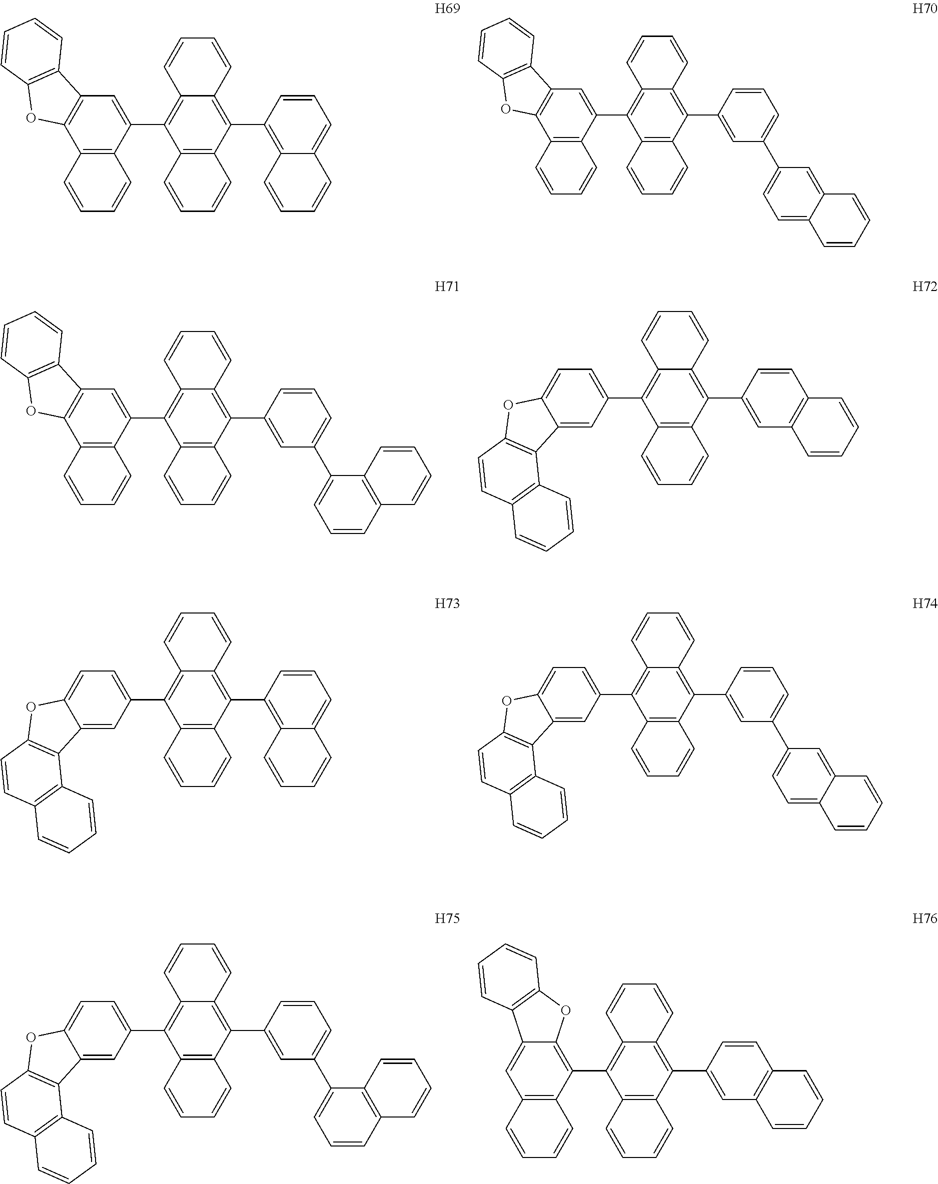

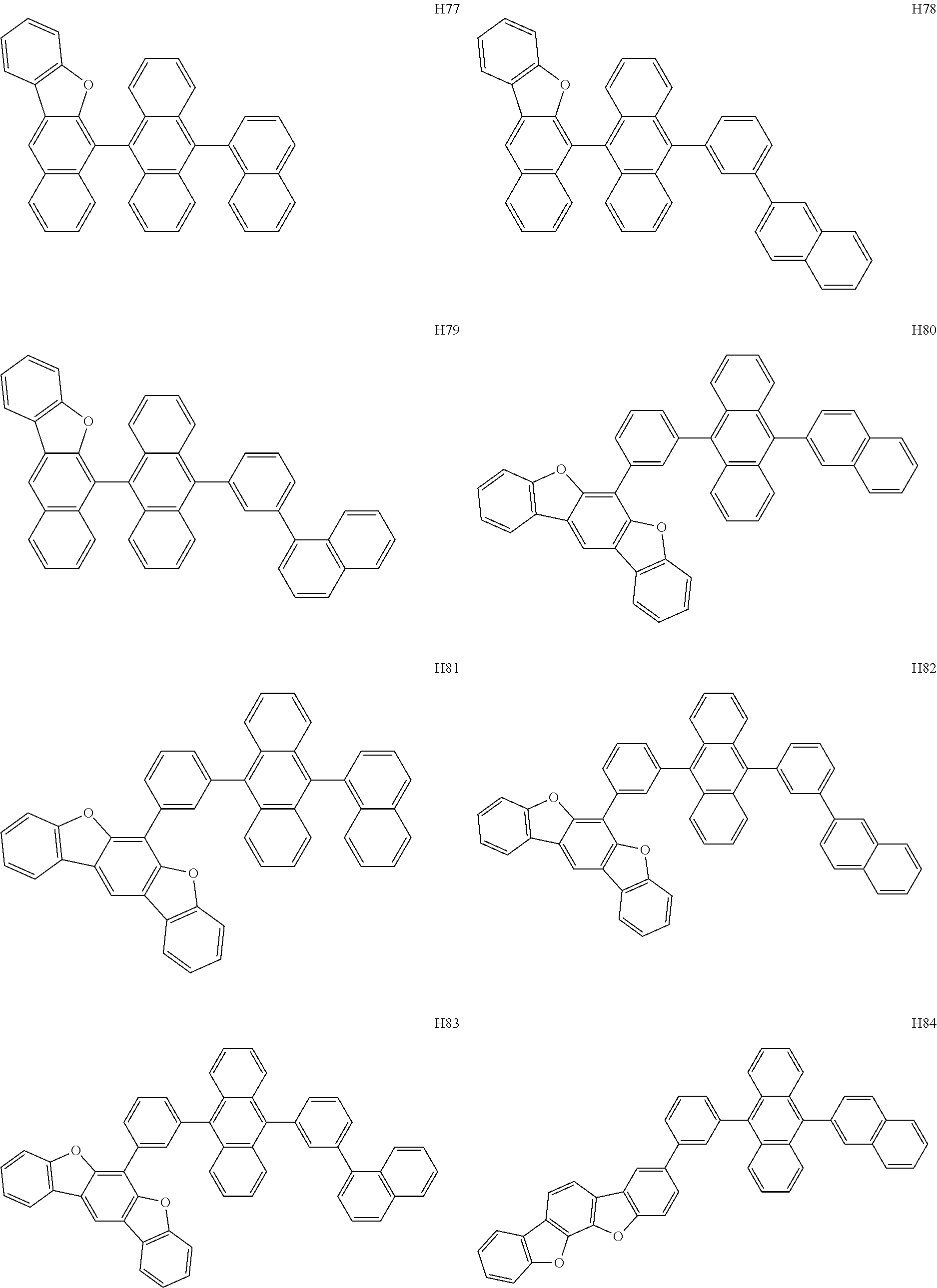

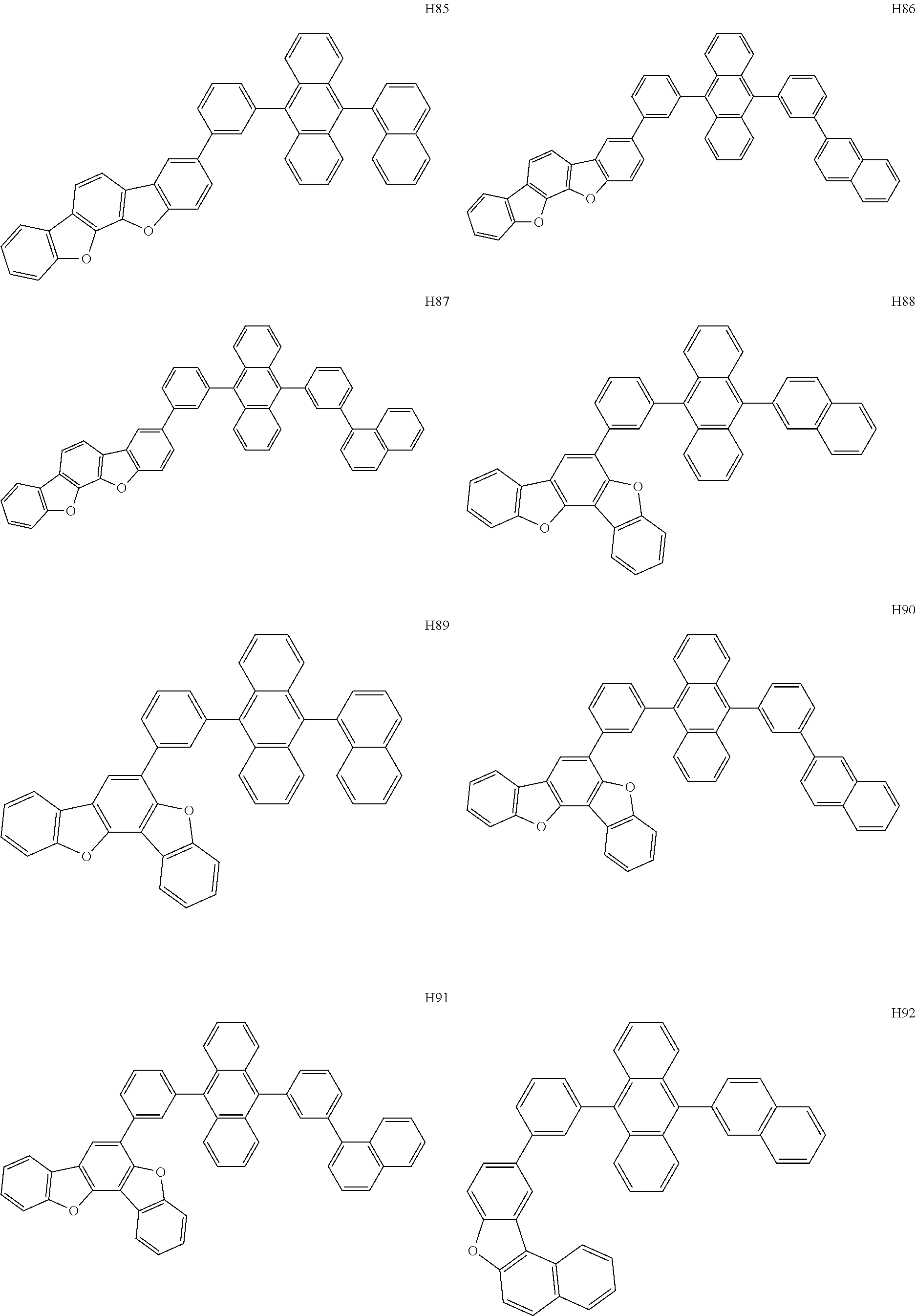

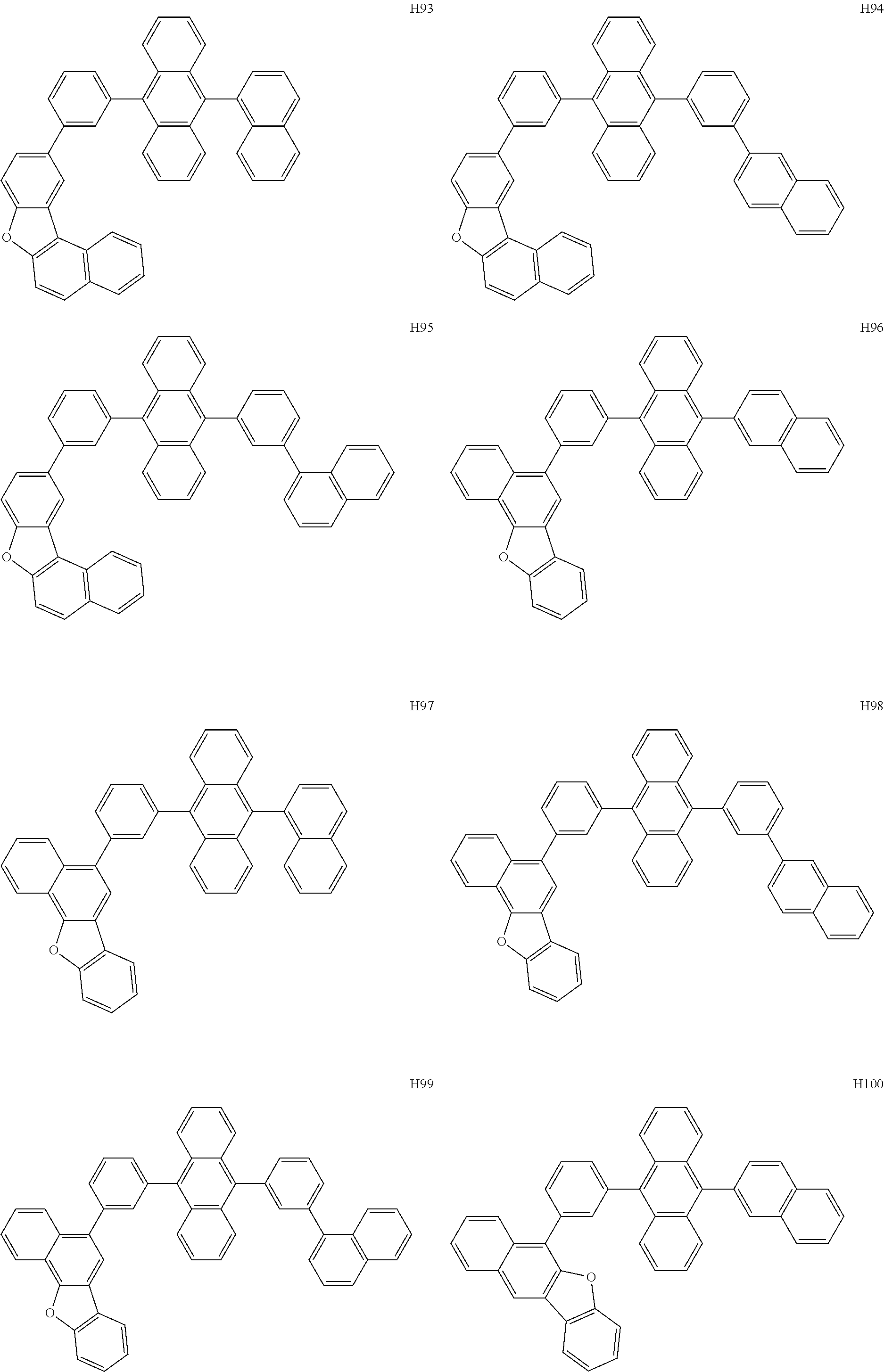

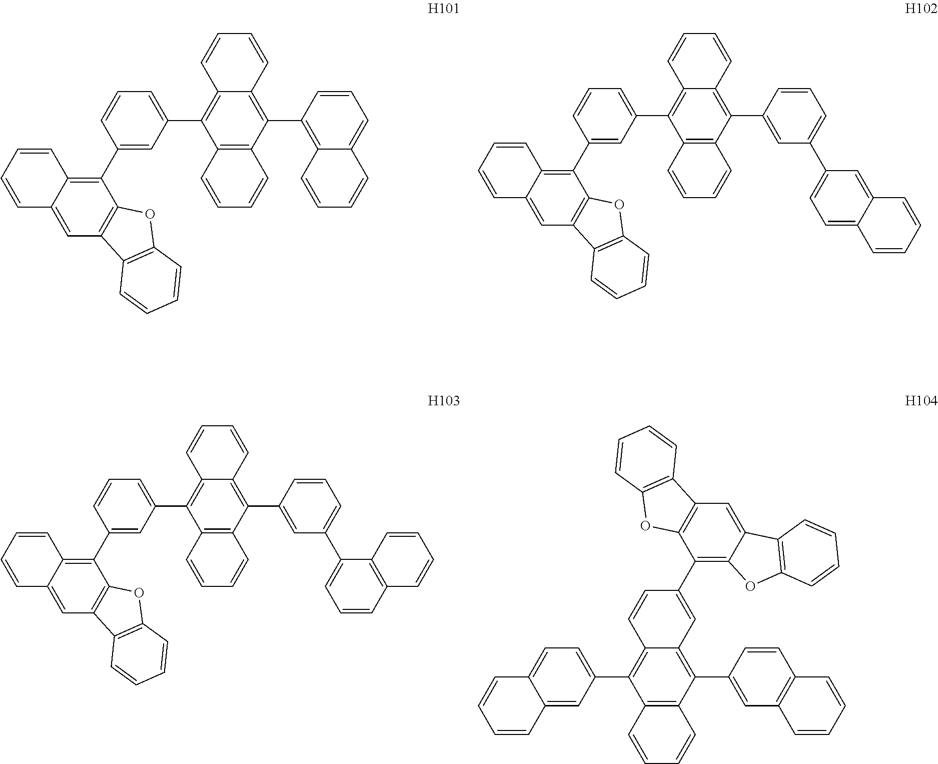 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

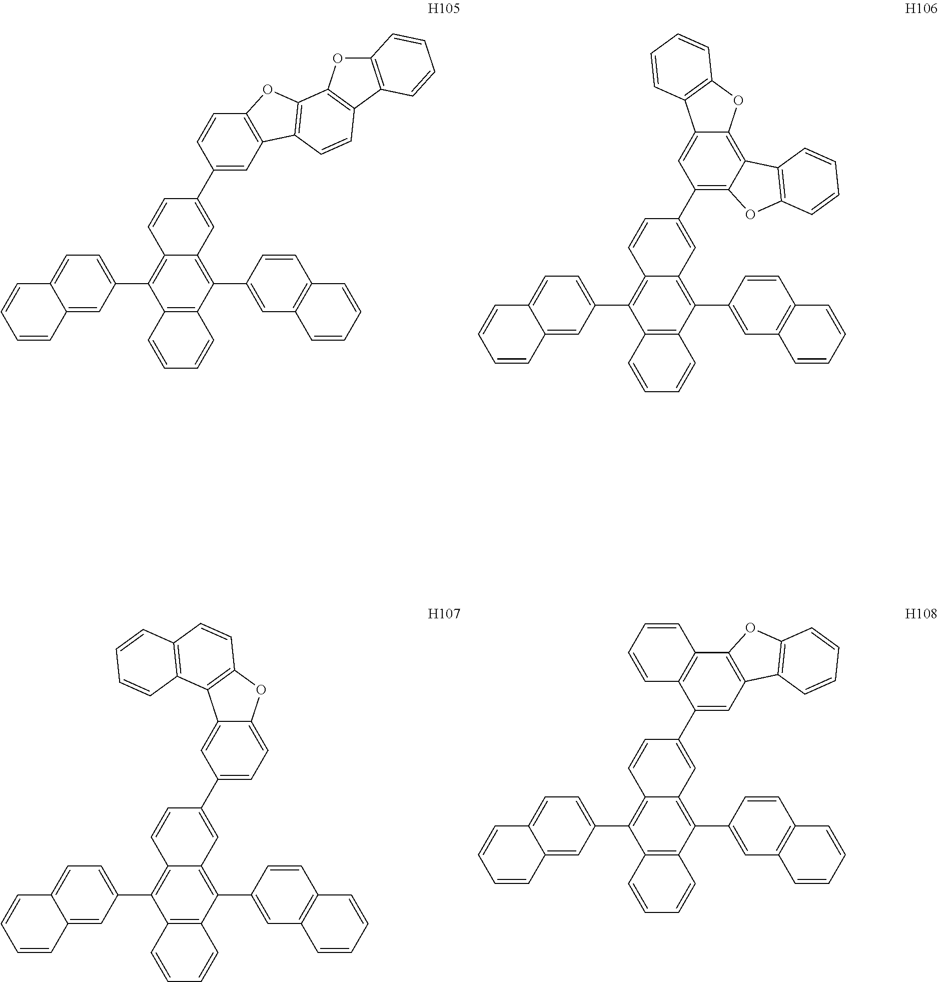 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

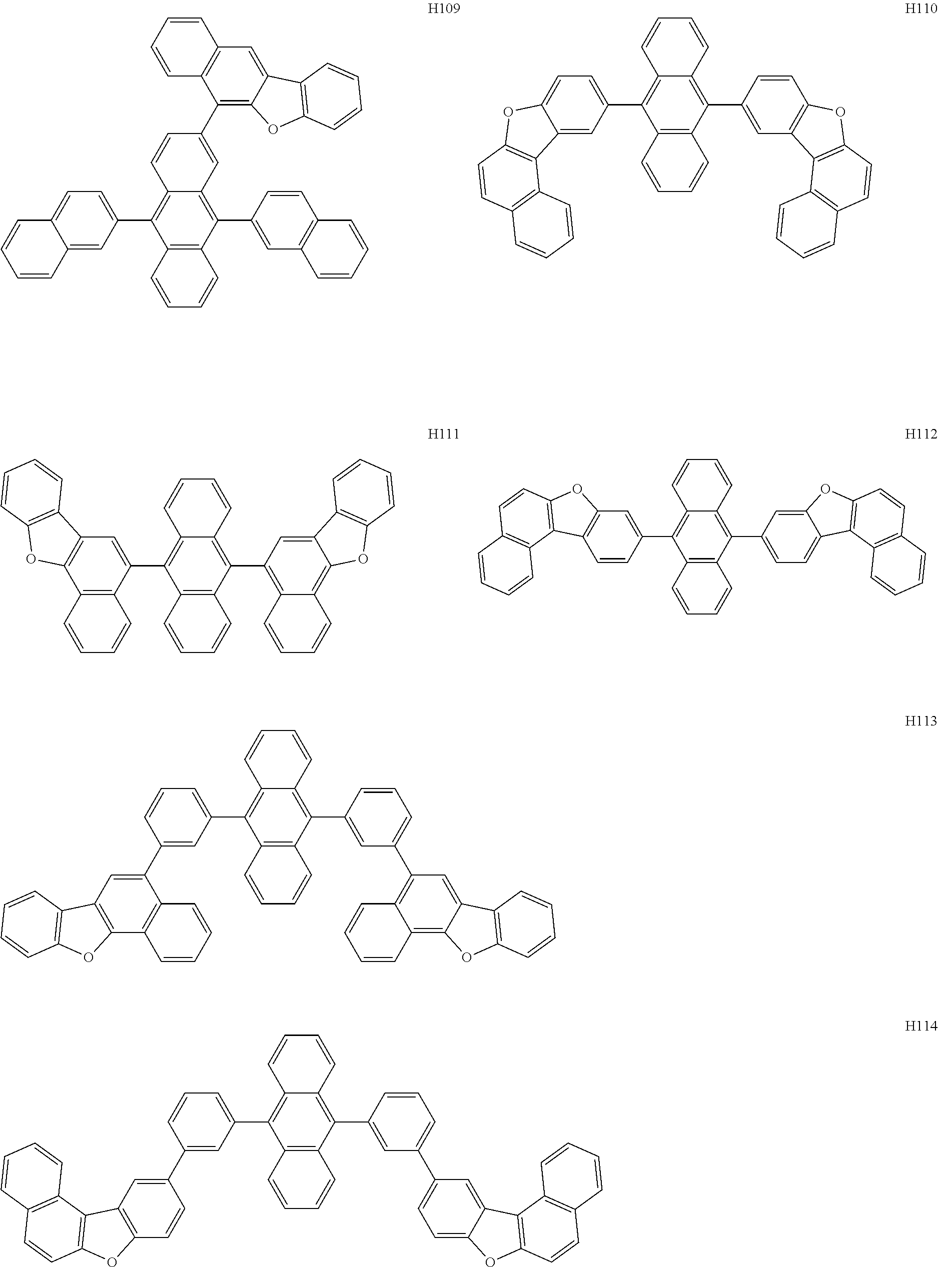

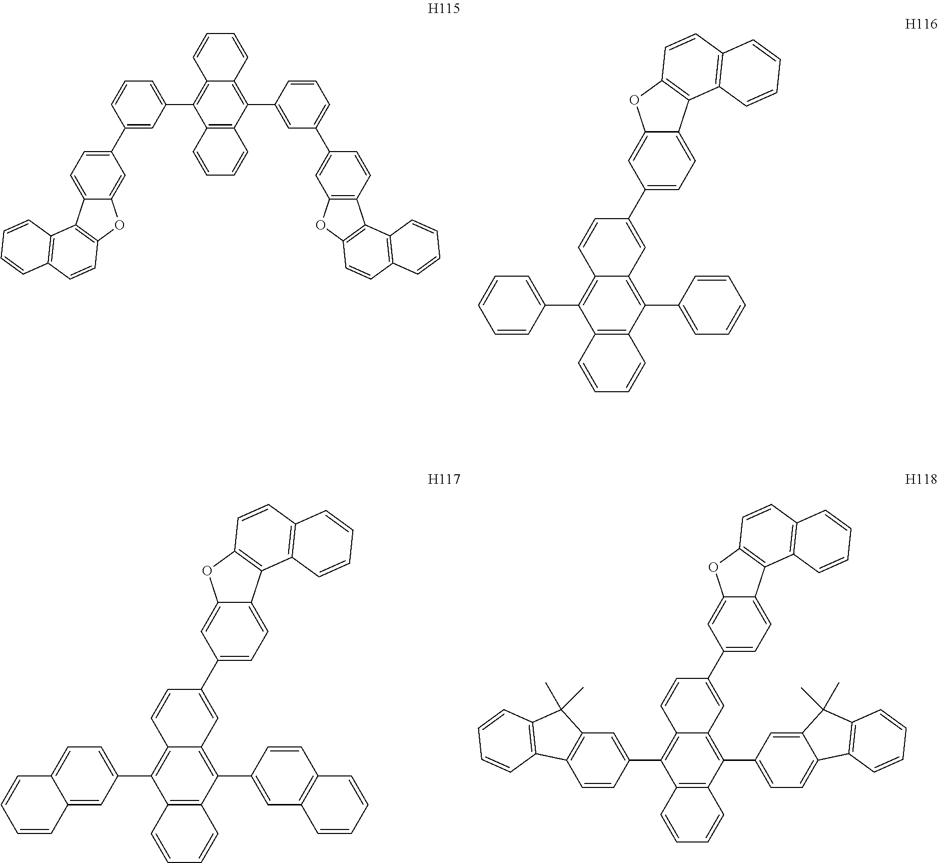 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

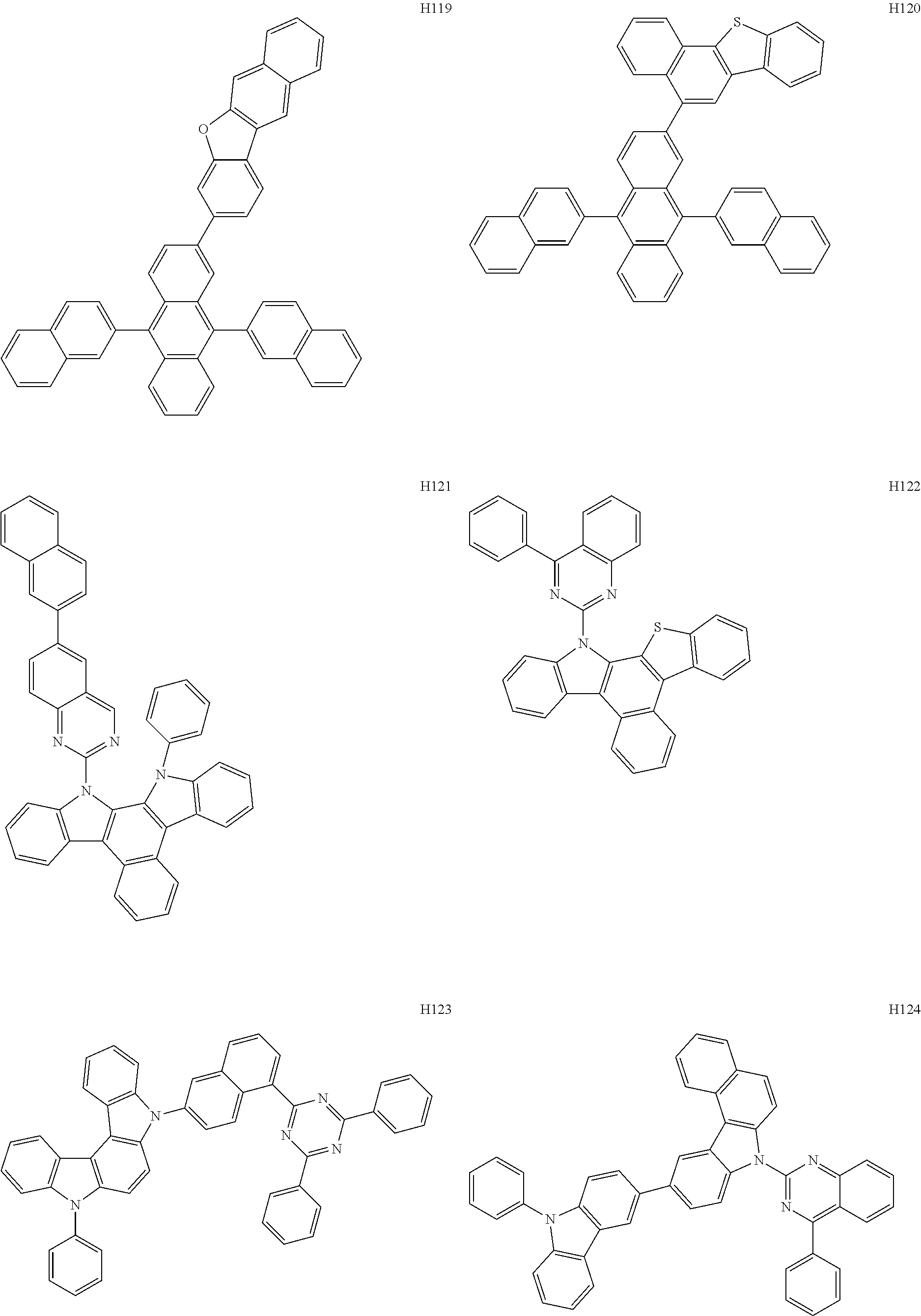

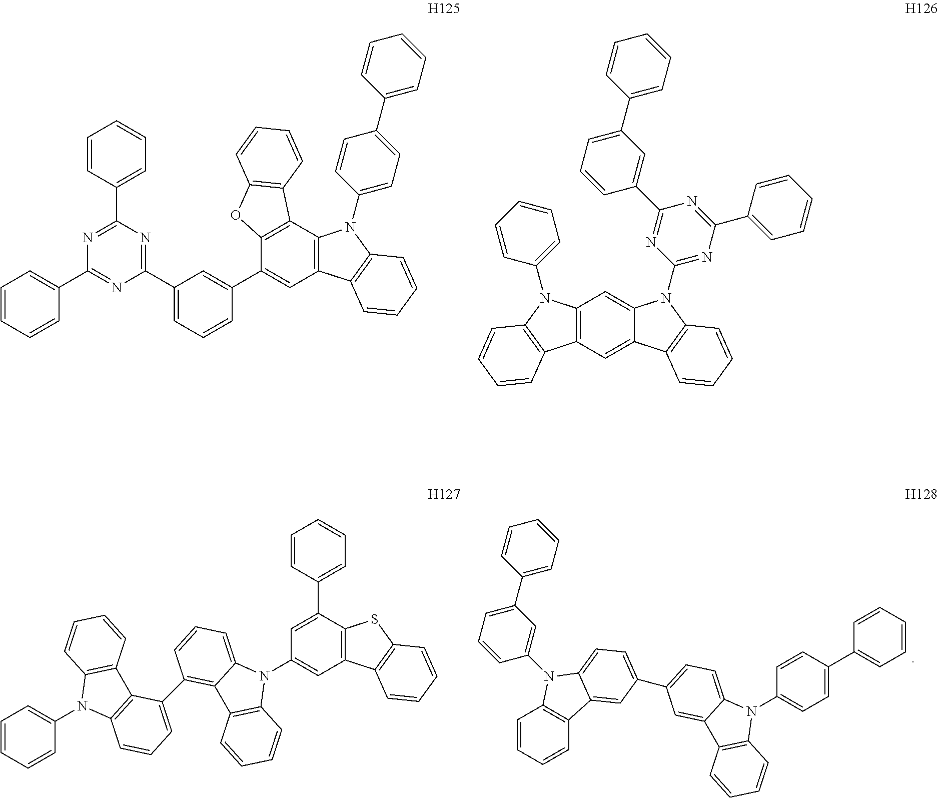 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

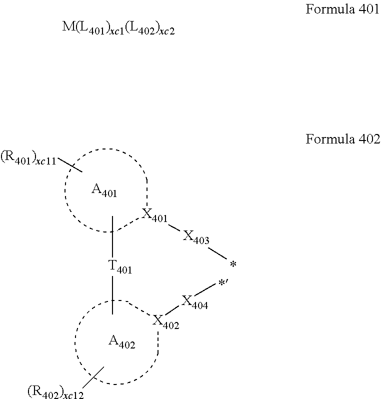

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *=C=*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁3)(Q₄₁₄),
Q₄₁₁ to Q₄₁₄ may each be as described herein with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be as described herein with respect to Q₁,
xc11 and xc12 may each independently be an integer from 0 to 10, and
* and *′ in Formula 402 each indicates a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁(s) in two or more of L₄₀₁(s) may not be linked or optionally may be linked to each other via T₄₀₂, which is a linking group, or two ring A₄₀₂(s) may be optionally linked to each other via T₄₀₃, which is a linking group (see Compounds PD1 to PD4 and PD7). T₄₀₂ and T₄₀₃ may each be as described herein with respect to T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, and/or the like), or any combination thereof.

The phosphorescent dopant may include, for example, at least one (e.g., one or more) selected from among compounds PD1 to PD39, or any combination thereof:

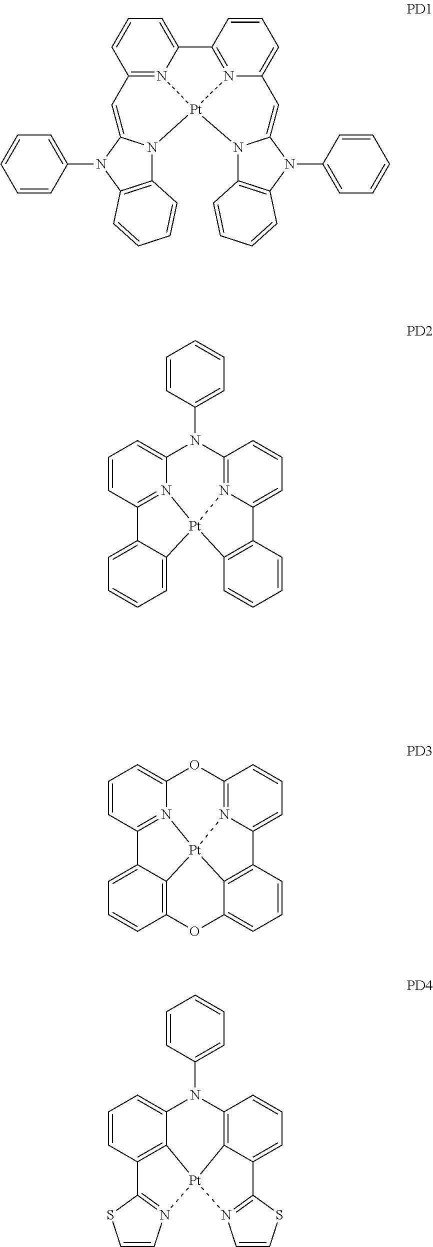

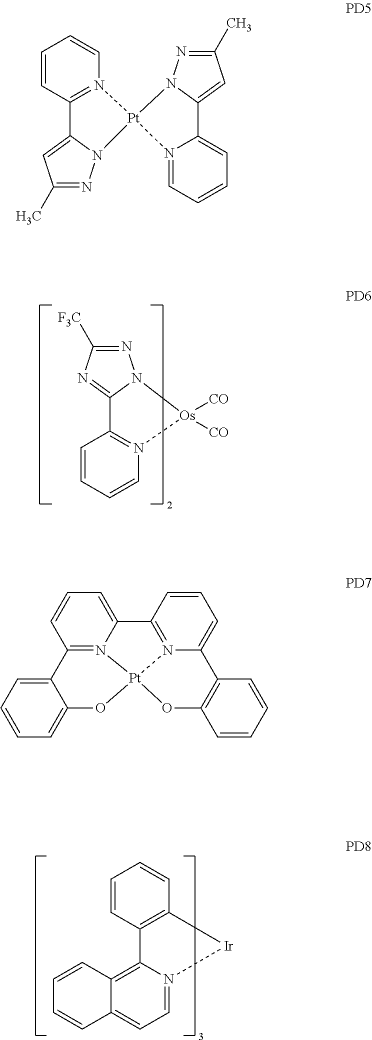

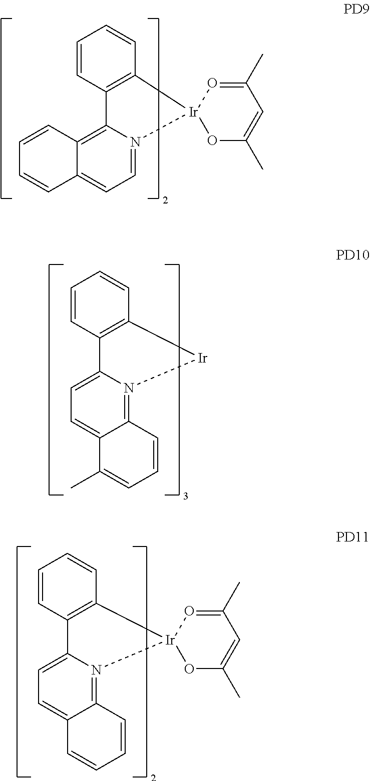

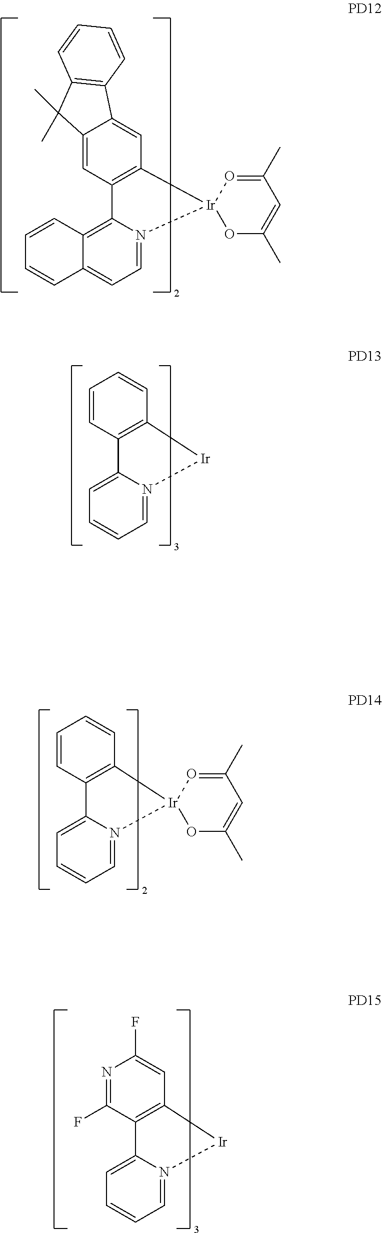

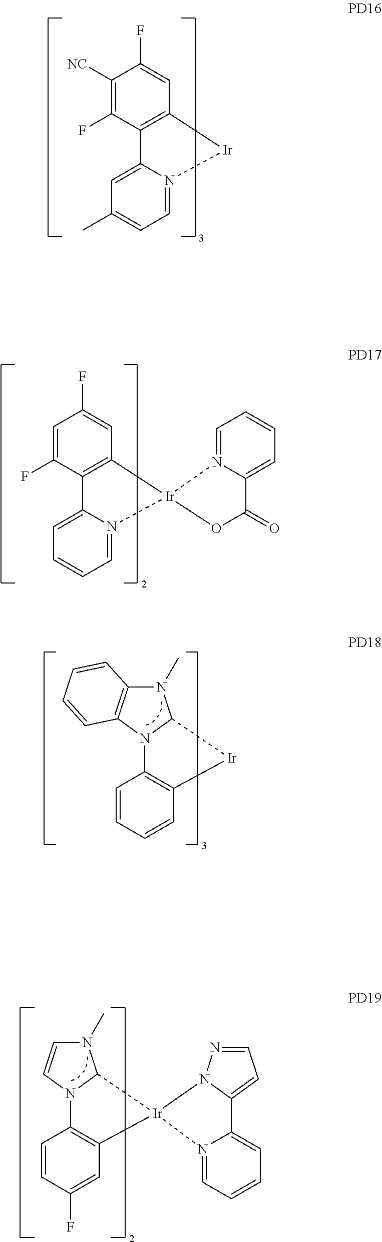

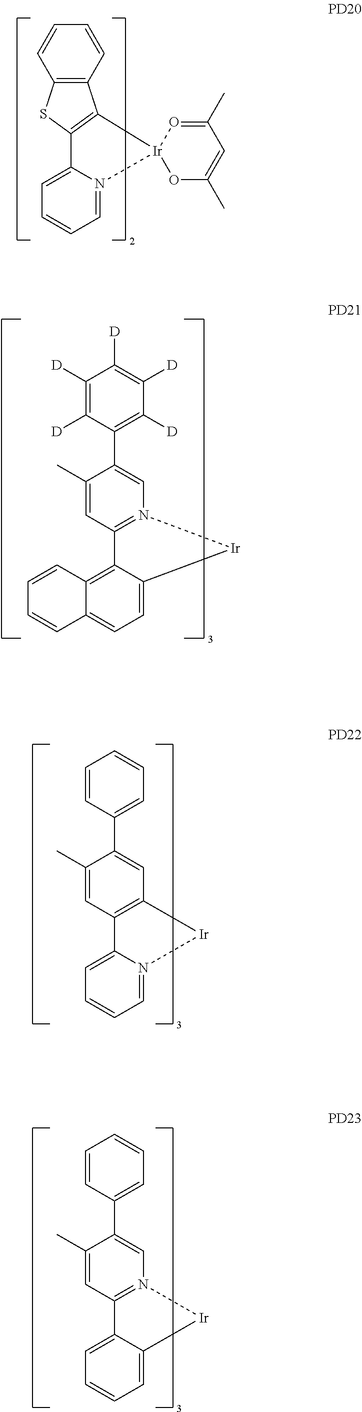

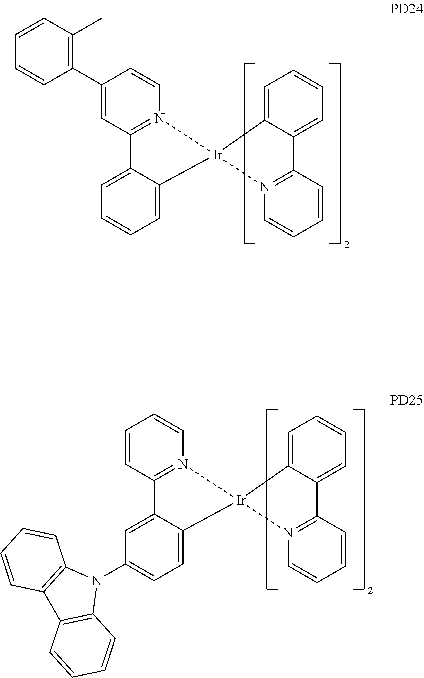

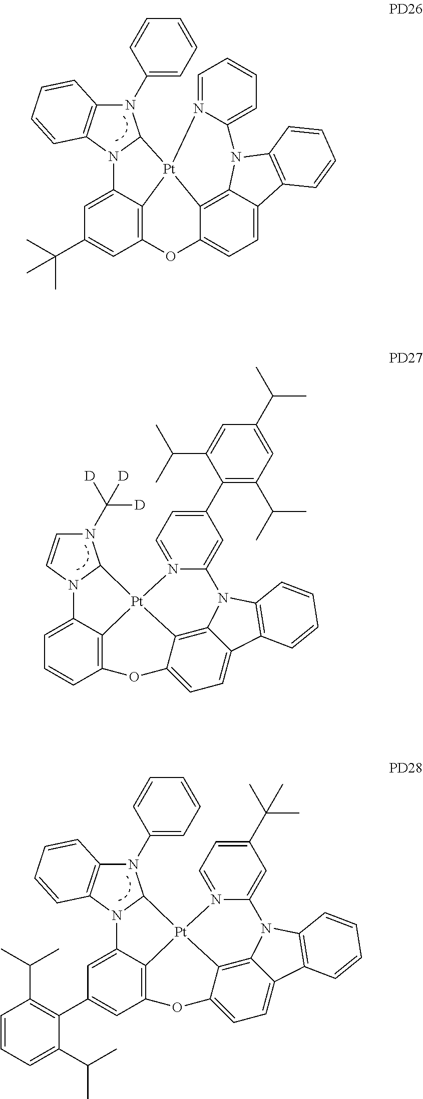

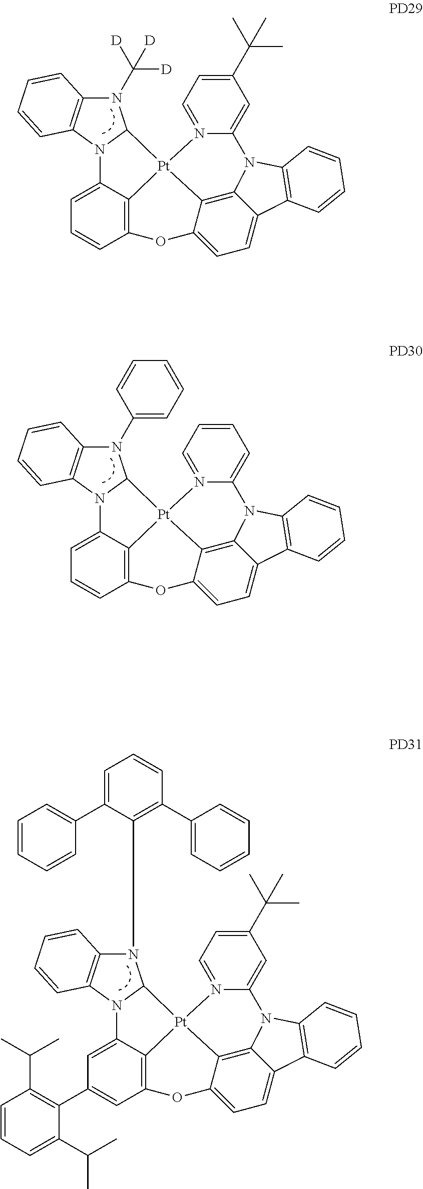

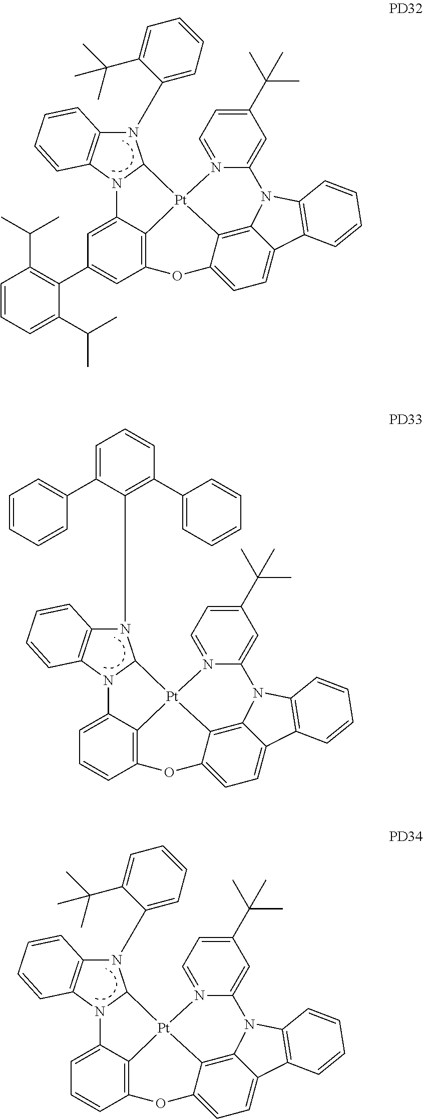

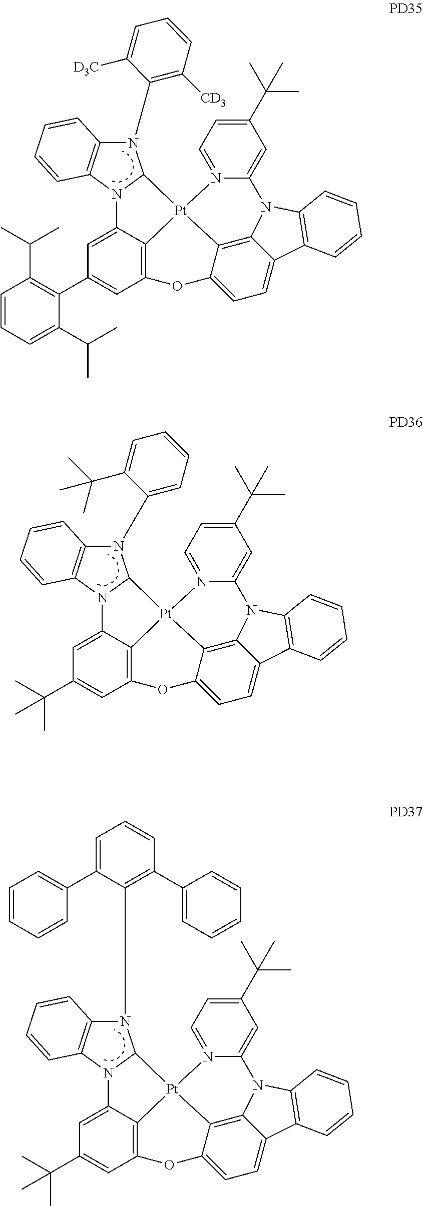

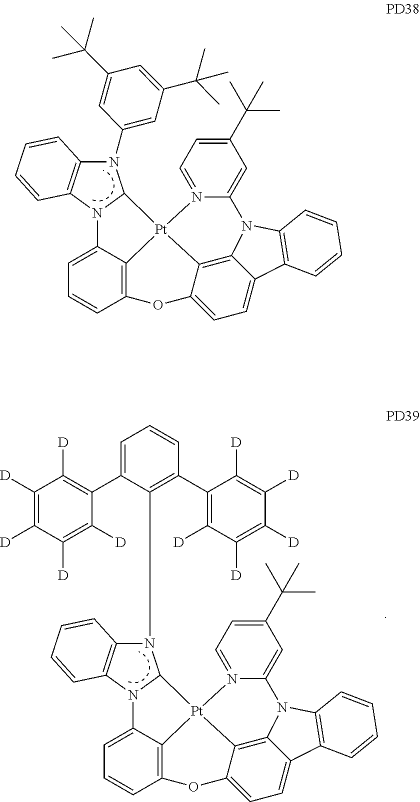

### Fluorescent Dopant

The fluorescent dopant may include an amine group-containing compound, a styryl group-containing compound, or any combination thereof.

For example, the fluorescent dopant may include at least one compound represented by Formula 501:

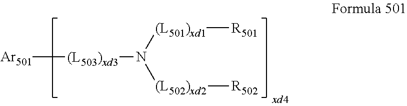

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

For example, the fluorescent dopant may include: at least one (e.g., one or more) selected from among Compounds FD1 to FD36; DPVBi; DPAVBi; or any combination thereof:

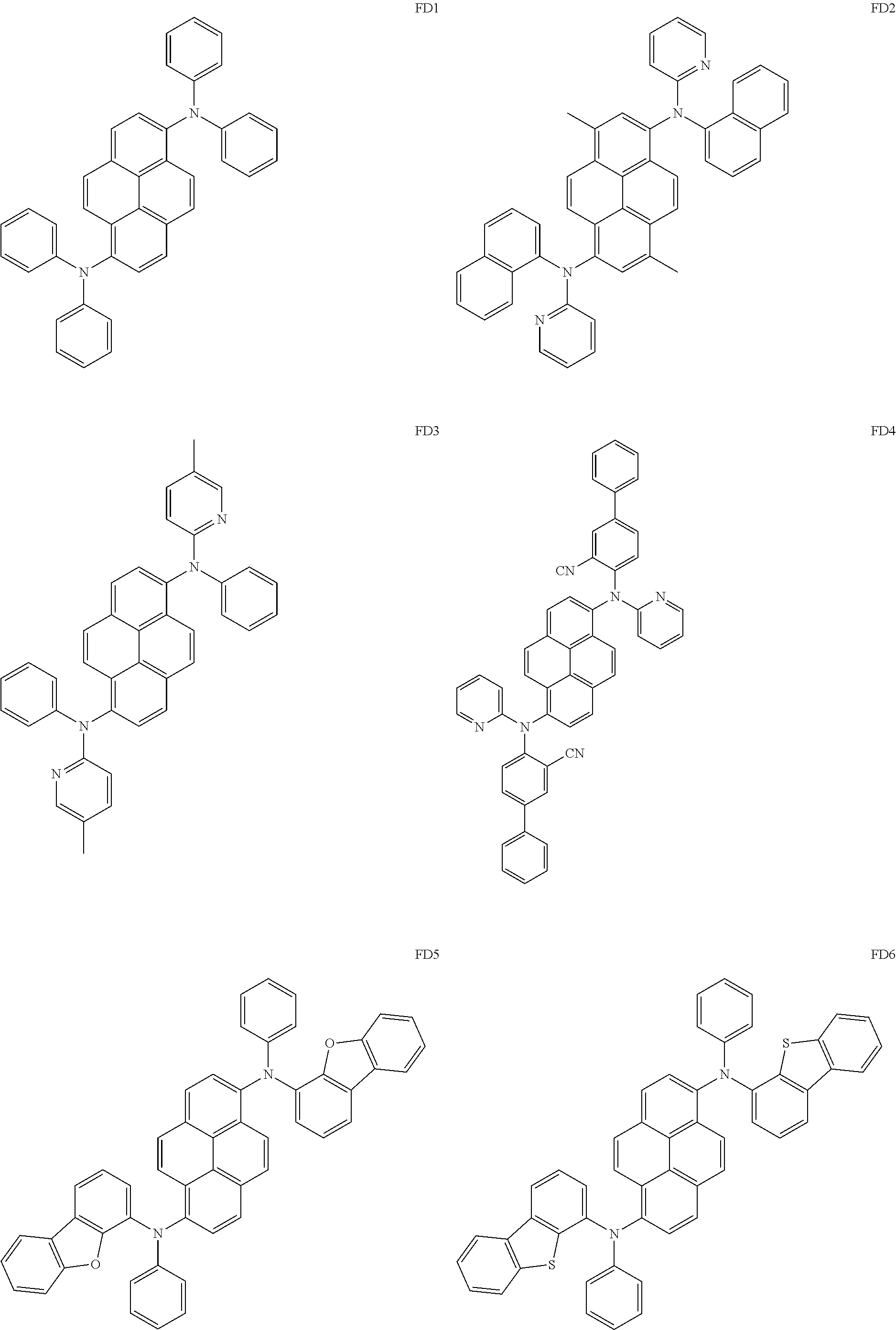

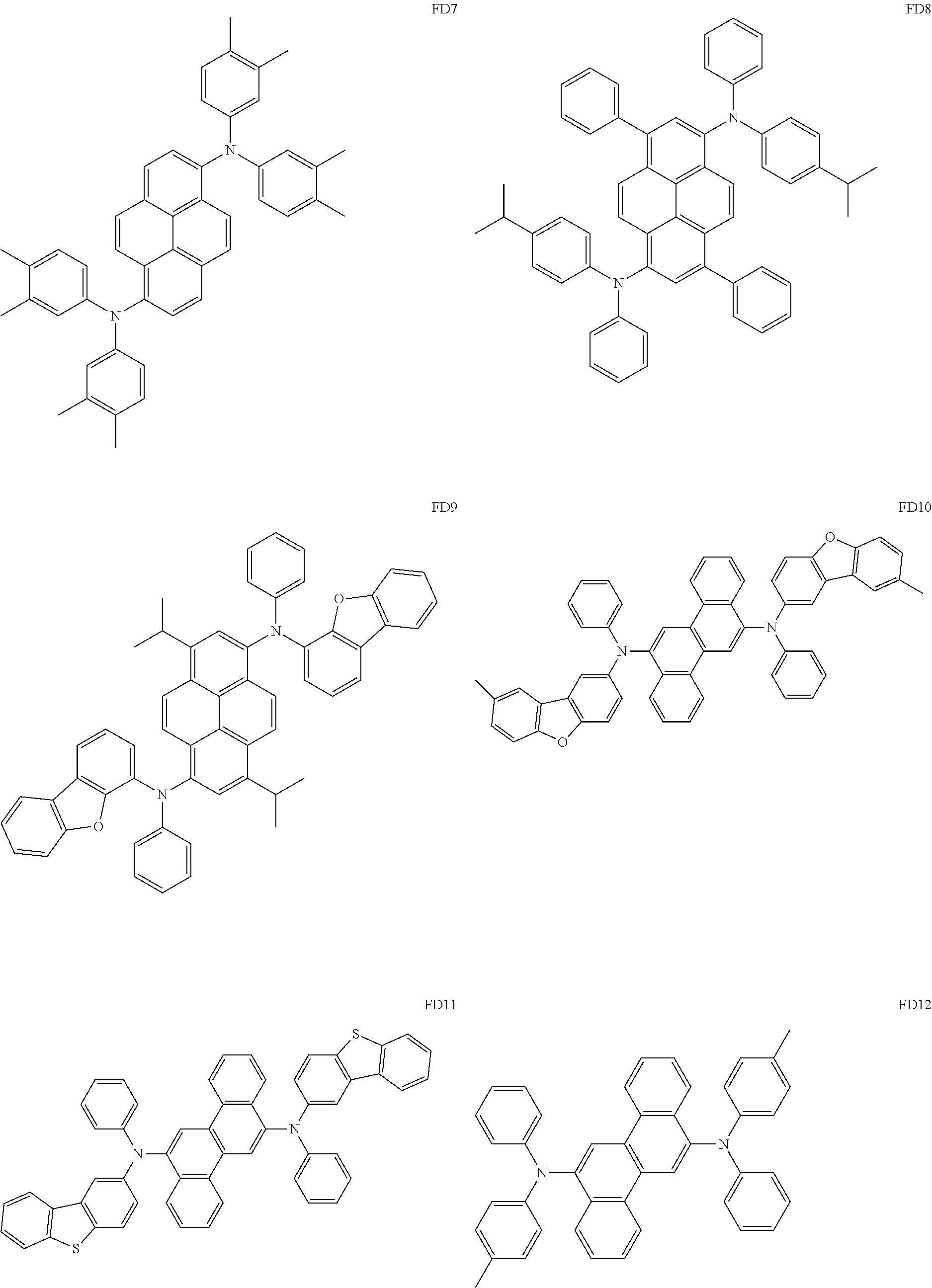

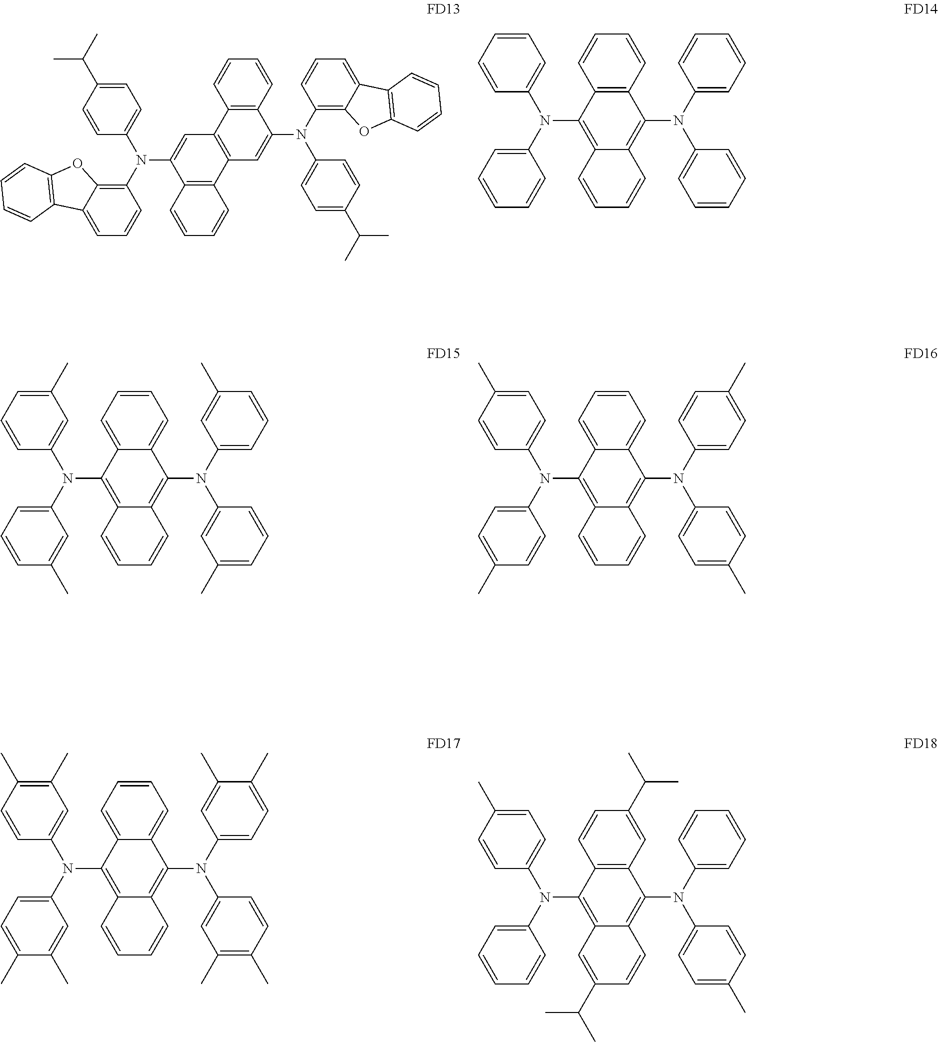

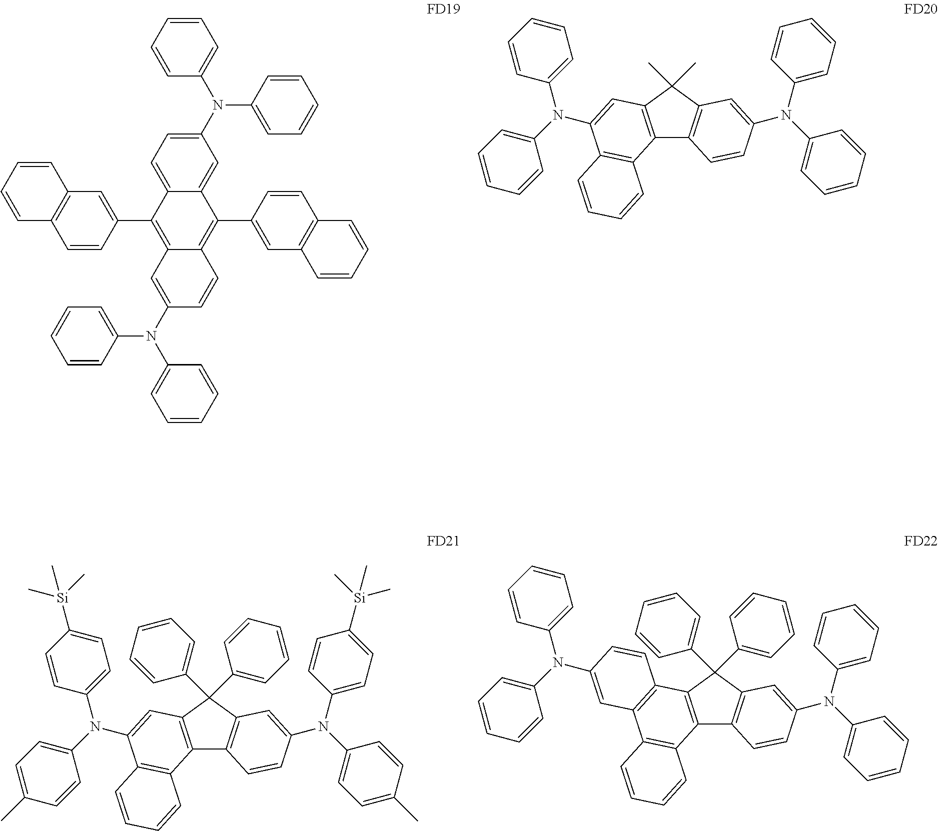

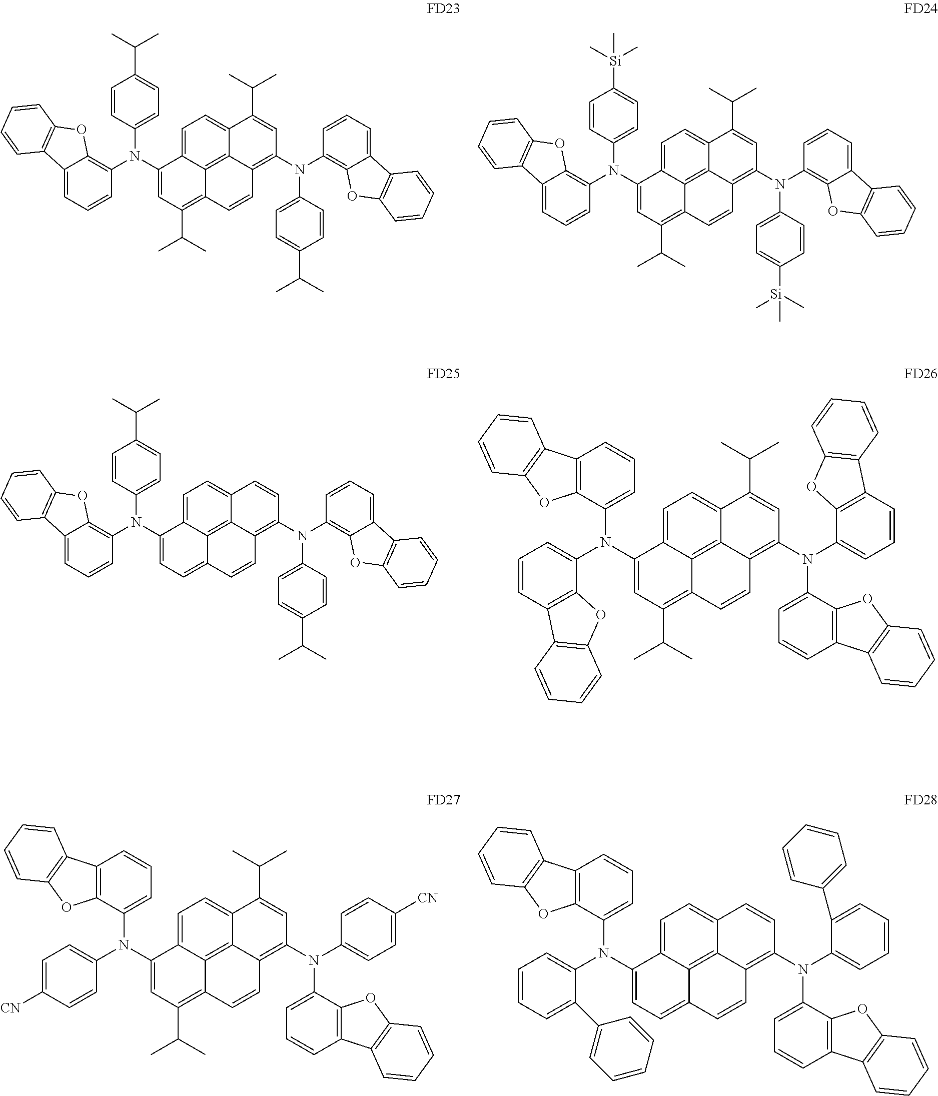

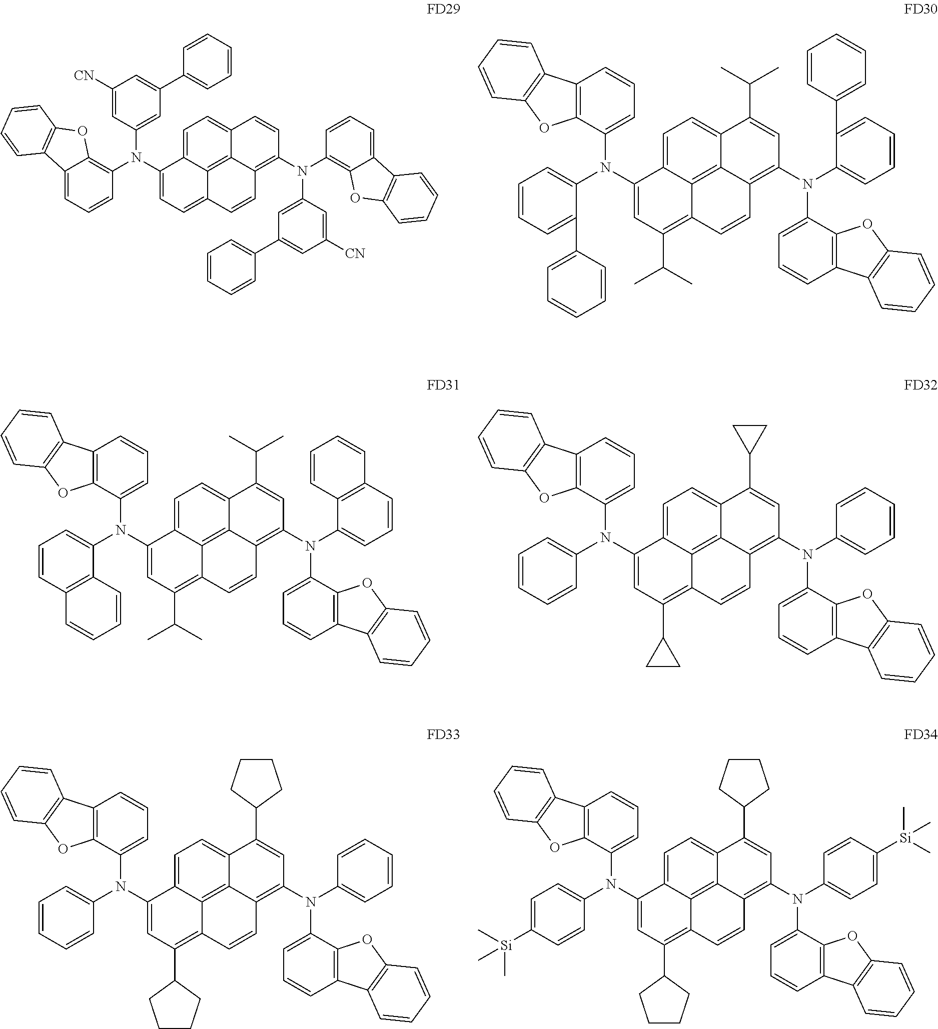

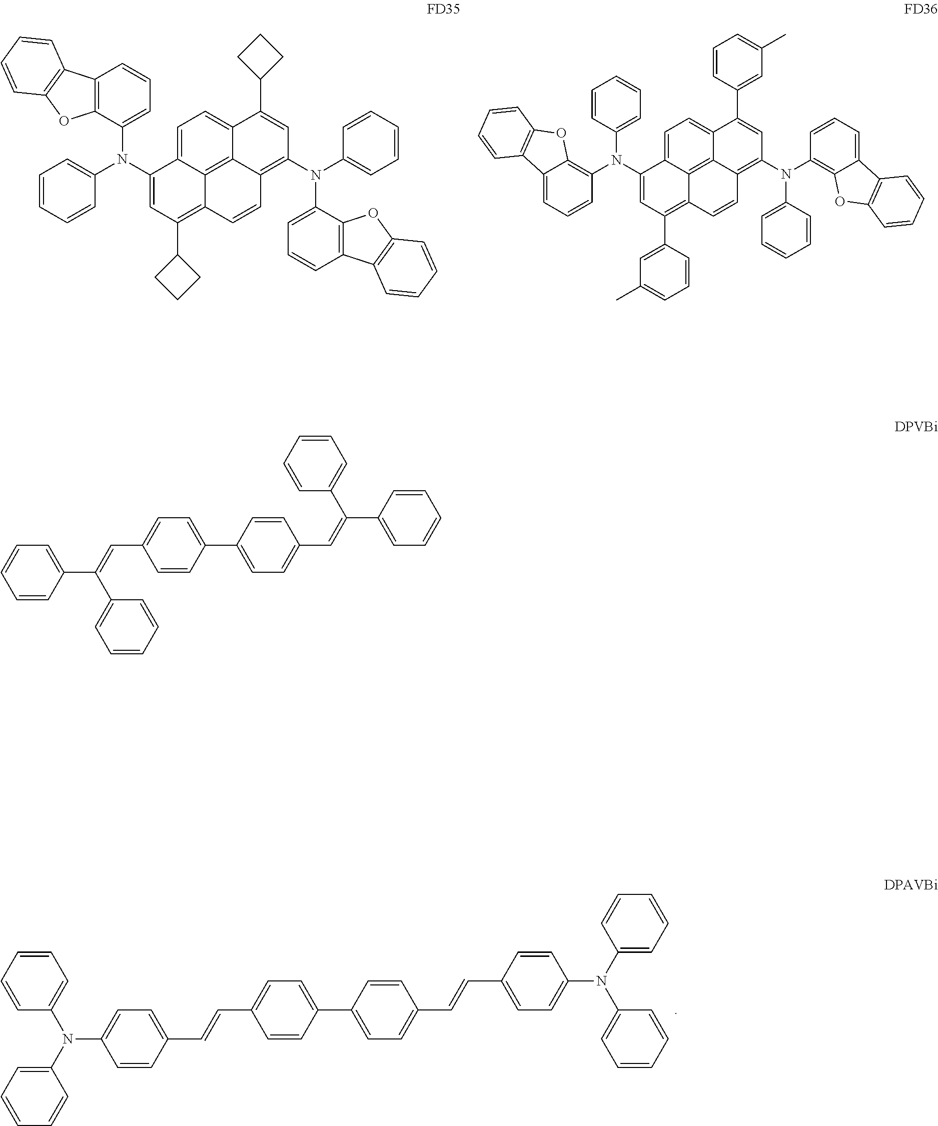

### Delayed Fluorescence Material

The emission layer may include a delayed fluorescence material.

In the specification, the delayed fluorescence material may be any one of (e.g., one selected from among) compounds capable of emitting delayed fluorescent light based on a delayed fluorescence emission mechanism.

The delayed fluorescence material included in the emission layer may act as a host or a dopant depending on the type or kind of other materials included in the emission layer.

According to one or more embodiments, the difference between the triplet energy level of the delayed fluorescence material and the singlet energy level of the delayed fluorescence material may be greater than or equal to about 0 eV and less than or equal to about 0.5 eV. When the difference between the triplet energy level (eV) of the delayed fluorescence material and the singlet energy level (eV) of the delayed fluorescence material satisfies the herein-described range, up-conversion from the triplet state to the singlet state of the delayed fluorescence materials may effectively occur, and thus, the luminescence efficiency of the light-emitting device 10 may be improved.

For example, the delayed fluorescence material may include: i) a material including at least one electron donor (for example, a π electron-rich C₃-C₆₀ cyclic group and/or the like, such as a carbazole group) and at least one electron acceptor (for example, a sulfoxide group, a cyano group, a π electron-deficient nitrogen-containing C₁-C₆₀ cyclic group, and/or the like), and/or ii) a material including a C₈-C₆₀ polycyclic group including at least two cyclic groups condensed to each other while sharing boron (B), and/or the like.

Examples of the delayed fluorescence material may include at least one of (e.g., one or more selected from among) Compounds DF1 to DF14:

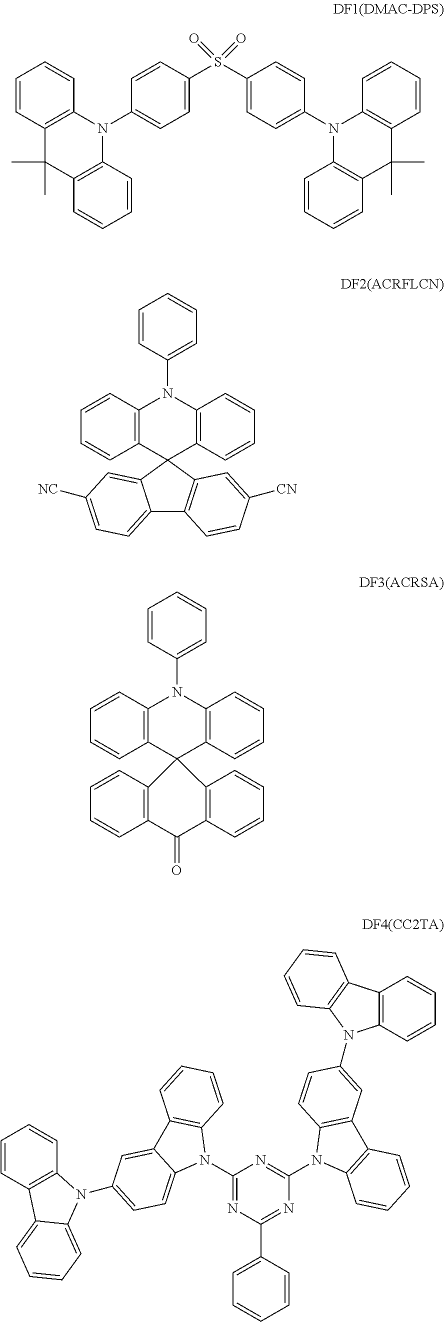

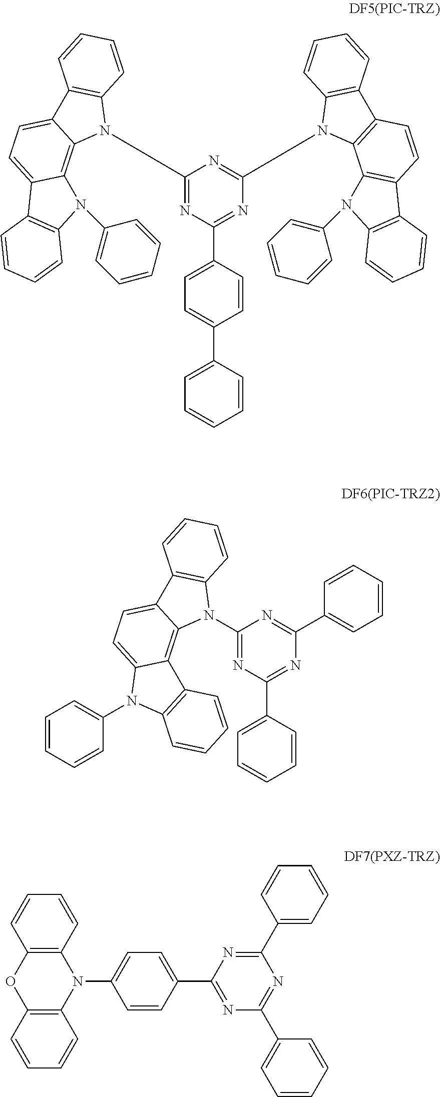 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

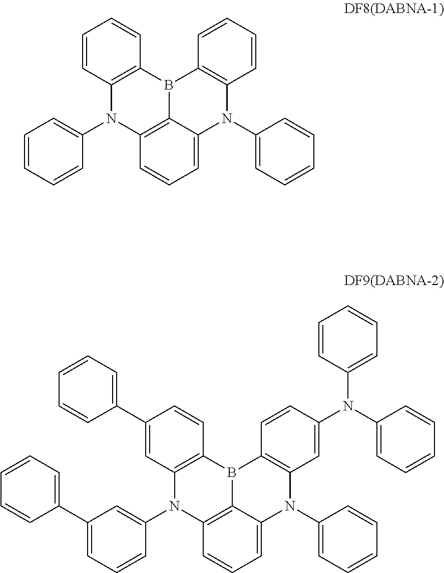 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

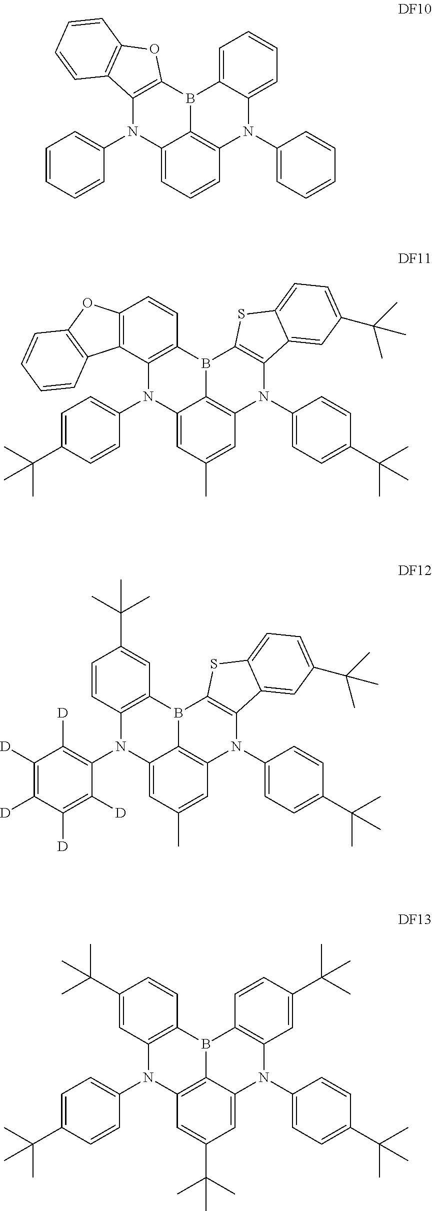

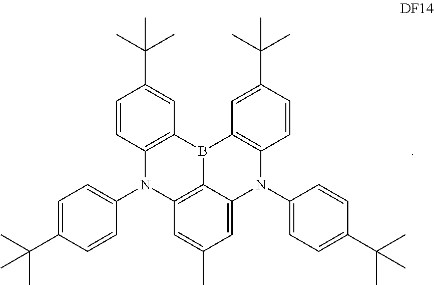 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer may include a quantum dot.

The term “quantum dots” as utilized herein refers to crystals of a semiconductor compound, and may include any material capable of emitting light of one or more suitable emission wavelengths according to the size of the crystals.

A diameter of the quantum dots may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE),

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element or compound, or any combination thereof.

Examples of the Group II-VI semiconductor compound are a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. In one or more embodiments, the Group III-V semiconductor compound may further include a Group II element. Examples of the Group III-V semiconductor compound further including a Group II element are InZnP, InGaZnP, InAlZnP, and/or the like.

Examples of the Group III-VI semiconductor compound are: a binary compound, such as GaS, GaSe, Ga₂Se₃, GaTe, InS, InSe, In₂S₃, In₂Se₃, or InTe; a ternary compound, such as InGaS₃, or InGaSe₃; and any combination thereof.

Examples of the Group I-III-VI semiconductor compound are: a ternary compound, such as AgInS, AgInS₂, CuInS, CuInS₂, CuGaO₂, AgGaO₂, or AgAlO₂; or any combination thereof.

Examples of the Group IV-VI semiconductor compound are: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

The Group IV element or compound may include: a single element, such as Si or Ge; a binary compound, such as SiC or SiGe; or any combination thereof.

Each element included in a multi-element compound such as the binary compound, the ternary compound, and the quaternary compound may be present at a substantially uniform concentration or non-substantially uniform concentration in a particle.

In one or more embodiments, the quantum dot may have a single structure in which the concentration of each element in the quantum dot is substantially uniform, or a core-shell dual structure. For example, the material included in the core and the material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be an oxide of metal, metalloid, or non-metal, a semiconductor compound, and any combination thereof. Examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, CO₃O₄, or NiO; a ternary compound, such as MgAl₂O₄, CoFe₂O₄, NiFe₂O₄, or CoMn₂O₄; and any combination thereof. Examples of the semiconductor compound are, as described herein, a Group II-VI semiconductor compound; a Group III-V semiconductor compound; a Group III-VI semiconductor compound; a Group I-III-VI semiconductor compound; a Group IV-VI semiconductor compound; and any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be about 45 nm or less, for example, about 40 nm or less, or, for example, about 30 nm or less, and within these ranges, color purity or color reproducibility may be increased. In one or more embodiments, because the light emitted through the quantum dot is emitted in all directions, the wide viewing angle may be improved.

In one or more embodiments, the quantum dot may be in the form of (or provide) a spherical nanoparticle, a pyramidal nanoparticle, a multi-arm nanoparticle, a cubic nanoparticle, a nanotube, a nanowire, a nanofiber, or a nanoplate.

Because the energy band gap may be adjusted by controlling the size of the quantum dot, light having one or more suitable wavelength bands may be obtained from the quantum dot emission layer. Accordingly, by utilizing quantum dots of different sizes, a light-emitting device that emits light of one or more suitable wavelengths may be implemented. In one or more embodiments, the size of the quantum dot may be selected to emit red, green and/or blue light. In one or more embodiments, the size of the quantum dot may be configured to emit white light by combination of light of one or more suitable colors.

### Electron Transport Region in Interlayer 130

The electron transport region may have: i) a single-layered structure consisting of a single layer consisting of a single material, ii) a single-layered structure consisting of a single layer including a plurality of different materials, or iii) a multi-layered structure including a plurality of layers including different materials.

The electron transport region may include a buffer layer, a hole-blocking layer, an electron control layer, an electron transport layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the constituting layers of each structure being sequentially stacked from an emission layer.

In one or more embodiments, the electron transport region (for example, the buffer layer, the hole-blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as described herein with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In other embodiments, Ar₆₀₁ in Formula 601 may be an anthracene group unsubstituted or substituted with at least one R₁₀ₐ.

In other embodiments, the electron transport region may include a compound represented by Formula 601-1:

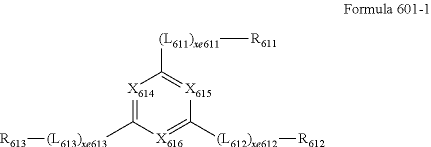

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be as described herein with respect to L₆₀₁,
xe611 to xe613 may each be as described herein with respect to xe1,
R₆₁₁ to R₆₁₃ may each be as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include at least one of (e.g., one or more selected from among) Compounds ET1 to ET45, 2,9-dimethyl-4,7-diphenyl-1,10-phenanthroline (BCP), 4,7-diphenyl-1,10-phenanthroline (Bphen), Alg3, BAlq, TAZ, NTAZ, TSPO1, TPBI, or any combination thereof:

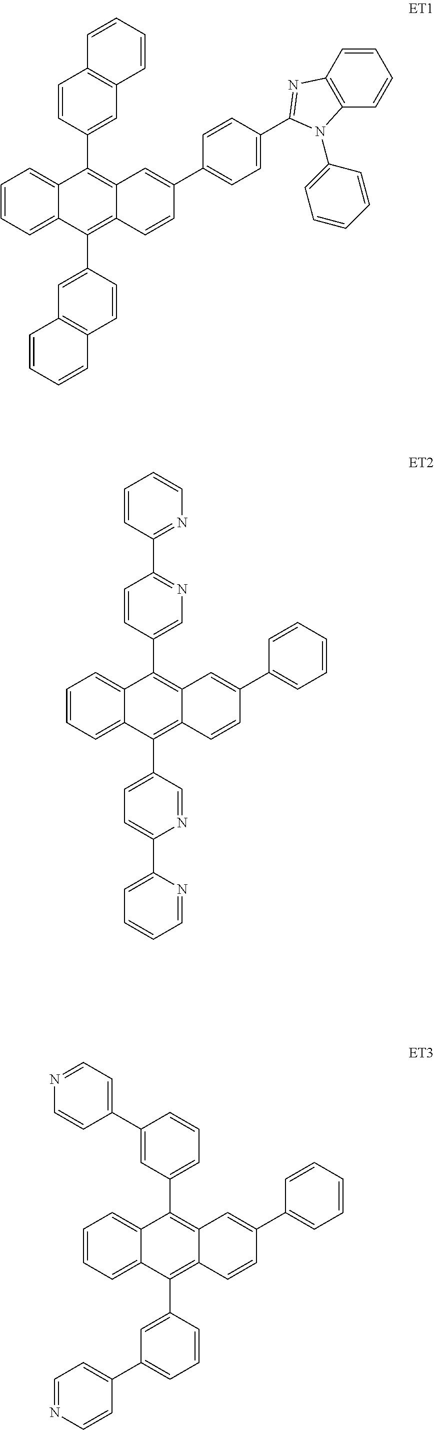 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

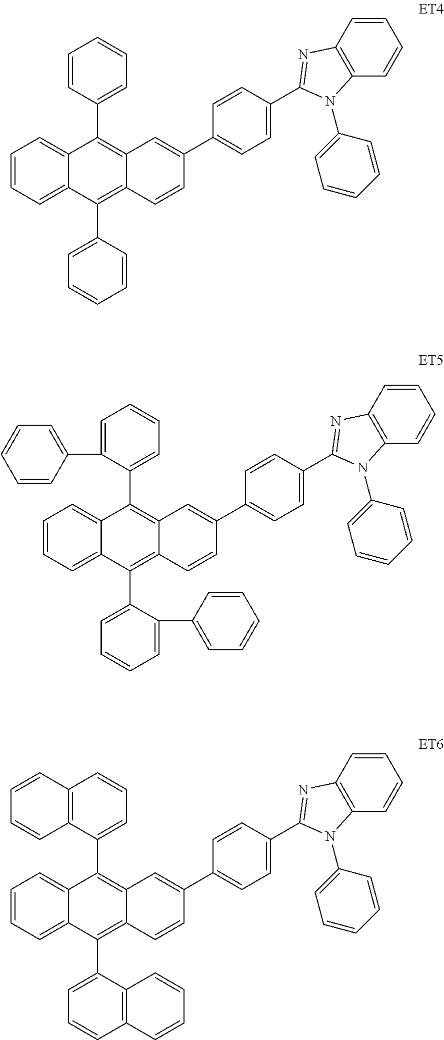 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

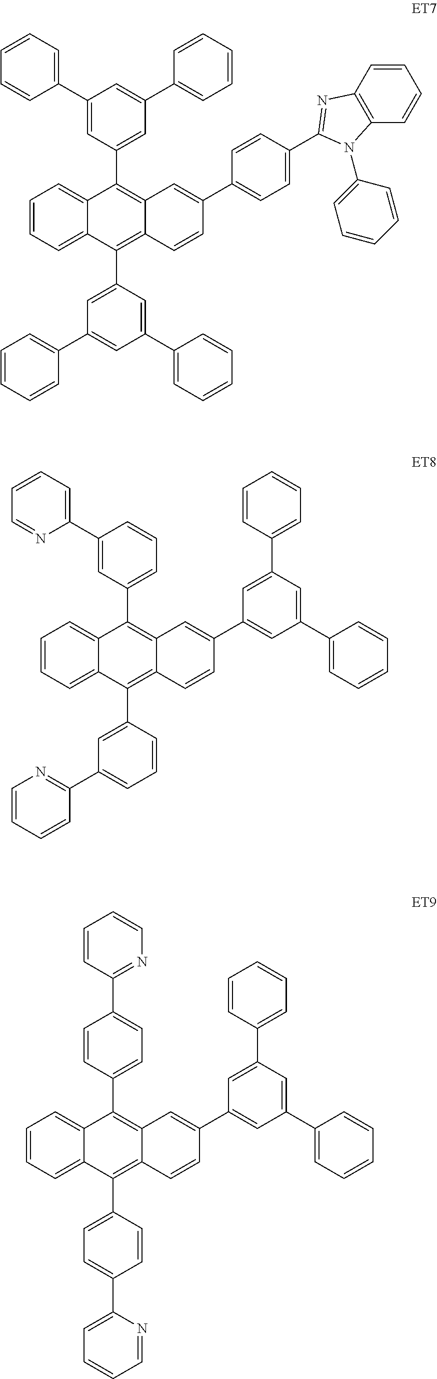 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

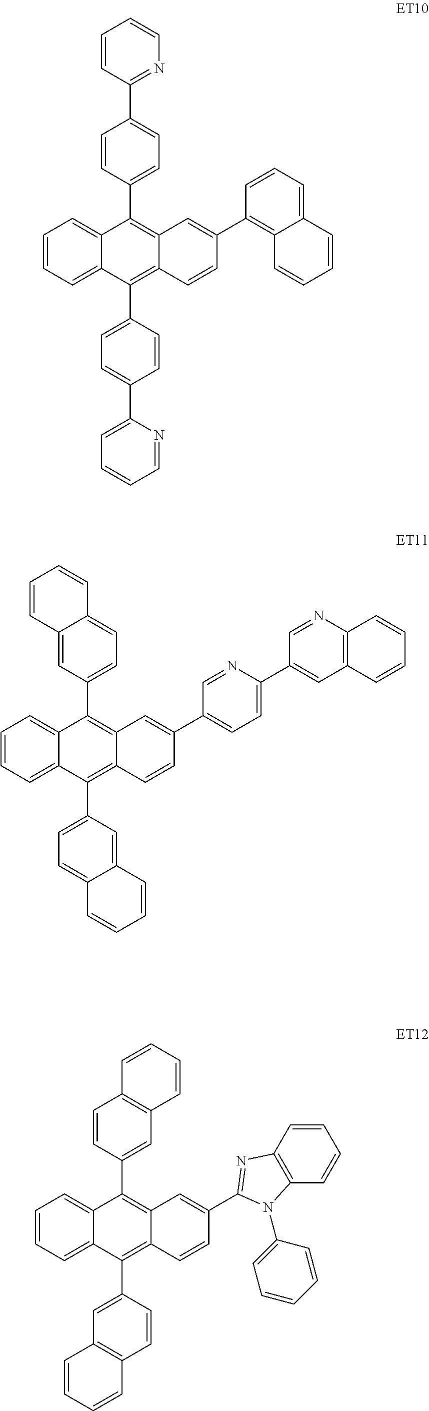 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

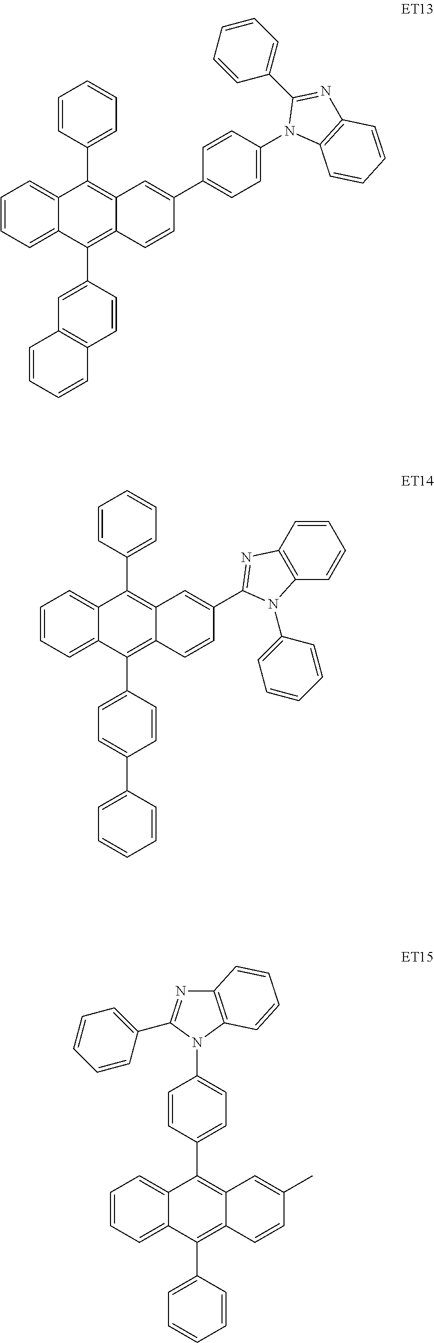 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

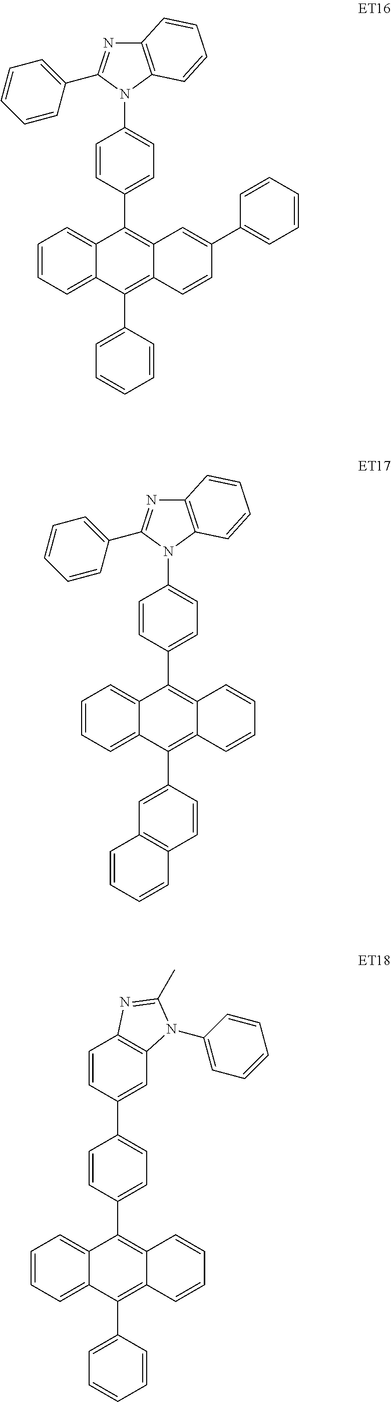 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

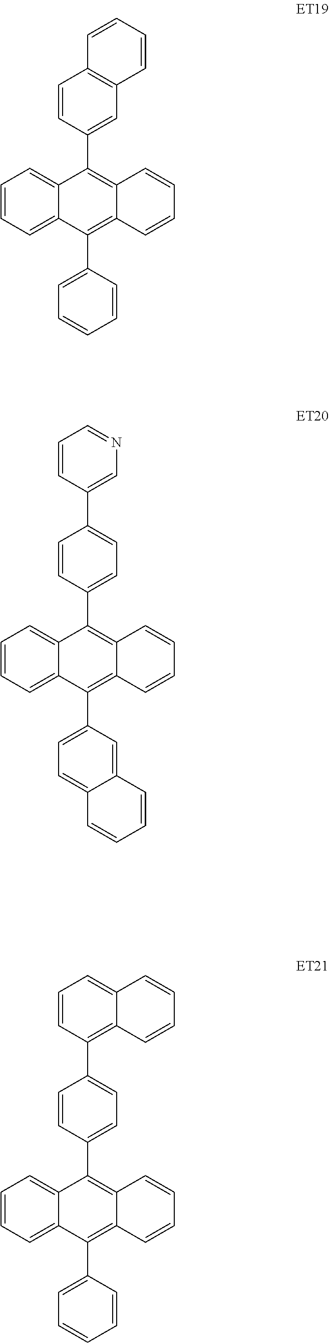

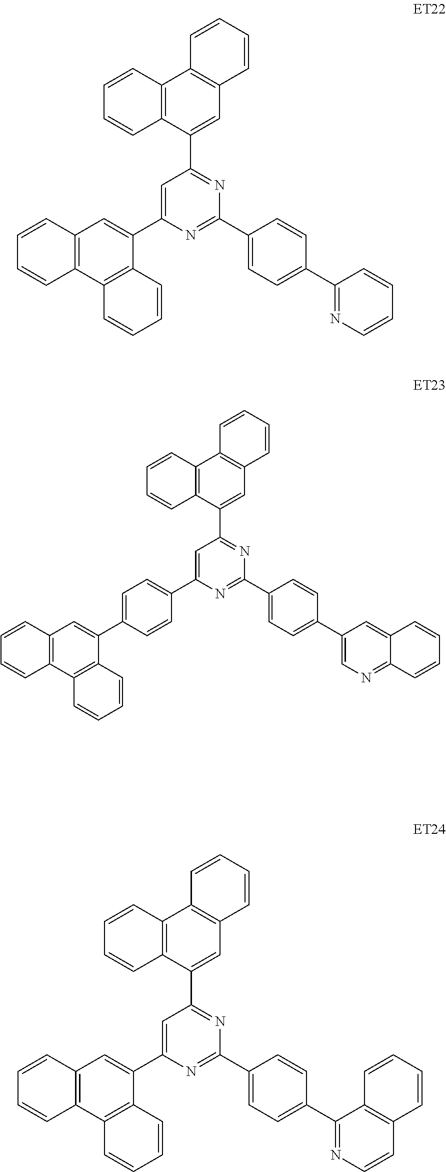 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

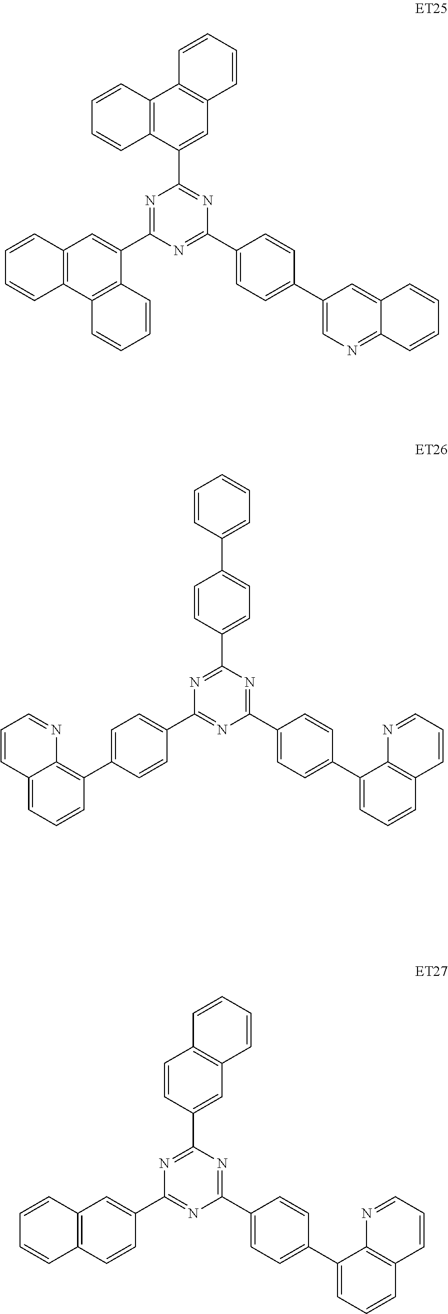 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

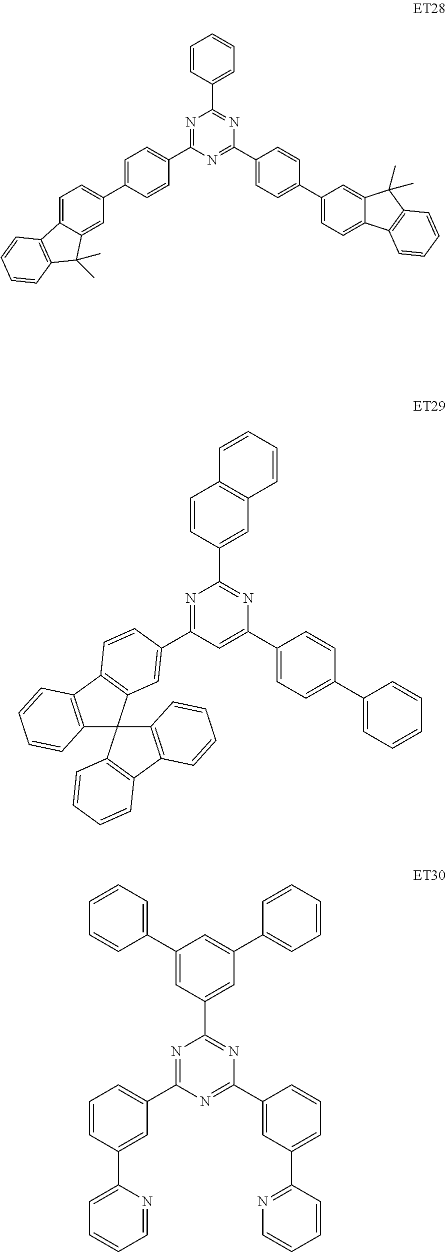 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

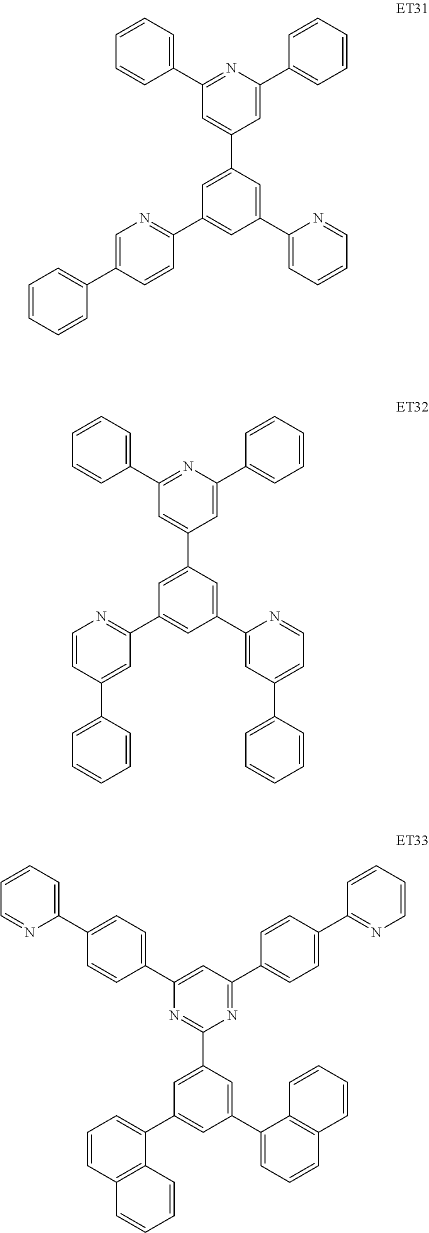 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

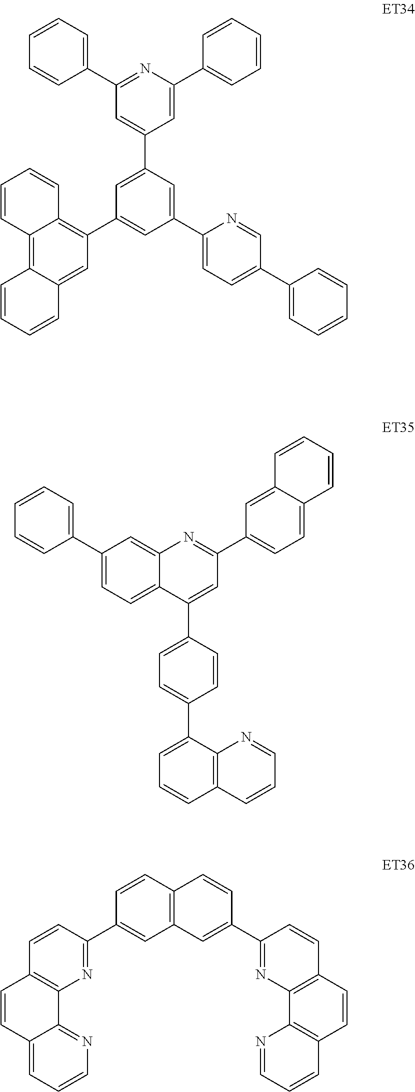 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

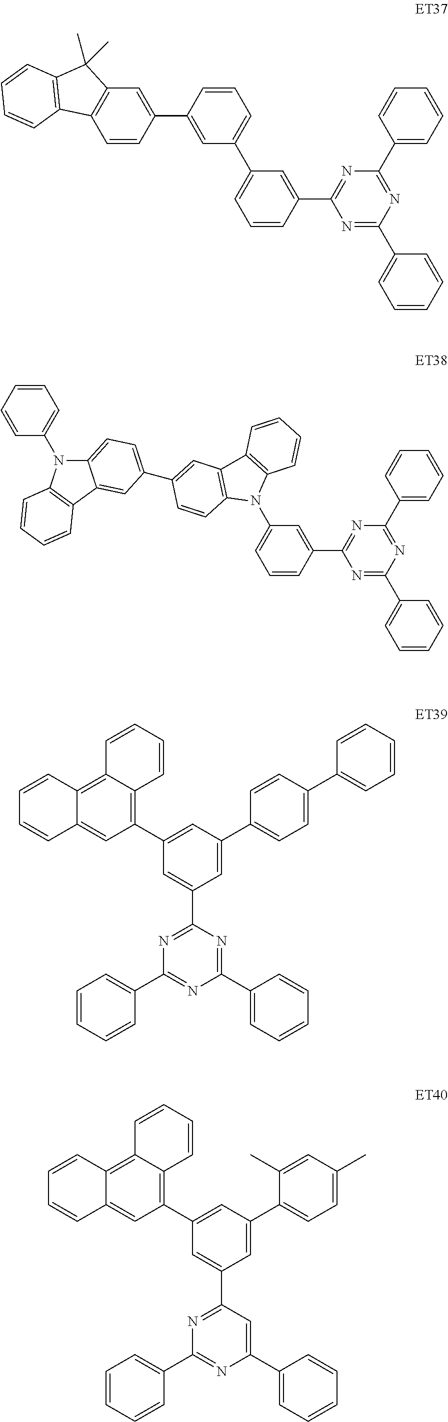 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

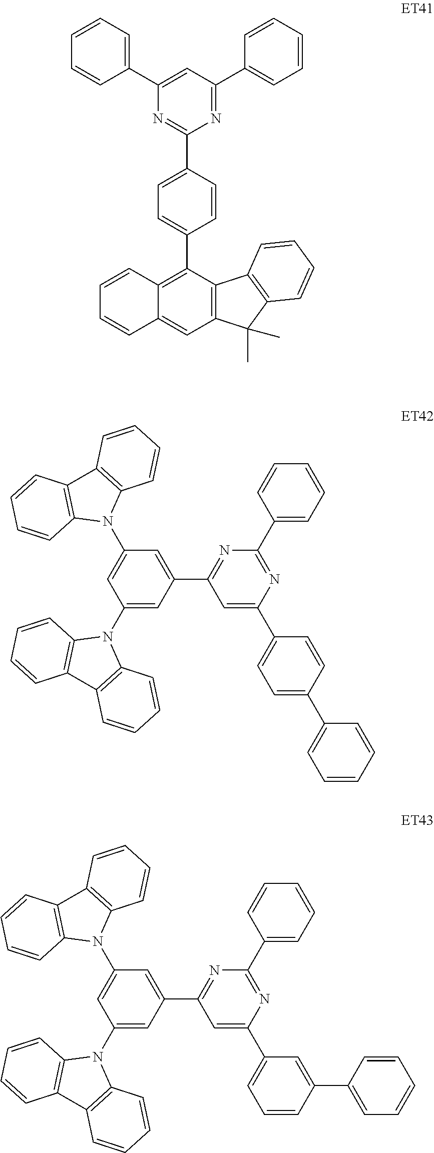 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

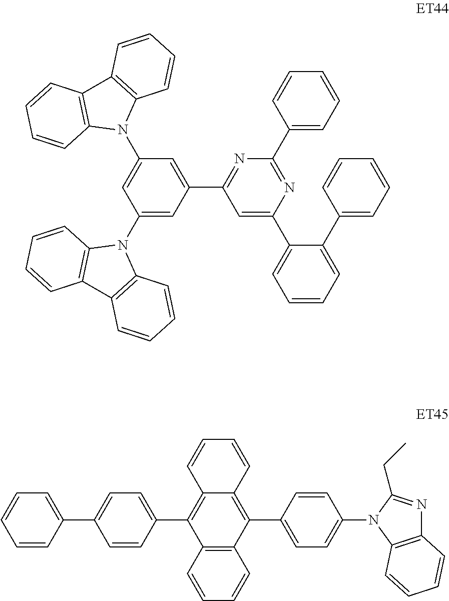 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

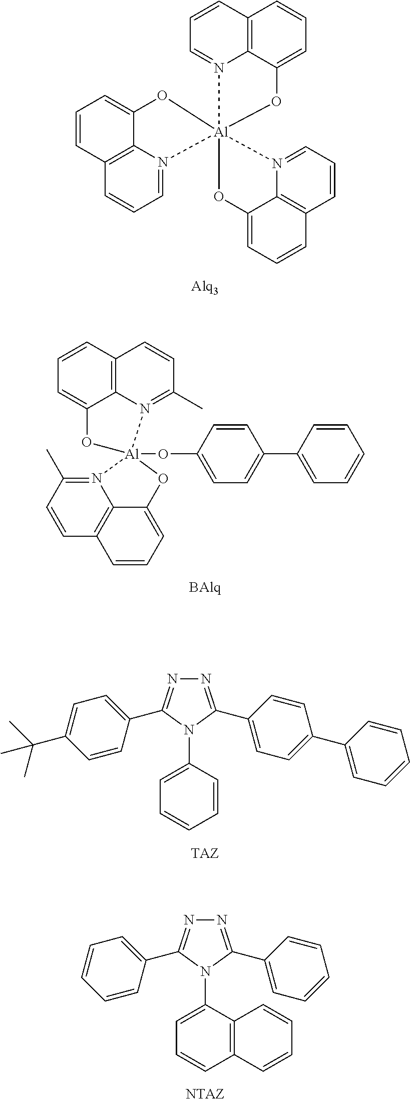

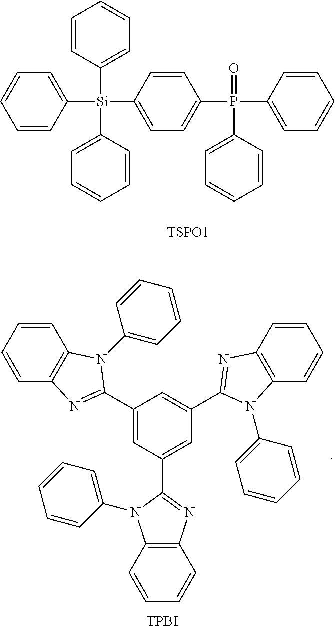

A thickness of the electron transport region may be from about 100 Å to about 5,000 Å, for example, from about 160 Å to about 4,000 Å. When the electron transport region includes a buffer layer, a hole-blocking layer, an electron control layer, an electron transport layer, or any combination thereof, the thickness of the buffer layer, the hole-blocking layer, or the electron control layer may each independently be from about 20 Å to about 1,000 Å, for example, about 30 Å to about 300 Å, and the thickness of the electron transport layer may be from about 100 Å to about 1,000 Å, for example, about 150 Å to about 500 Å. When the thickness of the buffer layer, the hole-blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described herein, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) and/or ET-D2:

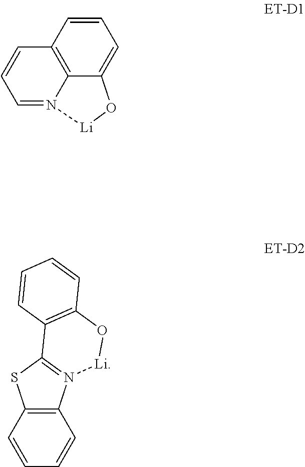

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of (e.g., selected from among) ions of the alkali metal, the alkaline earth metal, and the rare earth metal and ii), as a ligand bonded to the metal ion(s), (e.g., the selected metal ion(s)), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described herein. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly (substantially uniformly) or non-uniformly (substantially uniformly) dispersed in a matrix including the organic material.

A thickness of the electron injection layer may be in a range of about 1 Å to about 100 Å, and, for example, about 3 Å to about 90 Å. When the thickness of the electron injection layer is within the ranges described herein, satisfactory electron injection characteristics may be obtained without a substantial increase in driving voltage.

### Second Electrode 150

The second electrode 150 may be located on the interlayer 130 having a structure as described herein. The second electrode 150 may be a cathode, which is an electron injection electrode, and as the material for the second electrode 150, a metal, an alloy, an electrically conductive compound, or any combination thereof, each having a low-work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be located outside (and e.g., on) the first electrode 110, and/or a second capping layer may be located outside (and e.g., on) the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in an emission layer of the interlayer 130 of the light-emitting device 10 may be extracted toward the outside through the first electrode 110 which is a semi-transmissive electrode or a transmissive electrode, and the first capping layer. Light generated in an emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the aspect of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of (e.g., selected from among) the first capping layer and/or the second capping layer may each independently include carbocyclic compounds, heterocyclic compounds, amine group-containing compounds, porphine derivatives, phthalocyanine derivatives, a naphthalocyanine derivatives, alkali metal complexes, alkaline earth metal complexes, or any combination thereof. Optionally, the carbocyclic compound, the heterocyclic compound, and the amine group-containing compound may be substituted with a substituent including O, N, S, Se, Si, F, Cl, Br, I, or any combination thereof. In one or more embodiments, at least one of (e.g., selected from among) the first capping layer and/or the second capping layer may each independently include an amine group-containing compound.

For example, at least one of (e.g., selected from among) the first capping layer and/or the second capping layer may each independently include a compound represented by Formula 201, a compound represented by Formula 202, or any combination thereof.

In one or more embodiments, at least one of (e.g., selected from among) the first capping layer and/or the second capping layer may each independently include at least one of (e.g., one or more selected from among) Compounds HT28 to HT33, at least one of (e.g., one or more selected from among) Compounds CP1 to CP6, β-NPB, or any combination thereof:

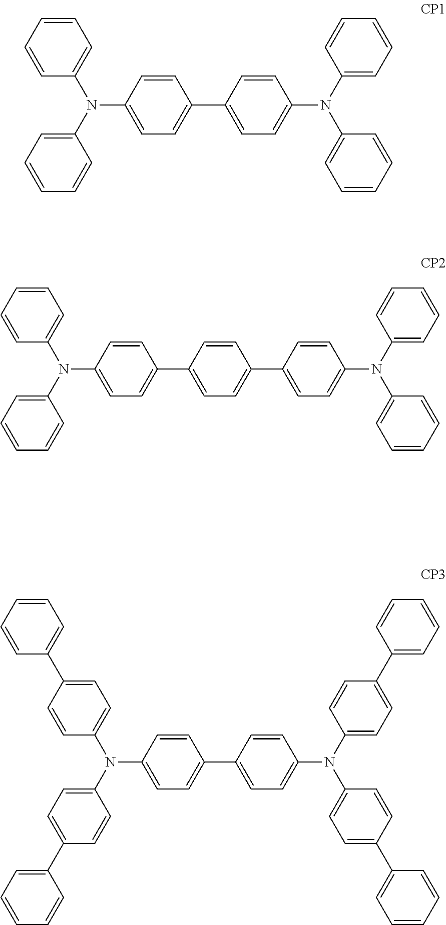 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

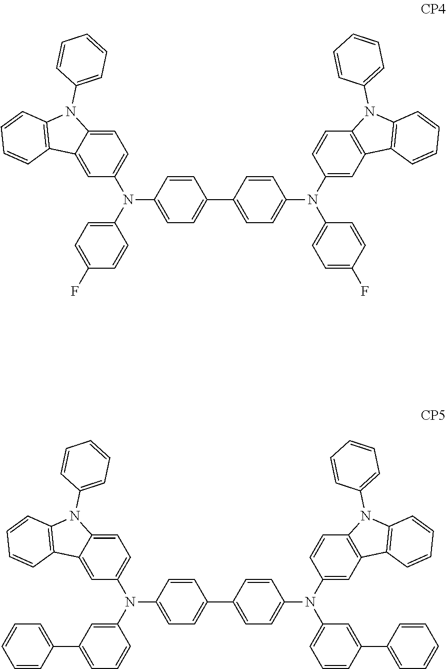 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

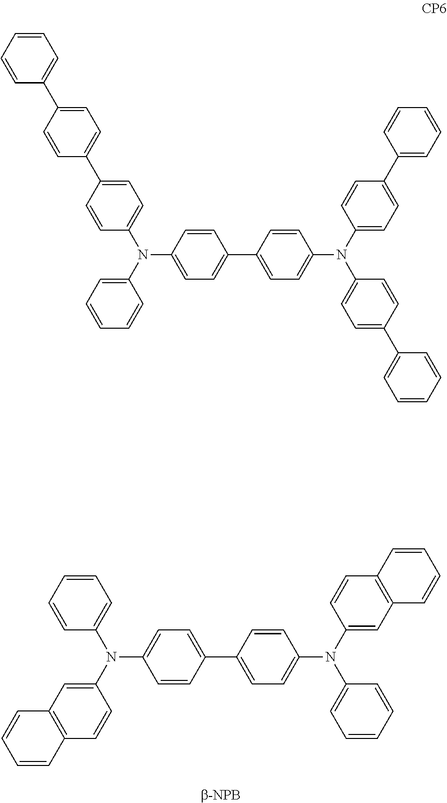 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The amine-containing compound represented by Formula 1 may be included in one or more suitable films. Accordingly, another aspect provides a film including the amine-containing compound represented by Formula 1. The film may be, for example, an optical member (or a light control refers to) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, and/or the like), a protective member (for example, an insulating layer, a dielectric layer, and/or the like).

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be located in at least one direction in which light emitted from the light-emitting device travels. For example, the light emitted from the light-emitting device may be blue light or white light. For details on the light-emitting device, related description provided herein may be referred to. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the plurality of subpixel areas, and the color conversion layer may include a plurality of color conversion areas respectively corresponding to the plurality of subpixel areas.

A pixel-defining layer may be located among the plurality of subpixel areas to define each of the plurality of subpixel areas.

The color filter may further include a plurality of color filter areas and light-shielding patterns located among the plurality of color filter areas, and the color conversion layer may further include a plurality of color conversion areas and light-shielding patterns located among the plurality of color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the plurality of color filter areas (or the plurality of color conversion areas) may include quantum dots. In particular, the first area may include a red quantum dot, the second area may include a green quantum dot, and the third area may not include (e.g., may exclude) a quantum dot. For details on the quantum dot, related descriptions provided herein may be referred to. The first area, the second area, and/or the third area may each include a scatter.

For example, the light-emitting device may be to emit first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum emission wavelengths. In particular, the first light may be blue light, the first-first color light may be red light, the second-first color light may be green light, and the third-first color light may be blue light.

The electronic apparatus may further include a thin-film transistor, in addition to the light-emitting device as described herein. The thin-film transistor may include a source electrode, a drain electrode, and an activation layer, wherein any one (e.g., one) selected from among the source electrode and the drain electrode may be electrically connected to any one (e.g., one) selected from among the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be located between the color filter and/or the color conversion layer and the light-emitting device. The sealing portion allows light from the light-emitting device to be extracted to the outside, and concurrently (e.g., simultaneously) prevents or reduces ambient air and/or moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally located on the sealing portion, in addition to the color filter and/or the color conversion layer, according to the utilize of the electronic apparatus. Examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, and/or the like).

The authentication apparatus may further include, in addition to the light-emitting device as described herein, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device may be included in one or more suitable electronic equipment.

For example, the electronic equipment including the light-emitting device may be at least one selected from among a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater or stadium screen, a phototherapy device, and a signboard.

The light-emitting device may have excellent or suitable effects in terms of luminescence efficiency long lifespan, and thus the electronic equipment including the light-emitting device may have characteristics, such as high luminance, high resolution, and relatively low power consumption.

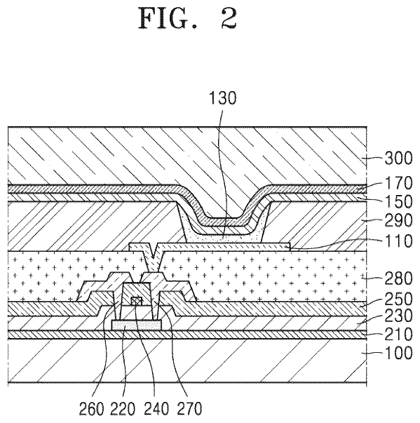

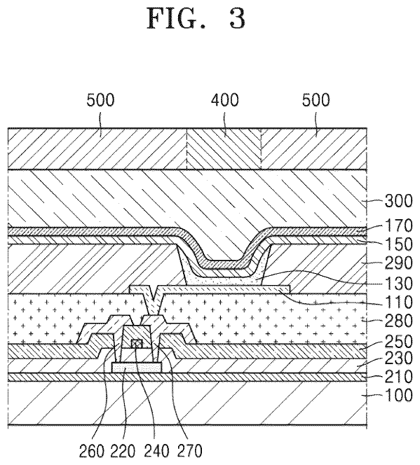

### Description of FIGS. 2 and 3

FIG. 2 is a cross-sectional view showing a light-emitting apparatus according to one or more embodiments of the disclosure.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located on the substrate 100. The buffer layer 210 may prevent or reduce penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be located on the activation layer 220, and the gate electrode 240 may be located on the gate insulating film 230.

An interlayer insulating film 250 may be located on the gate electrode 240. The interlayer insulating film 250 may be located between the gate electrode 240 and the source electrode 260 and between the gate electrode 240 and the drain electrode 270, to insulate from one another.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220.

The TFT may be electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be located on the passivation layer 280. The passivation layer 280 may be located to expose a portion of the drain electrode 270, not fully covering the drain electrode 270, and the first electrode 110 may be located to be connected to the exposed portion of the drain electrode 270.

A pixel defining layer 290 including an insulating material may be located on the first electrode 110. The pixel defining layer 290 may expose a certain region of the first electrode 110, and an interlayer 130 may be formed in the exposed region of the first electrode 110. The pixel defining layer 290 may be a polyimide or polyacrylic organic film. In one or more embodiments, at least some layers of the interlayer 130 may extend beyond the upper portion of the pixel defining layer 290 to be located in the form (or provide) of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture or oxygen. The encapsulation portion 300 may include: an inorganic film including silicon nitride (SiNx, where 0<x<1.8), silicon oxide (SiOx, where 0<x≤2), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 shows a cross-sectional view showing a light-emitting apparatus according to one or more embodiments of the disclosure.

The light-emitting apparatus of FIG. 3 is substantially the same as the light-emitting apparatus of FIG. 2, except that a light-shielding pattern 500 and a functional region 400 are additionally located on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 3 may be a tandem light-emitting device.

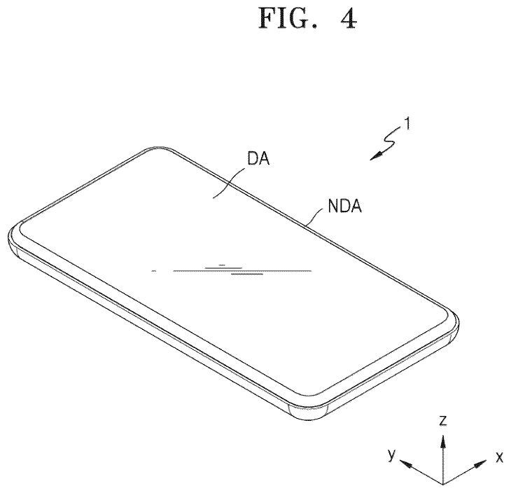

### Description of FIG. 4

FIG. 4 is a schematic perspective view of electronic equipment 1 including a light-emitting device according to one or more embodiments. The electronic equipment 1 may be, as a device apparatus, that displays a moving image or still image, a portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC) as well as one or more suitable products, such as a television, a laptop, a monitor, a billboards or an Internet of things (IOT). The electronic equipment 1 may be such a product herein or a part thereof. In one or more embodiments, the electronic equipment 1 may be a wearable device, such as a smart watch, a watch phone, a glasses-type or kind display, or a head mounted display (HMD), or a part of the wearable device. However, embodiments of the disclosure are not limited thereto. For example, the electronic equipment 1 may be a center information display (CID) on an instrument panel and a center fascia or dashboard of a vehicle, a room mirror display instead of a side mirror of a vehicle, an entertainment display for the rear seat of a car or a display placed on the back of the front seat, head up display (HUD) installed in front of a vehicle or projected on a front window glass, or a computer generated hologram augmented reality head up display (CGH AR HUD). FIG. 4 illustrates a case in which the electronic equipment 1 is a smartphone for convenience of explanation.

The electronic equipment 1 may include a display area DA and a non-display area NDA outside the display area DA. A display device may implement an image through an array of a plurality of pixels that are two-dimensionally arranged in the display area DA.

The non-display area NDA is an area that does not display an image, and may be around (e.g., entirely surround) the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

In the electronic equipment 1, a length in the x-axis direction and a length in the y-axis direction may be different from each other. For example, as shown in FIG. 4, the length in the x-axis direction may be shorter than the length in the y-axis direction. In one or more embodiments, the length in the x-axis direction may be substantially the same as the length in the y-axis direction. In one or more embodiments, the length in the x-axis direction may be longer than the length in the y-axis direction.

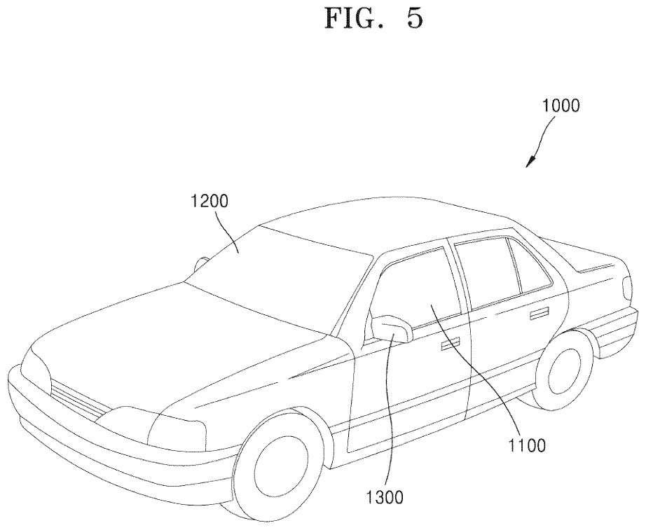

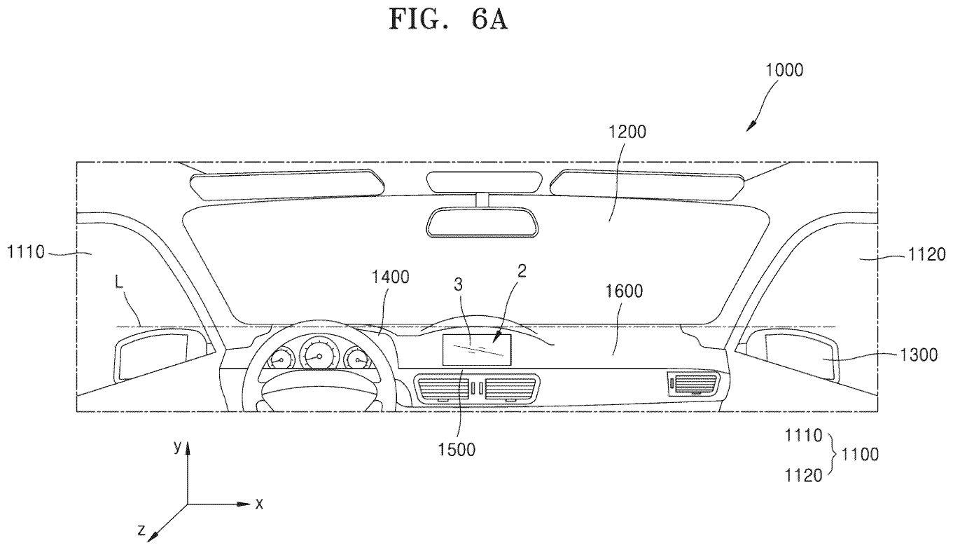

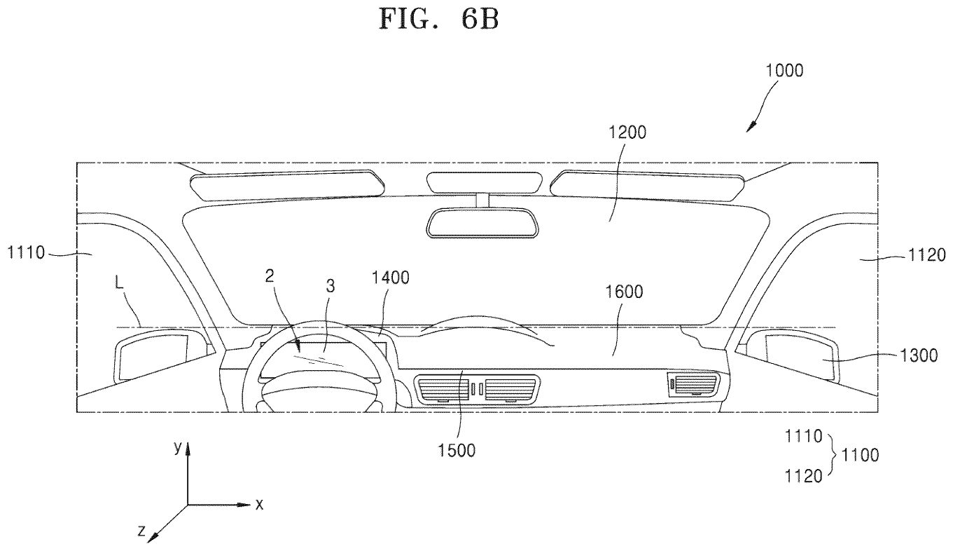

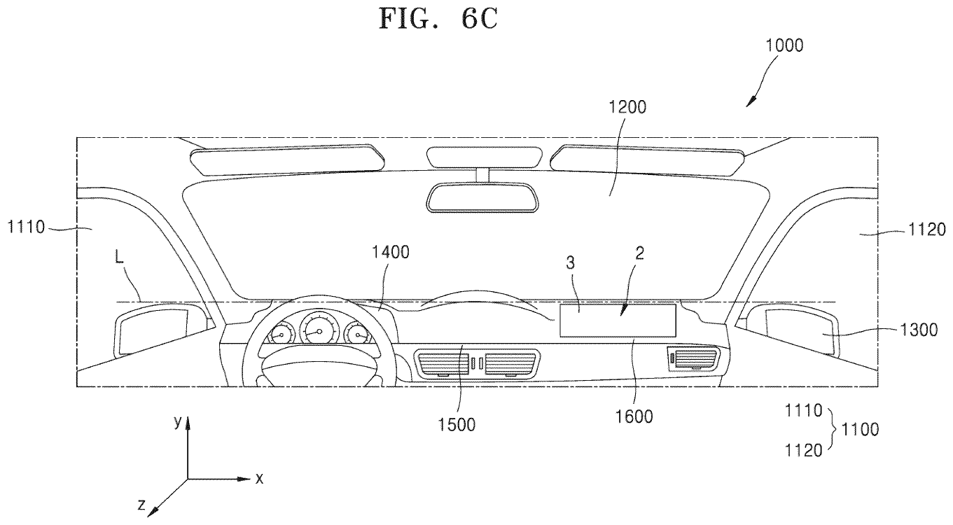

### Descriptions of FIGS. 5 and 6A to 6C

FIG. 5 is a diagram illustrating the exterior of a vehicle 1000 as electronic equipment including a light-emitting device according to one or more embodiments. FIGS. 6A to 6C are each a schematic view of the interior of the vehicle 1000 according to one or more embodiments.

Referring to FIGS. 5, 6A, 6B, and 6C, the vehicle 1000 may refer to one or more suitable apparatuses for moving a subject object to be transported, such as a human, an object, or an animal, from a departure point to a destination. The vehicle 1000 may include a vehicle traveling on a road or track, a vessel moving over a sea or river, an airplane flying in the sky utilizing the action of air, and/or the like.

The vehicle 1000 may travel on a road or a track. The vehicle 1000 may move in a set or predetermined direction according to the rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart from each other in the x-direction or the −x-direction. For example, the first side window glass 1110 and the second side window glass 1120 may be spaced and/or apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one or more embodiments, a plurality of side mirrors 1300 may be provided. Any one selected from among the plurality of side mirrors 1300 may be arranged outside the first side window glass 1110. The other one selected from among the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, a hodometer, an automatic shift selector indicator lamp, a door open warning lamp, an engine oil warning lamp, and/or a relatively low fuel warning light.

The center fascia 1500 may include a control panel on which a plurality of buttons for adjusting an audio device, an air conditioning device, and a heater of a seat are arranged. The center fascia 1500 may be arranged on one side of the cluster 1400.

A passenger seat dashboard 1600 may be spaced and/or apart from the cluster 1400 with the center fascia 1500 arranged therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In one or more embodiments, the display device 2 may be arranged between the side window glasses 1100 facing each other. The display device 2 may be arranged on at least one selected from among the cluster 1400, the center fascia 1500, and the passenger seat dashboard 1600.

The display device 2 may include an organic light-emitting display device, an inorganic electroluminescent (EL) display device, a quantum dot display device, and/or the like. Hereinafter, as the display device 2 according to one or more embodiments of the disclosure, an organic light-emitting display device including the light-emitting device according to the disclosure will be described as an example, but one or more suitable types (kinds) of display devices as described herein may be utilized in embodiments of the disclosure.

Referring to FIG. 6A, the display device 2 may be arranged on the center fascia 1500. In one or more embodiments, the display device 2 may display navigation information. In one or more embodiments, the display device 2 may display audio, video, or information regarding vehicle settings.

Referring to FIG. 6B, the display device 2 may be arranged on the cluster 1400. When the display device 2 is arranged on the cluster 1400, the cluster 1400 may display driving information and/or the like through the display device 2. For example, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, the display device 2 may be arranged on the passenger seat dashboard 1600. The display device 2 may be embedded in the passenger seat dashboard 1600 or arranged on the passenger seat dashboard 1600. In one or more embodiments, the display device 2 arranged on the passenger seat dashboard 1600 may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in a certain region by utilizing one or more suitable methods of (e.g., selected from among) vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group consisting of carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The term “cyclic group” as utilized herein may include both (e.g., simultaneously) the C₃-C₆₀ carbocyclic group and the C₁-C₆₀ heterocyclic group.

The term “π electron-rich C₃-C₆₀ cyclic group” as utilized herein refers to a cyclic group that has 3 to 60 carbon atoms and does not include *—N=*′ as a ring-forming moiety, and the term “π electron-deficient nitrogen-containing C₁-C₆₀ cyclic group” as utilized herein refers to a heterocyclic group that has 1 to 60 carbon atoms and includes *—N═*′ as a ring-forming moiety.

For example,
the C₃-C₆₀ carbocyclic group may be i) Group T1 or ii) a condensed cyclic group in which two or more Groups T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more Groups T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) Group T1, ii) a condensed cyclic group in which two or more Groups T1 are condensed with each other, iii) Group T3, iv) a condensed cyclic group in which two or more Groups T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ cyclic group may be i) Group T4, ii) a condensed cyclic group in which at least two Groups T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as utilized herein refer to a group condensed to any cyclic group, a monovalent group, or a polyvalent group (for example, a divalent group, a trivalent group, a tetravalent group, and/or the like) according to the structure of a formula for which the corresponding term is utilized. In one or more embodiments, “a benzene group” may be a benzo group, a phenyl group, a phenylene group, and/or the like, which may be easily understand by one of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

In some embodiments, examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as utilized herein refers to a linear or branched aliphatic hydrocarbon monovalent group that has one to sixty carbon atoms, and specific examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and examples thereof include an ethynyl group, a propynyl group, and/or the like. The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as utilized herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and specific examples are a 1,2,3,4-oxatriazolidinyl group, a tetrahydrofuranyl group, and a tetrahydrothiophenyl group. The term “C₁-C₁₀ heterocycloalkylene group” as utilized herein refers to a divalent group having the same structure as the C₁-C₁₀ heterocycloalkyl group.

The term C₃-C₁₀ cycloalkenyl group utilized herein refers to a monovalent cyclic group that has three to ten carbon atoms and at least one carbon-carbon double bond in the ring thereof and no aromaticity, and specific examples thereof are a cyclopentenyl group, a cyclohexenyl group, and a cycloheptenyl group. The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having the same structure as the monovalent non-aromatic condensed polycyclic group described herein.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having the same structure as the monovalent non-aromatic condensed heteropolycyclic group described herein.

The term “C₆-C₆₀ aryloxy group” as utilized herein indicates —OA₁₀₂ (wherein A₁₀₂ is a C₆-C₆₀ aryl group), and the term “C₆-C₆₀ arylthio group” as utilized herein indicates —SA₁₀₃ (wherein A₁₀₃ is a C₆-C₆₀ aryl group).

The term “C₇-C₆₀ arylalkyl group” utilized herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroarylalkyl group” utilized herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as utilized herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or [00405]—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom and a hydrogen atom. Examples of the heteroatom are O, S, N, P, Si, B, Ge, Se, and any combinations thereof.

The term “third-row transition metal” utilized herein includes hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “Bu^{t}” as utilized herein refers to a tert-butyl group, and the term “OMe” as utilized herein refers to a methoxy group.

The term “biphenyl group” as utilized herein refers to “a phenyl group substituted with a phenyl group.” In other words, the “biphenyl group” is a substituted phenyl group having a C₆-C₆₀ aryl group as a substituent.

The term “terphenyl group” as utilized herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as utilized herein, unless defined otherwise, each refer to a binding site to a neighboring atom in a corresponding formula or moiety.

In the specification, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting device, light emitting element, and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting device and/or light emitting element may be formed on one integrated circuit (IC) chip or on separate IC chips. Further, the various components of the light emitting device and/or light emitting element may be implemented on a flexible printed circuit film, a tape carrier package (TCP), a printed circuit board (PCB), or formed on one substrate. Further, the various components of the device and/or element may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hereinafter, compounds according to embodiments and light-emitting devices according to embodiments will be described in more detail with reference to the following synthesis examples and examples. The wording “B was utilized instead of A” utilized in describing Synthesis Examples refers to that an identical molar equivalent of B was utilized in place of A.

### EXAMPLES

### Synthesis Example 1: Synthesis of Compound 1

Compound 1 according to one or more embodiments may be synthesized by, for example, utilizing the following reaction.

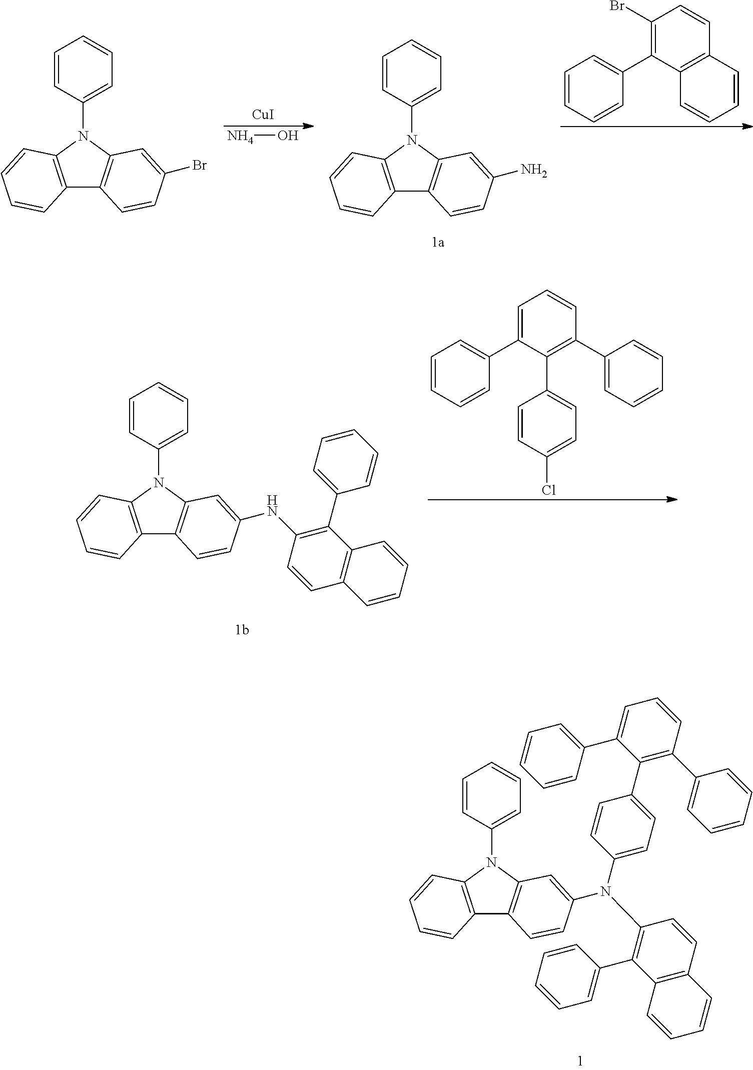

### Synthesis of Intermediate 1a

2-bromo-9-phenyl-9H-carbazole (1.0 eq.), copper(I)iodide (1.0 eq.), and ammonium hydroxide solution (25%, 5.0 eq.) were dissolved in 50 mL of N,N-dimethylformamide, and then stirred under a nitrogen atmosphere at 130° C. for 8 hours in a pressure reaction flask. After the reaction was completed, the reaction product was washed three times utilizing diethyl ether and water, and the obtained organic layer was dried utilizing MgSO₄ and then dried under reduced pressure. Intermediate 1a was obtained by column chromatography. (Yield: 68%)

### Synthesis of Intermediate 1b

Intermediate 1a (1.2 eq.), 2-bromo-1-phenylnaphthalene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 1 b was obtained by column chromatography. (Yield 71%)

### Synthesis of Compound 1

Intermediate 1b (1.0 eq.), 4″-chloro-3′-phenyl-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 1 was obtained by column chromatography. (Yield: 68%)

### Synthesis Example 2: Synthesis of Compound 6

Compound 6 according to one or more embodiments may be synthesized by, for example, utilizing the following reaction.

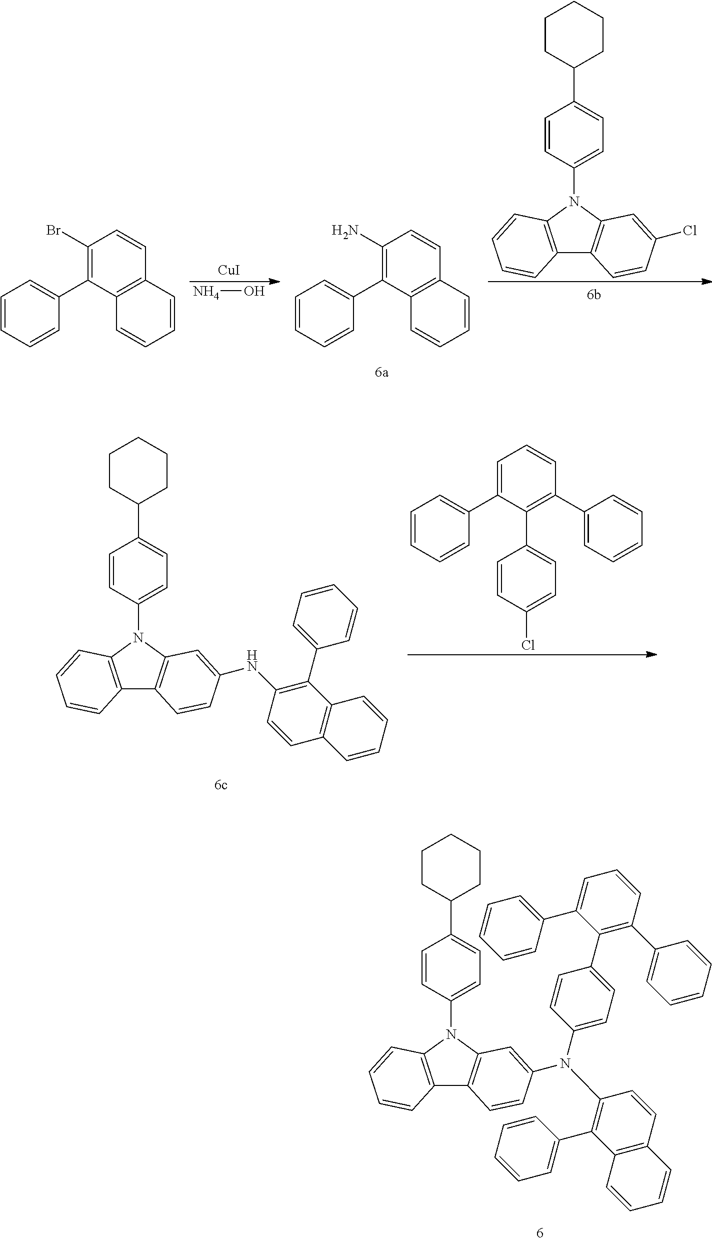

### Synthesis of Intermediate 6a

2-bromo-1-phenylnaphthalene (1.0 eq.), copper(I)iodide (1.0 eq.), and ammonium hydroxide solution (25%, 5.0 eq.) were dissolved in 50 mL of N,N-dimethylformamide, and then stirred under a nitrogen atmosphere at 130° C. for 8 hours in a pressure reaction flask. After the reaction was completed, the reaction product was washed three times utilizing diethyl ether and water, and the obtained organic layer was dried utilizing MgSO₄ and then dried under reduced pressure. Intermediate 6a was obtained by column chromatography. (Yield: 60%)

### Synthesis of Intermediate 6b

1-bromo-4-cyclohexylbenzene (1.0 eq.), 2-chloro-9H-carbazole (1.0 eq.), copper(I)iodide (0.5 eq.), potassium phosphate tribasic (3.0 eq.), and trans-1,2-diaminocyclohexane (2.0 eq.) were dissolved in 50 mL of toluene, followed by stirring under a nitrogen atmosphere at 110° C. for 5 hours. After the reaction was completed, the reaction product was filtered by utilizing celite and washed three times by utilizing water and diethyl ether to obtain an organic layer. The organic layer was dried utilizing MgSO₄ and then dried again under reduced pressure. Intermediate 6b was obtained by column chromatography. (Yield: 80%)

### Synthesis of Intermediate 6c

Intermediate 6a (1.2 eq.), Intermediate 6b (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 6c was obtained by column chromatography. (Yield 63%)

### Synthesis of Compound 6

Intermediate 6c (1.0 eq.), 4″-chloro-3′-phenyl-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 6 was obtained by column chromatography. (Yield: 64%)

### Synthesis Example 3: Synthesis of Compound 33

Compound 33 according to one or more embodiments may be synthesized by, for example, utilizing the following reaction.

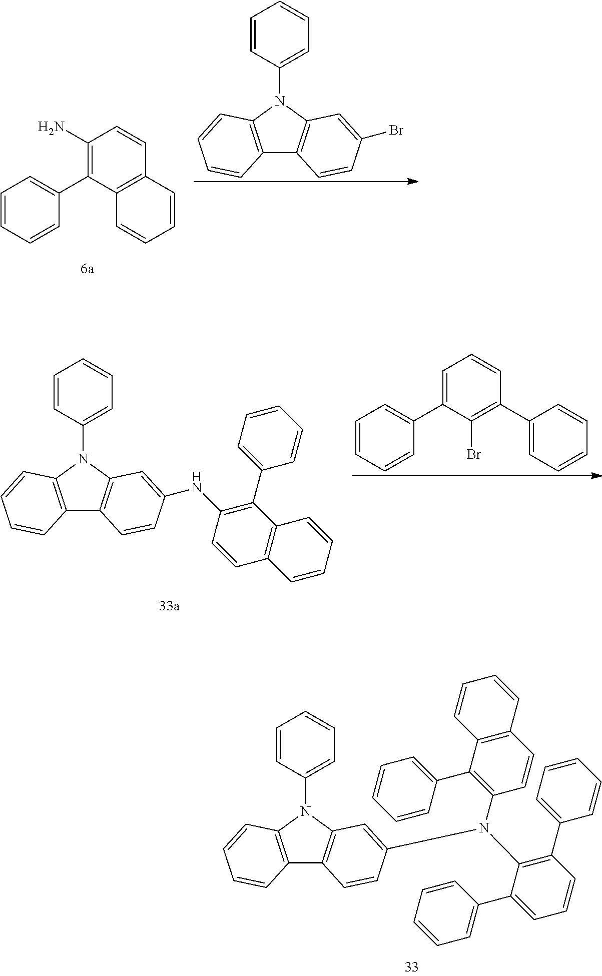

### Synthesis of Intermediate 33a

Intermediate 6a (1.2 eq.), 2-bromo-9-phenyl-9H-carbazole (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 33a was obtained by column chromatography. (Yield: 71%)

### Synthesis of Compound 33

Intermediate 33a (1.0 eq.), 2′-bromo-1,1′:3′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 33 was obtained by column chromatography. (Yield: 45 Synthesis Example 4: Synthesis of Compound 43

Compound 43 according to one or more embodiments may be synthesized by, for example, utilizing the following reaction.

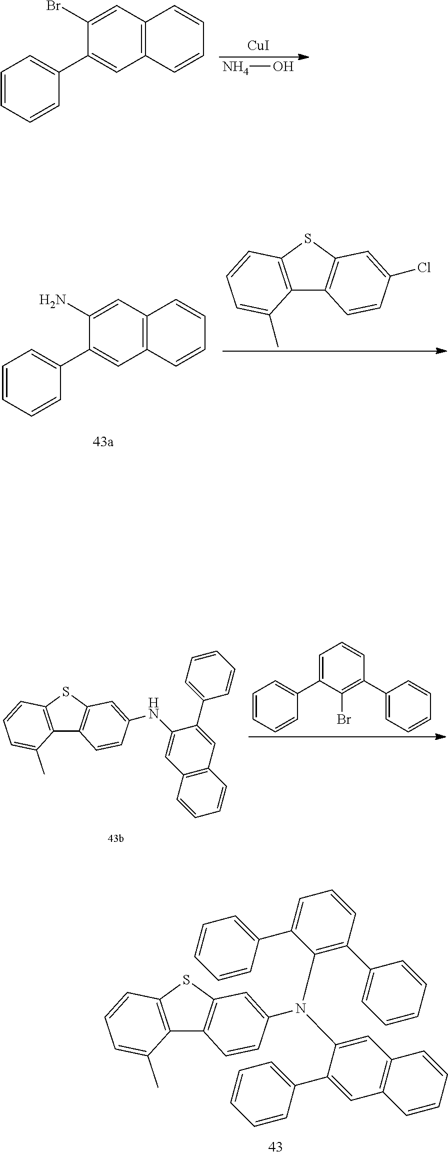

### Synthesis of Intermediate 43a

2-bromo-3-phenylnaphthalene (1.0 eq.), copper(I)iodide (1.0 eq.), and ammonium hydroxide solution (25%, 5.0 eq.) were dissolved in 50 mL of N,N-dimethylformamide, and then stirred under a nitrogen atmosphere at 130° C. for 8 hours in a pressure reaction flask. After the reaction was completed, the reaction product washed three times utilizing diethyl ether and water, and the obtained organic layer was dried utilizing MgSO₄ and then dried under reduced pressure. Intermediate 43a was obtained by column chromatography. (Yield: 61%)

### Synthesis of Intermediate 43b

Intermediate 43a (1.2 eq.), 7-chloro-1-methyldibenzo[b,d]thiophene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Intermediate 43b was obtained by column chromatography. (Yield 65%)

### Synthesis of Compound 43

Intermediate 43b (1.0 eq.), 2′-bromo-1,1′:3′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 43 was obtained by column chromatography. (Yield: 52%)

### Synthesis Example 5: Synthesis of Compound 76

Compound 76 according to one or more embodiments may be synthesized by, for example, utilizing the following reaction.

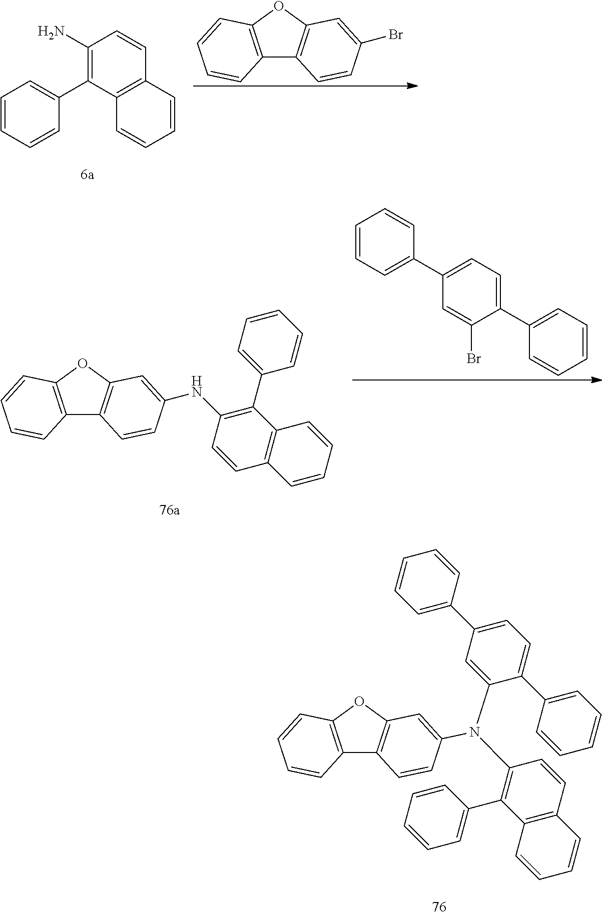

### Synthesis of Intermediate 76a

Intermediate 6a (1.2 eq.), 3-bromodibenzo[b,d]furan (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 76a was obtained by column chromatography. (Yield: 68%)

### Synthesis of Compound 76

Intermediate 76a (1.0 eq.), 2′-bromo-1,1′:4′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 76 was obtained by column chromatography. (Yield: 58%)

### Synthesis Example 6: Synthesis of Compound 147

Compound 147 according to one or more embodiments may be synthesized by, for example, utilizing the following reaction.

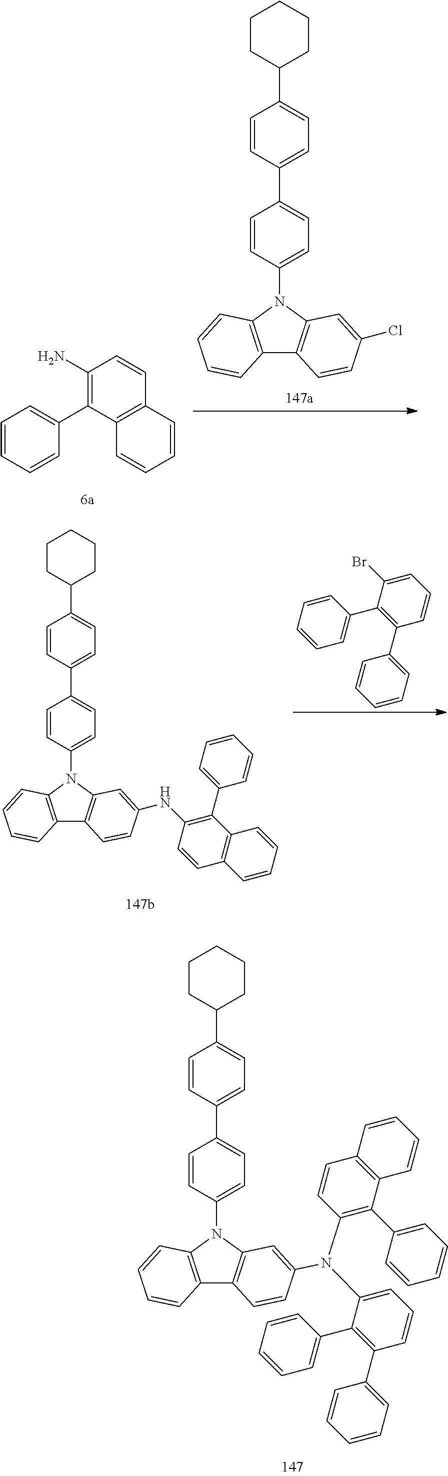 [SMILES: Nc1ccc2ccccc2c1-c1ccccc1 ; Clc1ccc2c3ccccc3n(-c3ccc(-c4ccc(C5CCCCC5)cc4)cc3)c2c1 ; c1ccc(-c2c(Nc3ccc4c5ccccc5n(-c5ccc(-c6ccc(C7CCCCC7)cc6)cc5)c4c3)ccc3ccccc23)cc1 ; Brc1cccc(-c2ccccc2)c1-c1ccccc1 ; c1ccc(-c2cccc(N(c3ccc4c5ccccc5n(-c5ccc(-c6ccc(C7CCCCC7)cc6)cc5)c4c3)c3ccc4ccccc4c3-c3ccccc3)c2-c2ccccc2)cc1]

### Synthesis of Intermediate 147a

4-bromo-4′-cyclohexyl-1,1′-biphenyl (1.0 eq.), 2-chloro-9H-carbazole (1.0 eq.), copper(I)iodide (0.5 eq.), potassium phosphate tribasic (3.0 eq.), and trans-1,2-diaminocyclohexane (2.0 eq.) were dissolved in 50 mL of toluene, followed by stirring under a nitrogen atmosphere at 110° C. for 5 hours. After the reaction was completed, the reaction product was filtered by utilizing celite and washed three times by utilizing water and diethyl ether to obtain an organic layer. The organic layer was dried utilizing MgSO₄ and then dried again under reduced pressure. Intermediate 147a was obtained by column chromatography. (Yield: 77%)

### Synthesis of Intermediate 147b

Intermediate 6a (1.2 eq.), Intermediate 147a (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Intermediate 147b was obtained by column chromatography. (Yield 66%)

### Synthesis of Compound 147

Intermediate 147b (1.0 eq.), 3′-bromo-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 147 was obtained by column chromatography. (Yield 54%)

### Synthesis Example 7: Synthesis of Compound 169

Compound 169 according to one or more embodiments may be synthesized by, for example, utilizing the following reaction.

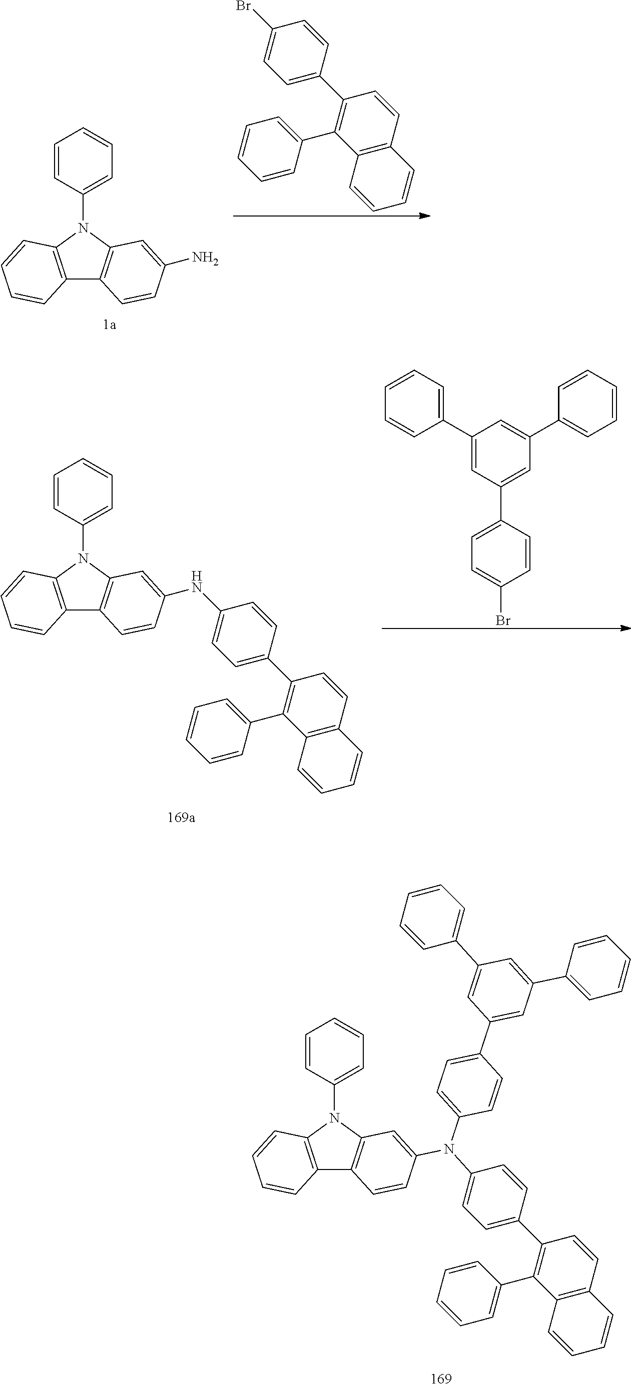 [SMILES: Nc1ccc2c3ccccc3n(-c3ccccc3)c2c1 ; Brc1ccc(-c2ccc3ccccc3c2-c2ccccc2)cc1 ; c1ccc(-c2c(-c3ccc(Nc4ccc5c6ccccc6n(-c6ccccc6)c5c4)cc3)ccc3ccccc23)cc1 ; Brc1ccc(-c2cc(-c3ccccc3)cc(-c3ccccc3)c2)cc1 ; c1ccc(-c2cc(-c3ccccc3)cc(-c3ccc(N(c4ccc(-c5ccc6ccccc6c5-c5ccccc5)cc4)c4ccc5c6ccccc6n(-c6ccccc6)c5c4)cc3)c2)cc1]

### Synthesis of Intermediate 169a

Intermediate 1a (1.2 eq.), 2-(4-bromophenyl)-1-phenylnaphthalene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 169a was obtained by column chromatography. (Yield: 68%)

### Synthesis of Compound 169

Intermediate 169a (1.0 eq.), 4-bromo-5′-phenyl-1,1′:3′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 100° C. for 1 hour. After the reaction was completed, the reaction product was washed three times utilizing diethyl ether and water, and the obtained organic layer was dried utilizing MgSO₄ and then dried under reduced pressure. Compound 169 was obtained by column chromatography. (Yield 64%)

### Synthesis Example 8: Synthesis of Compound 191

Compound 191 according to one or more embodiments may be synthesized by, for example, utilizing the following reaction.

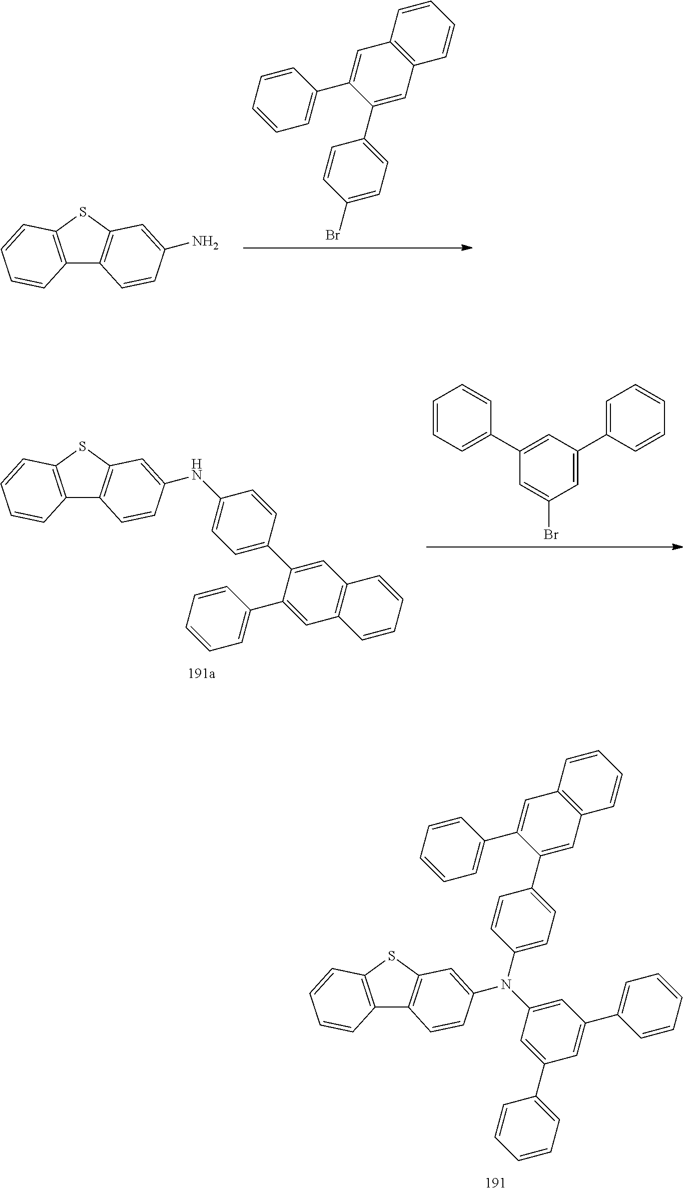 [SMILES: Nc1ccc2c(c1)sc1ccccc12 ; Brc1ccc(-c2cc3ccccc3cc2-c2ccccc2)cc1 ; c1ccc(-c2cc3ccccc3cc2-c2ccc(Nc3ccc4c(c3)sc3ccccc34)cc2)cc1 ; Brc1cc(-c2ccccc2)cc(-c2ccccc2)c1 ; c1ccc(-c2cc(-c3ccccc3)cc(N(c3ccc(-c4cc5ccccc5cc4-c4ccccc4)cc3)c3ccc4c(c3)sc3ccccc34)c2)cc1]

### Synthesis of Intermediate 191a

dibenzo[b,d]thiophen-3-amine (1.2 eq.), 2-(4-bromophenyl)-1-phenylnaphthalene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 191a was obtained by column chromatography. (Yield: 66%)

### Synthesis of Compound 191

Intermediate 191a (1.0 eq.), 5′-bromo-1,1′:3′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 100° C. for 1 hour. After the reaction was completed, the reaction product was washed three times utilizing diethyl ether and water, and the obtained organic layer was dried utilizing MgSO₄ and then dried under reduced pressure. Compound 191 was obtained by column chromatography. (Yield: 54%)

### Synthesis Example 9: Synthesis of Compound 211

Compound 211 according to one or more embodiments may be synthesized by, for example, utilizing the following reaction.

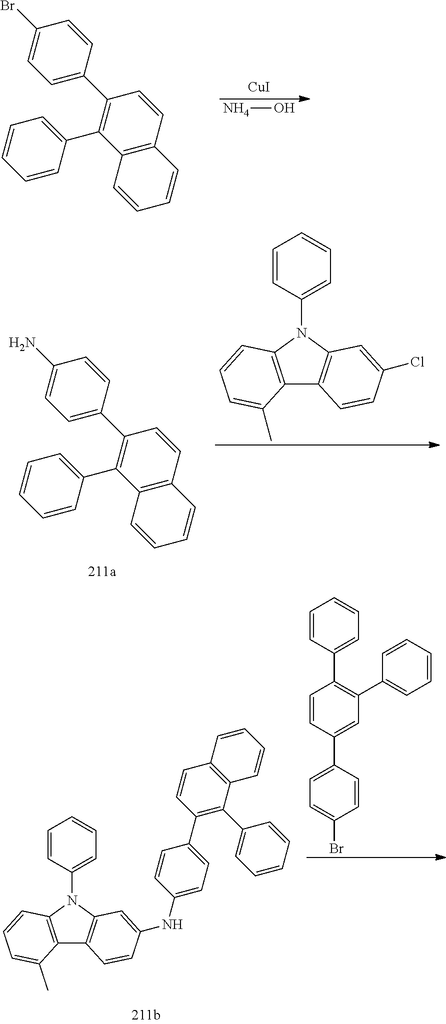

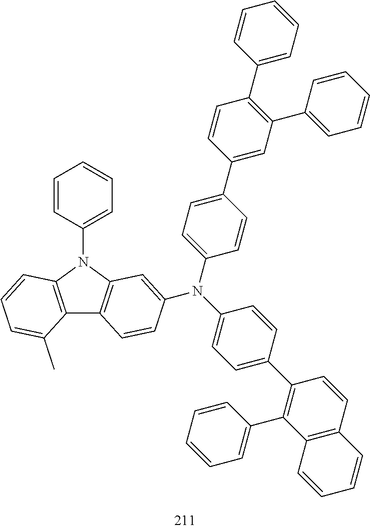 [SMILES: Cc1cccc2c1c1ccc(N(c3ccc(-c4ccc(-c5ccccc5)c(-c5ccccc5)c4)cc3)c3ccc(-c4ccc5ccccc5c4-c4ccccc4)cc3)cc1n2-c1ccccc1]

### Synthesis of Intermediate 211a

2-(4-bromophenyl)-1-phenylnaphthalene (1.0 eq.), copper(I)iodide (1.0 eq.), and ammonium hydroxide solution (25%, 5.0 eq.) were dissolved in 50 mL of N,N-dimethylformamide, and then stirred under a nitrogen atmosphere at 130° C. for 8 hours in a pressure reaction flask. After the reaction was completed, the reaction product was washed three times utilizing diethyl ether and water, and the obtained organic layer was dried utilizing MgSO₄ and then dried under reduced pressure. Intermediate 211a was obtained by column chromatography. (Yield: 65%)

### Synthesis of Intermediate 211b

Intermediate 211a (1.2 eq.), 2-chloro-5-methyl-9-phenyl-9H-carbazole (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Intermediate 211 b was obtained by column chromatography. (Yield 62%)

### Synthesis of Compound 211

Intermediate 211b (1.0 eq.), 4′-(4-bromophenyl)-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 211 was obtained by column chromatography. (Yield 68%)

### Synthesis Example 10: Synthesis of Compound 226

Compound 226 according to one or more embodiments may be synthesized by, for example, utilizing the following reaction.

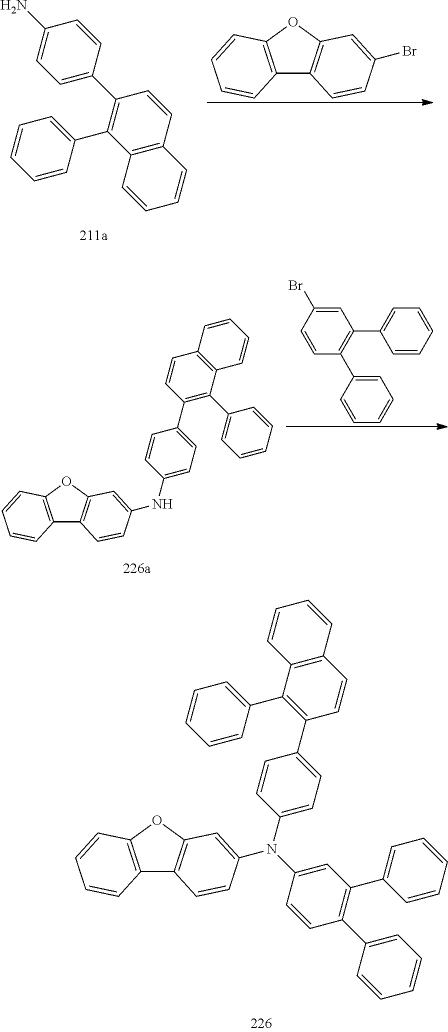 [SMILES: Nc1ccc(-c2ccc3ccccc3c2-c2ccccc2)cc1 ; Brc1ccc2c(c1)oc1ccccc12 ; c1ccc(-c2c(-c3ccc(Nc4ccc5c(c4)oc4ccccc45)cc3)ccc3ccccc23)cc1 ; Brc1ccc(-c2ccccc2)c(-c2ccccc2)c1 ; c1ccc(-c2ccc(N(c3ccc(-c4ccc5ccccc5c4-c4ccccc4)cc3)c3ccc4c(c3)oc3ccccc34)cc2-c2ccccc2)cc1]

### Synthesis of Intermediate 226a

Intermediate 211a (1.2 eq.), 3-bromodibenzo[b,d]furan (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 226a was obtained by column chromatography. (Yield 60%)

### Synthesis of Compound 226

Intermediate 226a (1.0 eq.), 4′-bromo-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 226 was obtained by column chromatography. (Yield 58%)

### Synthesis Example 11: Synthesis of Compound 229

Compound 229 according to one or more embodiments may be synthesized by, for example, utilizing the following reaction.

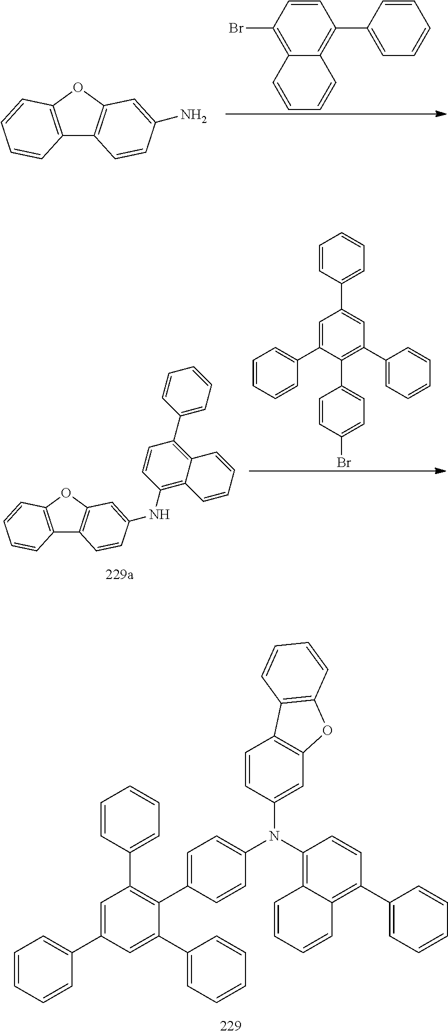

### Synthesis of Intermediate 229a

dibenzo[b,d]furan-3-amine (1.2 eq.), 1-bromo-4-phenylnaphthalene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 229a was obtained by column chromatography. (Yield 63%)

### Synthesis of Compound 229

Intermediate 229a (1.0 eq.), 4″-bromo-3′,5′-diphenyl-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 229 was obtained by column chromatography. (Yield 61%)

### Synthesis Example 12: Synthesis of Compound 230

Compound 230 according to one or more embodiments may be synthesized by, for example, utilizing the following reaction described.

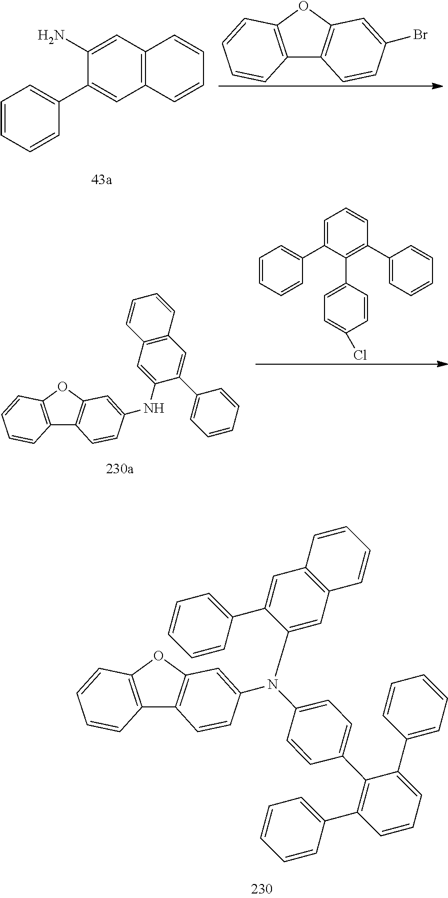

### Synthesis of Intermediate 230a

Intermediate 43a (1.2 eq.), 3-bromodibenzo[b,d]furan (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene, followed by stirring under a nitrogen atmosphere at 80° C. for 1 hour. After the reaction was completed, the reaction product was washed three times utilizing diethyl ether and water, and the obtained organic layer was dried utilizing MgSO₄ and then dried under reduced pressure. Intermediate 230a was obtained by column chromatography. (Yield 61%)

### Synthesis of Compound 230

Intermediate 230a (1.0 eq.), 4″-chloro-3′-phenyl-1,1′:2′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 230 was obtained by column chromatography. (Yield: 64%)

### Synthesis Example 13: Synthesis of Compound 231

Compound 231 according to one or more embodiments may be synthesized by, for example, utilizing the following reaction.

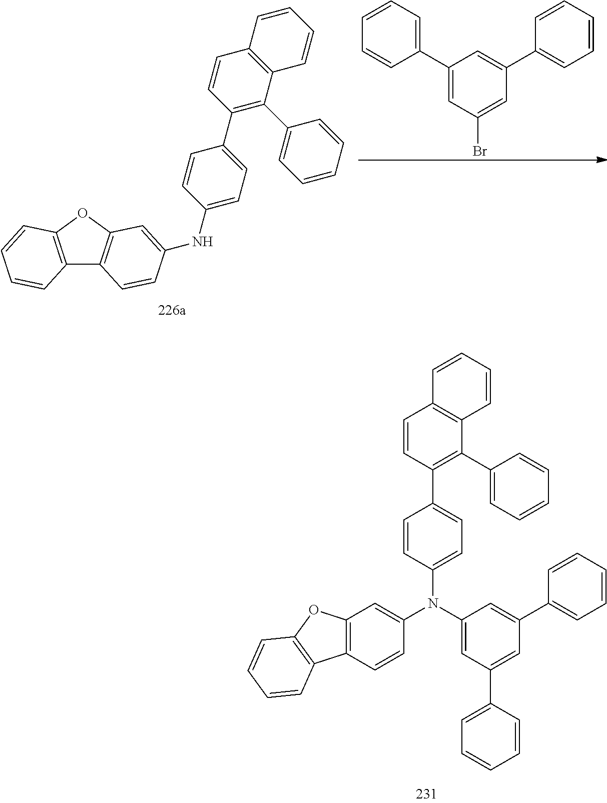 [SMILES: c1ccc(-c2c(-c3ccc(Nc4ccc5c(c4)oc4ccccc45)cc3)ccc3ccccc23)cc1 ; Brc1cc(-c2ccccc2)cc(-c2ccccc2)c1 ; c1ccc(-c2cc(-c3ccccc3)cc(N(c3ccc(-c4ccc5ccccc5c4-c4ccccc4)cc3)c3ccc4c(c3)oc3ccccc34)c2)cc1]

### Synthesis of Compound 231

Intermediate 226a (1.0 eq.), 5′-bromo-1,1′:3′,1″-terphenyl (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of o-xylene, followed by stirring under a nitrogen atmosphere at 150° C. for 1 hour. After the reaction was completed, the reaction product was washed three times utilizing diethyl ether and water, and the obtained organic layer was dried utilizing MgSO₄ and then dried under reduced pressure. Compound 231 was obtained by column chromatography. (Yield 55%).

Data for ¹H nuclear magnetic resonance (NMR) spectroscopy and mass spectroscopy using fast-atom bombardment (MS/FAB) of the compounds synthesized according to Synthesis Examples are shown in Table 1.

TABLE 1

| | ¹H NMR | MS/FAB | |
|---|---|---|---|
| Compound | Chemical shift (δ) | Calc. | Found |
| Compound 1 | 8.80(d, 1H), 8.55(d, 1H), 8.35(d, 2H), 8.24 (d, 1H), | 764.32 | 764.25 |
| | 7.89(d, 1H), 7.81-7.76(m, 6H), 7.66-7.41 (m, 20H), | | |
| | 7.27-7.25 (m, 4H), 7.16-7.11 (m, 4H) | | |
| Compound 6 | 8.80(d, 1H), 8.55(d, 1H), 8.32(d, 2H), 8.24(d, 1H), | 846.40 | 846.31 |
| | 7.89(d, 1H), 7.81-7.76(m, 6H), 7.66(d, 1H), 7.79-7.41 | | |
| | (m, 16H), 7.29-7.25 (m, 6H), 7.16-7.14 (m, 4H), 2.72 | | |
| | (m, 1H), 1.85-1.60 (m, 4H), 1.53-1.43 (m, 6H) | | |
| Compound | 8.80(d, 1H), 8.55(d, 1H), 8.2-8.20(m, 3H), 7.89(d, | 688.29 | 688.15 |
| 33 | 1H), 7.76(d, 1H), 7.66-7.41(m, 19H), 7.27-7.25(m, | | |
| | 2H), 7.16-7.08(m, 8H) | | |
| Compound | 8.20(d, 2H), 7.86(d, 1H), 7.77-7.70(m, 4H), 7.64 (s, | 643.24 | 643.17 |
| 43 | 1H), 7.43-7.37(m, 15H), 7.29(d, 1H), 7.08(m, 6H), | | |
| | 2.58(s, 3H) | | |
| Compound | 8.80(d, 1H), 8.21(d, 1H), 8.03 (s, 1H), 7.98(d, 1H), | 613.24 | 613.11 |
| 76 | 7.89(d, 1H), 7.75-7.43(m, 3H), 7.66(d, 1H), 7.54- | | |
| | 7.41(m, 16H), 7.16-7.08(m, 4H), 6.91(d, 2H) | | |
| Compound | 8.80(d, 1H), 8.55(d, 1H), 8.24-8.20(m, 3H), 7.92- | 846.40 | 846.33 |
| 147 | 7.89(m, 4H), 7.79-7.76(m, 3H), 7.66-7.65(m, 2H), | | |
| | 7.52-7.27(m, 19H), 7.27-7.25(m, 2H), 7.16-7.11(m, | | |
| | 4H), 2.72(m, 1H), 1.85-1.60(m, 4H), 1.53-1.43(m, | | |
| | 6H) | | |
| Compound | 8.93(d, 1H), 8.55(d, 1H), 8.24(d, 1H), 8.04-8.03(m, | 840.35 | 840.21 |
| 169 | 4H), 7.95(d, 1H), 7.75(d, 4H), 7.62-7.36(m, 24H), | | |
| | 7.27-7.25(m, 6H), 7.16-7.11(m, 2H) | | |
| Compound | 8.57(s, 2H), 8.45(d, 1H), 8.15(d, 2H), 7.86(d, 1H), | 705.25 | 705.10 |
| 191 | 7.79-7.74(m, 7H), 7.64-7.27(m, 22H) | | |
| Compound | 8.93(d, 1H), 8.44(d, 1H), 8.13(s, 1H), 8.03(d, 1H), | 854.37 | 854.35 |
| 211 | 7.96-7.95(m, 2H), 7.79(d, 4H), 7.62-7.30(m, 29H), | | |
| | 7.18-7.13(m, 2H), 6.91(d, 1H), 6.40(d, 1H), 2.58(s, | | |
| | 3H) | | |
| Compound | 8.93(d, 1H), 8.21(d, 1H), 8.03-7.95(m, 4H), 7.79(m, | 689.27 | 689.12 |
| 226 | 4H), 7.65(d, 2H), 7.55-7.31(m, 22H), 6.91(d, 1H) | | |
| Compound | 8.95(d, 1H), 8.27(d, 1H), 8.23(s, 2H), 8.03-7.98(m, | 765.30 | 765.23 |
| 229 | 2H), 7.79-7.75(m, 8H), 7.54-7.31(m, 24H), 6.91(d, | | |
| | 1H) | | |
| Compound | 8.35(d, 2H), 8.03(s, 1H), 7.98(d, 1H), 7.86-7.77(m, | 689.27 | 689.15 |
| 230 | 7H), 7.70(s, 1H), 7.54-7.27(m, 20H), 7.08(d, 2H), | | |
| | 6.94(d, 1H) | | |
| Compound | 8.93(d, 1H), 8.03-7.95(m, 4H), 7.75(d, 4H), 7.55- | 689.27 | 689.24 |
| 231 | 7.31(m, 25H), 6.91(d, 1H) | | |

### Example 1

As an anode, a glass substrate (product of Corning Inc.) with a 15 ohm per square centimeter (Q/cm²) (1,200 angstrom (A)) ITO electrode formed thereon was cut to a size of 50 millimeter (mm)×50 mm×0.7 mm, sonicated utilizing isopropyl alcohol and pure water each for 5 minutes, and then cleaned by irradiation of ultraviolet rays and exposure of ozone thereto for 30 minutes. Then, the resultant glass substrate was mounted on a vacuum deposition apparatus.

NPD was deposited on the anode to form (or provide) a hole injection layer having a thickness of 300 Å, Compound 1 was deposited on the hole injection layer to form (or provide) a hole transport layer having a thickness of 200 Å, and CzSi was deposited on the hole transport layer to form (or provide) an emission auxiliary layer having a thickness of 100 Å.

HT-3 and ET-2 (Exiplex hosts), PS-2 (phosphorescent sensitizer), and t-DABNA boron dopant were co-deposited on the emission auxiliary layer at a weight ratio of 42:42:15:1 to form (or provide) an emission layer having a thickness of 200 Å, TSPO1 was deposited on the emission layer to form (or provide) a hole-blocking layer having a thickness of 200 Å, TPBI was deposited on the hole-blocking layer to form (or provide) an electron transport layer having a thickness of 300 Å, LiF was deposited on the electron transport layer to form (or provide) an electron injection layer having a thickness of 10 Å, and Al was deposited on the electron injection layer to form (or provide) a cathode having a thickness of 3,000 Å, thereby completing manufacture of a light-emitting device.

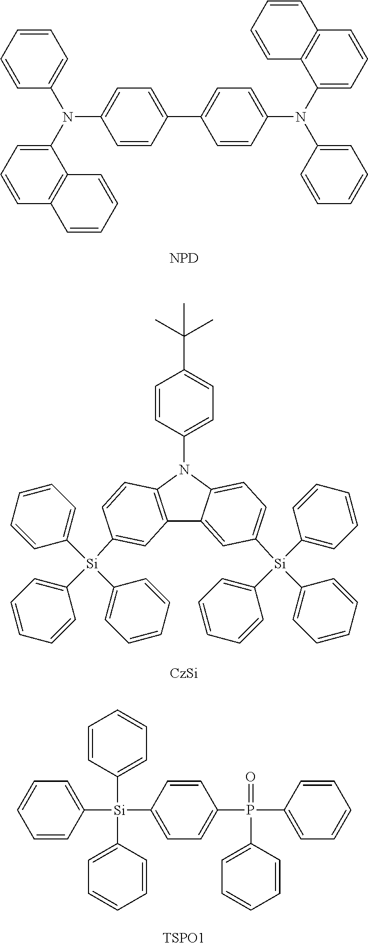

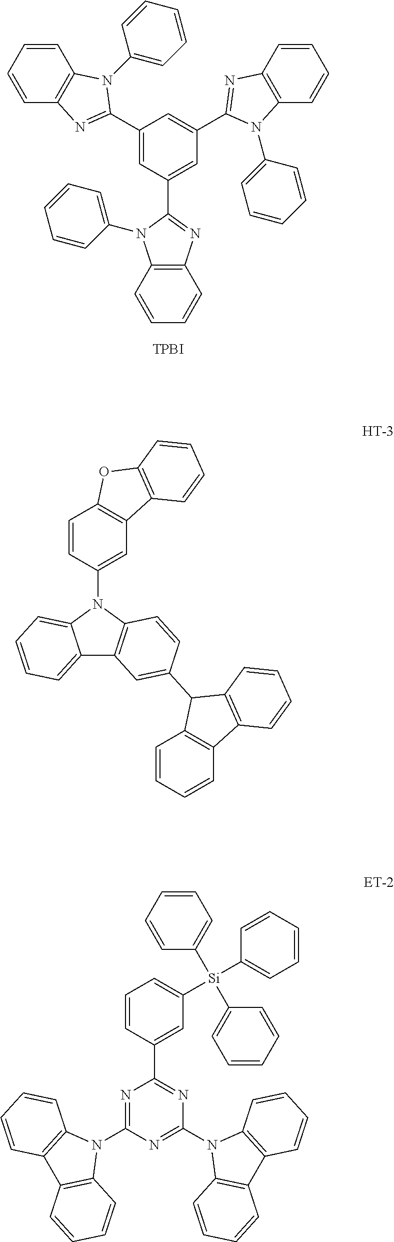

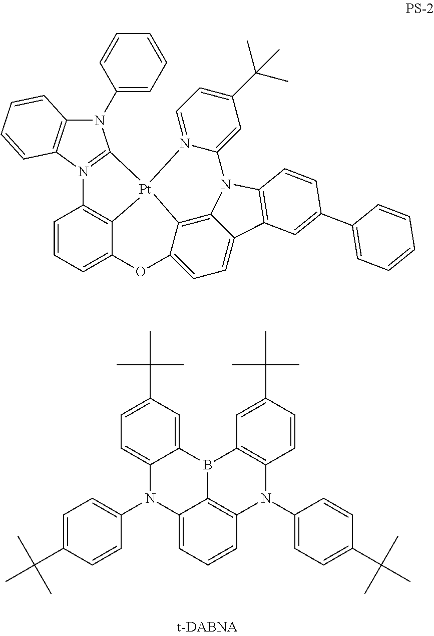

### Examples 2 to 13 and Comparative Examples 1 to 9

Light-emitting devices were manufactured in substantially the same manner as in Example 1, except that, in forming (or providing) the hole transport layer, the compounds shown in Table 2 were each utilized instead of Compound 1.

TABLE 2

| | HTL | | Phos- | Boron |
|---|---|---|---|---|
| | (Hole transport | Exciplex host | phorescent | dopant |
| | layer) | (HT:ET = 5:5) | sensitizer | (t-DABNA1) |
| Example 1 | Compound 1 | HT-3/ET-2 | PS-2 | t-DABNA |
| Example 2 | Compound 6 | HT-3/ET-2 | PS-2 | t-DABNA |
| Example 3 | Compound 33 | HT-3/ET-2 | PS-2 | t-DABNA |
| Example 4 | Compound 43 | HT-3/ET-2 | PS-2 | t-DABNA |
| Example 5 | Compound 76 | HT-3/ET-2 | PS-2 | t-DABNA |
| Example 6 | Compound 147 | HT-3/ET-2 | PS-2 | t-DABNA |
| Example 7 | Compound 169 | HT-3/ET-2 | PS-2 | t-DABNA |
| Example 8 | Compound 191 | HT-3/ET-2 | PS-2 | t-DABNA |
| Example 9 | Compound 211 | HT-3/ET-2 | PS-2 | t-DABNA |
| Example 10 | Compound 226 | HT-3/ET-2 | PS-2 | t-DABNA |
| Example 11 | Compound 229 | HT-3/ET-2 | PS-2 | t-DABNA |
| Example 12 | Compound 230 | HT-3/ET-2 | PS-2 | t-DABNA |
| Example 13 | Compound 231 | HT-3/ET-2 | PS-2 | t-DABNA |
| Comparative | HT3 | HT-3/ET-2 | PS-2 | t-DABNA |
| Example 1 | | | | |
| Comparative | Compound A | HT-3/ET-2 | PS-2 | t-DABNA |
| Example 2 | | | | |
| Comparative | Compound B | HT-3/ET-2 | PS-2 | t-DABNA |
| Example 3 | | | | |
| Comparative | Compound C | HT-3/ET-2 | PS-2 | t-DABNA |
| Example 4 | | | | |
| Comparative | Compound D | HT-3/ET-2 | PS-2 | t-DABNA |
| Example 5 | | | | |
| Comparative | Compound E | HT-3/ET-2 | PS-2 | t-DABNA |
| Example 6 | | | | |
| Comparative | compound F | HT-3/ET-2 | PS-2 | t-DABNA |
| Example 7 | | | | |
| Comparative | Compound G | HT-3/ET-2 | PS-2 | t-DABNA |
| Example 8 | | | | |
| Comparative | Compound H | HT-3/ET-2 | PS-2 | t-DABNA |
| Example 9 | | | | |

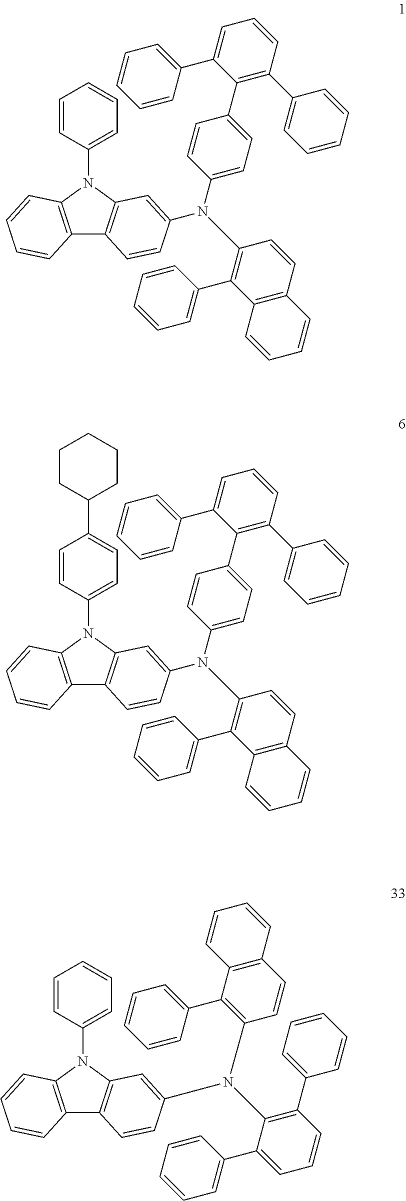 [SMILES: c1ccc(-c2cccc(-c3ccccc3)c2-c2ccc(N(c3ccc4c5ccccc5n(-c5ccccc5)c4c3)c3ccc4ccccc4c3-c3ccccc3)cc2)cc1 ; c1ccc(-c2cccc(-c3ccccc3)c2-c2ccc(N(c3ccc4c5ccccc5n(-c5ccc(C6CCCCC6)cc5)c4c3)c3ccc4ccccc4c3-c3ccccc3)cc2)cc1 ; c1ccc(-c2cccc(-c3ccccc3)c2N(c2ccc3c4ccccc4n(-c4ccccc4)c3c2)c2ccc3ccccc3c2-c2ccccc2)cc1]

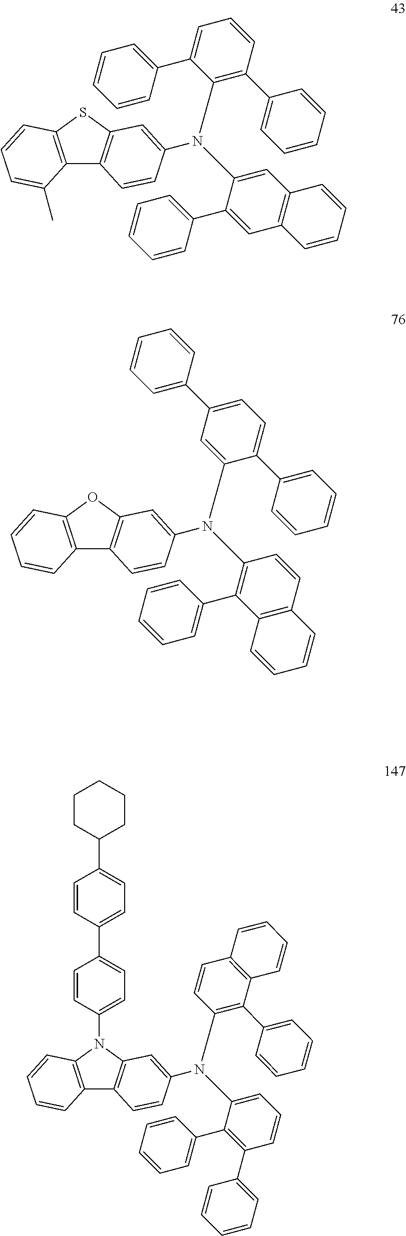 [SMILES: Cc1cccc2sc3cc(N(c4cc5ccccc5cc4-c4ccccc4)c4c(-c5ccccc5)cccc4-c4ccccc4)ccc3c12 ; c1ccc(-c2ccc(-c3ccccc3)c(N(c3ccc4c(c3)oc3ccccc34)c3ccc4ccccc4c3-c3ccccc3)c2)cc1 ; c1ccc(-c2cccc(N(c3ccc4c5ccccc5n(-c5ccc(-c6ccc(C7CCCCC7)cc6)cc5)c4c3)c3ccc4ccccc4c3-c3ccccc3)c2-c2ccccc2)cc1]

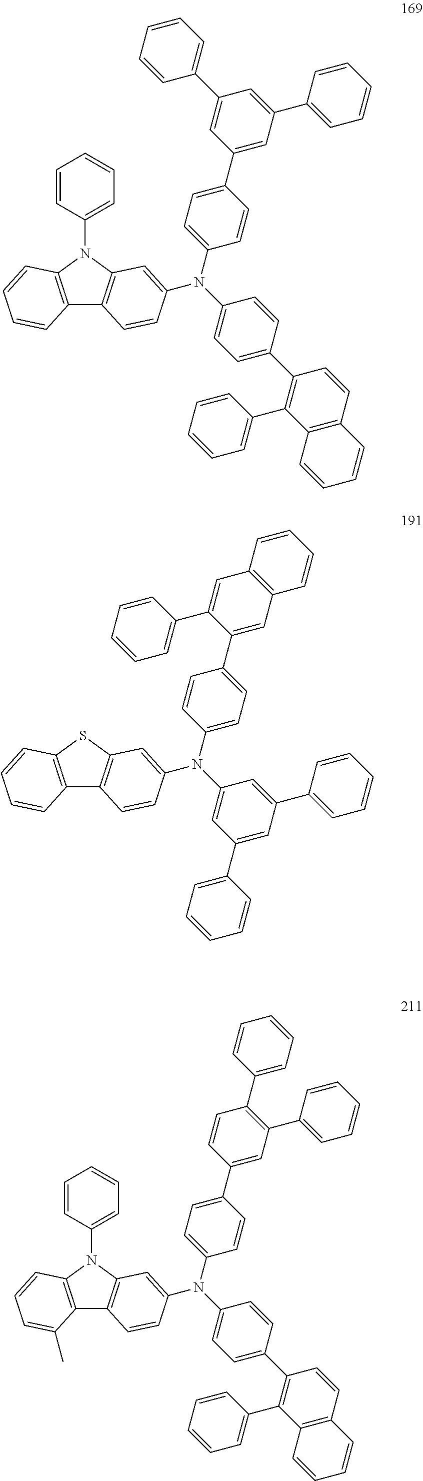 [SMILES: c1ccc(-c2cc(-c3ccccc3)cc(-c3ccc(N(c4ccc(-c5ccc6ccccc6c5-c5ccccc5)cc4)c4ccc5c6ccccc6n(-c6ccccc6)c5c4)cc3)c2)cc1 ; c1ccc(-c2cc(-c3ccccc3)cc(N(c3ccc(-c4cc5ccccc5cc4-c4ccccc4)cc3)c3ccc4c(c3)sc3ccccc34)c2)cc1 ; Cc1cccc2c1c1ccc(N(c3ccc(-c4ccc(-c5ccccc5)c(-c5ccccc5)c4)cc3)c3ccc(-c4ccc5ccccc5c4-c4ccccc4)cc3)cc1n2-c1ccccc1]

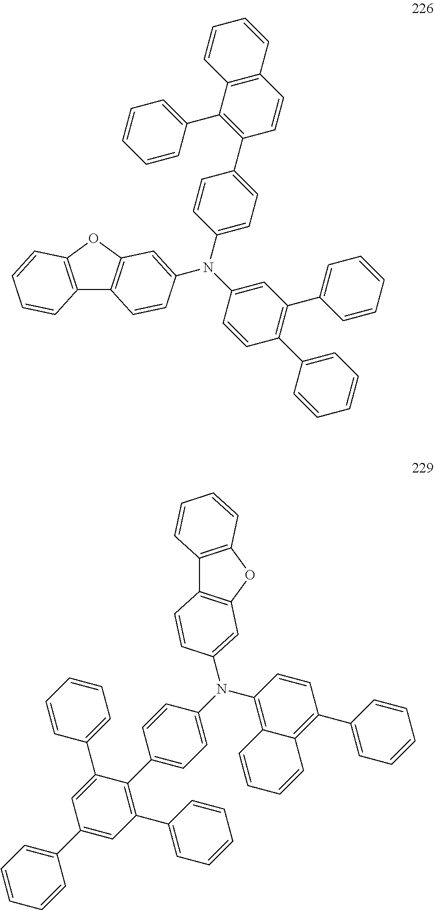 [SMILES: c1ccc(-c2ccc(N(c3ccc(-c4ccc5ccccc5c4-c4ccccc4)cc3)c3ccc4c(c3)oc3ccccc34)cc2-c2ccccc2)cc1 ; c1ccc(-c2cc(-c3ccccc3)c(-c3ccc(N(c4ccc5c(c4)oc4ccccc45)c4ccc(-c5ccccc5)c5ccccc45)cc3)c(-c3ccccc3)c2)cc1]

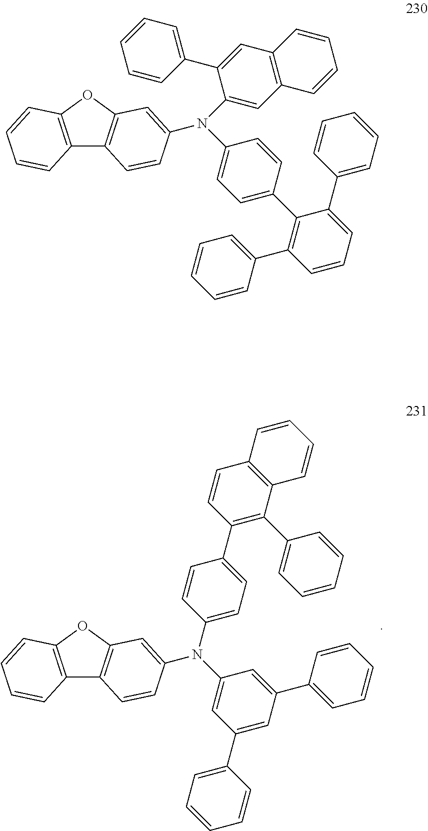 [SMILES: c1ccc(-c2cc3ccccc3cc2N(c2ccc(-c3c(-c4ccccc4)cccc3-c3ccccc3)cc2)c2ccc3c(c2)oc2ccccc23)cc1 ; c1ccc(-c2cc(-c3ccccc3)cc(N(c3ccc(-c4ccc5ccccc5c4-c4ccccc4)cc3)c3ccc4c(c3)oc3ccccc34)c2)cc1]

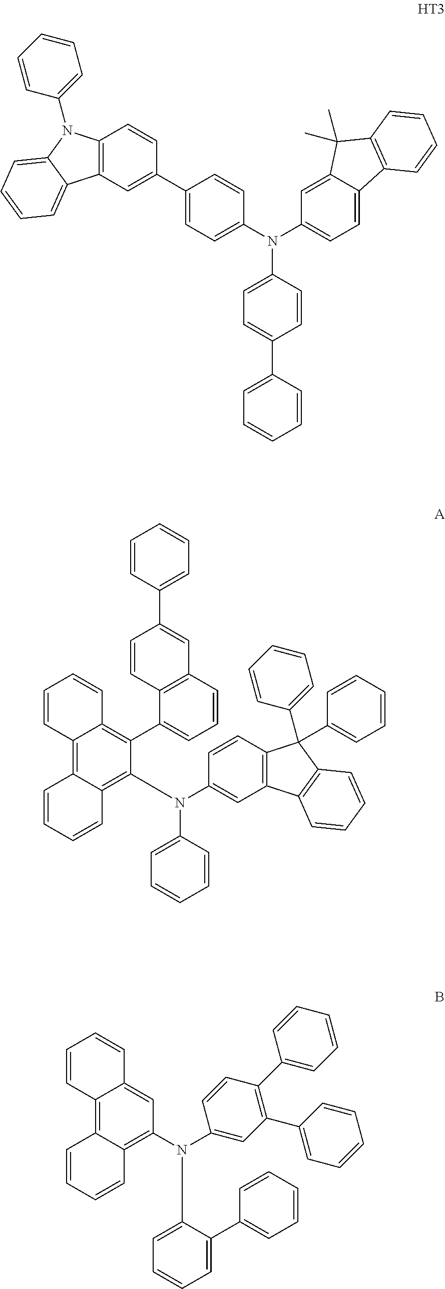 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc3c(-c4c(N(c5ccccc5)c5ccc6c(c5)-c5ccccc5C6(c5ccccc5)c5ccccc5)c5ccccc5c5ccccc45)cccc3c2)cc1 ; c1ccc(-c2ccc(N(c3ccccc3-c3ccccc3)c3cc4ccccc4c4ccccc34)cc2-c2ccccc2)cc1]

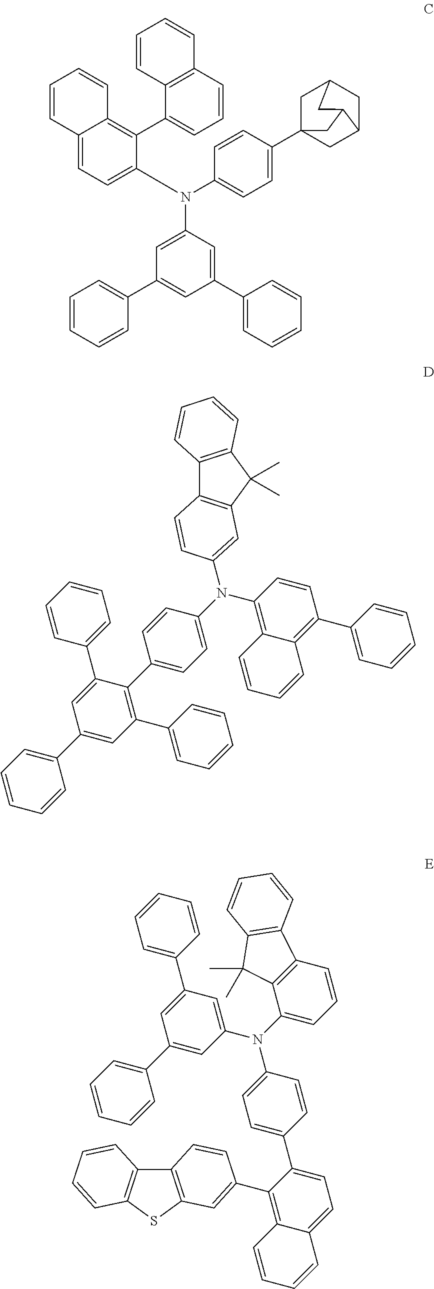 [SMILES: c1ccc(-c2cc(-c3ccccc3)cc(N(c3ccc(C45CC6CC(C4)C(C6)C5)cc3)c3ccc4ccccc4c3-c3cccc4ccccc34)c2)cc1 ; CC1(C)c2ccccc2-c2ccc(N(c3ccc(-c4c(-c5ccccc5)cc(-c5ccccc5)cc4-c4ccccc4)cc3)c3ccc(-c4ccccc4)c4ccccc34)cc21 ; CC1(C)c2ccccc2-c2cccc(N(c3ccc(-c4ccc5ccccc5c4-c4ccc5c(c4)sc4ccccc45)cc3)c3cc(-c4ccccc4)cc(-c4ccccc4)c3)c21]

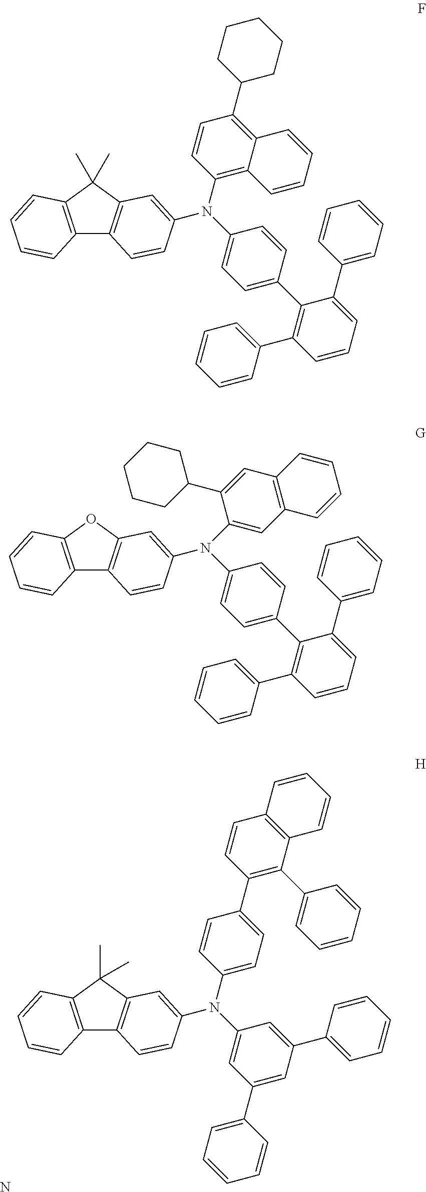

### Evaluation Example

Evaluation of the characteristics of the light-emitting devices including driving voltage (V), current density (milliampere per square centimeter (mA/cm²)), luminance (candela per square meter (cd/m²)), luminescence efficiency (candela per ampere (cd/A)), emission color, and half lifespan (hr @100 mA/cm²) were each measured by utilizing Keithley MU 236 and luminance meter PR650. The luminescence efficiency was measured at the current density of 50 mA/cm². The lifespan ratio was calculated by dividing the T95 values of Examples and Comparative Examples by the T95 value of Comparative Example 1.

When a light-emitting device has a relatively low driving voltage (V) value, the light-emitting device may have relatively higher power efficiency than a light-emitting device having a high driving voltage (V) value even when a device or material having substantially the same quantum efficiency is utilized in both (e.g., simultaneously) devices. The lifespan (T95) value represents time taken for the luminance of the light-emitting device to decline to 95%. The time taken for the luminance of the light-emitting device of Comparative Example 1 to decline to 95% was defined as 1, and the times taken for the luminance of the other light-emitting devices to decline to 95% were shown in relative values, i.e., lifespan ratio (T95).

The driving voltage (V), luminescence efficiency (cd/A), emission wavelength (nanometer (nm)), and lifespan (T95) of the manufactured light-emitting devices are shown in Table 3.

TABLE 3

| | | | Emission | Lifespan |
|---|---|---|---|---|
| | Driving voltage | Efficiency | wavelength | ratio |
| | (V) | (Cd/A) | (nm) | (T₉₅) |
| Example 1 | 3.62 | 32.5 | 460 | 2.4 |
| Example 2 | 3.85 | 33.0 | 461 | 2.0 |
| Example 3 | 3.72 | 32.0 | 461 | 1.9 |
| Example 4 | 3.75 | 28.8 | 460 | 1.8 |
| Example 5 | 3.87 | 27.5 | 461 | 1.6 |
| Example 6 | 3.61 | 33.1 | 462 | 2.1 |
| Example 7 | 3.91 | 31.5 | 461 | 2.0 |
| Example 8 | 3.65 | 29.9 | 460 | 1.8 |
| Example 9 | 3.74 | 30.8 | 460 | 1.3 |
| Example 10 | 3.87 | 27.4 | 461 | 1.5 |
| Example 11 | 3.81 | 27.7 | 462 | 1.4 |
| Example 12 | 3.95 | 29.1 | 461 | 1.5 |
| Example 13 | 3.88 | 27.9 | 460 | 1.4 |
| Comparative | 4.46 | 25.8 | 462 | 1 |
| Example 1 | | | | |
| Comparative | 4.20 | 27.3 | 461 | 1.1 |
| Example 2 | | | | |
| Comparative | 4.12 | 26.4 | 460 | 1.3 |
| Example 3 | | | | |
| Comparative | 4.54 | 24.8 | 463 | 1.2 |
| Example 4 | | | | |
| Comparative | 4.10 | 26.3 | 461 | 1 |
| Example 5 | | | | |
| Comparative | 4.44 | 27.5 | 462 | 1.1 |
| Example 6 | | | | |
| Comparative | 4.05 | 28.9 | 460 | 1.1 |
| Example 7 | | | | |
| Comparative | 4.35 | 27.1 | 461 | 0.9 |
| Example 8 | | | | |
| Comparative | 4.08 | 26.5 | 460 | 1.1 |
| Example 9 | | | | |

As shown in Table 3, the light-emitting devices of Examples 1 to 13 were each found to have a lower driving voltage, compared to the light-emitting devices of each of Comparative Examples 1 to 9. In one or more embodiments, it was confirmed that the light-emitting devices of Examples 1 to 13 each had, substantially the same, or an improved efficiency and lifespan in comparison with the light-emitting devices of Comparative Examples 1 to 9.

The amine-containing compound represented by Formula 1 may have excellent or suitable hole transport characteristics. A light-emitting device including the amine-containing compound represented by Formula 1 may have a relatively low driving voltage, high efficiency, and a long lifespan. The display quality of an electronic apparatus including the light-emitting device and electronic equipment including the light-emitting device may be improved.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form (or provide) and details may be made therein without departing from the spirit and scope as defined by the following claims and equivalents thereof.

1. An amine-containing compound represented by Formula 1: 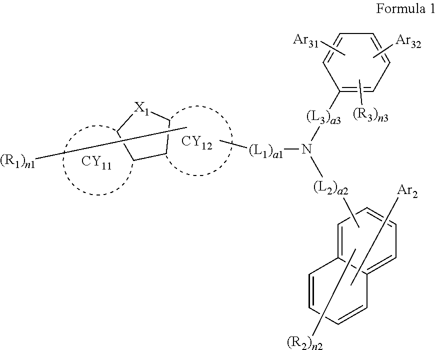
in Formula 1,
X₁ is N(R₄), O, or S,
CY₁₁ and CY₁₂ are each independently a C₃-C₆₀ carbocyclic group or a C₁-C₆₀ heterocyclic group,
Ar₂, Ar₃₁, and Ar₃₂ are each independently a C₃-C₆₀ aryl group unsubstituted or substituted with at least one R₁₀ₐ or a C₁-C₆₀ heteroaryl group unsubstituted or substituted with at least one R₁₀ₐ,
L₁ to L₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 are each independently an integer from 0 to 5, and i) when a1 is 0, a group represented by *-(L₁)ₐ₁-*′ is a single bond, ii) when a2 is 0, a group represented by *-(L₂)ₐ₂-*′ is a single bond, iii) when a3 is 0, a group represented by *-(L₃)ₐ₃-*′ is a single bond, iv) when a1 is an integer from 2 to 5, a plurality of L₁(s) are identical to or different from each other, v) when a2 is an integer from 2 to 5, a plurality of L₂(s) are identical to or different from each other, and vi) when a3 is an integer from 2 to 5, a plurality of L₃(s) are identical to or different from each other,
R₁ to R₄ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
neighboring two or more of R₁ to R₃ are not bonded or are bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
n1 is an integer from 0 to 20,
n2 is an integer from 0 to 6,
n3 is an integer from 0 to 3,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group, and
* and *′ each indicate a binding site to a neighboring atom.

2. The amine-containing compound of claim 1, wherein CY₁₁ and CY₁₂ are each independently a benzene group, a naphthalene group, a phenanthrene group, a fluoranthene group, a triphenylene group, a pyrene group, a chrysene group, an indene group, a fluorene group, a spiro-bifluorene group, a benzofluorene group, a dibenzofluorene group, an indole group, a pyridine group, a pyrimidine group, a carbazole group, a benzocarbazole group, a dibenzocarbazole group, a furan group, a benzofuran group, a dibenzofuran group, a naphthofuran group, a benzonaphthofuran group, a dinaphthofuran group, a thiophene group, a benzothiophene group, a dibenzothiophene group, a naphthothiophene group, a benzonaphthothiophene group, or a dinaphthothiophene group.

3. The amine-containing compound of claim 1, wherein the amine-containing compound represented by Formula 1 is a compound represented by any one selected from among Formula 1-1 to Formula 1-4: 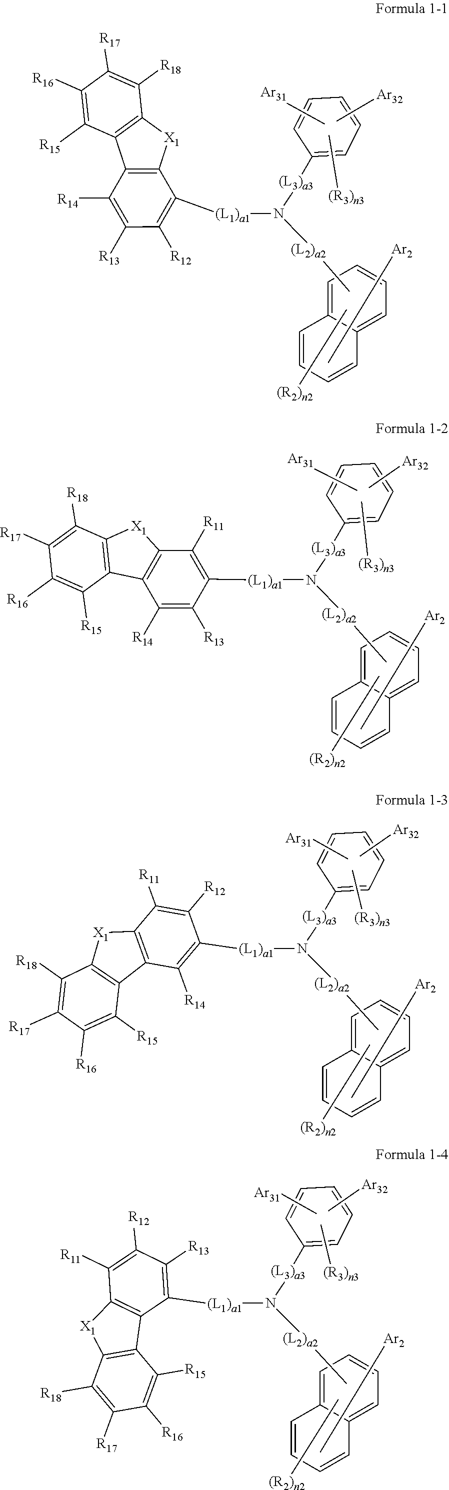
in Formulae 1-1 to 1-4,
X₁, Ar₂, Ar₃₁, Ar₃₂, L₁ to L₃, a1 to a3, R₂, R₃, n2, and n3 are each as described for Formula 1, and
R₁₁ to R₁₈ are each as described in connection with R₁ in Formula 1.

4. The amine-containing compound of claim 1, wherein Ar₂, Ar₃₁, and Ar₃₂ are each independently a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a dimethylfluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphthosilolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indeno carbazolyl group, an indolocarbazolyl group, a naphthacenyl group, a picenyl group, a hexacenyl group, a pentaphenyl group, a rubicenyl group, a coronenyl group, an ovalenyl group, a naphthopyrrolyl group, a naphthofuranyl group, a naphthothiophenyl group, a naphthosilolyl group, a benzofurocarbazolyl group, a benzothienocarbazolyl group, a benzosilolocarbazolyl group, a triindolophenyl group, a pyrrolophenanthrenyl group, a furanophenanthrenyl group, a thienophenanthrenyl group, a benzonaphthofuranyl group, a benzonaphthothiophenyl group, an (indolo)phenanthrenyl group, a (benzofurano)phenanthrenyl group, or a (benzothieno)phenanthrenyl group, each unsubstituted or substituted with at least one R₁₀ₐ.

5. The amine-containing compound of claim 1, wherein Ar₂, Ar₃₁, and Ar₃₂ are each independently 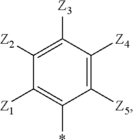
Z₁ to Z₅ are each independently hydrogen or as described in connection with R₁₀ₐ for Formula 1, and
* indicates a binding site to a neighboring atom.

6. The amine-containing compound of claim 1, wherein a group represented by 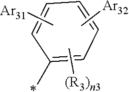
is a group represented by any one selected from among Formula 3A to Formula 3F: 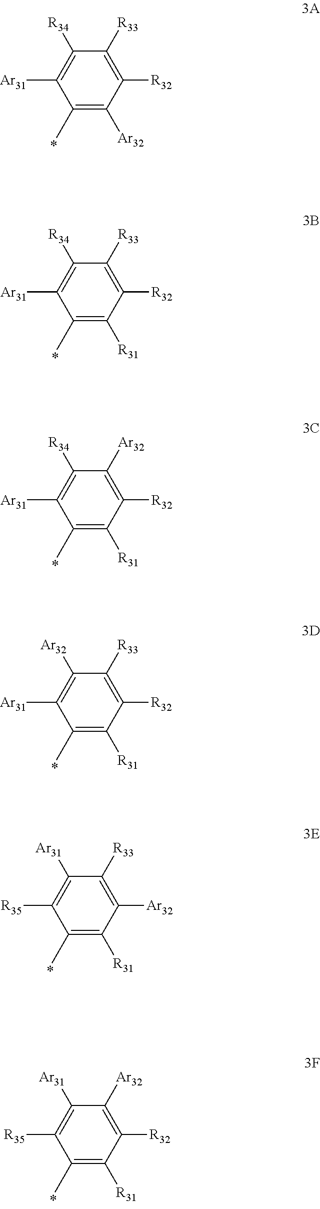
in Formula 3A to Formula 3F,
Ar₃₁ and Ar₃₂ are each as described for Formula 1,
R₃₁ to R₃₅ are each as described in connection with R₃ for Formula 1, and
indicates a binding site to a neighboring atom.

7. The amine-containing compound of claim 1, wherein R₃ is hydrogen or deuterium.

8. The amine-containing compound of claim 1, wherein a group represented by 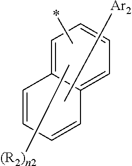
is a group represented by any one selected from among Formula 2A to Formula 2N: 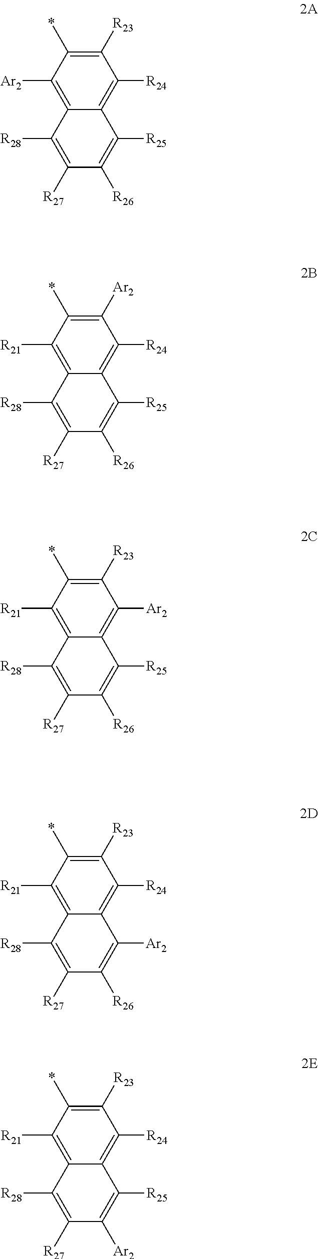 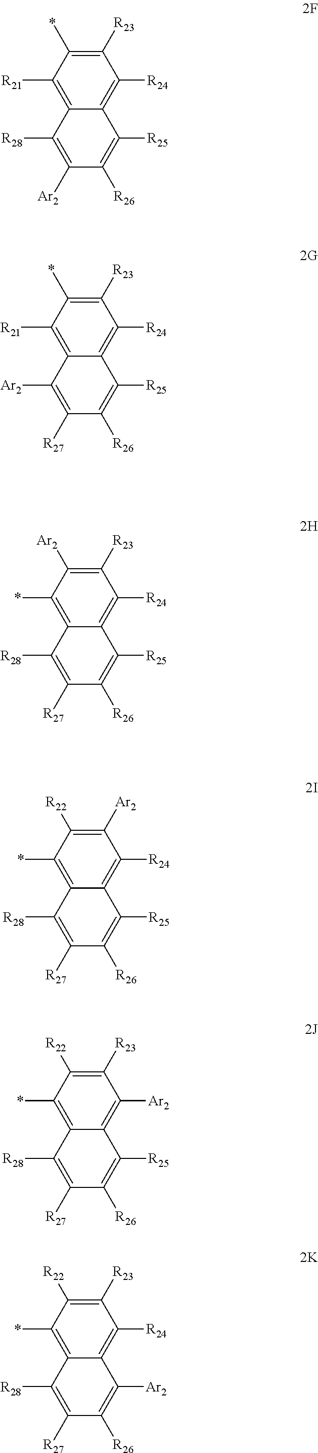 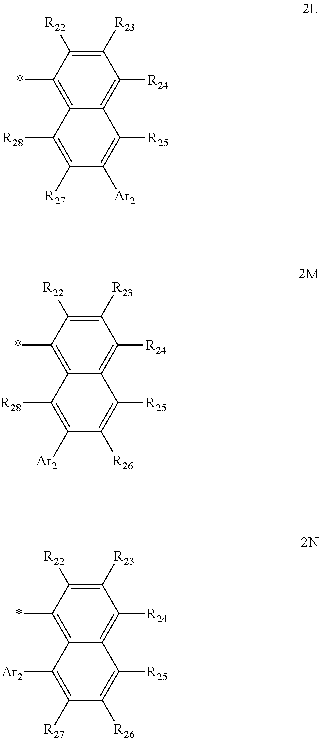
wherein, in Formula 2A to Formula 2N,
Ar₂ is as described for Formula 1,
R₂₁ to R₂₈ are each as described in connection with R₂ for Formula 1, and
* indicates a binding site to a neighboring atom.

9. The amine-containing compound of claim 1, wherein L₁ to L₃ are each independently a benzene group, a naphthalene group, an anthracene group, a phenanthrene group, a triphenylene group, a pyrene group, or a chrysene group, each unsubstituted or substituted with at least one R₁₀ₐ, and
R₁₀ₐ is as described in for Formula 1.

10. The amine-containing compound of claim 1, wherein L₁ to L₃ are each a group represented by one selected from among Formula 4A to Formula 4C: 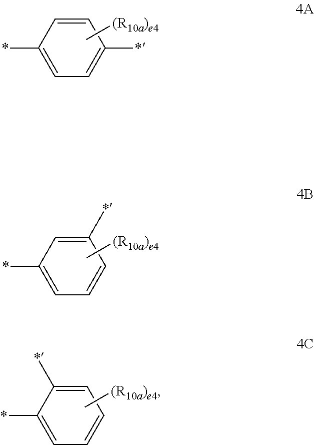
in Formulae 4A to 4C,
R₁₀ₐ is as described for Formula 1,
e4 is an integer from 0 to 4, and
* and *′ each indicate a binding site to a neighboring atom.

11. The amine-containing compound of claim 1, wherein the amine-containing compound represented by Formula 1 is any one selected from among Compounds 1 to 231:

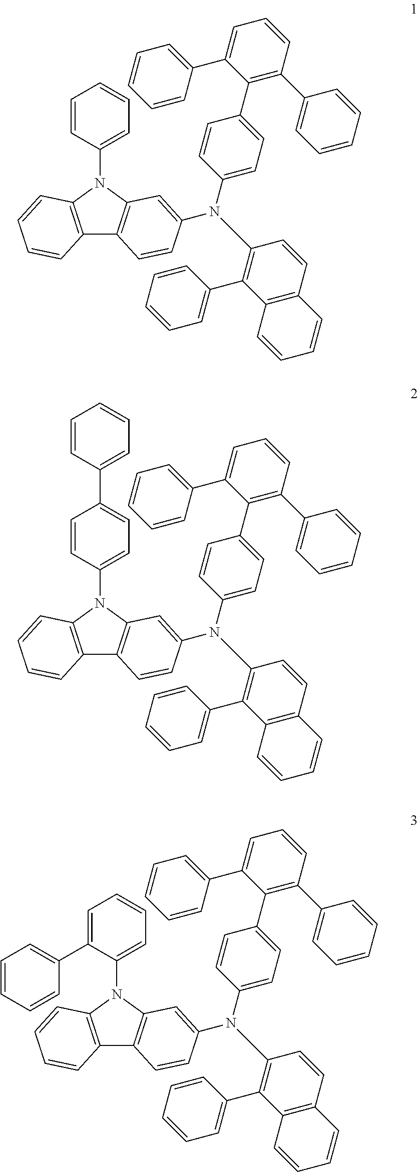 [SMILES: c1ccc(-c2cccc(-c3ccccc3)c2-c2ccc(N(c3ccc4c5ccccc5n(-c5ccccc5)c4c3)c3ccc4ccccc4c3-c3ccccc3)cc2)cc1 ; c1ccc(-c2ccc(-n3c4ccccc4c4ccc(N(c5ccc(-c6c(-c7ccccc7)cccc6-c6ccccc6)cc5)c5ccc6ccccc6c5-c5ccccc5)cc43)cc2)cc1 ; c1ccc(-c2ccccc2-n2c3ccccc3c3ccc(N(c4ccc(-c5c(-c6ccccc6)cccc5-c5ccccc5)cc4)c4ccc5ccccc5c4-c4ccccc4)cc32)cc1]

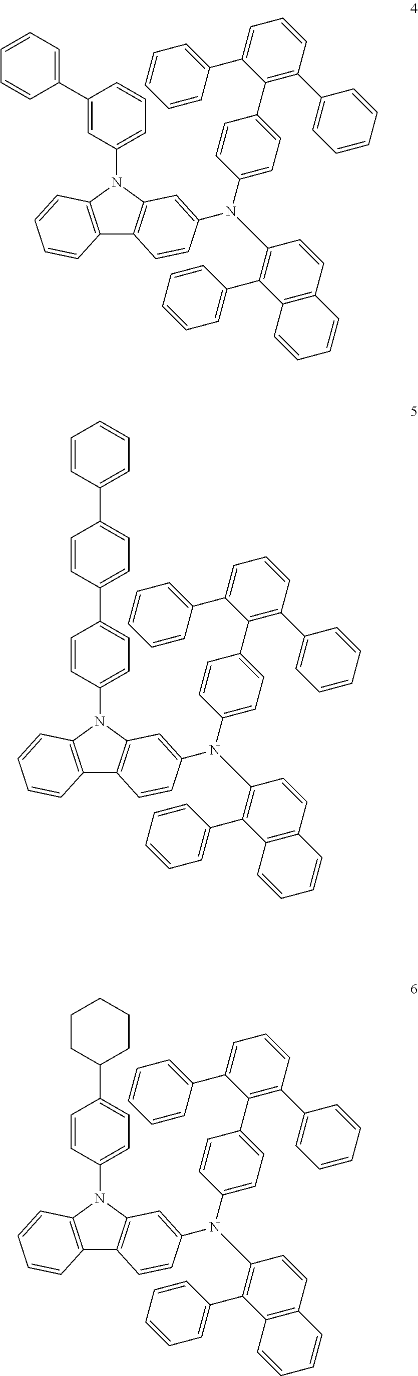 [SMILES: c1ccc(-c2cccc(-n3c4ccccc4c4ccc(N(c5ccc(-c6c(-c7ccccc7)cccc6-c6ccccc6)cc5)c5ccc6ccccc6c5-c5ccccc5)cc43)c2)cc1 ; c1ccc(-c2ccc(-c3ccc(-n4c5ccccc5c5ccc(N(c6ccc(-c7c(-c8ccccc8)cccc7-c7ccccc7)cc6)c6ccc7ccccc7c6-c6ccccc6)cc54)cc3)cc2)cc1 ; c1ccc(-c2cccc(-c3ccccc3)c2-c2ccc(N(c3ccc4c5ccccc5n(-c5ccc(C6CCCCC6)cc5)c4c3)c3ccc4ccccc4c3-c3ccccc3)cc2)cc1]

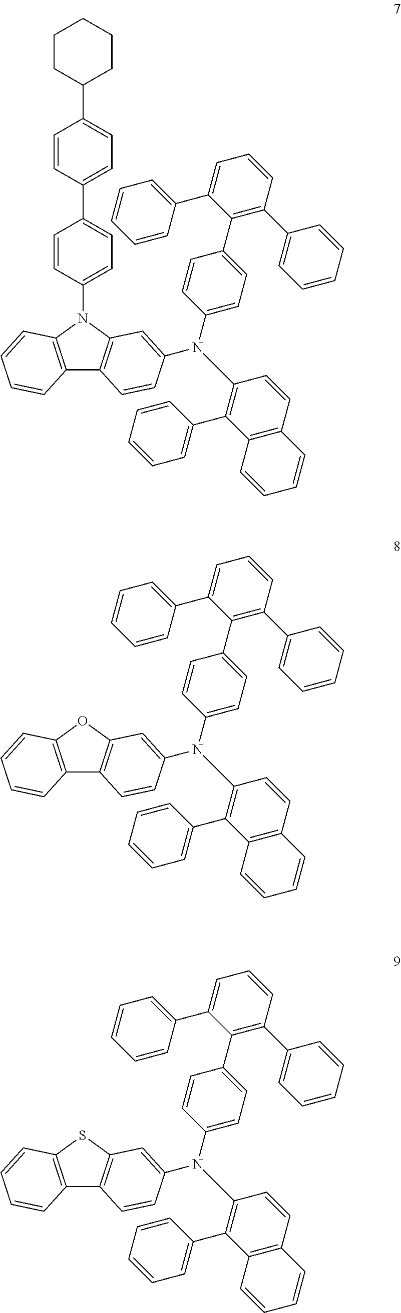 [SMILES: c1ccc(-c2cccc(-c3ccccc3)c2-c2ccc(N(c3ccc4c5ccccc5n(-c5ccc(-c6ccc(C7CCCCC7)cc6)cc5)c4c3)c3ccc4ccccc4c3-c3ccccc3)cc2)cc1 ; c1ccc(-c2cccc(-c3ccccc3)c2-c2ccc(N(c3ccc4c(c3)oc3ccccc34)c3ccc4ccccc4c3-c3ccccc3)cc2)cc1 ; c1ccc(-c2cccc(-c3ccccc3)c2-c2ccc(N(c3ccc4c(c3)sc3ccccc34)c3ccc4ccccc4c3-c3ccccc3)cc2)cc1]

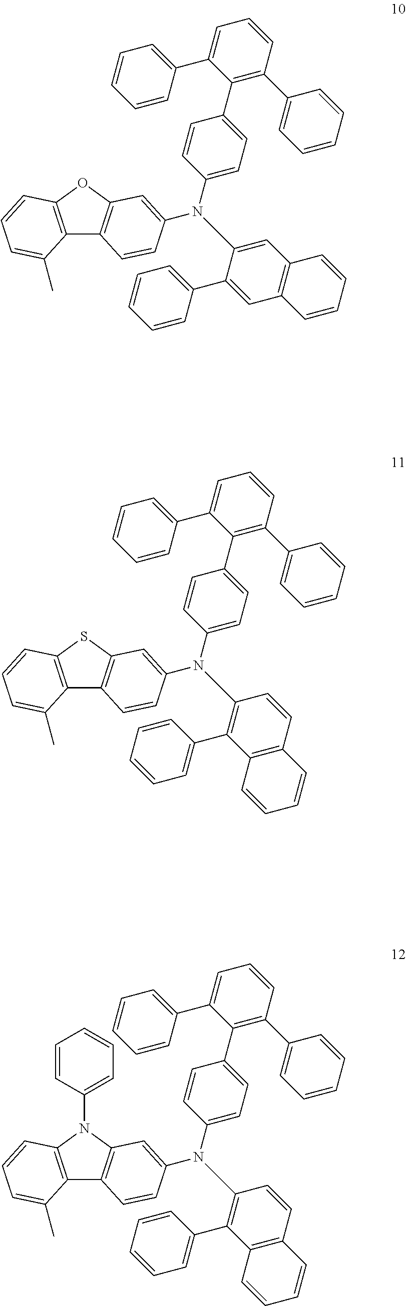 [SMILES: Cc1cccc2oc3cc(N(c4ccc(-c5c(-c6ccccc6)cccc5-c5ccccc5)cc4)c4cc5ccccc5cc4-c4ccccc4)ccc3c12 ; Cc1cccc2sc3cc(N(c4ccc(-c5c(-c6ccccc6)cccc5-c5ccccc5)cc4)c4ccc5ccccc5c4-c4ccccc4)ccc3c12 ; Cc1cccc2c1c1ccc(N(c3ccc(-c4c(-c5ccccc5)cccc4-c4ccccc4)cc3)c3ccc4ccccc4c3-c3ccccc3)cc1n2-c1ccccc1]

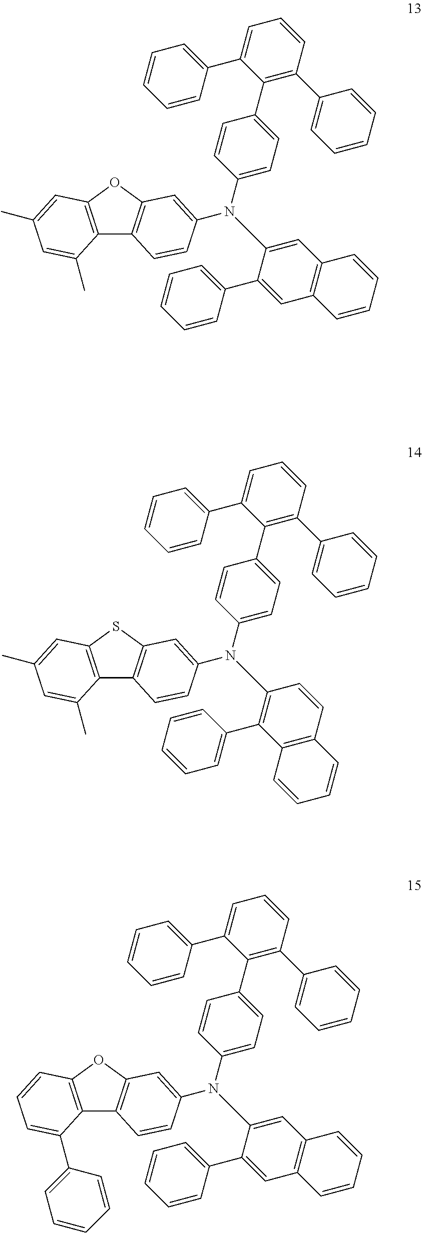 [SMILES: Cc1cc(C)c2c(c1)oc1cc(N(c3ccc(-c4c(-c5ccccc5)cccc4-c4ccccc4)cc3)c3cc4ccccc4cc3-c3ccccc3)ccc12 ; Cc1cc(C)c2c(c1)sc1cc(N(c3ccc(-c4c(-c5ccccc5)cccc4-c4ccccc4)cc3)c3ccc4ccccc4c3-c3ccccc3)ccc12 ; c1ccc(-c2cc3ccccc3cc2N(c2ccc(-c3c(-c4ccccc4)cccc3-c3ccccc3)cc2)c2ccc3c(c2)oc2cccc(-c4ccccc4)c23)cc1]

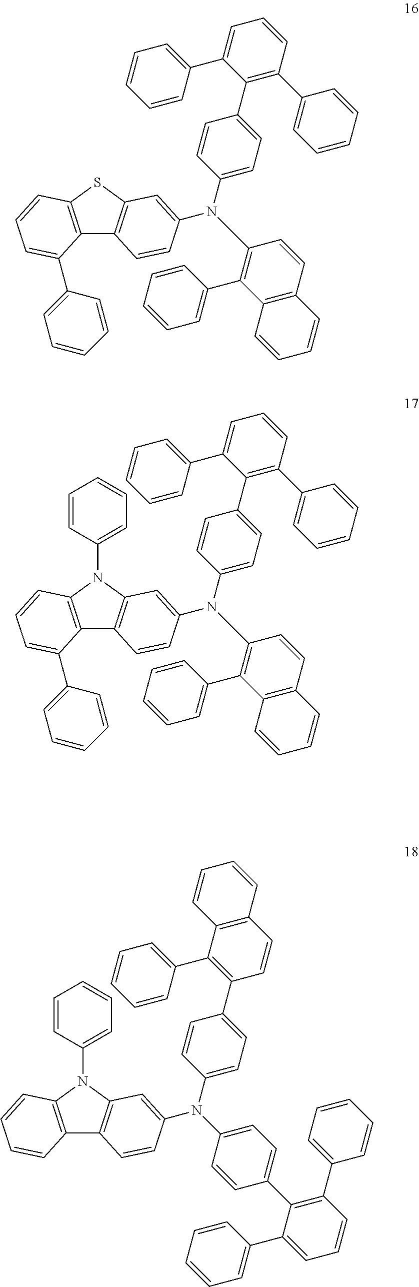 [SMILES: c1ccc(-c2cccc(-c3ccccc3)c2-c2ccc(N(c3ccc4c(c3)sc3cccc(-c5ccccc5)c34)c3ccc4ccccc4c3-c3ccccc3)cc2)cc1 ; c1ccc(-c2cccc(-c3ccccc3)c2-c2ccc(N(c3ccc4c5c(-c6ccccc6)cccc5n(-c5ccccc5)c4c3)c3ccc4ccccc4c3-c3ccccc3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccccc5)c4c3)cc2)cc1]

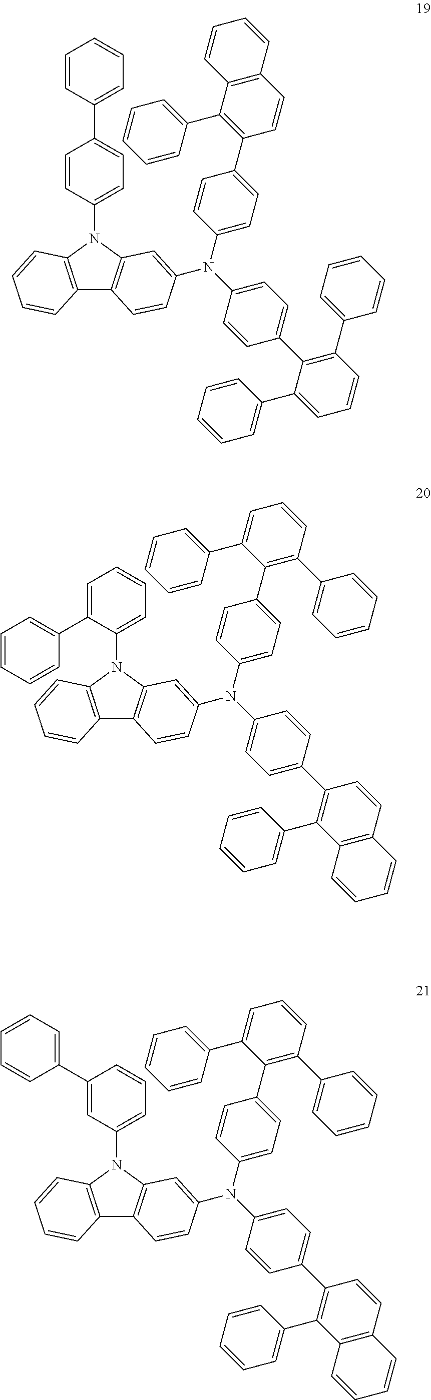 [SMILES: c1ccc(-c2ccc(-n3c4ccccc4c4ccc(N(c5ccc(-c6ccc7ccccc7c6-c6ccccc6)cc5)c5ccc(-c6c(-c7ccccc7)cccc6-c6ccccc6)cc5)cc43)cc2)cc1 ; c1ccc(-c2ccccc2-n2c3ccccc3c3ccc(N(c4ccc(-c5ccc6ccccc6c5-c5ccccc5)cc4)c4ccc(-c5c(-c6ccccc6)cccc5-c5ccccc5)cc4)cc32)cc1 ; c1ccc(-c2cccc(-n3c4ccccc4c4ccc(N(c5ccc(-c6ccc7ccccc7c6-c6ccccc6)cc5)c5ccc(-c6c(-c7ccccc7)cccc6-c6ccccc6)cc5)cc43)c2)cc1]

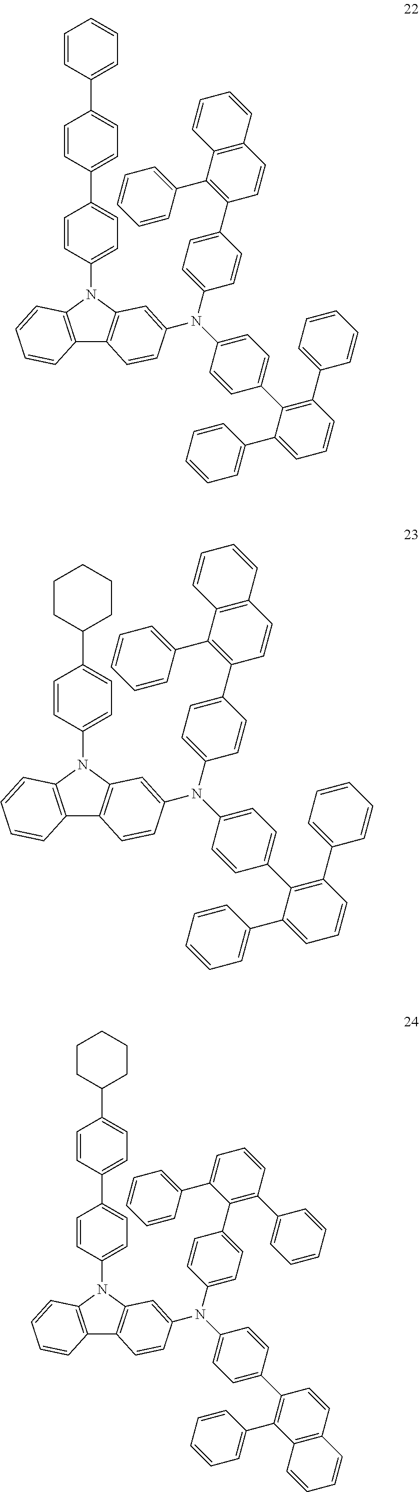 [SMILES: c1ccc(-c2ccc(-c3ccc(-n4c5ccccc5c5ccc(N(c6ccc(-c7ccc8ccccc8c7-c7ccccc7)cc6)c6ccc(-c7c(-c8ccccc8)cccc7-c7ccccc7)cc6)cc54)cc3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccc(C6CCCCC6)cc5)c4c3)cc2)cc1 ; c1ccc(-c2cccc(-c3ccccc3)c2-c2ccc(N(c3ccc(-c4ccc5ccccc5c4-c4ccccc4)cc3)c3ccc4c5ccccc5n(-c5ccc(-c6ccc(C7CCCCC7)cc6)cc5)c4c3)cc2)cc1]

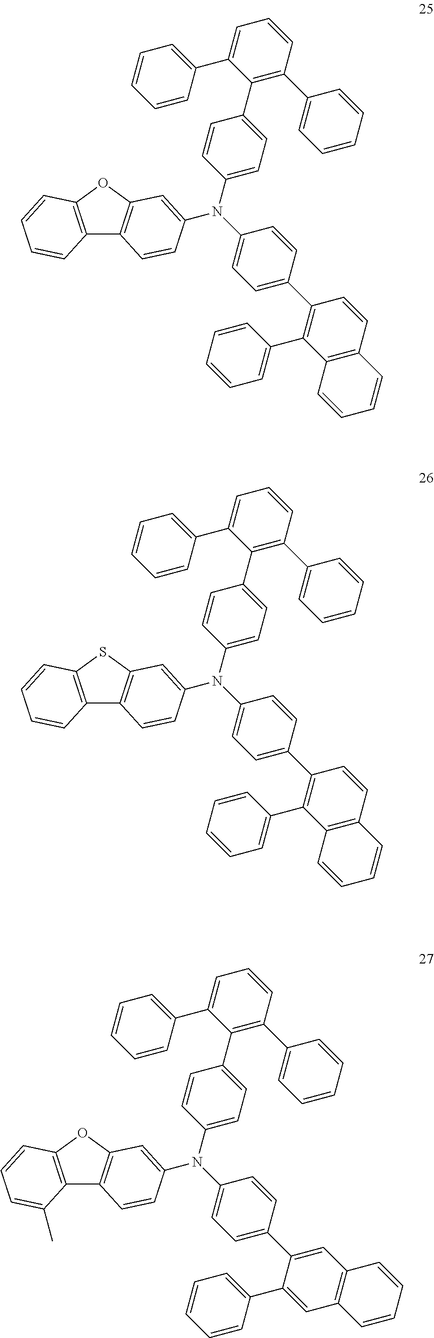 [SMILES: c1ccc(-c2cccc(-c3ccccc3)c2-c2ccc(N(c3ccc(-c4ccc5ccccc5c4-c4ccccc4)cc3)c3ccc4c(c3)oc3ccccc34)cc2)cc1 ; c1ccc(-c2cccc(-c3ccccc3)c2-c2ccc(N(c3ccc(-c4ccc5ccccc5c4-c4ccccc4)cc3)c3ccc4c(c3)sc3ccccc34)cc2)cc1 ; Cc1cccc2oc3cc(N(c4ccc(-c5cc6ccccc6cc5-c5ccccc5)cc4)c4ccc(-c5c(-c6ccccc6)cccc5-c5ccccc5)cc4)ccc3c12]

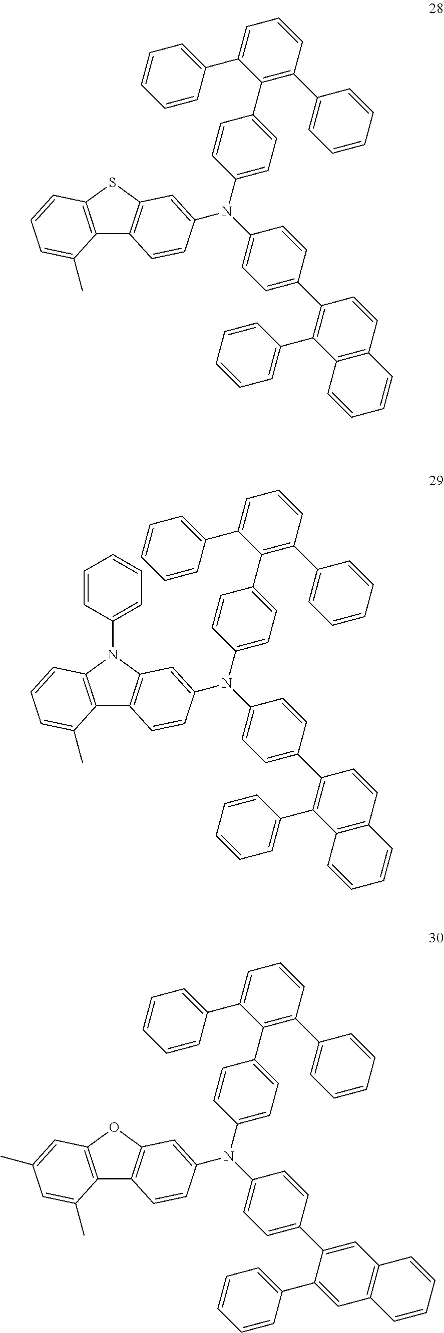 [SMILES: Cc1cccc2sc3cc(N(c4ccc(-c5ccc6ccccc6c5-c5ccccc5)cc4)c4ccc(-c5c(-c6ccccc6)cccc5-c5ccccc5)cc4)ccc3c12 ; Cc1cccc2c1c1ccc(N(c3ccc(-c4ccc5ccccc5c4-c4ccccc4)cc3)c3ccc(-c4c(-c5ccccc5)cccc4-c4ccccc4)cc3)cc1n2-c1ccccc1 ; Cc1cc(C)c2c(c1)oc1cc(N(c3ccc(-c4cc5ccccc5cc4-c4ccccc4)cc3)c3ccc(-c4c(-c5ccccc5)cccc4-c4ccccc4)cc3)ccc12]

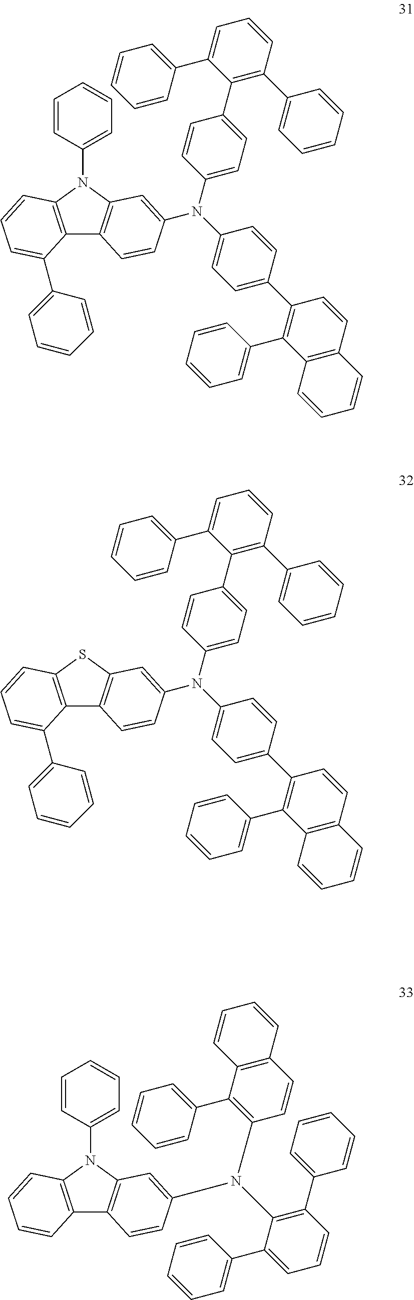 [SMILES: c1ccc(-c2cccc(-c3ccccc3)c2-c2ccc(N(c3ccc(-c4ccc5ccccc5c4-c4ccccc4)cc3)c3ccc4c5c(-c6ccccc6)cccc5n(-c5ccccc5)c4c3)cc2)cc1 ; c1ccc(-c2cccc(-c3ccccc3)c2-c2ccc(N(c3ccc(-c4ccc5ccccc5c4-c4ccccc4)cc3)c3ccc4c(c3)sc3cccc(-c5ccccc5)c34)cc2)cc1 ; c1ccc(-c2cccc(-c3ccccc3)c2N(c2ccc3c4ccccc4n(-c4ccccc4)c3c2)c2ccc3ccccc3c2-c2ccccc2)cc1]

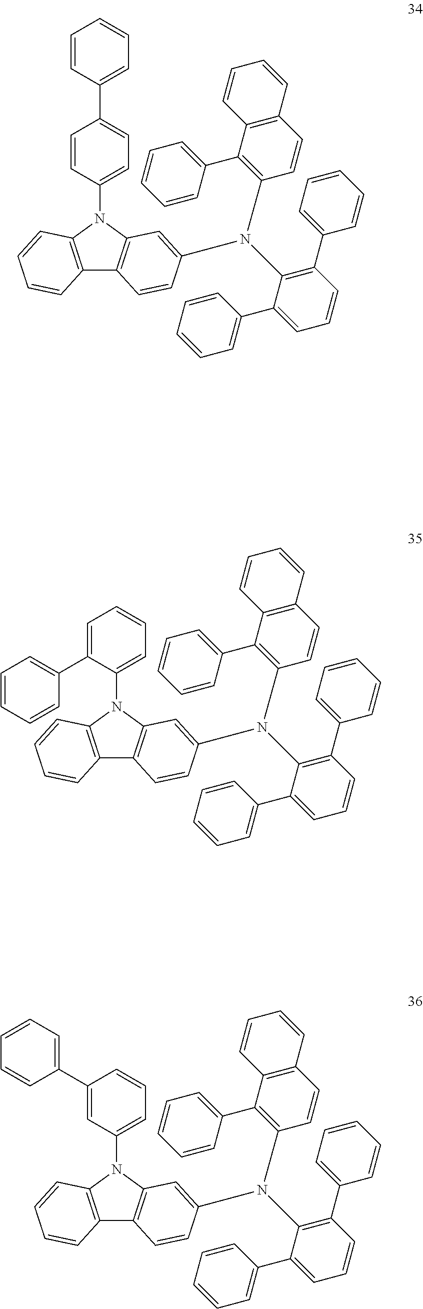 [SMILES: c1ccc(-c2ccc(-n3c4ccccc4c4ccc(N(c5ccc6ccccc6c5-c5ccccc5)c5c(-c6ccccc6)cccc5-c5ccccc5)cc43)cc2)cc1 ; c1ccc(-c2ccccc2-n2c3ccccc3c3ccc(N(c4ccc5ccccc5c4-c4ccccc4)c4c(-c5ccccc5)cccc4-c4ccccc4)cc32)cc1 ; c1ccc(-c2cccc(-n3c4ccccc4c4ccc(N(c5ccc6ccccc6c5-c5ccccc5)c5c(-c6ccccc6)cccc5-c5ccccc5)cc43)c2)cc1]

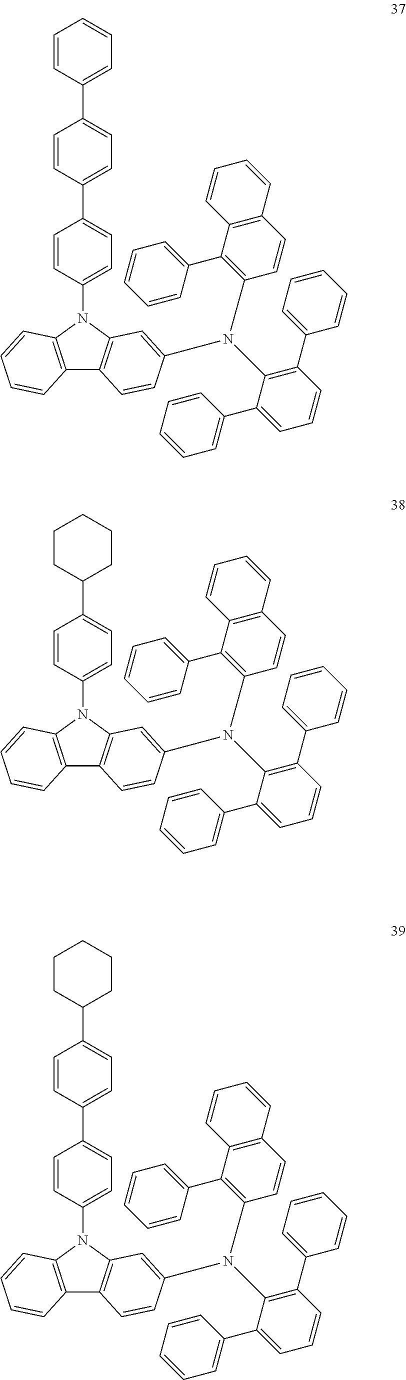 [SMILES: c1ccc(-c2ccc(-c3ccc(-n4c5ccccc5c5ccc(N(c6ccc7ccccc7c6-c6ccccc6)c6c(-c7ccccc7)cccc6-c6ccccc6)cc54)cc3)cc2)cc1 ; c1ccc(-c2cccc(-c3ccccc3)c2N(c2ccc3c4ccccc4n(-c4ccc(C5CCCCC5)cc4)c3c2)c2ccc3ccccc3c2-c2ccccc2)cc1 ; c1ccc(-c2cccc(-c3ccccc3)c2N(c2ccc3c4ccccc4n(-c4ccc(-c5ccc(C6CCCCC6)cc5)cc4)c3c2)c2ccc3ccccc3c2-c2ccccc2)cc1]

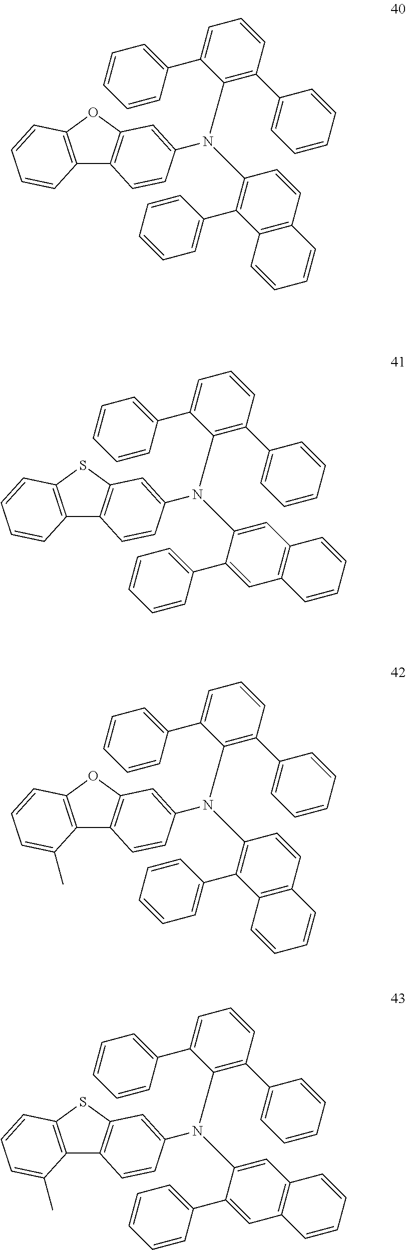 [SMILES: c1ccc(-c2cccc(-c3ccccc3)c2N(c2ccc3c(c2)oc2ccccc23)c2ccc3ccccc3c2-c2ccccc2)cc1 ; c1ccc(-c2cc3ccccc3cc2N(c2ccc3c(c2)sc2ccccc23)c2c(-c3ccccc3)cccc2-c2ccccc2)cc1 ; Cc1cccc2oc3cc(N(c4ccc5ccccc5c4-c4ccccc4)c4c(-c5ccccc5)cccc4-c4ccccc4)ccc3c12 ; Cc1cccc2sc3cc(N(c4cc5ccccc5cc4-c4ccccc4)c4c(-c5ccccc5)cccc4-c4ccccc4)ccc3c12]

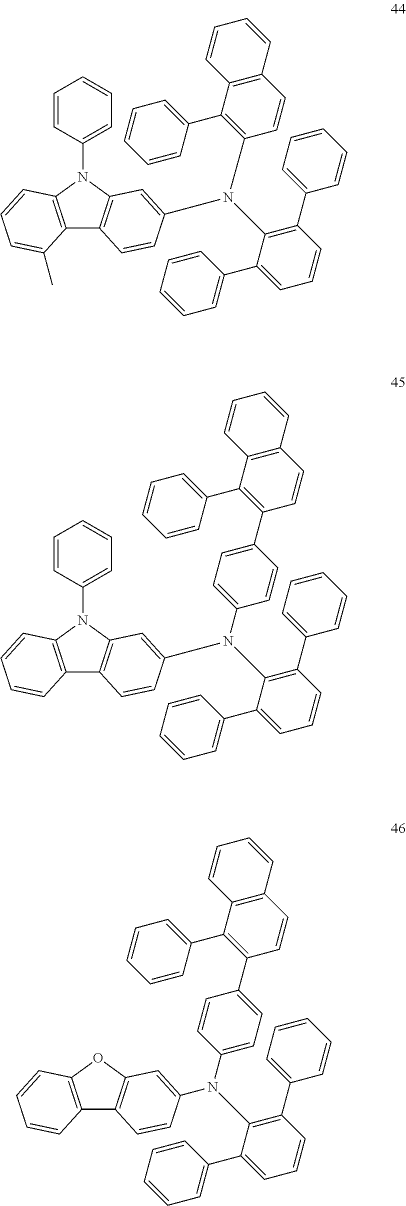 [SMILES: Cc1cccc2c1c1ccc(N(c3ccc4ccccc4c3-c3ccccc3)c3c(-c4ccccc4)cccc3-c3ccccc3)cc1n2-c1ccccc1 ; c1ccc(-c2cccc(-c3ccccc3)c2N(c2ccc(-c3ccc4ccccc4c3-c3ccccc3)cc2)c2ccc3c4ccccc4n(-c4ccccc4)c3c2)cc1 ; c1ccc(-c2cccc(-c3ccccc3)c2N(c2ccc(-c3ccc4ccccc4c3-c3ccccc3)cc2)c2ccc3c(c2)oc2ccccc23)cc1]

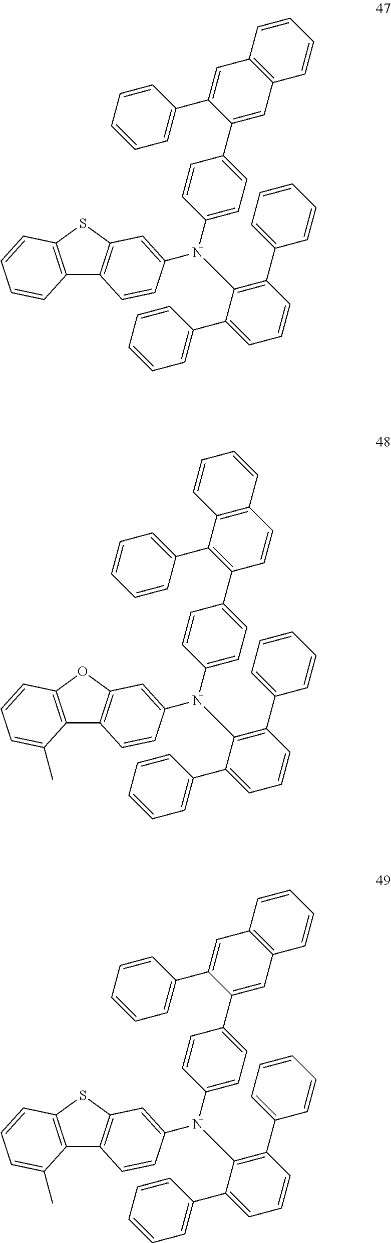 [SMILES: c1ccc(-c2cc3ccccc3cc2-c2ccc(N(c3ccc4c(c3)sc3ccccc34)c3c(-c4ccccc4)cccc3-c3ccccc3)cc2)cc1 ; Cc1cccc2oc3cc(N(c4ccc(-c5ccc6ccccc6c5-c5ccccc5)cc4)c4c(-c5ccccc5)cccc4-c4ccccc4)ccc3c12 ; Cc1cccc2sc3cc(N(c4ccc(-c5cc6ccccc6cc5-c5ccccc5)cc4)c4c(-c5ccccc5)cccc4-c4ccccc4)ccc3c12]

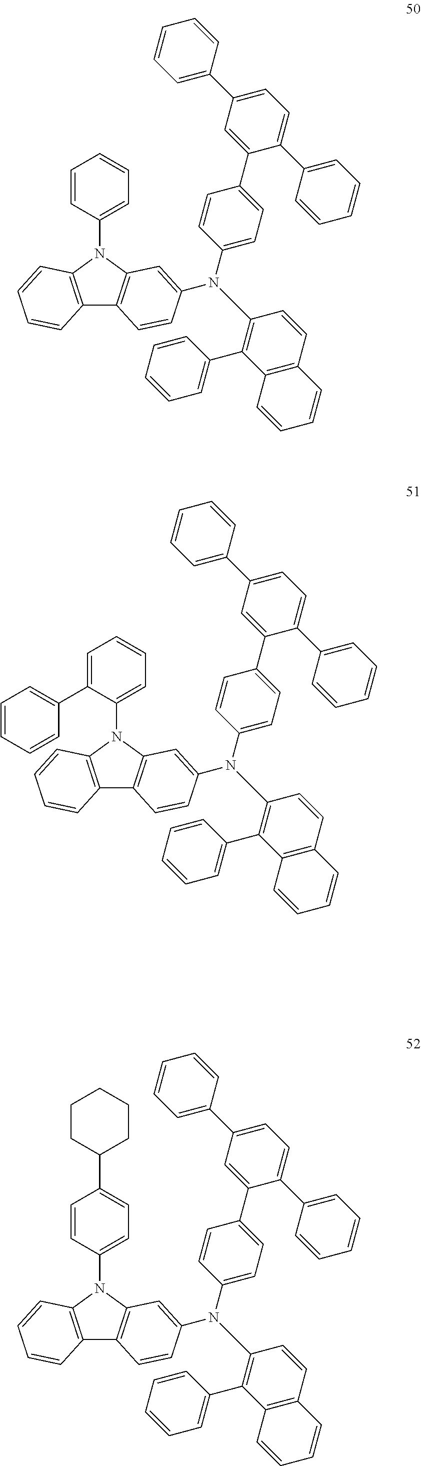 [SMILES: c1ccc(-c2ccc(-c3ccccc3)c(-c3ccc(N(c4ccc5c6ccccc6n(-c6ccccc6)c5c4)c4ccc5ccccc5c4-c4ccccc4)cc3)c2)cc1 ; c1ccc(-c2ccc(-c3ccccc3)c(-c3ccc(N(c4ccc5c6ccccc6n(-c6ccccc6-c6ccccc6)c5c4)c4ccc5ccccc5c4-c4ccccc4)cc3)c2)cc1 ; c1ccc(-c2ccc(-c3ccccc3)c(-c3ccc(N(c4ccc5c6ccccc6n(-c6ccc(C7CCCCC7)cc6)c5c4)c4ccc5ccccc5c4-c4ccccc4)cc3)c2)cc1]

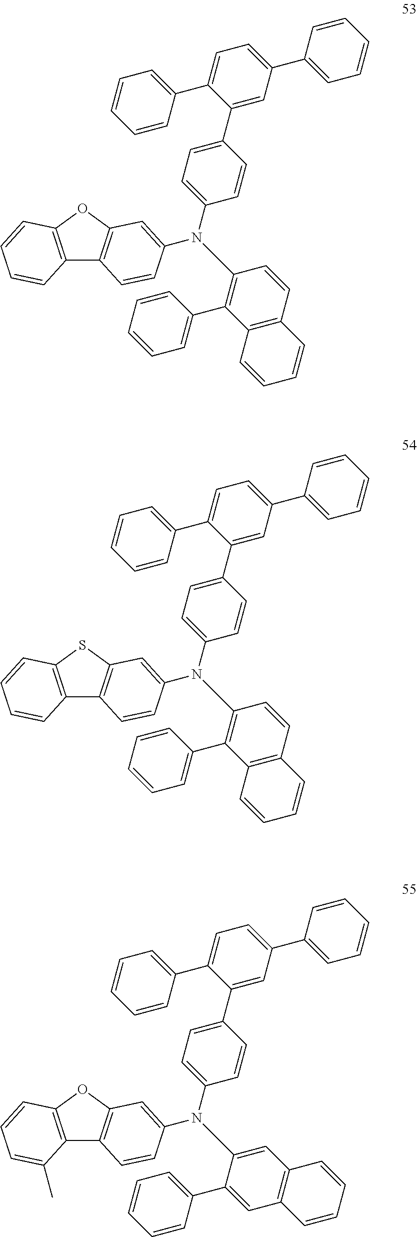 [SMILES: c1ccc(-c2ccc(-c3ccccc3)c(-c3ccc(N(c4ccc5c(c4)oc4ccccc45)c4ccc5ccccc5c4-c4ccccc4)cc3)c2)cc1 ; c1ccc(-c2ccc(-c3ccccc3)c(-c3ccc(N(c4ccc5c(c4)sc4ccccc45)c4ccc5ccccc5c4-c4ccccc4)cc3)c2)cc1 ; Cc1cccc2oc3cc(N(c4ccc(-c5cc(-c6ccccc6)ccc5-c5ccccc5)cc4)c4cc5ccccc5cc4-c4ccccc4)ccc3c12]

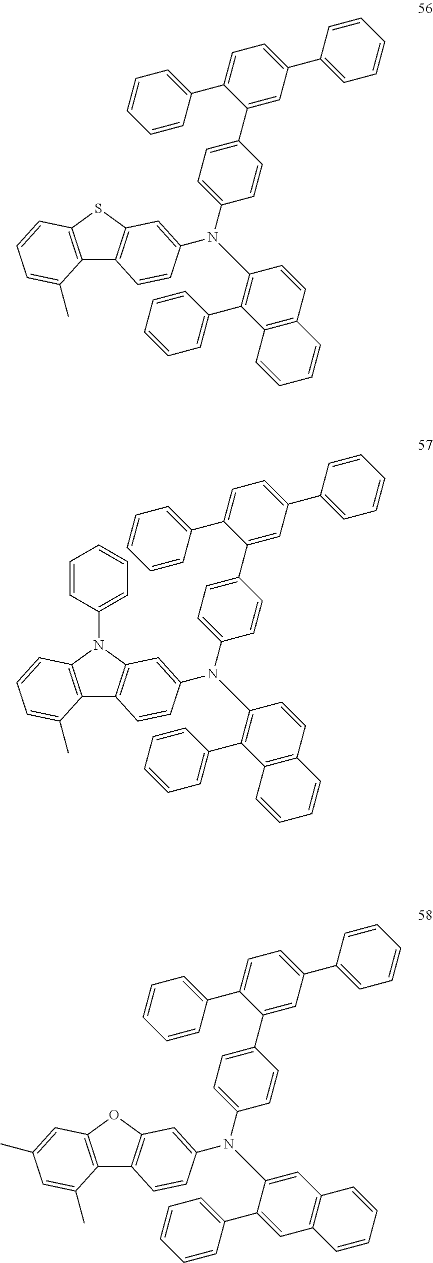 [SMILES: Cc1cccc2sc3cc(N(c4ccc(-c5cc(-c6ccccc6)ccc5-c5ccccc5)cc4)c4ccc5ccccc5c4-c4ccccc4)ccc3c12 ; Cc1cccc2c1c1ccc(N(c3ccc(-c4cc(-c5ccccc5)ccc4-c4ccccc4)cc3)c3ccc4ccccc4c3-c3ccccc3)cc1n2-c1ccccc1 ; Cc1cc(C)c2c(c1)oc1cc(N(c3ccc(-c4cc(-c5ccccc5)ccc4-c4ccccc4)cc3)c3cc4ccccc4cc3-c3ccccc3)ccc12]

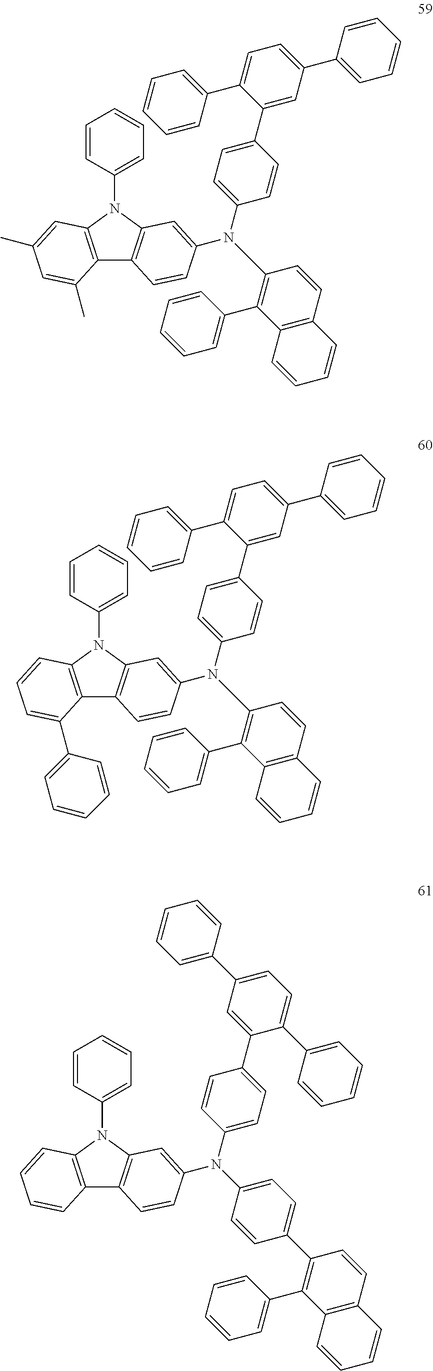 [SMILES: Cc1cc(C)c2c3ccc(N(c4ccc(-c5cc(-c6ccccc6)ccc5-c5ccccc5)cc4)c4ccc5ccccc5c4-c4ccccc4)cc3n(-c3ccccc3)c2c1 ; c1ccc(-c2ccc(-c3ccccc3)c(-c3ccc(N(c4ccc5c6c(-c7ccccc7)cccc6n(-c6ccccc6)c5c4)c4ccc5ccccc5c4-c4ccccc4)cc3)c2)cc1 ; c1ccc(-c2ccc(-c3ccccc3)c(-c3ccc(N(c4ccc(-c5ccc6ccccc6c5-c5ccccc5)cc4)c4ccc5c6ccccc6n(-c6ccccc6)c5c4)cc3)c2)cc1]

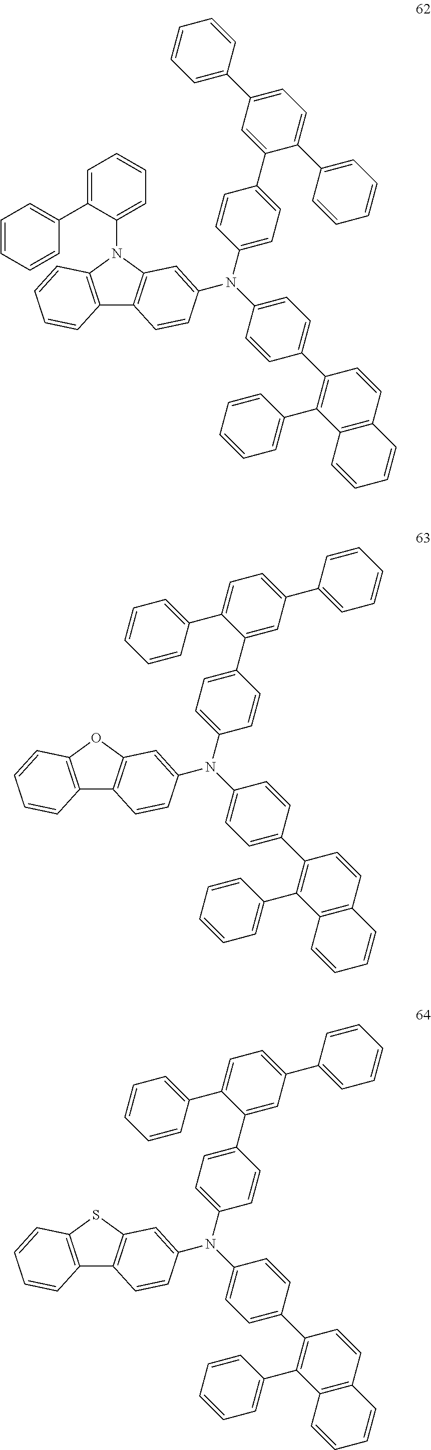 [SMILES: c1ccc(-c2ccc(-c3ccccc3)c(-c3ccc(N(c4ccc(-c5ccc6ccccc6c5-c5ccccc5)cc4)c4ccc5c6ccccc6n(-c6ccccc6-c6ccccc6)c5c4)cc3)c2)cc1 ; c1ccc(-c2ccc(-c3ccccc3)c(-c3ccc(N(c4ccc(-c5ccc6ccccc6c5-c5ccccc5)cc4)c4ccc5c(c4)oc4ccccc45)cc3)c2)cc1 ; c1ccc(-c2ccc(-c3ccccc3)c(-c3ccc(N(c4ccc(-c5ccc6ccccc6c5-c5ccccc5)cc4)c4ccc5c(c4)sc4ccccc45)cc3)c2)cc1]

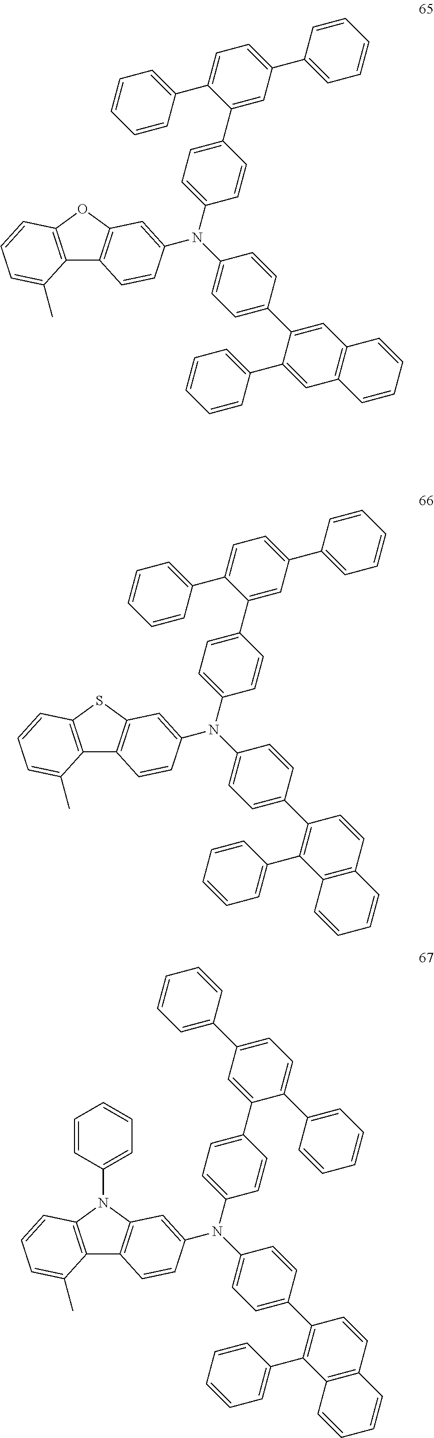 [SMILES: Cc1cccc2oc3cc(N(c4ccc(-c5cc(-c6ccccc6)ccc5-c5ccccc5)cc4)c4ccc(-c5cc6ccccc6cc5-c5ccccc5)cc4)ccc3c12 ; Cc1cccc2sc3cc(N(c4ccc(-c5cc(-c6ccccc6)ccc5-c5ccccc5)cc4)c4ccc(-c5ccc6ccccc6c5-c5ccccc5)cc4)ccc3c12 ; Cc1cccc2c1c1ccc(N(c3ccc(-c4cc(-c5ccccc5)ccc4-c4ccccc4)cc3)c3ccc(-c4ccc5ccccc5c4-c4ccccc4)cc3)cc1n2-c1ccccc1]

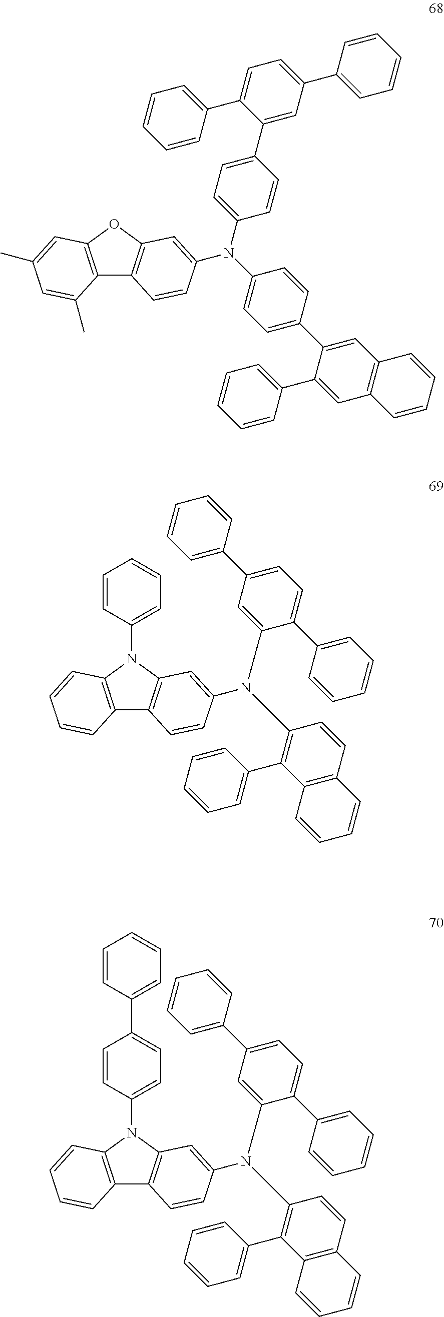 [SMILES: Cc1cc(C)c2c(c1)oc1cc(N(c3ccc(-c4cc(-c5ccccc5)ccc4-c4ccccc4)cc3)c3ccc(-c4cc5ccccc5cc4-c4ccccc4)cc3)ccc12 ; c1ccc(-c2ccc(-c3ccccc3)c(N(c3ccc4c5ccccc5n(-c5ccccc5)c4c3)c3ccc4ccccc4c3-c3ccccc3)c2)cc1 ; c1ccc(-c2ccc(-n3c4ccccc4c4ccc(N(c5cc(-c6ccccc6)ccc5-c5ccccc5)c5ccc6ccccc6c5-c5ccccc5)cc43)cc2)cc1]

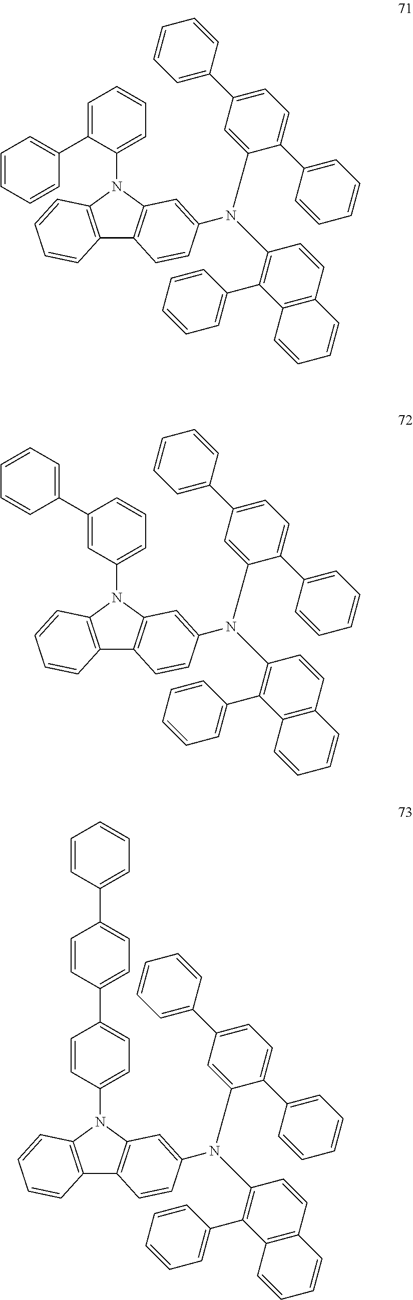 [SMILES: c1ccc(-c2ccc(-c3ccccc3)c(N(c3ccc4c5ccccc5n(-c5ccccc5-c5ccccc5)c4c3)c3ccc4ccccc4c3-c3ccccc3)c2)cc1 ; c1ccc(-c2cccc(-n3c4ccccc4c4ccc(N(c5cc(-c6ccccc6)ccc5-c5ccccc5)c5ccc6ccccc6c5-c5ccccc5)cc43)c2)cc1 ; c1ccc(-c2ccc(-c3ccc(-n4c5ccccc5c5ccc(N(c6cc(-c7ccccc7)ccc6-c6ccccc6)c6ccc7ccccc7c6-c6ccccc6)cc54)cc3)cc2)cc1]

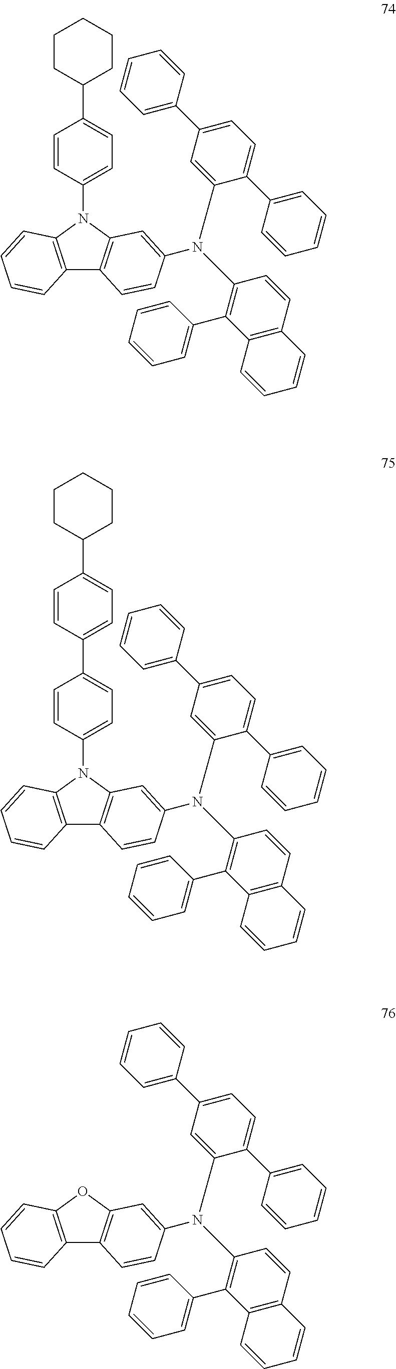 [SMILES: c1ccc(-c2ccc(-c3ccccc3)c(N(c3ccc4c5ccccc5n(-c5ccc(C6CCCCC6)cc5)c4c3)c3ccc4ccccc4c3-c3ccccc3)c2)cc1 ; c1ccc(-c2ccc(-c3ccccc3)c(N(c3ccc4c5ccccc5n(-c5ccc(-c6ccc(C7CCCCC7)cc6)cc5)c4c3)c3ccc4ccccc4c3-c3ccccc3)c2)cc1 ; c1ccc(-c2ccc(-c3ccccc3)c(N(c3ccc4c(c3)oc3ccccc34)c3ccc4ccccc4c3-c3ccccc3)c2)cc1]

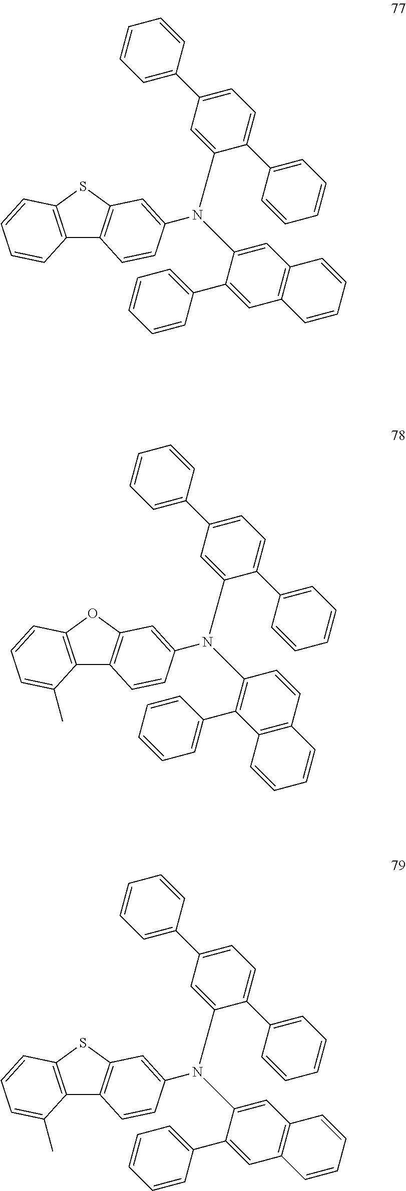 [SMILES: c1ccc(-c2ccc(-c3ccccc3)c(N(c3ccc4c(c3)sc3ccccc34)c3cc4ccccc4cc3-c3ccccc3)c2)cc1 ; Cc1cccc2oc3cc(N(c4cc(-c5ccccc5)ccc4-c4ccccc4)c4ccc5ccccc5c4-c4ccccc4)ccc3c12 ; Cc1cccc2sc3cc(N(c4cc(-c5ccccc5)ccc4-c4ccccc4)c4cc5ccccc5cc4-c4ccccc4)ccc3c12]

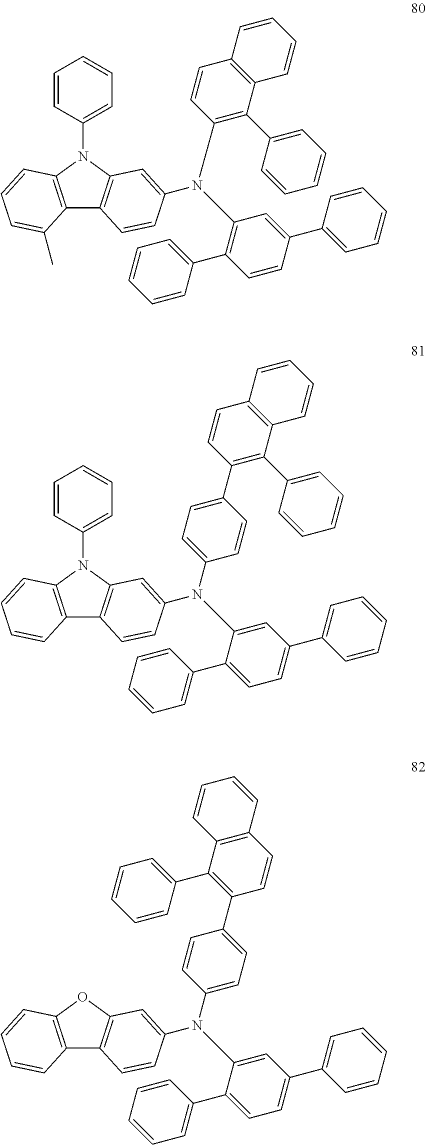 [SMILES: Cc1cccc2c1c1ccc(N(c3cc(-c4ccccc4)ccc3-c3ccccc3)c3ccc4ccccc4c3-c3ccccc3)cc1n2-c1ccccc1 ; c1ccc(-c2ccc(-c3ccccc3)c(N(c3ccc(-c4ccc5ccccc5c4-c4ccccc4)cc3)c3ccc4c5ccccc5n(-c5ccccc5)c4c3)c2)cc1 ; c1ccc(-c2ccc(-c3ccccc3)c(N(c3ccc(-c4ccc5ccccc5c4-c4ccccc4)cc3)c3ccc4c(c3)oc3ccccc34)c2)cc1]

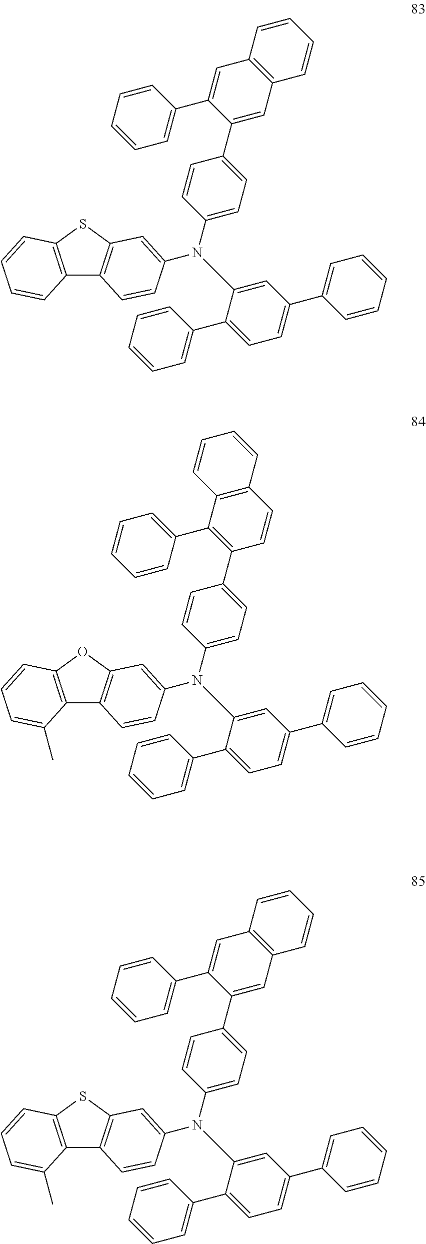 [SMILES: c1ccc(-c2ccc(-c3ccccc3)c(N(c3ccc(-c4cc5ccccc5cc4-c4ccccc4)cc3)c3ccc4c(c3)sc3ccccc34)c2)cc1 ; Cc1cccc2oc3cc(N(c4ccc(-c5ccc6ccccc6c5-c5ccccc5)cc4)c4cc(-c5ccccc5)ccc4-c4ccccc4)ccc3c12 ; Cc1cccc2sc3cc(N(c4ccc(-c5cc6ccccc6cc5-c5ccccc5)cc4)c4cc(-c5ccccc5)ccc4-c4ccccc4)ccc3c12]

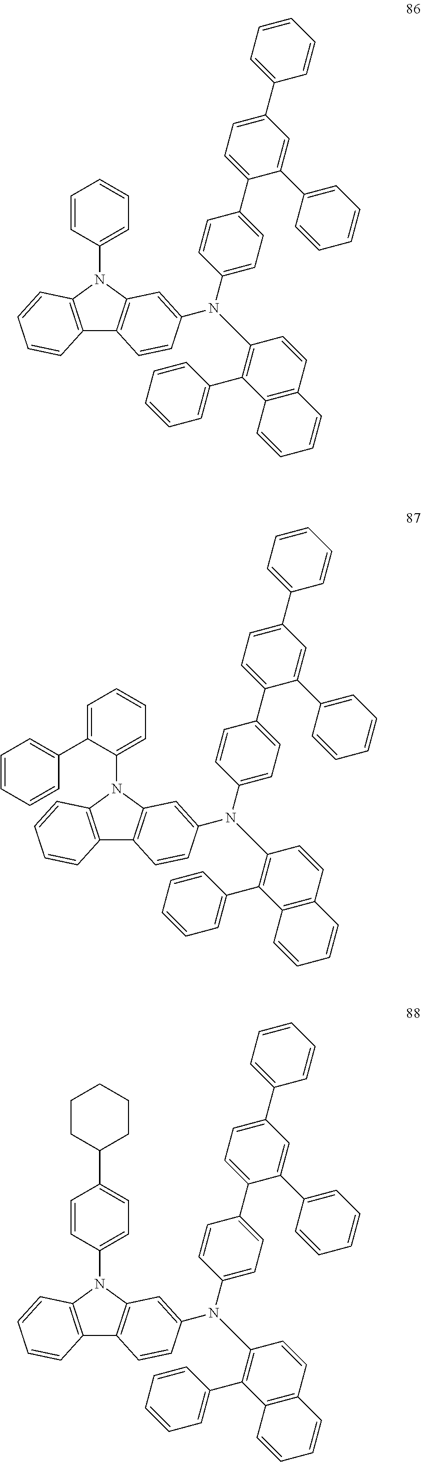 [SMILES: c1ccc(-c2ccc(-c3ccc(N(c4ccc5c6ccccc6n(-c6ccccc6)c5c4)c4ccc5ccccc5c4-c4ccccc4)cc3)c(-c3ccccc3)c2)cc1 ; c1ccc(-c2ccc(-c3ccc(N(c4ccc5c6ccccc6n(-c6ccccc6-c6ccccc6)c5c4)c4ccc5ccccc5c4-c4ccccc4)cc3)c(-c3ccccc3)c2)cc1 ; c1ccc(-c2ccc(-c3ccc(N(c4ccc5c6ccccc6n(-c6ccc(C7CCCCC7)cc6)c5c4)c4ccc5ccccc5c4-c4ccccc4)cc3)c(-c3ccccc3)c2)cc1]

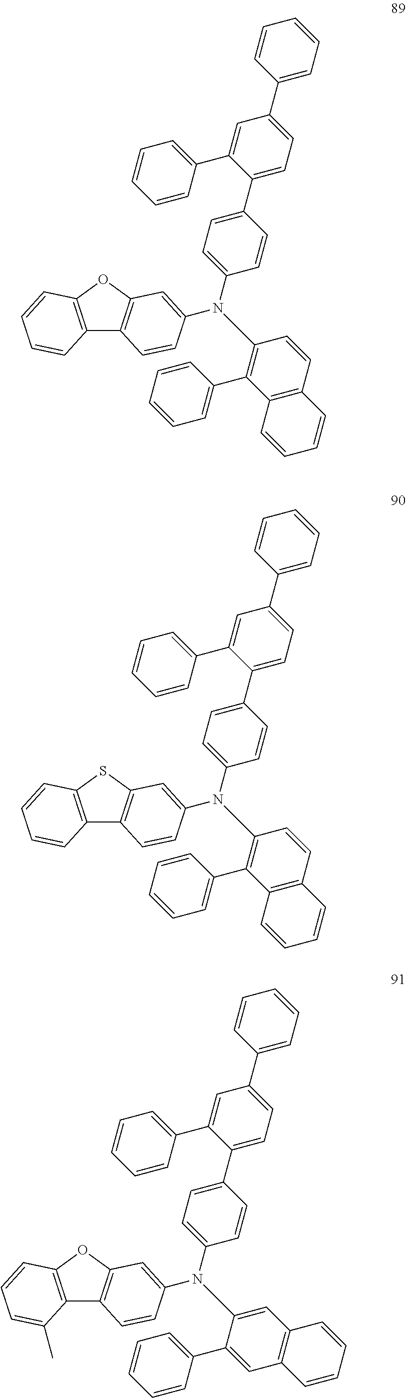

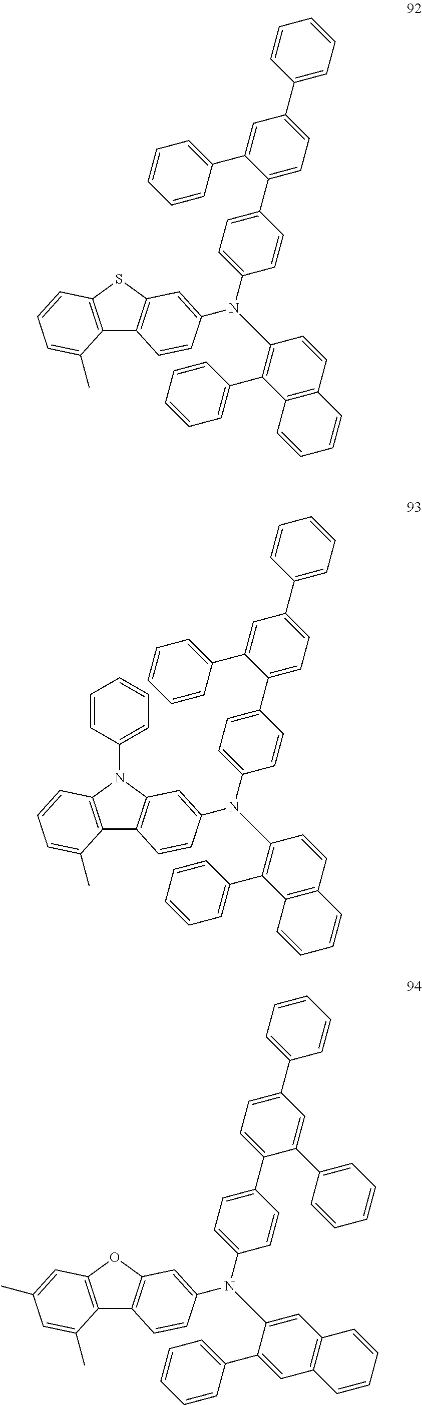

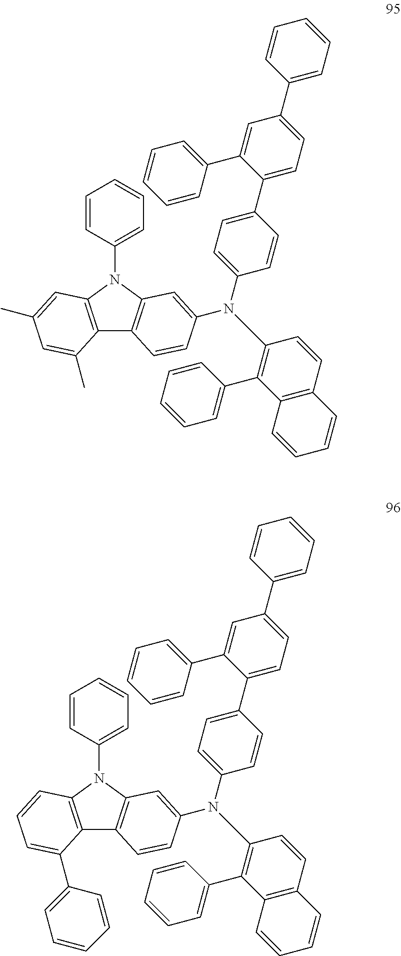 [SMILES: Cc1cc(C)c2c3ccc(N(c4ccc(-c5ccc(-c6ccccc6)cc5-c5ccccc5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)c(-c3ccccc3)c2)cc1]

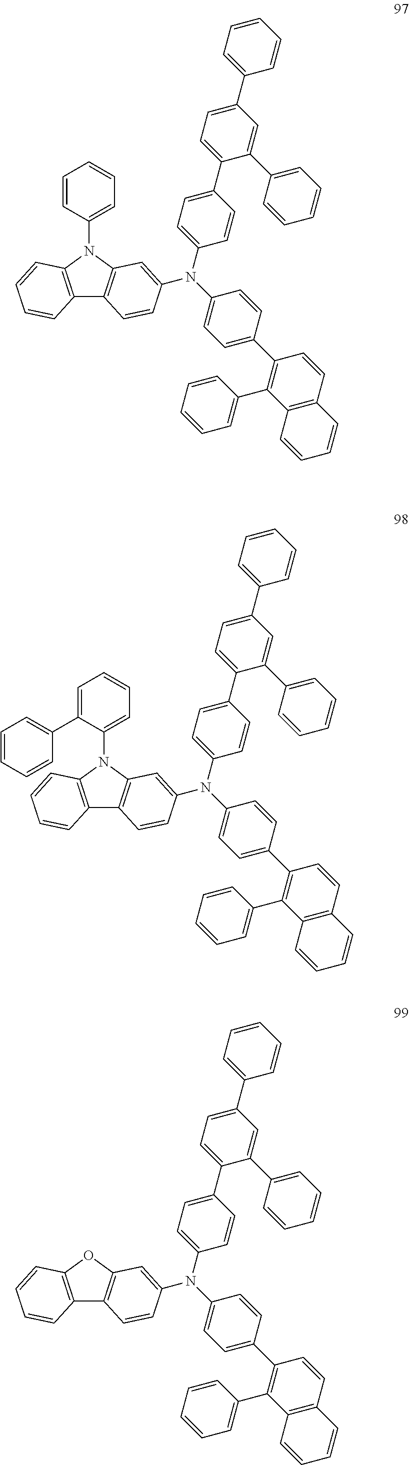 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c(c4)oc4ccccc45)cc3)c(-c3ccccc3)c2)cc1]

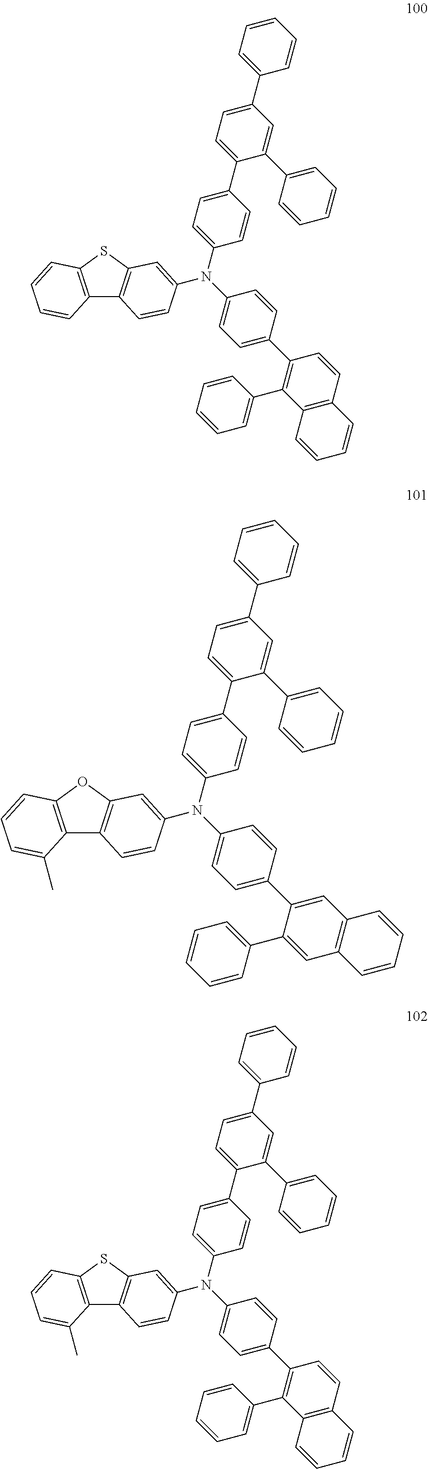 [SMILES: c1ccc(-c2ccc(-c3ccc(N(c4ccc(-c5ccc6ccccc6c5-c5ccccc5)cc4)c4ccc5c(c4)sc4ccccc45)cc3)c(-c3ccccc3)c2)cc1 ; Cc1cccc2oc3cc(N(c4ccc(-c5ccc(-c6ccccc6)cc5-c5ccccc5)cc4)c4ccc(-c5cc6ccccc6cc5-c5ccccc5)cc4)ccc3c12 ; Cc1cccc2sc3cc(N(c4ccc(-c5ccc(-c6ccccc6)cc5-c5ccccc5)cc4)c4ccc(-c5ccc6ccccc6c5-c5ccccc5)cc4)ccc3c12]

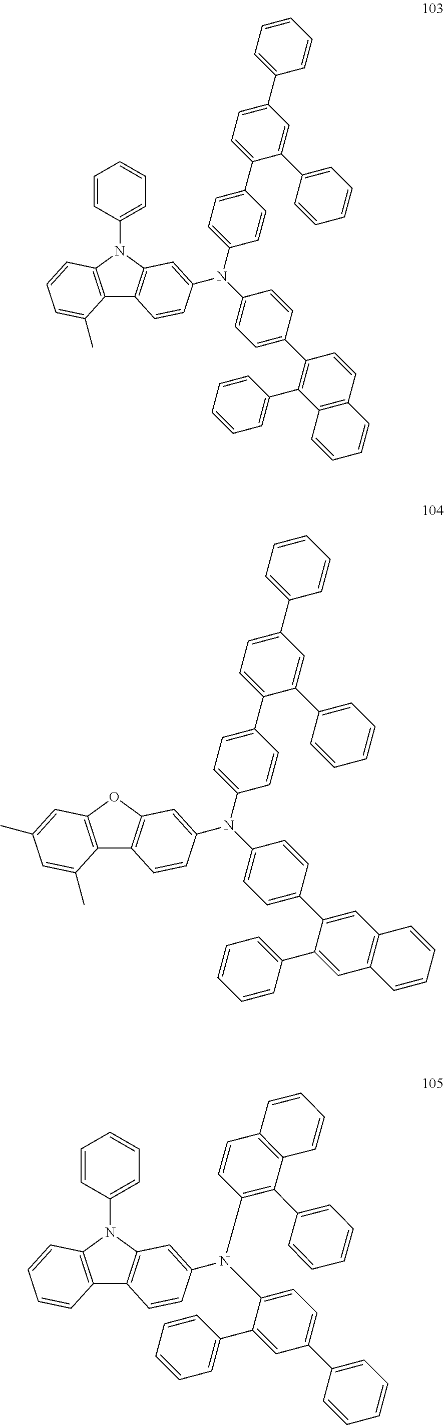 [SMILES: Cc1cccc2c1c1ccc(N(c3ccc(-c4ccc(-c5ccccc5)cc4-c4ccccc4)cc3)c3ccc(-c4ccc5ccccc5c4-c4ccccc4)cc3)cc1n2-c1ccccc1 ; Cc1cc(C)c2c(c1)oc1cc(N(c3ccc(-c4ccc(-c5ccccc5)cc4-c4ccccc4)cc3)c3ccc(-c4cc5ccccc5cc4-c4ccccc4)cc3)ccc12 ; c1ccc(-c2ccc(N(c3ccc4c5ccccc5n(-c5ccccc5)c4c3)c3ccc4ccccc4c3-c3ccccc3)c(-c3ccccc3)c2)cc1]

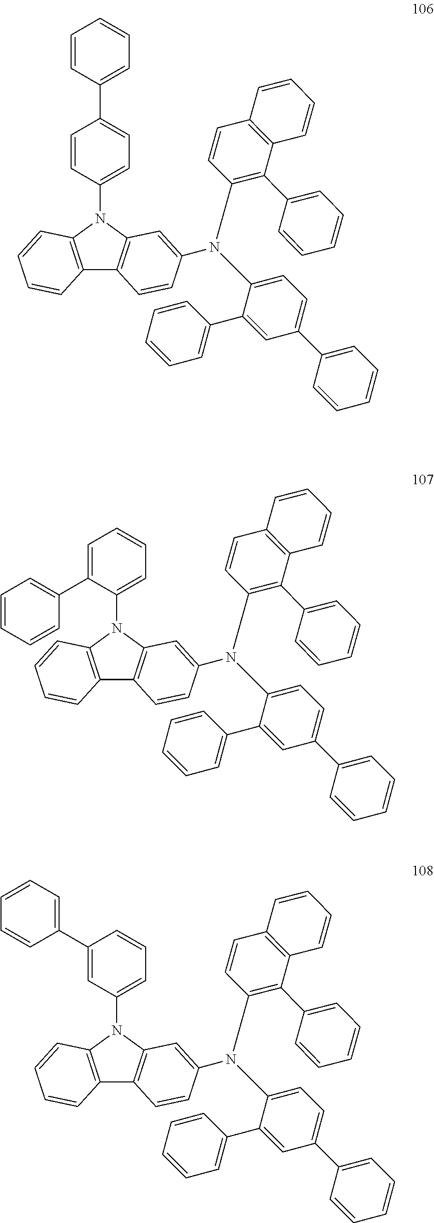 [SMILES: c1ccc(-c2ccc(-n3c4ccccc4c4ccc(N(c5ccc(-c6ccccc6)cc5-c5ccccc5)c5ccc6ccccc6c5-c5ccccc5)cc43)cc2)cc1 ; c1ccc(-c2ccc(N(c3ccc4c5ccccc5n(-c5ccccc5-c5ccccc5)c4c3)c3ccc4ccccc4c3-c3ccccc3)c(-c3ccccc3)c2)cc1 ; c1ccc(-c2cccc(-n3c4ccccc4c4ccc(N(c5ccc(-c6ccccc6)cc5-c5ccccc5)c5ccc6ccccc6c5-c5ccccc5)cc43)c2)cc1]

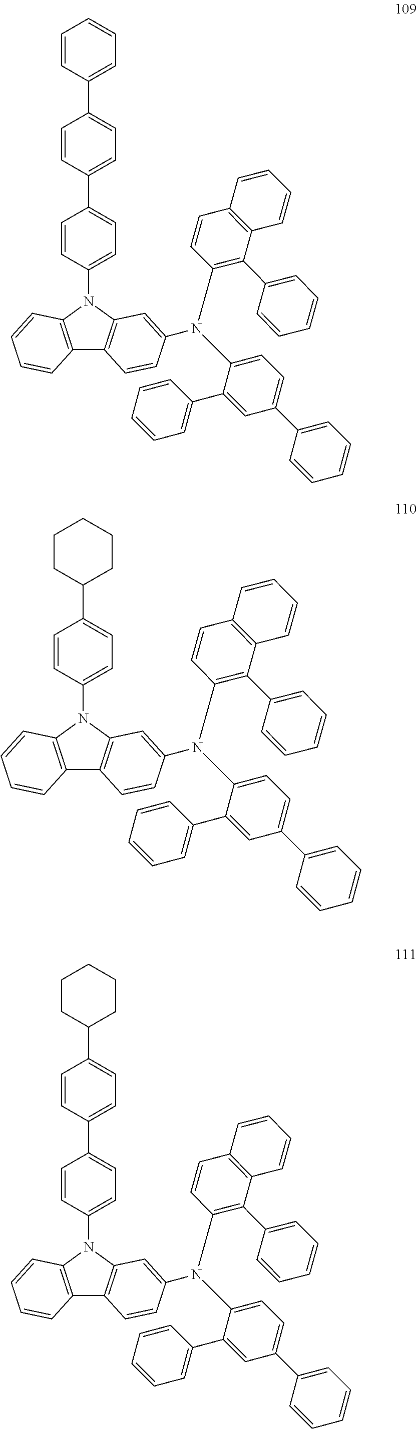 [SMILES: c1ccc(-c2ccc(-c3ccc(-n4c5ccccc5c5ccc(N(c6ccc(-c7ccccc7)cc6-c6ccccc6)c6ccc7ccccc7c6-c6ccccc6)cc54)cc3)cc2)cc1 ; c1ccc(-c2ccc(N(c3ccc4c5ccccc5n(-c5ccc(C6CCCCC6)cc5)c4c3)c3ccc4ccccc4c3-c3ccccc3)c(-c3ccccc3)c2)cc1 ; c1ccc(-c2ccc(N(c3ccc4c5ccccc5n(-c5ccc(-c6ccc(C7CCCCC7)cc6)cc5)c4c3)c3ccc4ccccc4c3-c3ccccc3)c(-c3ccccc3)c2)cc1]

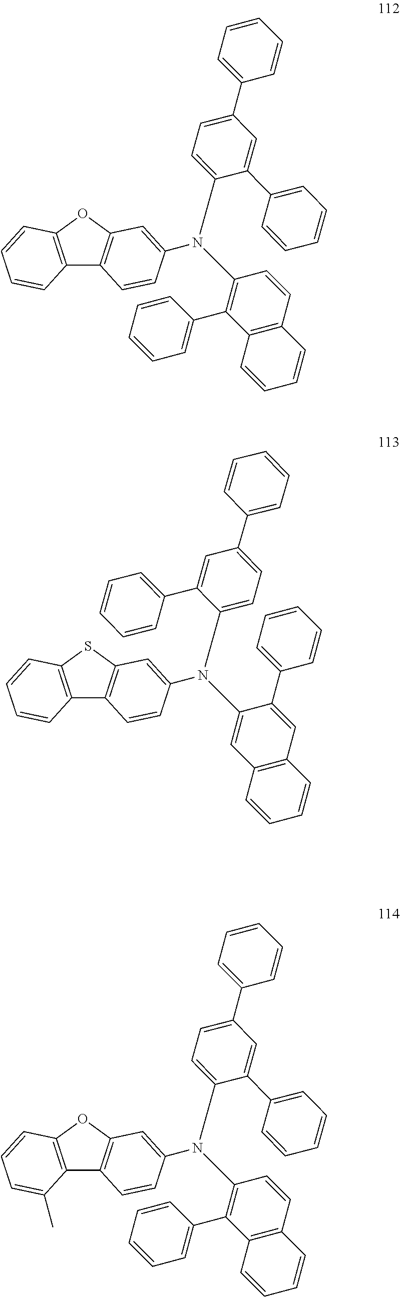 [SMILES: c1ccc(-c2ccc(N(c3ccc4c(c3)oc3ccccc34)c3ccc4ccccc4c3-c3ccccc3)c(-c3ccccc3)c2)cc1 ; c1ccc(-c2ccc(N(c3ccc4c(c3)sc3ccccc34)c3cc4ccccc4cc3-c3ccccc3)c(-c3ccccc3)c2)cc1 ; Cc1cccc2oc3cc(N(c4ccc(-c5ccccc5)cc4-c4ccccc4)c4ccc5ccccc5c4-c4ccccc4)ccc3c12]

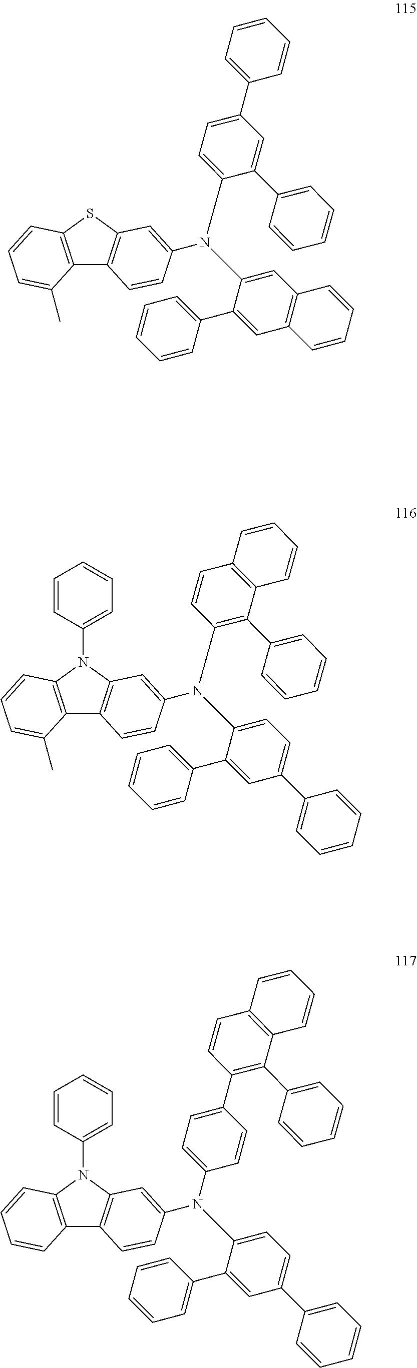 [SMILES: Cc1cccc2sc3cc(N(c4ccc(-c5ccccc5)cc4-c4ccccc4)c4cc5ccccc5cc4-c4ccccc4)ccc3c12 ; Cc1cccc2c1c1ccc(N(c3ccc(-c4ccccc4)cc3-c3ccccc3)c3ccc4ccccc4c3-c3ccccc3)cc1n2-c1ccccc1 ; c1ccc(-c2ccc(N(c3ccc(-c4ccc5ccccc5c4-c4ccccc4)cc3)c3ccc4c5ccccc5n(-c5ccccc5)c4c3)c(-c3ccccc3)c2)cc1]

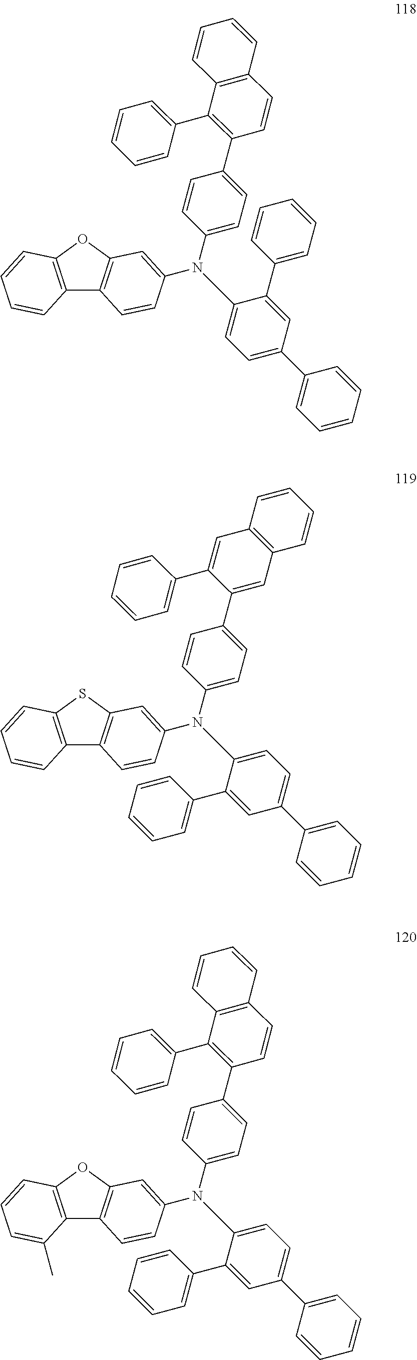 [SMILES: c1ccc(-c2ccc(N(c3ccc(-c4ccc5ccccc5c4-c4ccccc4)cc3)c3ccc4c(c3)oc3ccccc34)c(-c3ccccc3)c2)cc1 ; c1ccc(-c2ccc(N(c3ccc(-c4cc5ccccc5cc4-c4ccccc4)cc3)c3ccc4c(c3)sc3ccccc34)c(-c3ccccc3)c2)cc1 ; Cc1cccc2oc3cc(N(c4ccc(-c5ccc6ccccc6c5-c5ccccc5)cc4)c4ccc(-c5ccccc5)cc4-c4ccccc4)ccc3c12]

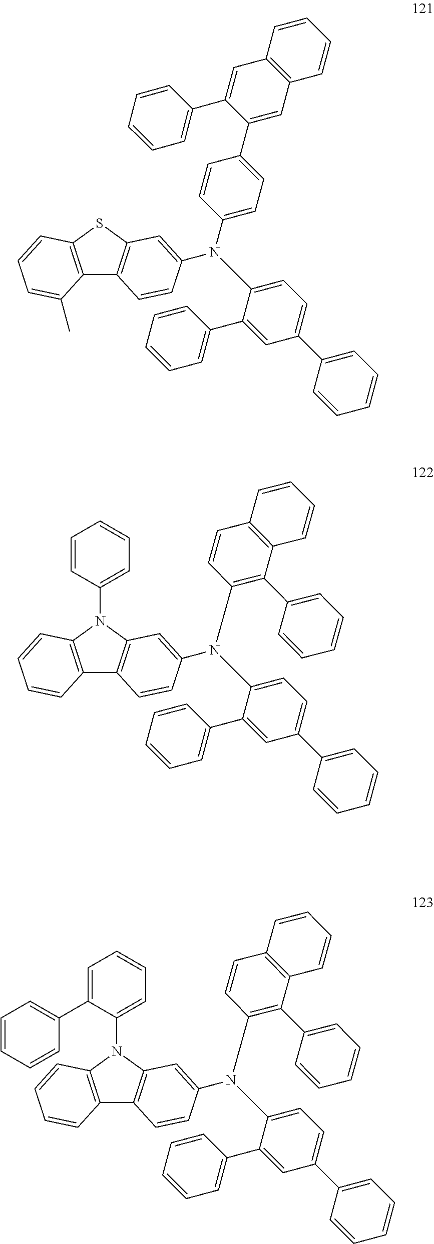 [SMILES: Cc1cccc2sc3cc(N(c4ccc(-c5cc6ccccc6cc5-c5ccccc5)cc4)c4ccc(-c5ccccc5)cc4-c4ccccc4)ccc3c12 ; c1ccc(-c2ccc(N(c3ccc4c5ccccc5n(-c5ccccc5)c4c3)c3ccc4ccccc4c3-c3ccccc3)c(-c3ccccc3)c2)cc1 ; c1ccc(-c2ccc(N(c3ccc4c5ccccc5n(-c5ccccc5-c5ccccc5)c4c3)c3ccc4ccccc4c3-c3ccccc3)c(-c3ccccc3)c2)cc1]

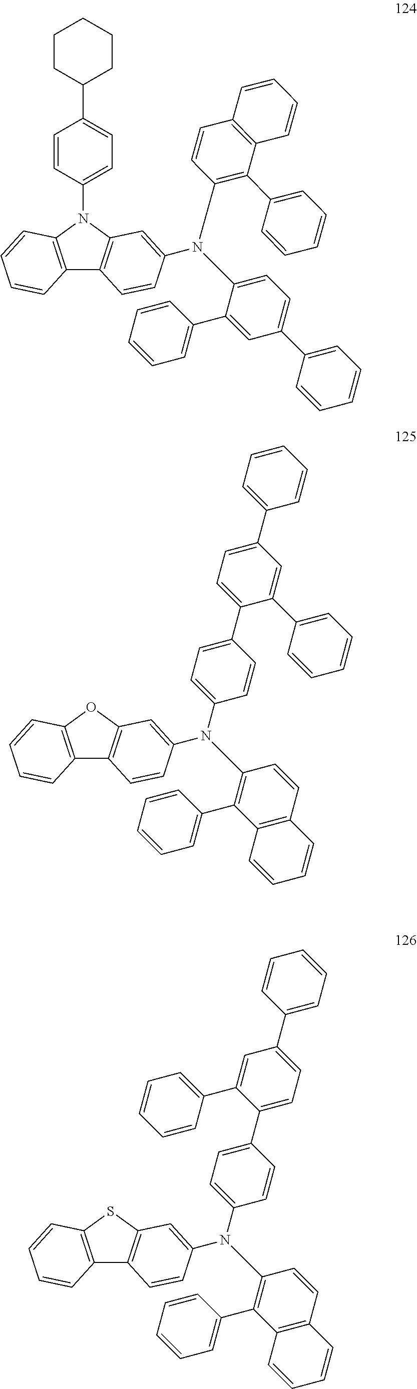 [SMILES: c1ccc(-c2ccc(N(c3ccc4c5ccccc5n(-c5ccc(C6CCCCC6)cc5)c4c3)c3ccc4ccccc4c3-c3ccccc3)c(-c3ccccc3)c2)cc1 ; c1ccc(-c2ccc(-c3ccc(N(c4ccc5c(c4)oc4ccccc45)c4ccc5ccccc5c4-c4ccccc4)cc3)c(-c3ccccc3)c2)cc1 ; c1ccc(-c2ccc(-c3ccc(N(c4ccc5c(c4)sc4ccccc45)c4ccc5ccccc5c4-c4ccccc4)cc3)c(-c3ccccc3)c2)cc1]

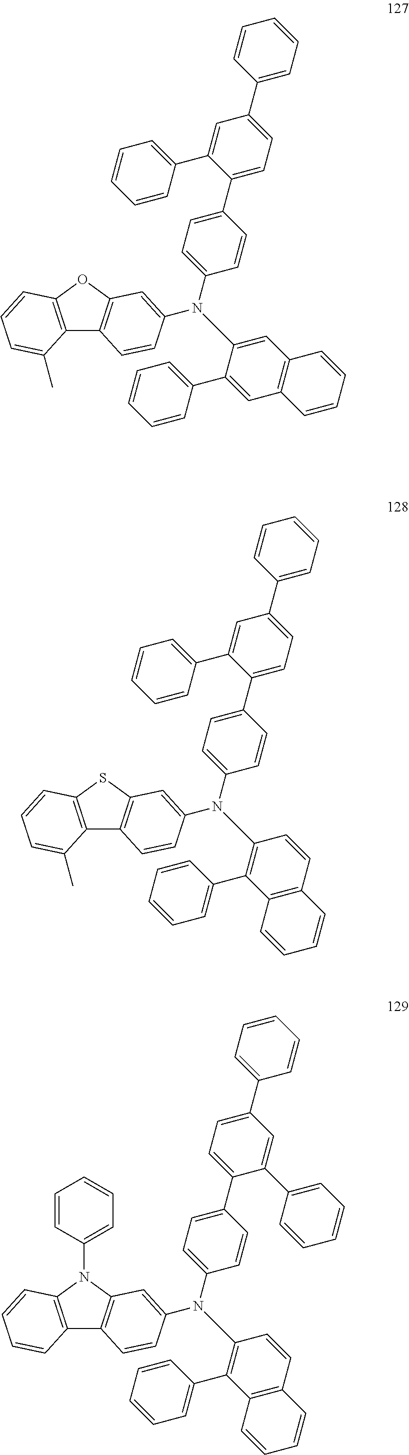 [SMILES: Cc1cccc2oc3cc(N(c4ccc(-c5ccc(-c6ccccc6)cc5-c5ccccc5)cc4)c4cc5ccccc5cc4-c4ccccc4)ccc3c12 ; Cc1cccc2sc3cc(N(c4ccc(-c5ccc(-c6ccccc6)cc5-c5ccccc5)cc4)c4ccc5ccccc5c4-c4ccccc4)ccc3c12 ; c1ccc(-c2ccc(-c3ccc(N(c4ccc5c6ccccc6n(-c6ccccc6)c5c4)c4ccc5ccccc5c4-c4ccccc4)cc3)c(-c3ccccc3)c2)cc1]

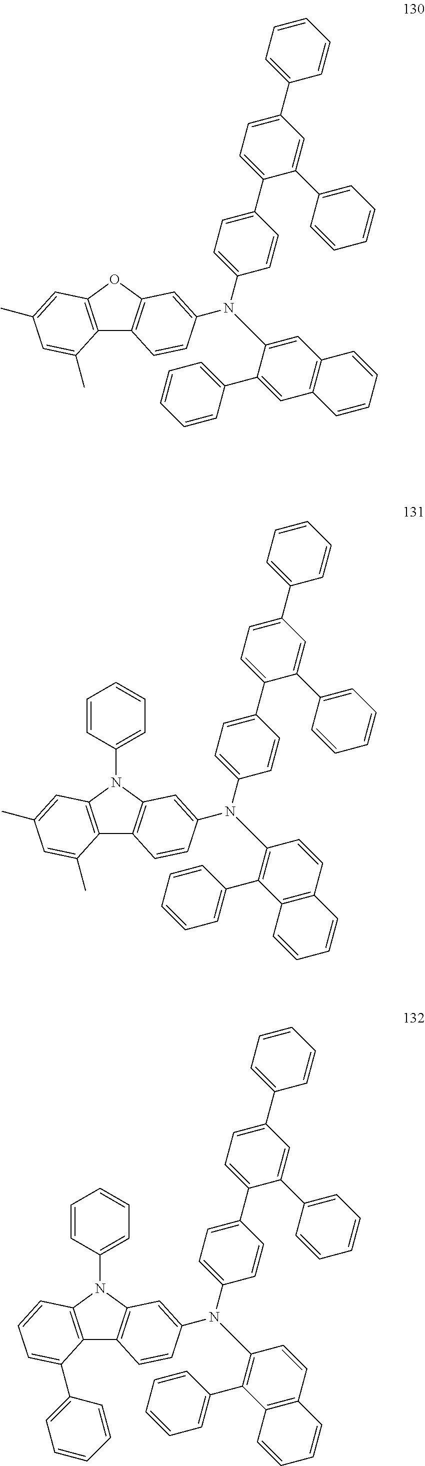 [SMILES: Cc1cc(C)c2c(c1)oc1cc(N(c3ccc(-c4ccc(-c5ccccc5)cc4-c4ccccc4)cc3)c3cc4ccccc4cc3-c3ccccc3)ccc12 ; Cc1cc(C)c2c3ccc(N(c4ccc(-c5ccc(-c6ccccc6)cc5-c5ccccc5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)c(-c3ccccc3)c2)cc1]

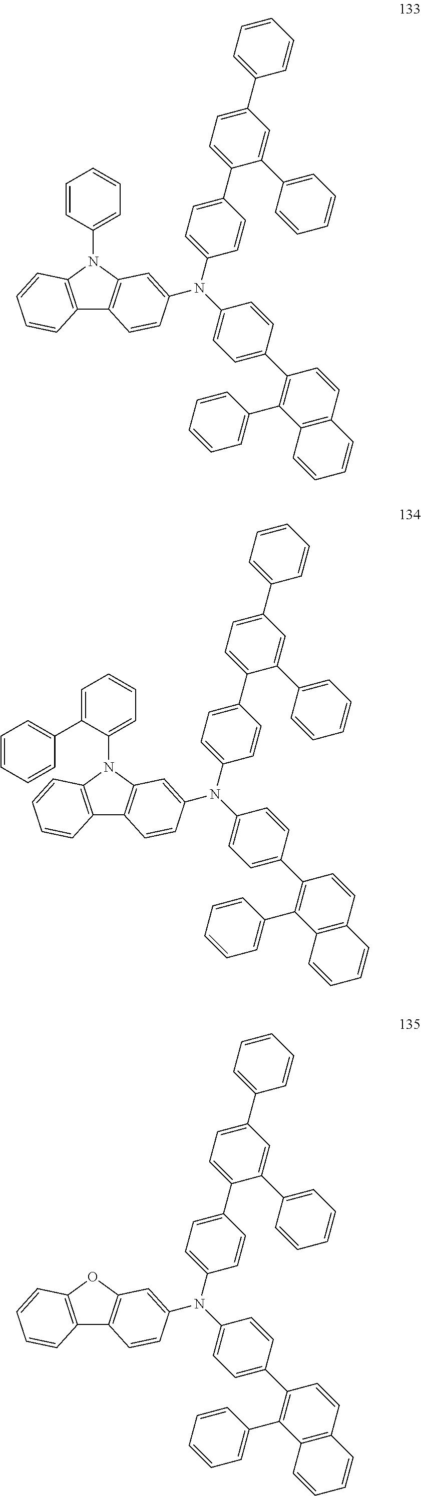 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)c(-c3ccccc3)c2)cc1 ; c1ccc(-c2ccc(-c3ccc(N(c4ccc(-c5ccc6ccccc6c5-c5ccccc5)cc4)c4ccc5c(c4)oc4ccccc45)cc3)c(-c3ccccc3)c2)cc1]

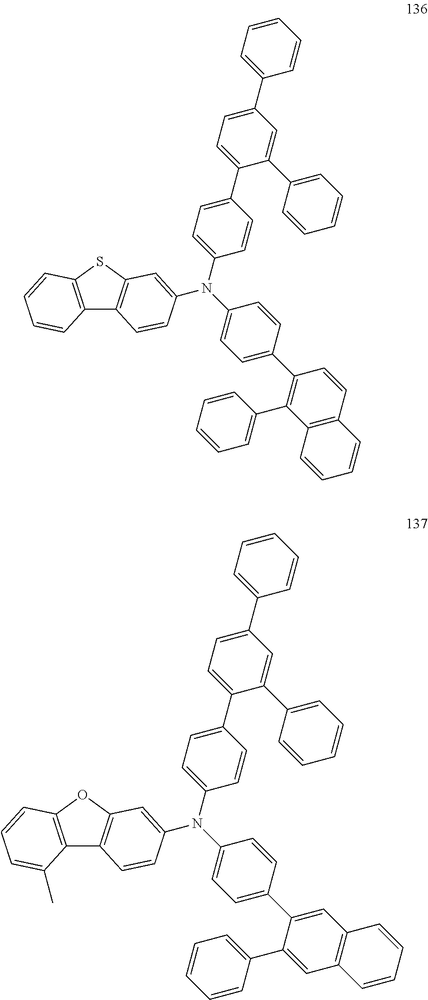 [SMILES: c1ccc(-c2ccc(-c3ccc(N(c4ccc(-c5ccc6ccccc6c5-c5ccccc5)cc4)c4ccc5c(c4)sc4ccccc45)cc3)c(-c3ccccc3)c2)cc1 ; Cc1cccc2oc3cc(N(c4ccc(-c5ccc(-c6ccccc6)cc5-c5ccccc5)cc4)c4ccc(-c5cc6ccccc6cc5-c5ccccc5)cc4)ccc3c12]

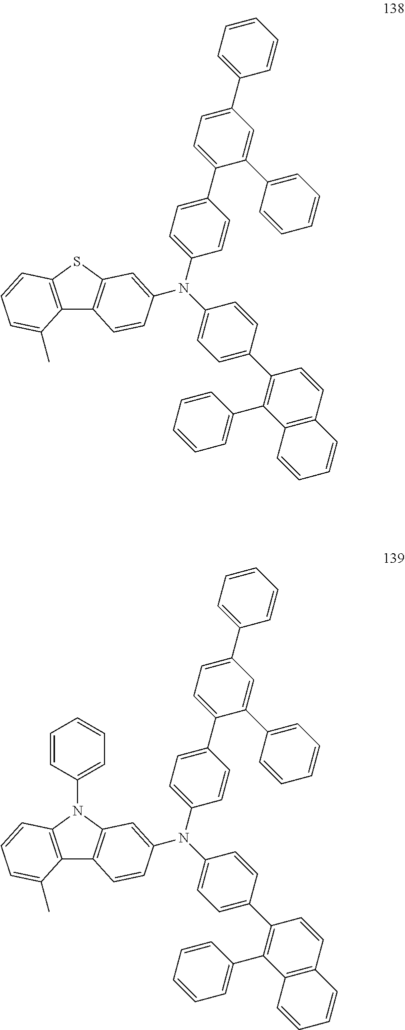 [SMILES: Cc1cccc2sc3cc(N(c4ccc(-c5ccc(-c6ccccc6)cc5-c5ccccc5)cc4)c4ccc(-c5ccc6ccccc6c5-c5ccccc5)cc4)ccc3c12 ; Cc1cccc2c1c1ccc(N(c3ccc(-c4ccc(-c5ccccc5)cc4-c4ccccc4)cc3)c3ccc(-c4ccc5ccccc5c4-c4ccccc4)cc3)cc1n2-c1ccccc1]

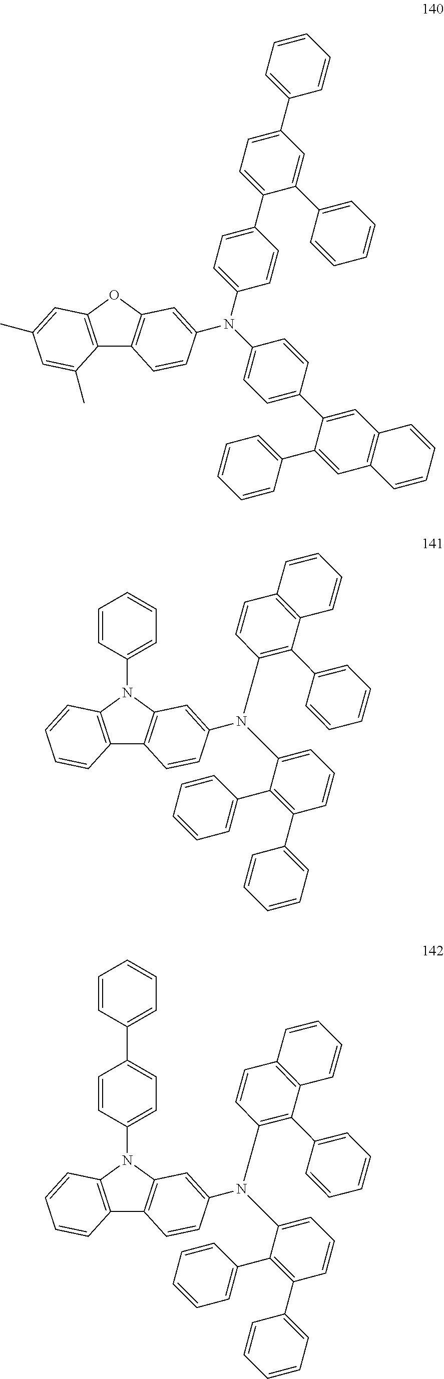 [SMILES: Cc1cc(C)c2c(c1)oc1cc(N(c3ccc(-c4ccc(-c5ccccc5)cc4-c4ccccc4)cc3)c3ccc(-c4cc5ccccc5cc4-c4ccccc4)cc3)ccc12 ; c1ccc(-c2cccc(N(c3ccc4c5ccccc5n(-c5ccccc5)c4c3)c3ccc4ccccc4c3-c3ccccc3)c2-c2ccccc2)cc1 ; c1ccc(-c2ccc(-n3c4ccccc4c4ccc(N(c5cccc(-c6ccccc6)c5-c5ccccc5)c5ccc6ccccc6c5-c5ccccc5)cc43)cc2)cc1]

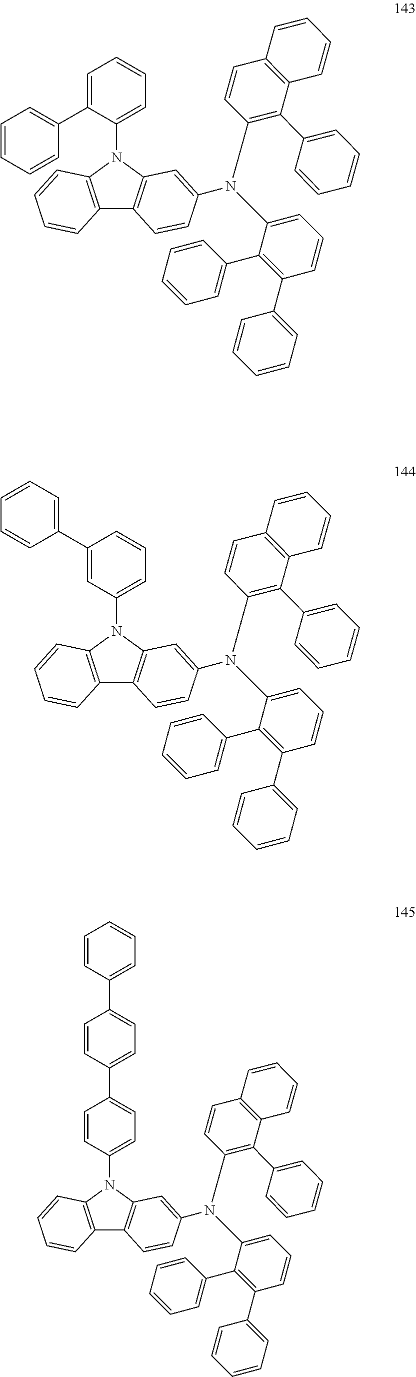 [SMILES: c1ccc(-c2ccccc2-n2c3ccccc3c3ccc(N(c4cccc(-c5ccccc5)c4-c4ccccc4)c4ccc5ccccc5c4-c4ccccc4)cc32)cc1 ; c1ccc(-c2cccc(-n3c4ccccc4c4ccc(N(c5cccc(-c6ccccc6)c5-c5ccccc5)c5ccc6ccccc6c5-c5ccccc5)cc43)c2)cc1 ; c1ccc(-c2ccc(-c3ccc(-n4c5ccccc5c5ccc(N(c6cccc(-c7ccccc7)c6-c6ccccc6)c6ccc7ccccc7c6-c6ccccc6)cc54)cc3)cc2)cc1]

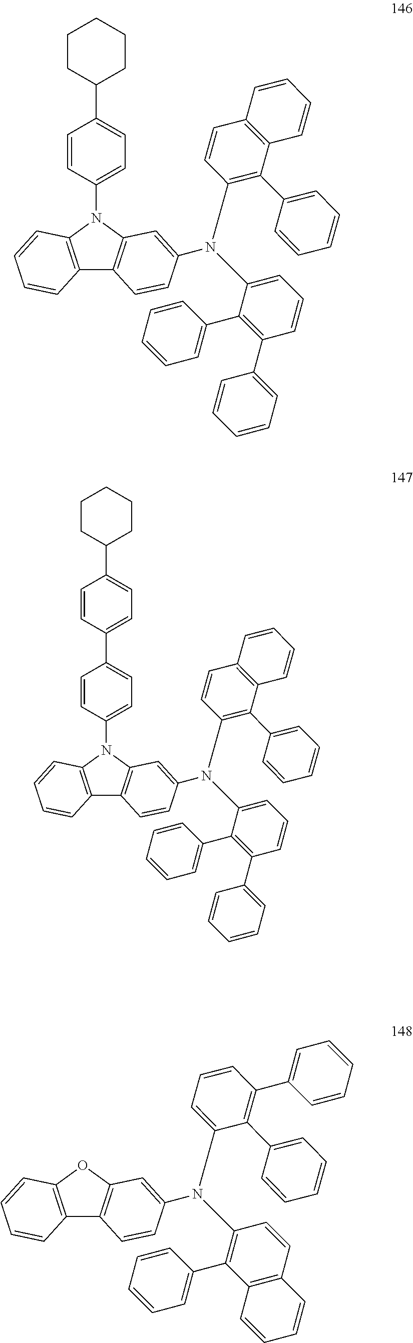 [SMILES: c1ccc(-c2cccc(N(c3ccc4c5ccccc5n(-c5ccc(C6CCCCC6)cc5)c4c3)c3ccc4ccccc4c3-c3ccccc3)c2-c2ccccc2)cc1 ; c1ccc(-c2cccc(N(c3ccc4c5ccccc5n(-c5ccc(-c6ccc(C7CCCCC7)cc6)cc5)c4c3)c3ccc4ccccc4c3-c3ccccc3)c2-c2ccccc2)cc1 ; c1ccc(-c2cccc(N(c3ccc4c(c3)oc3ccccc34)c3ccc4ccccc4c3-c3ccccc3)c2-c2ccccc2)cc1]

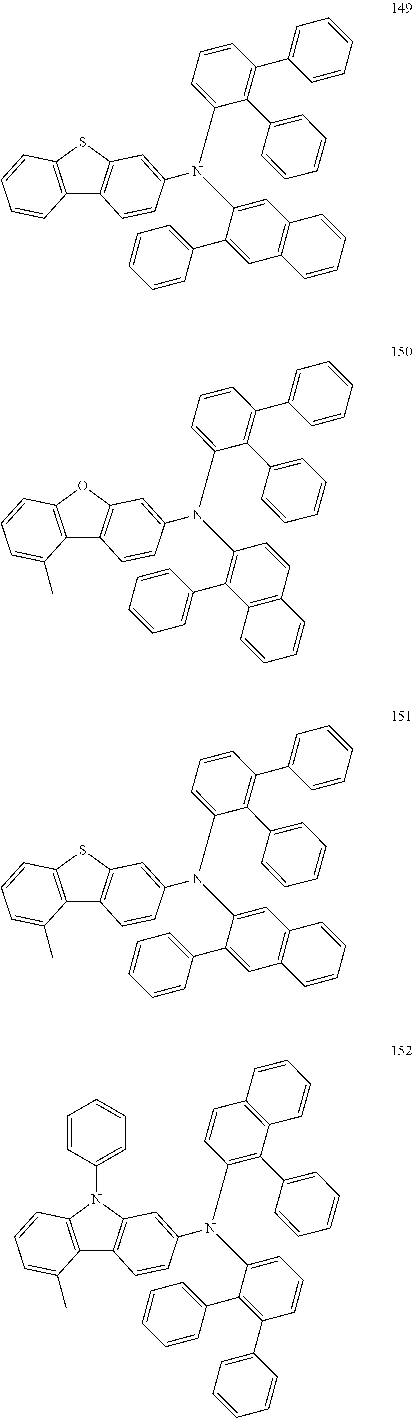 [SMILES: c1ccc(-c2cc3ccccc3cc2N(c2ccc3c(c2)sc2ccccc23)c2cccc(-c3ccccc3)c2-c2ccccc2)cc1 ; Cc1cccc2oc3cc(N(c4cccc(-c5ccccc5)c4-c4ccccc4)c4ccc5ccccc5c4-c4ccccc4)ccc3c12 ; Cc1cccc2sc3cc(N(c4cc5ccccc5cc4-c4ccccc4)c4cccc(-c5ccccc5)c4-c4ccccc4)ccc3c12 ; Cc1cccc2c1c1ccc(N(c3cccc(-c4ccccc4)c3-c3ccccc3)c3ccc4ccccc4c3-c3ccccc3)cc1n2-c1ccccc1]

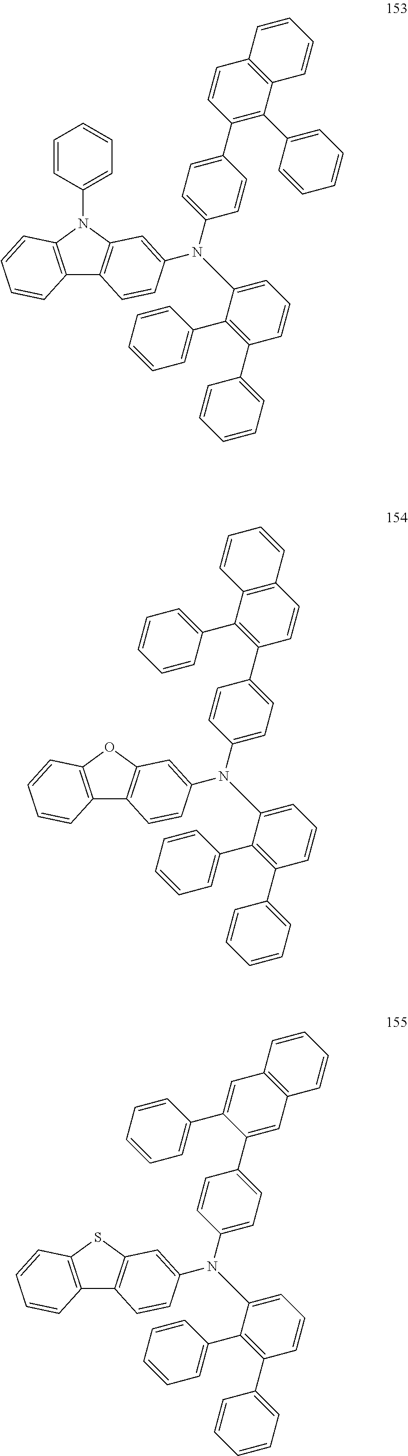 [SMILES: c1ccc(-c2cccc(N(c3ccc(-c4ccc5ccccc5c4-c4ccccc4)cc3)c3ccc4c5ccccc5n(-c5ccccc5)c4c3)c2-c2ccccc2)cc1 ; c1ccc(-c2cccc(N(c3ccc(-c4ccc5ccccc5c4-c4ccccc4)cc3)c3ccc4c(c3)oc3ccccc34)c2-c2ccccc2)cc1 ; c1ccc(-c2cc3ccccc3cc2-c2ccc(N(c3ccc4c(c3)sc3ccccc34)c3cccc(-c4ccccc4)c3-c3ccccc3)cc2)cc1]

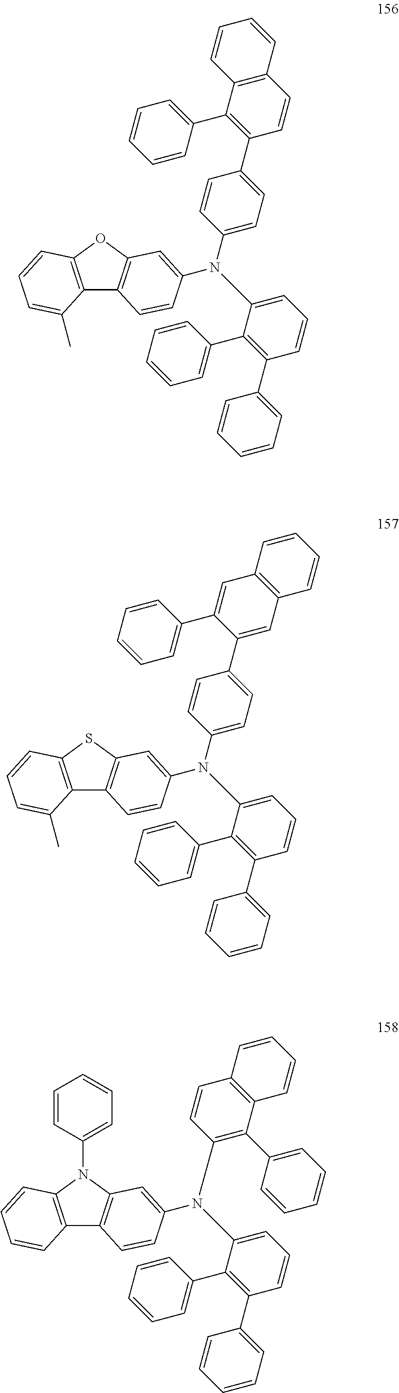 [SMILES: Cc1cccc2oc3cc(N(c4ccc(-c5ccc6ccccc6c5-c5ccccc5)cc4)c4cccc(-c5ccccc5)c4-c4ccccc4)ccc3c12 ; Cc1cccc2sc3cc(N(c4ccc(-c5cc6ccccc6cc5-c5ccccc5)cc4)c4cccc(-c5ccccc5)c4-c4ccccc4)ccc3c12 ; c1ccc(-c2cccc(N(c3ccc4c5ccccc5n(-c5ccccc5)c4c3)c3ccc4ccccc4c3-c3ccccc3)c2-c2ccccc2)cc1]

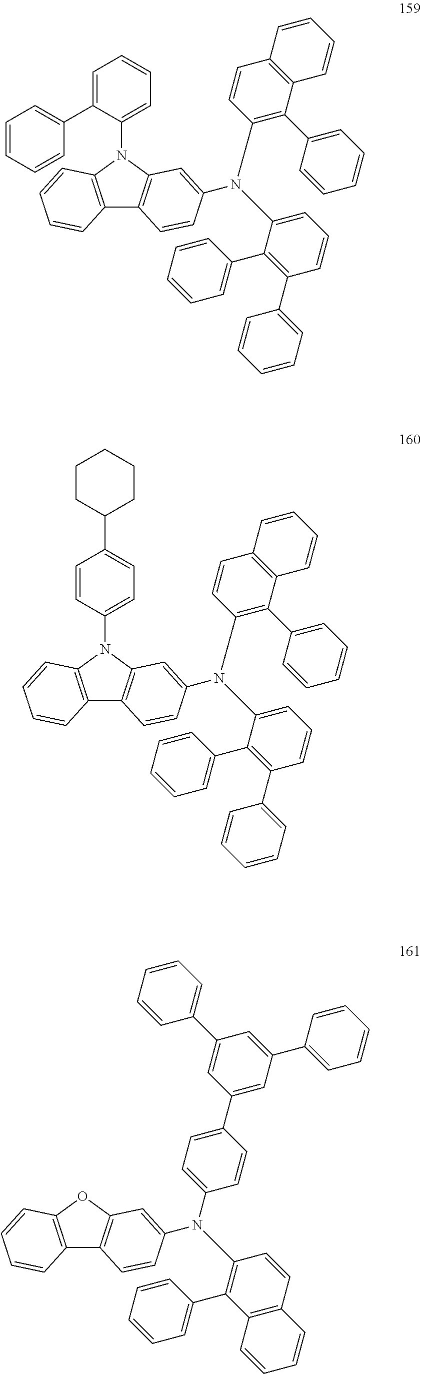 [SMILES: c1ccc(-c2ccccc2-n2c3ccccc3c3ccc(N(c4cccc(-c5ccccc5)c4-c4ccccc4)c4ccc5ccccc5c4-c4ccccc4)cc32)cc1 ; c1ccc(-c2cccc(N(c3ccc4c5ccccc5n(-c5ccc(C6CCCCC6)cc5)c4c3)c3ccc4ccccc4c3-c3ccccc3)c2-c2ccccc2)cc1 ; c1ccc(-c2cc(-c3ccccc3)cc(-c3ccc(N(c4ccc5c(c4)oc4ccccc45)c4ccc5ccccc5c4-c4ccccc4)cc3)c2)cc1]

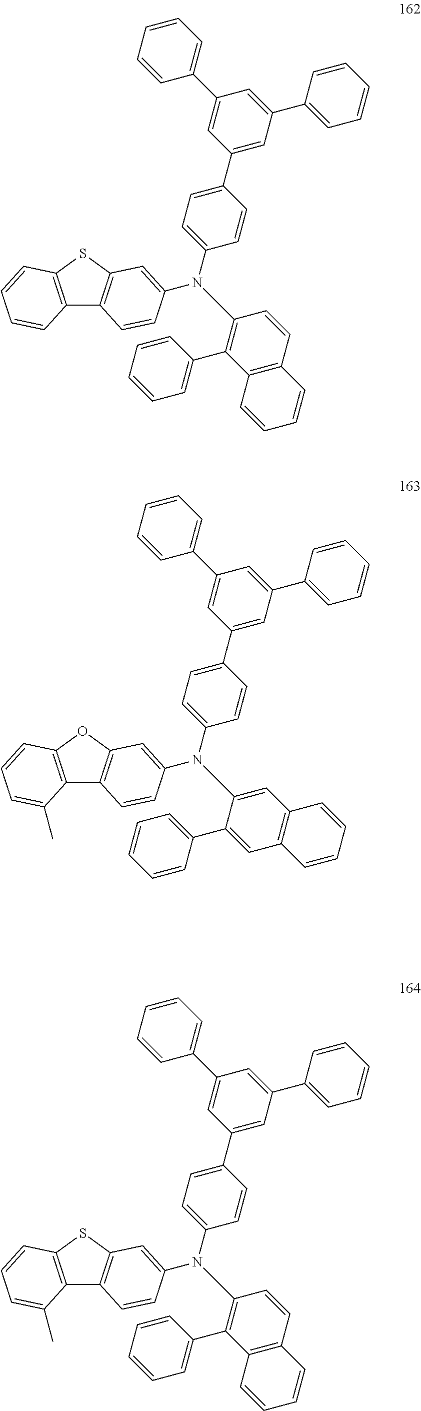 [SMILES: c1ccc(-c2cc(-c3ccccc3)cc(-c3ccc(N(c4ccc5c(c4)sc4ccccc45)c4ccc5ccccc5c4-c4ccccc4)cc3)c2)cc1 ; Cc1cccc2oc3cc(N(c4ccc(-c5cc(-c6ccccc6)cc(-c6ccccc6)c5)cc4)c4cc5ccccc5cc4-c4ccccc4)ccc3c12 ; Cc1cccc2sc3cc(N(c4ccc(-c5cc(-c6ccccc6)cc(-c6ccccc6)c5)cc4)c4ccc5ccccc5c4-c4ccccc4)ccc3c12]

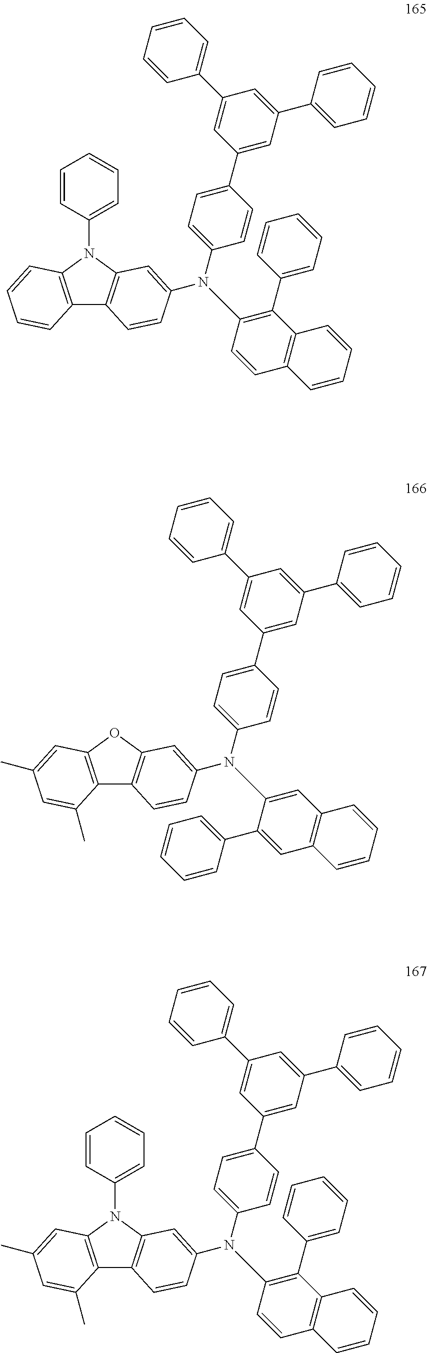 [SMILES: c1ccc(-c2cc(-c3ccccc3)cc(-c3ccc(N(c4ccc5c6ccccc6n(-c6ccccc6)c5c4)c4ccc5ccccc5c4-c4ccccc4)cc3)c2)cc1 ; Cc1cc(C)c2c(c1)oc1cc(N(c3ccc(-c4cc(-c5ccccc5)cc(-c5ccccc5)c4)cc3)c3cc4ccccc4cc3-c3ccccc3)ccc12 ; Cc1cc(C)c2c3ccc(N(c4ccc(-c5cc(-c6ccccc6)cc(-c6ccccc6)c5)cc4)c4ccc5ccccc5c4-c4ccccc4)cc3n(-c3ccccc3)c2c1]

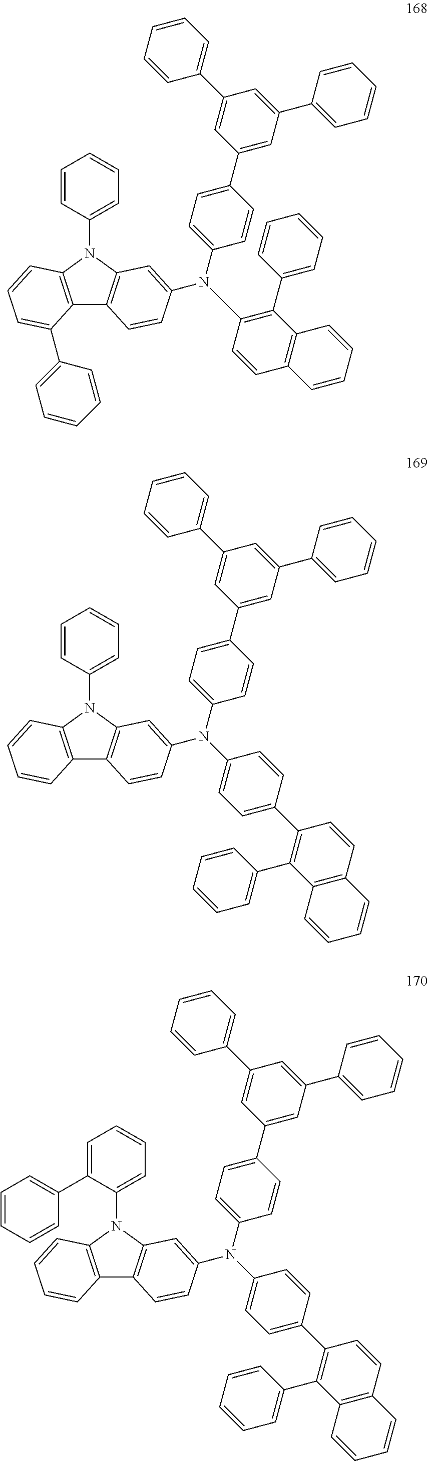 [SMILES: c1ccc(-c2cc(-c3ccccc3)cc(-c3ccc(N(c4ccc5c6c(-c7ccccc7)cccc6n(-c6ccccc6)c5c4)c4ccc5ccccc5c4-c4ccccc4)cc3)c2)cc1 ; c1ccc(-c2cc(-c3ccccc3)cc(-c3ccc(N(c4ccc(-c5ccc6ccccc6c5-c5ccccc5)cc4)c4ccc5c6ccccc6n(-c6ccccc6)c5c4)cc3)c2)cc1 ; c1ccc(-c2cc(-c3ccccc3)cc(-c3ccc(N(c4ccc(-c5ccc6ccccc6c5-c5ccccc5)cc4)c4ccc5c6ccccc6n(-c6ccccc6-c6ccccc6)c5c4)cc3)c2)cc1]

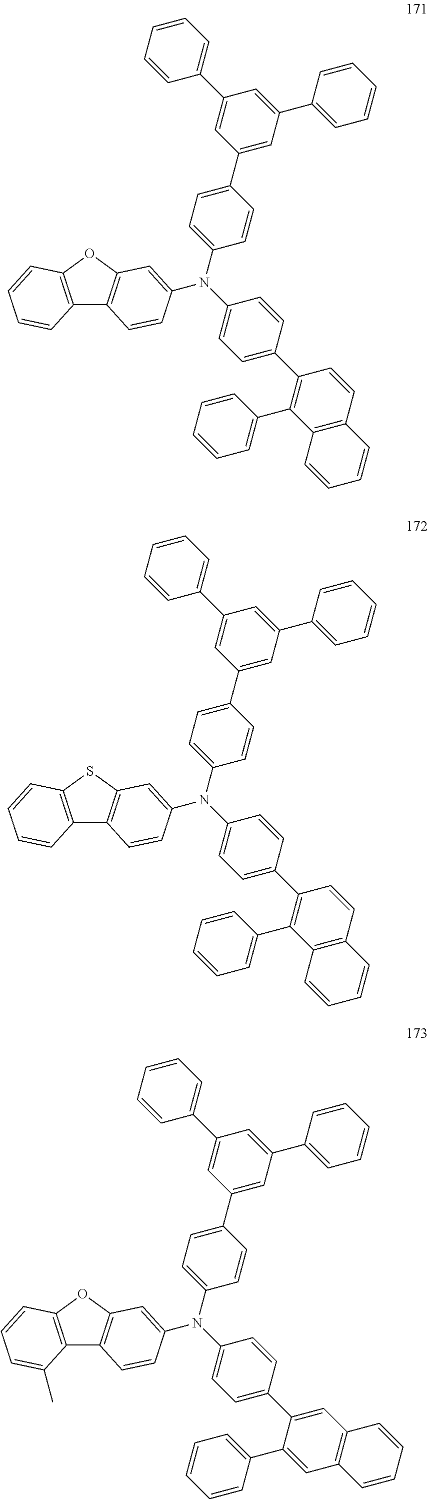 [SMILES: c1ccc(-c2cc(-c3ccccc3)cc(-c3ccc(N(c4ccc(-c5ccc6ccccc6c5-c5ccccc5)cc4)c4ccc5c(c4)oc4ccccc45)cc3)c2)cc1 ; c1ccc(-c2cc(-c3ccccc3)cc(-c3ccc(N(c4ccc(-c5ccc6ccccc6c5-c5ccccc5)cc4)c4ccc5c(c4)sc4ccccc45)cc3)c2)cc1 ; Cc1cccc2oc3cc(N(c4ccc(-c5cc(-c6ccccc6)cc(-c6ccccc6)c5)cc4)c4ccc(-c5cc6ccccc6cc5-c5ccccc5)cc4)ccc3c12]

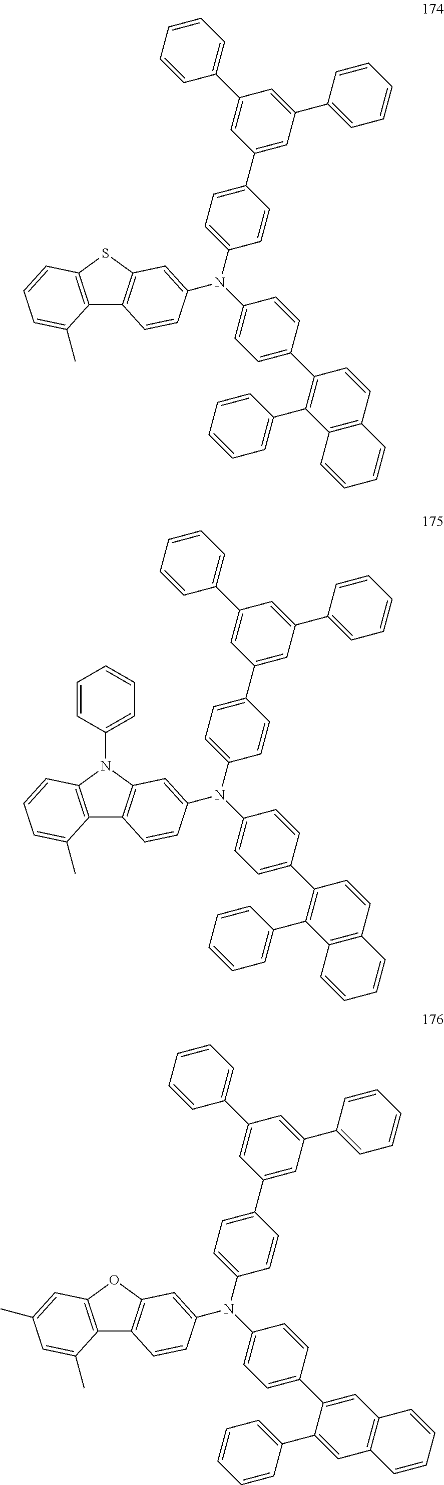 [SMILES: Cc1cccc2sc3cc(N(c4ccc(-c5cc(-c6ccccc6)cc(-c6ccccc6)c5)cc4)c4ccc(-c5ccc6ccccc6c5-c5ccccc5)cc4)ccc3c12 ; Cc1cccc2c1c1ccc(N(c3ccc(-c4cc(-c5ccccc5)cc(-c5ccccc5)c4)cc3)c3ccc(-c4ccc5ccccc5c4-c4ccccc4)cc3)cc1n2-c1ccccc1 ; Cc1cc(C)c2c(c1)oc1cc(N(c3ccc(-c4cc(-c5ccccc5)cc(-c5ccccc5)c4)cc3)c3ccc(-c4cc5ccccc5cc4-c4ccccc4)cc3)ccc12]

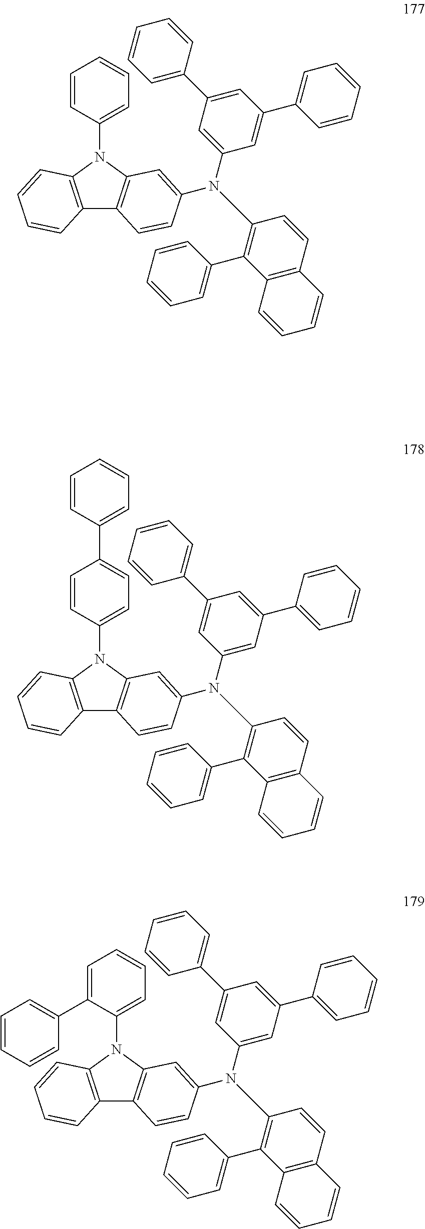 [SMILES: c1ccc(-c2cc(-c3ccccc3)cc(N(c3ccc4c5ccccc5n(-c5ccccc5)c4c3)c3ccc4ccccc4c3-c3ccccc3)c2)cc1 ; c1ccc(-c2ccc(-n3c4ccccc4c4ccc(N(c5cc(-c6ccccc6)cc(-c6ccccc6)c5)c5ccc6ccccc6c5-c5ccccc5)cc43)cc2)cc1 ; c1ccc(-c2cc(-c3ccccc3)cc(N(c3ccc4c5ccccc5n(-c5ccccc5-c5ccccc5)c4c3)c3ccc4ccccc4c3-c3ccccc3)c2)cc1]

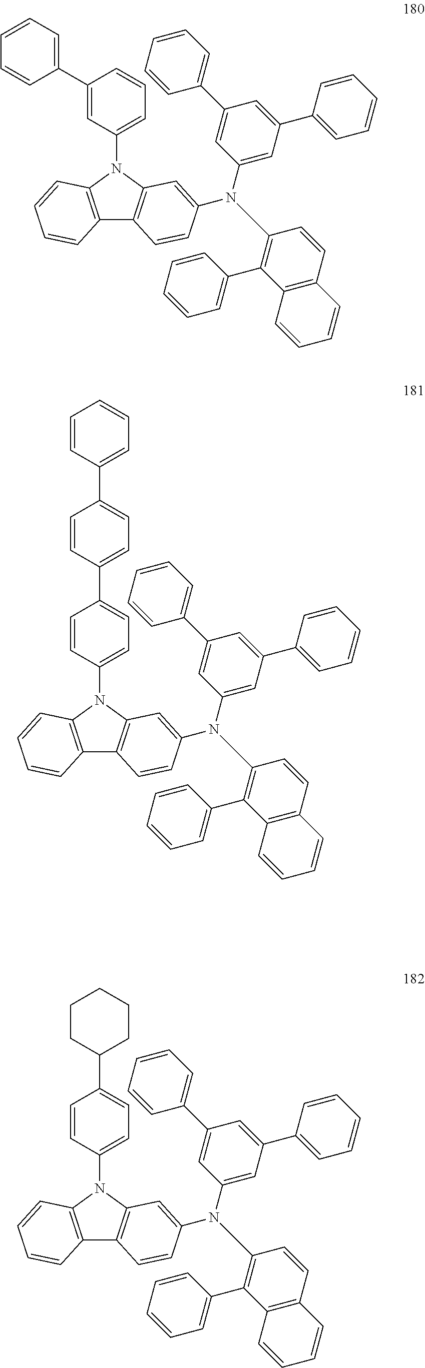 [SMILES: c1ccc(-c2cc(-c3ccccc3)cc(N(c3ccc4c5ccccc5n(-c5cccc(-c6ccccc6)c5)c4c3)c3ccc4ccccc4c3-c3ccccc3)c2)cc1 ; c1ccc(-c2ccc(-c3ccc(-n4c5ccccc5c5ccc(N(c6cc(-c7ccccc7)cc(-c7ccccc7)c6)c6ccc7ccccc7c6-c6ccccc6)cc54)cc3)cc2)cc1 ; c1ccc(-c2cc(-c3ccccc3)cc(N(c3ccc4c5ccccc5n(-c5ccc(C6CCCCC6)cc5)c4c3)c3ccc4ccccc4c3-c3ccccc3)c2)cc1]

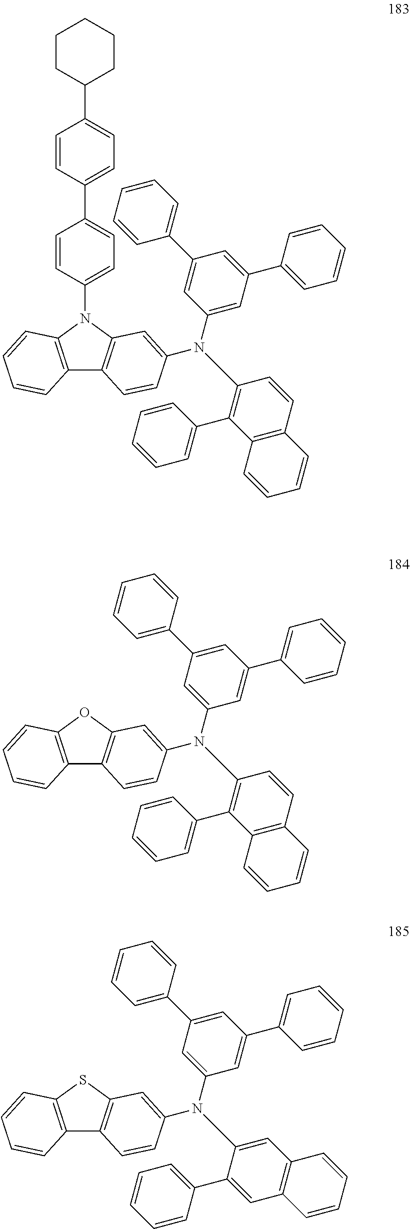 [SMILES: c1ccc(-c2cc(-c3ccccc3)cc(N(c3ccc4c5ccccc5n(-c5ccc(-c6ccc(C7CCCCC7)cc6)cc5)c4c3)c3ccc4ccccc4c3-c3ccccc3)c2)cc1 ; c1ccc(-c2cc(-c3ccccc3)cc(N(c3ccc4c(c3)oc3ccccc34)c3ccc4ccccc4c3-c3ccccc3)c2)cc1 ; c1ccc(-c2cc(-c3ccccc3)cc(N(c3ccc4c(c3)sc3ccccc34)c3cc4ccccc4cc3-c3ccccc3)c2)cc1]

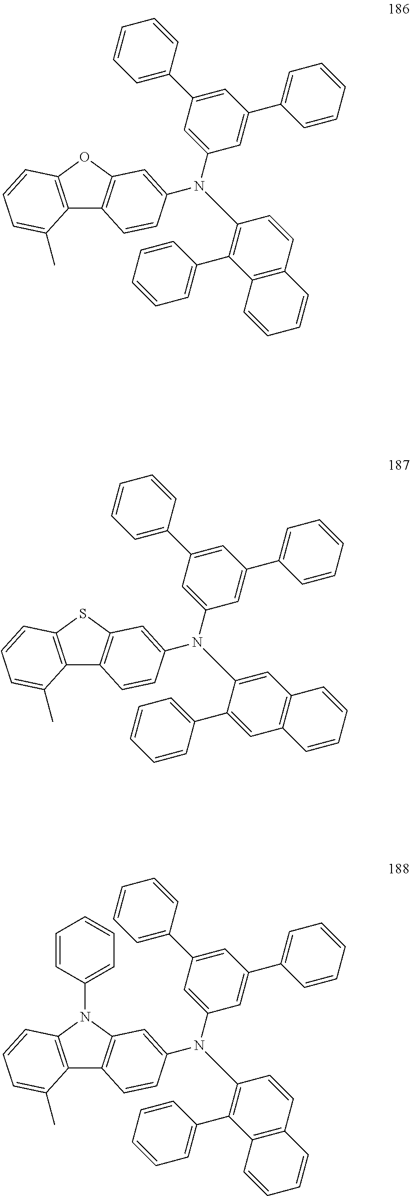 [SMILES: Cc1cccc2oc3cc(N(c4cc(-c5ccccc5)cc(-c5ccccc5)c4)c4ccc5ccccc5c4-c4ccccc4)ccc3c12 ; Cc1cccc2sc3cc(N(c4cc(-c5ccccc5)cc(-c5ccccc5)c4)c4cc5ccccc5cc4-c4ccccc4)ccc3c12 ; Cc1cccc2c1c1ccc(N(c3cc(-c4ccccc4)cc(-c4ccccc4)c3)c3ccc4ccccc4c3-c3ccccc3)cc1n2-c1ccccc1]

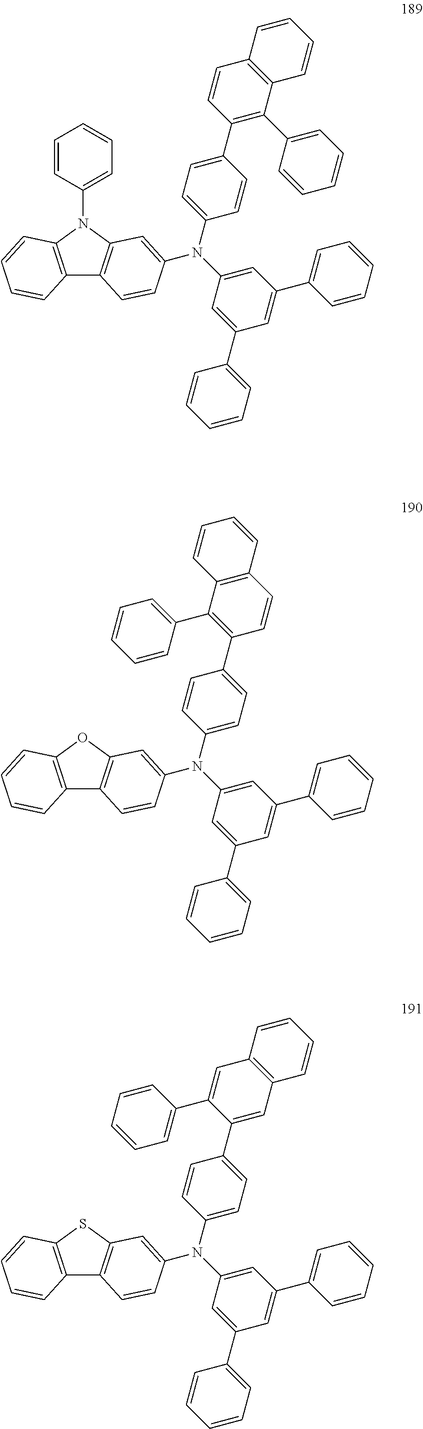 [SMILES: c1ccc(-c2cc(-c3ccccc3)cc(N(c3ccc(-c4ccc5ccccc5c4-c4ccccc4)cc3)c3ccc4c5ccccc5n(-c5ccccc5)c4c3)c2)cc1 ; c1ccc(-c2cc(-c3ccccc3)cc(N(c3ccc(-c4ccc5ccccc5c4-c4ccccc4)cc3)c3ccc4c(c3)oc3ccccc34)c2)cc1 ; c1ccc(-c2cc(-c3ccccc3)cc(N(c3ccc(-c4cc5ccccc5cc4-c4ccccc4)cc3)c3ccc4c(c3)sc3ccccc34)c2)cc1]

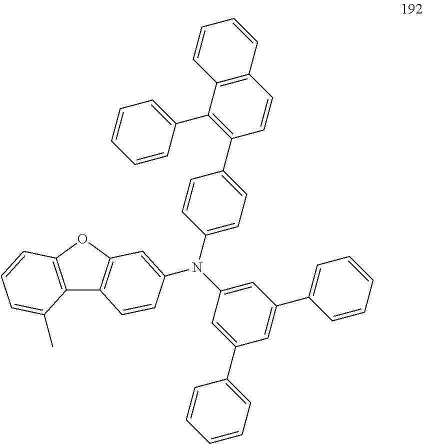 [SMILES: Cc1cccc2oc3cc(N(c4ccc(-c5ccc6ccccc6c5-c5ccccc5)cc4)c4cc(-c5ccccc5)cc(-c5ccccc5)c4)ccc3c12]

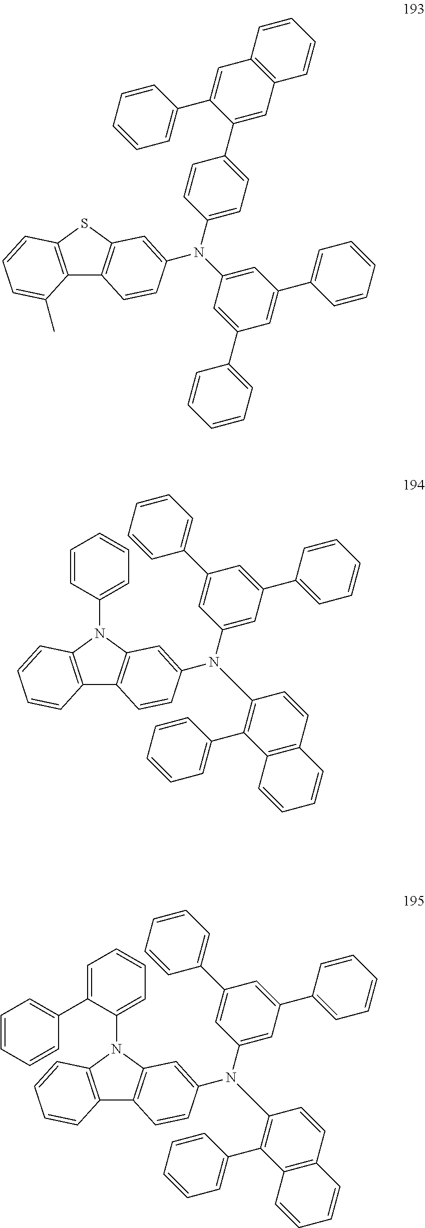 [SMILES: Cc1cccc2sc3cc(N(c4ccc(-c5cc6ccccc6cc5-c5ccccc5)cc4)c4cc(-c5ccccc5)cc(-c5ccccc5)c4)ccc3c12 ; c1ccc(-c2cc(-c3ccccc3)cc(N(c3ccc4c5ccccc5n(-c5ccccc5)c4c3)c3ccc4ccccc4c3-c3ccccc3)c2)cc1 ; c1ccc(-c2cc(-c3ccccc3)cc(N(c3ccc4c5ccccc5n(-c5ccccc5-c5ccccc5)c4c3)c3ccc4ccccc4c3-c3ccccc3)c2)cc1]

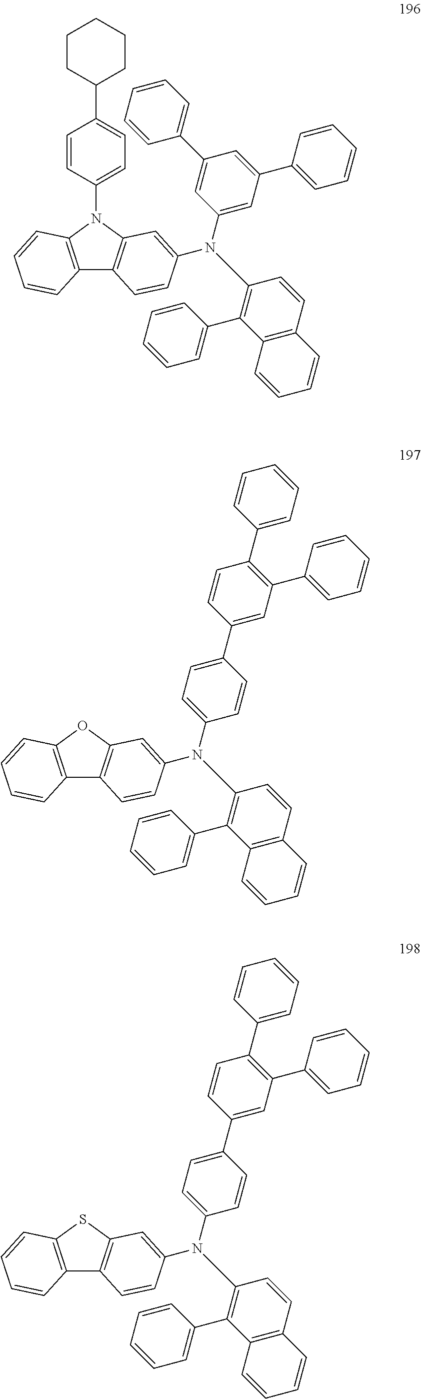 [SMILES: c1ccc(-c2cc(-c3ccccc3)cc(N(c3ccc4c5ccccc5n(-c5ccc(C6CCCCC6)cc5)c4c3)c3ccc4ccccc4c3-c3ccccc3)c2)cc1 ; c1ccc(-c2ccc(-c3ccc(N(c4ccc5c(c4)oc4ccccc45)c4ccc5ccccc5c4-c4ccccc4)cc3)cc2-c2ccccc2)cc1 ; c1ccc(-c2ccc(-c3ccc(N(c4ccc5c(c4)sc4ccccc45)c4ccc5ccccc5c4-c4ccccc4)cc3)cc2-c2ccccc2)cc1]

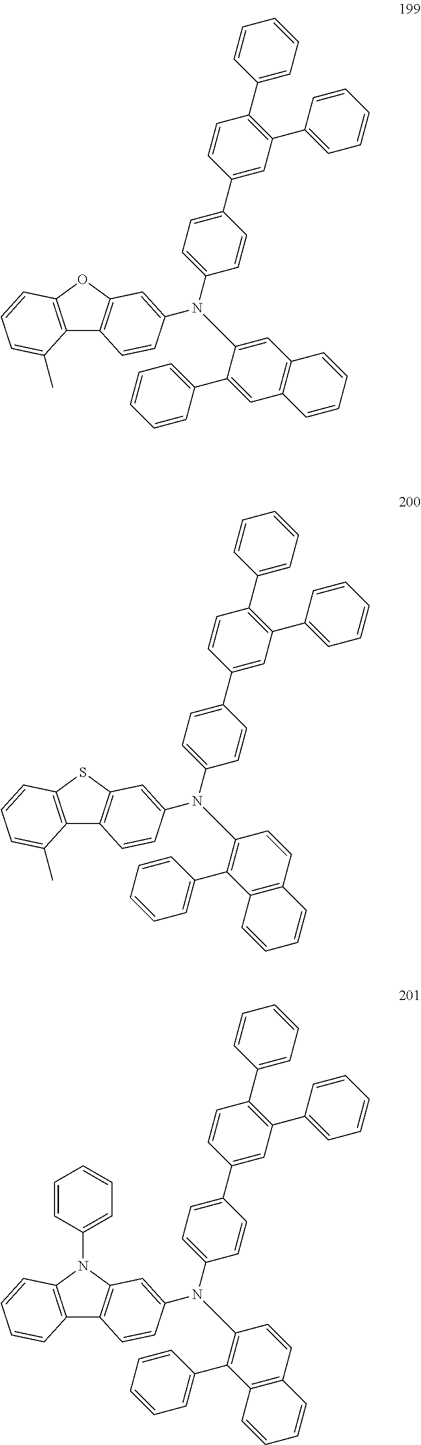 [SMILES: Cc1cccc2oc3cc(N(c4ccc(-c5ccc(-c6ccccc6)c(-c6ccccc6)c5)cc4)c4cc5ccccc5cc4-c4ccccc4)ccc3c12 ; Cc1cccc2sc3cc(N(c4ccc(-c5ccc(-c6ccccc6)c(-c6ccccc6)c5)cc4)c4ccc5ccccc5c4-c4ccccc4)ccc3c12 ; c1ccc(-c2ccc(-c3ccc(N(c4ccc5c6ccccc6n(-c6ccccc6)c5c4)c4ccc5ccccc5c4-c4ccccc4)cc3)cc2-c2ccccc2)cc1]

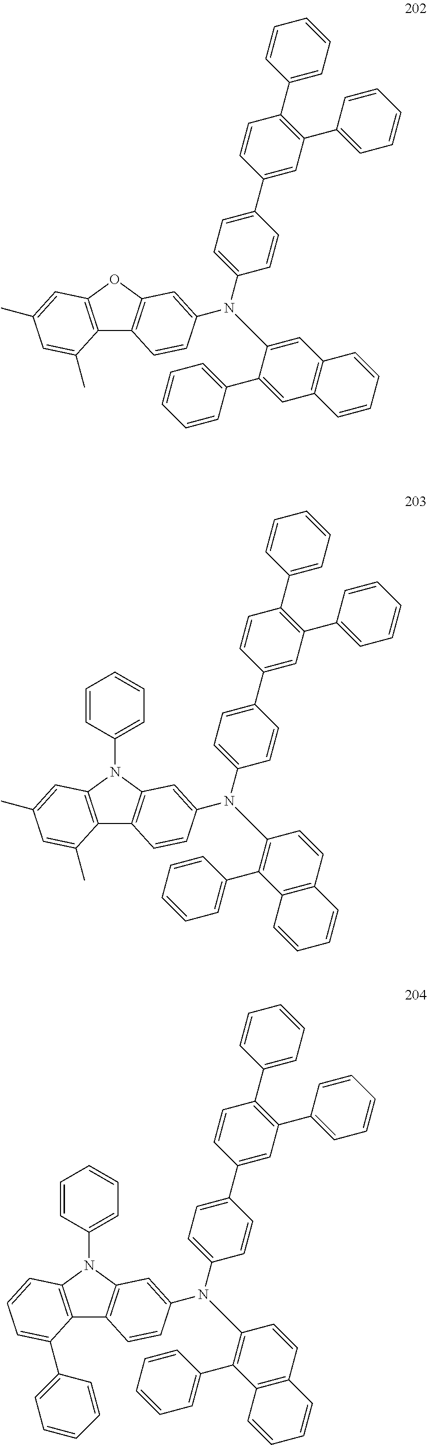 [SMILES: Cc1cc(C)c2c(c1)oc1cc(N(c3ccc(-c4ccc(-c5ccccc5)c(-c5ccccc5)c4)cc3)c3cc4ccccc4cc3-c3ccccc3)ccc12 ; Cc1cc(C)c2c3ccc(N(c4ccc(-c5ccc(-c6ccccc6)c(-c6ccccc6)c5)cc4)c4ccc5ccccc5c4-c4ccccc4)cc3n(-c3ccccc3)c2c1 ; c1ccc(-c2ccc(-c3ccc(N(c4ccc5c6c(-c7ccccc7)cccc6n(-c6ccccc6)c5c4)c4ccc5ccccc5c4-c4ccccc4)cc3)cc2-c2ccccc2)cc1]

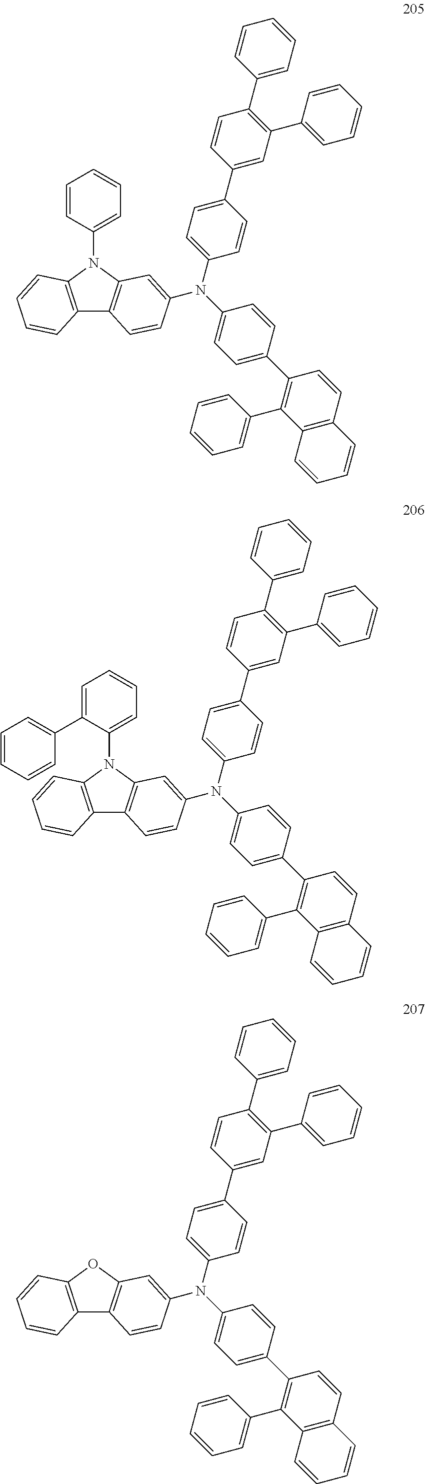 [SMILES: c1ccc(-c2ccc(-c3ccc(N(c4ccc(-c5ccc6ccccc6c5-c5ccccc5)cc4)c4ccc5c6ccccc6n(-c6ccccc6)c5c4)cc3)cc2-c2ccccc2)cc1 ; c1ccc(-c2ccc(-c3ccc(N(c4ccc(-c5ccc6ccccc6c5-c5ccccc5)cc4)c4ccc5c6ccccc6n(-c6ccccc6-c6ccccc6)c5c4)cc3)cc2-c2ccccc2)cc1 ; c1ccc(-c2ccc(-c3ccc(N(c4ccc(-c5ccc6ccccc6c5-c5ccccc5)cc4)c4ccc5c(c4)oc4ccccc45)cc3)cc2-c2ccccc2)cc1]

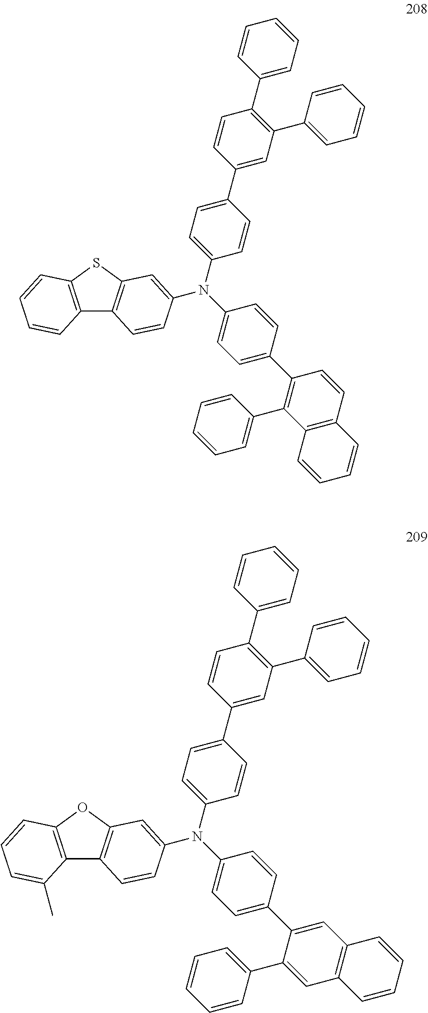 [SMILES: c1ccc(-c2ccc(-c3ccc(N(c4ccc(-c5ccc6ccccc6c5-c5ccccc5)cc4)c4ccc5c(c4)sc4ccccc45)cc3)cc2-c2ccccc2)cc1 ; Cc1cccc2oc3cc(N(c4ccc(-c5ccc(-c6ccccc6)c(-c6ccccc6)c5)cc4)c4ccc(-c5cc6ccccc6cc5-c5ccccc5)cc4)ccc3c12]

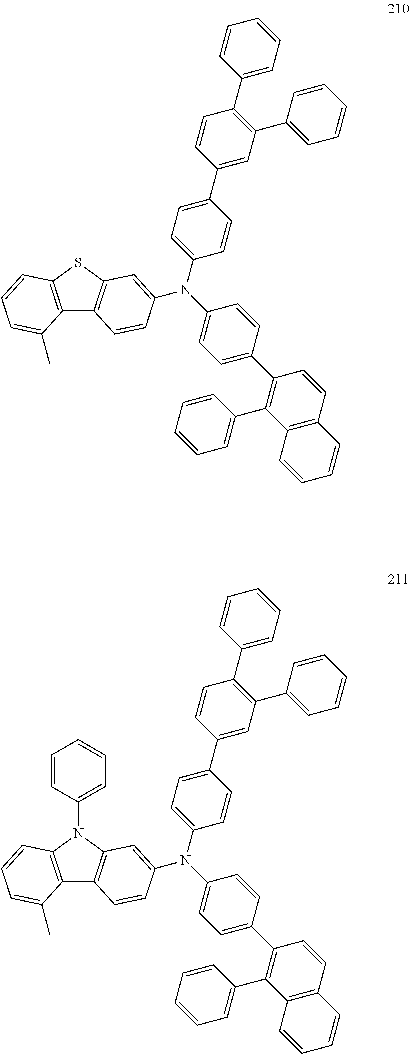 [SMILES: Cc1cccc2sc3cc(N(c4ccc(-c5ccc(-c6ccccc6)c(-c6ccccc6)c5)cc4)c4ccc(-c5ccc6ccccc6c5-c5ccccc5)cc4)ccc3c12 ; Cc1cccc2c1c1ccc(N(c3ccc(-c4ccc(-c5ccccc5)c(-c5ccccc5)c4)cc3)c3ccc(-c4ccc5ccccc5c4-c4ccccc4)cc3)cc1n2-c1ccccc1]

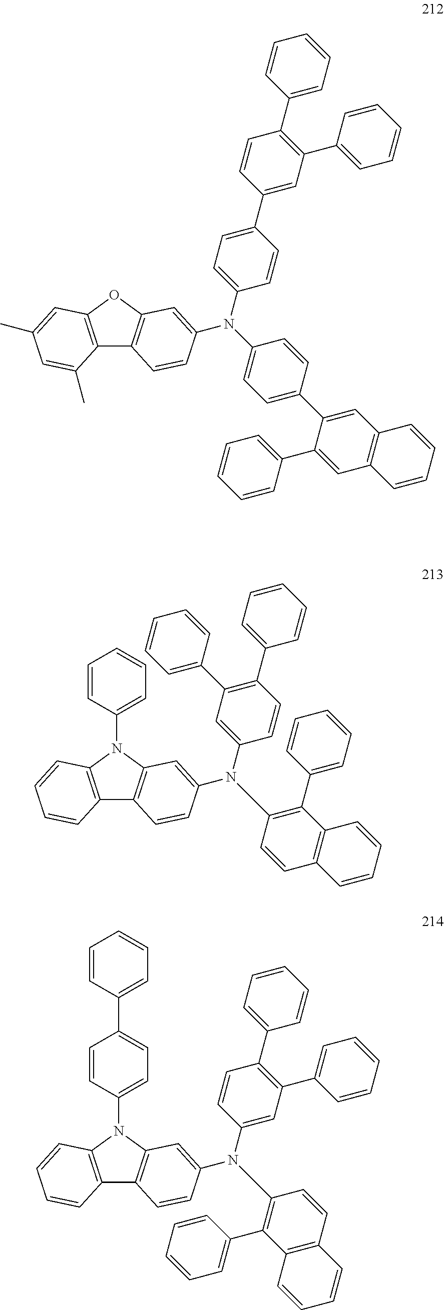 [SMILES: Cc1cc(C)c2c(c1)oc1cc(N(c3ccc(-c4ccc(-c5ccccc5)c(-c5ccccc5)c4)cc3)c3ccc(-c4cc5ccccc5cc4-c4ccccc4)cc3)ccc12 ; c1ccc(-c2ccc(N(c3ccc4c5ccccc5n(-c5ccccc5)c4c3)c3ccc4ccccc4c3-c3ccccc3)cc2-c2ccccc2)cc1 ; c1ccc(-c2ccc(-n3c4ccccc4c4ccc(N(c5ccc(-c6ccccc6)c(-c6ccccc6)c5)c5ccc6ccccc6c5-c5ccccc5)cc43)cc2)cc1]

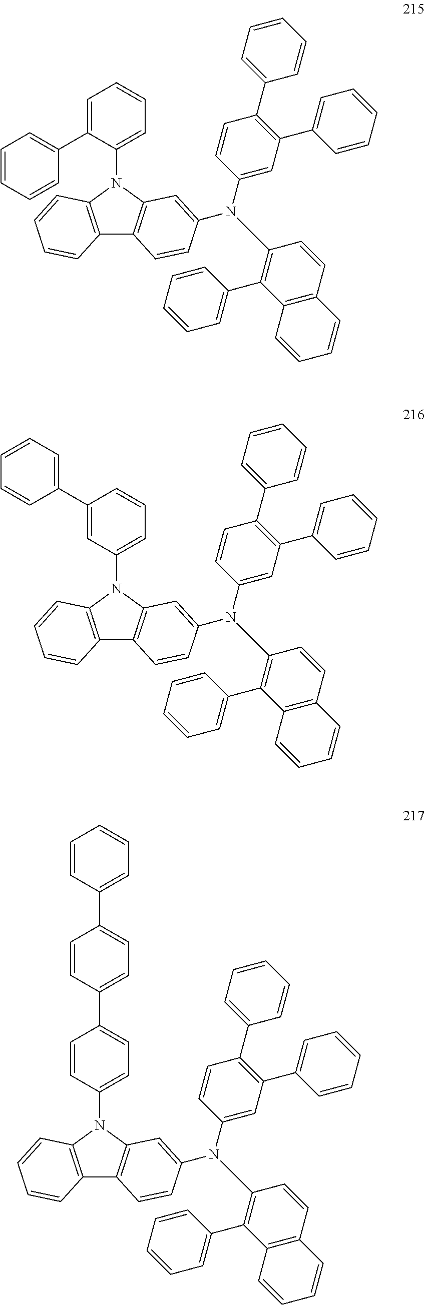 [SMILES: c1ccc(-c2ccc(N(c3ccc4c5ccccc5n(-c5ccccc5-c5ccccc5)c4c3)c3ccc4ccccc4c3-c3ccccc3)cc2-c2ccccc2)cc1 ; c1ccc(-c2cccc(-n3c4ccccc4c4ccc(N(c5ccc(-c6ccccc6)c(-c6ccccc6)c5)c5ccc6ccccc6c5-c5ccccc5)cc43)c2)cc1 ; c1ccc(-c2ccc(-c3ccc(-n4c5ccccc5c5ccc(N(c6ccc(-c7ccccc7)c(-c7ccccc7)c6)c6ccc7ccccc7c6-c6ccccc6)cc54)cc3)cc2)cc1]

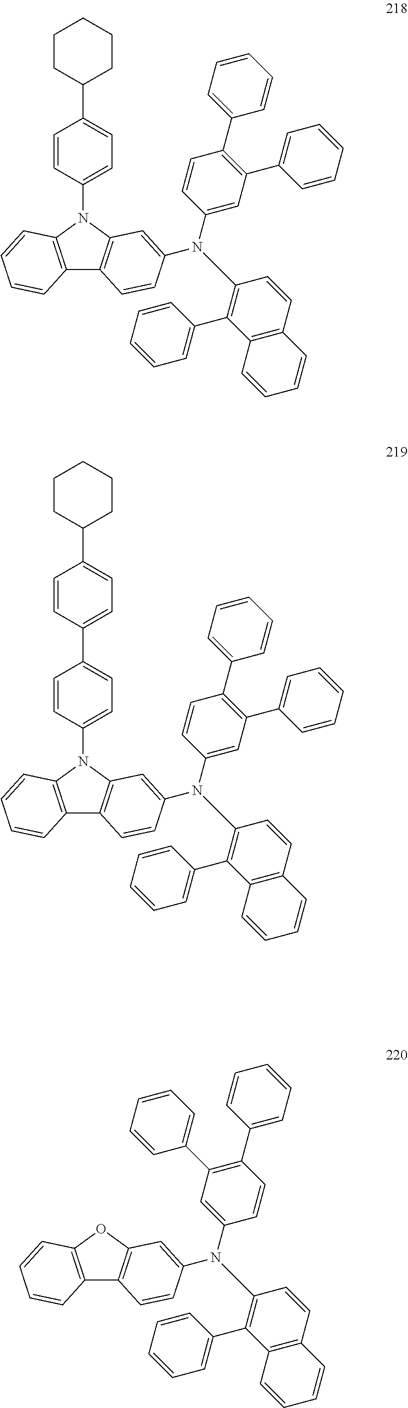 [SMILES: c1ccc(-c2ccc(N(c3ccc4c5ccccc5n(-c5ccc(C6CCCCC6)cc5)c4c3)c3ccc4ccccc4c3-c3ccccc3)cc2-c2ccccc2)cc1 ; c1ccc(-c2ccc(N(c3ccc4c5ccccc5n(-c5ccc(-c6ccc(C7CCCCC7)cc6)cc5)c4c3)c3ccc4ccccc4c3-c3ccccc3)cc2-c2ccccc2)cc1 ; c1ccc(-c2ccc(N(c3ccc4c(c3)oc3ccccc34)c3ccc4ccccc4c3-c3ccccc3)cc2-c2ccccc2)cc1]

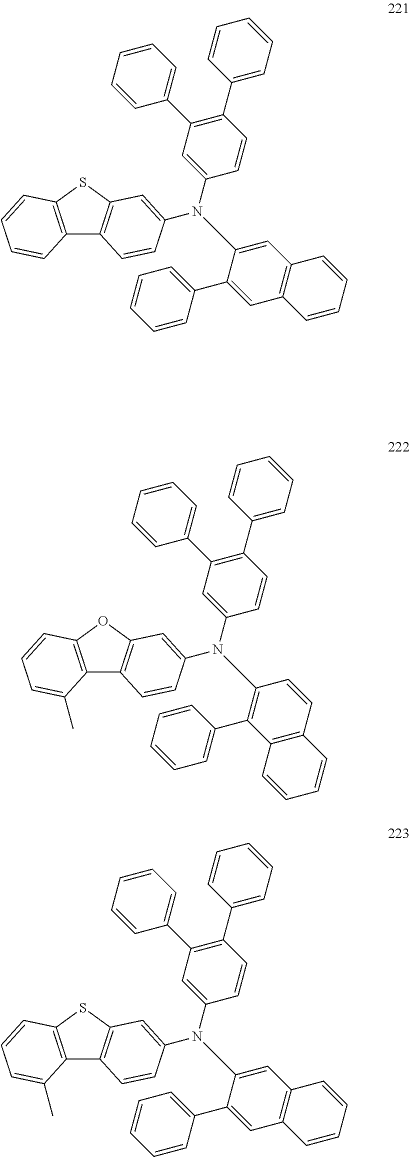 [SMILES: c1ccc(-c2ccc(N(c3ccc4c(c3)sc3ccccc34)c3cc4ccccc4cc3-c3ccccc3)cc2-c2ccccc2)cc1 ; Cc1cccc2oc3cc(N(c4ccc(-c5ccccc5)c(-c5ccccc5)c4)c4ccc5ccccc5c4-c4ccccc4)ccc3c12 ; Cc1cccc2sc3cc(N(c4ccc(-c5ccccc5)c(-c5ccccc5)c4)c4cc5ccccc5cc4-c4ccccc4)ccc3c12]

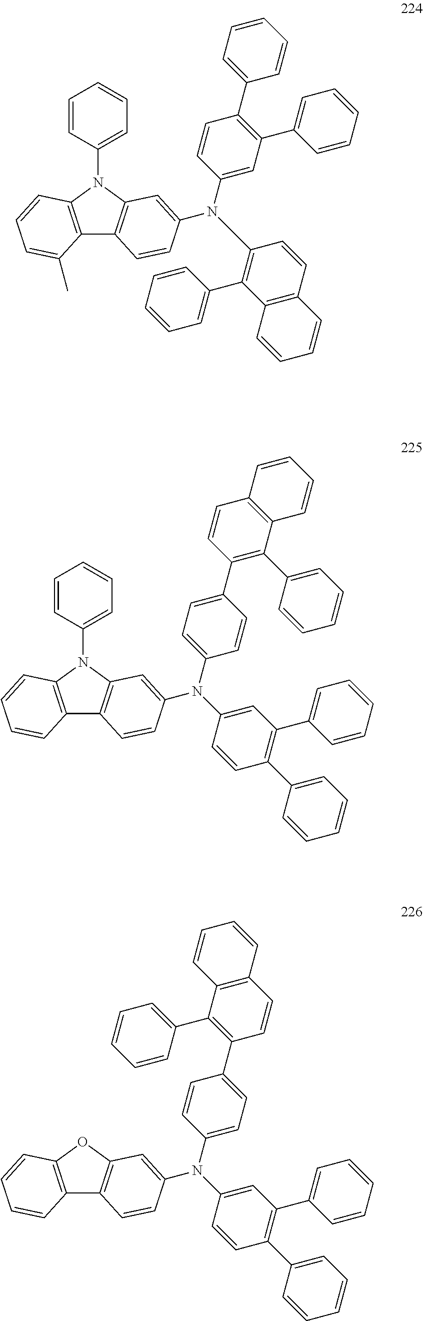 [SMILES: Cc1cccc2c1c1ccc(N(c3ccc(-c4ccccc4)c(-c4ccccc4)c3)c3ccc4ccccc4c3-c3ccccc3)cc1n2-c1ccccc1 ; c1ccc(-c2ccc(N(c3ccc(-c4ccc5ccccc5c4-c4ccccc4)cc3)c3ccc4c5ccccc5n(-c5ccccc5)c4c3)cc2-c2ccccc2)cc1 ; c1ccc(-c2ccc(N(c3ccc(-c4ccc5ccccc5c4-c4ccccc4)cc3)c3ccc4c(c3)oc3ccccc34)cc2-c2ccccc2)cc1]

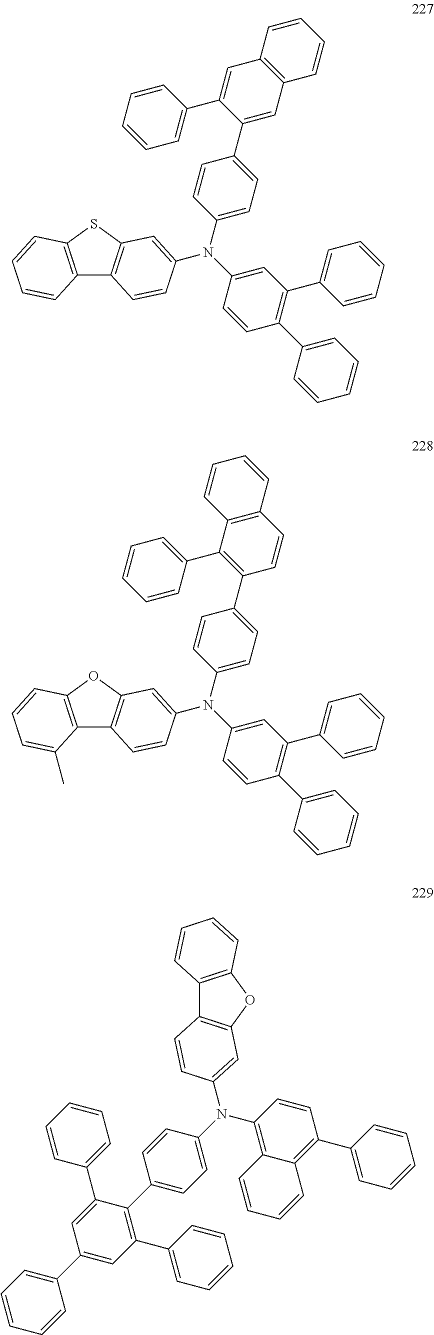 [SMILES: c1ccc(-c2ccc(N(c3ccc(-c4cc5ccccc5cc4-c4ccccc4)cc3)c3ccc4c(c3)sc3ccccc34)cc2-c2ccccc2)cc1 ; Cc1cccc2oc3cc(N(c4ccc(-c5ccc6ccccc6c5-c5ccccc5)cc4)c4ccc(-c5ccccc5)c(-c5ccccc5)c4)ccc3c12 ; c1ccc(-c2cc(-c3ccccc3)c(-c3ccc(N(c4ccc5c(c4)oc4ccccc45)c4ccc(-c5ccccc5)c5ccccc45)cc3)c(-c3ccccc3)c2)cc1]

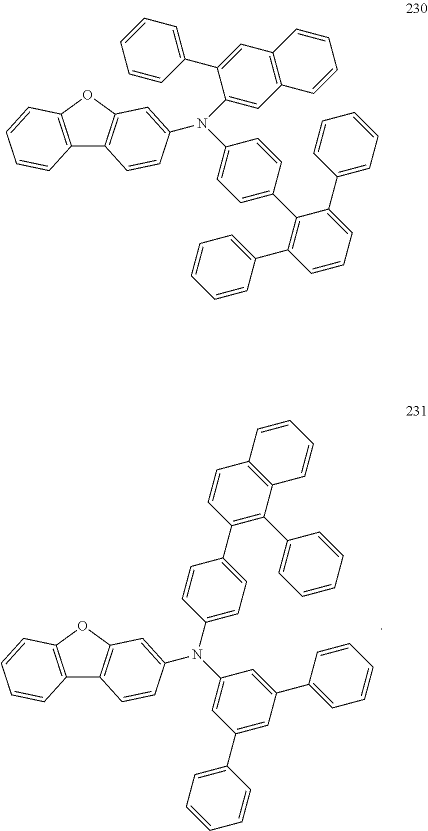 [SMILES: c1ccc(-c2cc3ccccc3cc2N(c2ccc(-c3c(-c4ccccc4)cccc3-c3ccccc3)cc2)c2ccc3c(c2)oc2ccccc23)cc1 ; c1ccc(-c2cc(-c3ccccc3)cc(N(c3ccc(-c4ccc5ccccc5c4-c4ccccc4)cc3)c3ccc4c(c3)oc3ccccc34)c2)cc1]

12. A light-emitting device comprising:
a first electrode;
a second electrode facing the first electrode;
an interlayer between the first electrode and the second electrode and comprising an emission layer; and
the amine-containing compound of claim 1.

13. The light-emitting device of claim 12, wherein the interlayer further comprises a hole transport region between the first electrode and the emission layer and an electron transport region between the emission layer and the second electrode,
the hole transport region comprises a hole injection layer, a hole transport layer, an emission auxiliary layer, an electron-blocking layer, or any combination thereof, and
the electron transport region comprises a buffer layer, a hole-blocking layer, an electron control layer, an electron transport layer, an electron injection layer, or any combination thereof.

14. The light-emitting device of claim 12, wherein the interlayer comprises the amine-containing compound.

15. The light-emitting device of claim 13, wherein the hole transport region comprises the amine-containing compound.

16. The light-emitting device of claim 13, wherein the hole transport layer comprises the amine-containing compound, and
the hole transport layer is in direct contact with the emission layer.

17. The light-emitting device of claim 12, further comprising a capping layer located outside the second electrode,
wherein the capping layer comprises the amine-containing compound.

18. An electronic apparatus comprising the light-emitting device of claim 12.

19. The electronic apparatus of claim 18, further comprising a thin-film transistor,
wherein the thin-film transistor comprises a source electrode and a drain electrode, and
the first electrode of the light-emitting device is electrically connected to at least one of the source electrode and the drain electrode of the thin-film transistor.

20. An electronic equipment comprising the light-emitting device of claim 12, wherein the electronic equipment is at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, and combinations thereof.
